(19) United States
(12) Patent Application Publication
(10) Pub. No.: US 2021/0389308 A1
(43) Pub. Date: Dec. 16, 2021

(54) DETECTING ADAPTIVE IMMUNITY TO CORONAVIRUS

(71) Applicant: NONIGENEX, INC., LA JOLLA, CA (US)

(72) Inventors: Jerome P. LAPOINTE, LA JOLLA, CA (US); Andrew SENYEI, LA JOLLA, CA (US)

(21) Appl. No.: 17/408,096

(22) Filed: Aug. 20, 2021

Related U.S. Application Data
(63) Continuation of application No. PCT/US21/25409, filed on Apr. 1, 2021.
(60) Provisional application No. 63/161,869, filed on Mar. 16, 2021.
(60) Provisional application No. 63/119,854, filed on Dec. 1, 2020.
(60) Provisional application No. 63/052,922, filed on Jul. 16, 2020.
(60) Provisional application No. 63/042,394, filed on Jun. 22, 2020.
(60) Provisional application No. 63/013,422, filed on Apr. 21, 2020.
(60) Provisional application No. 63/009,992, filed on Apr. 14, 2020.
(60) Provisional application No. 63/005,168, filed on Apr. 3, 2020.

(51) Int. Cl. G01N 33/536; G01N 33/543; G01N 33/68

(52) U.S. Cl. CPC G01N 33/536; G01N 2333/165; G01N 33/6854; G01N 33/543

(57) ABSTRACT

Provided are devices, systems, methods and kits for determining whether a subject is immune to an infection by a disease-causing pathogen by measuring neutralizing antibodies against the disease-causing pathogen in a biological sample from the subject. The devices, systems, methods, and kits described herein are useful for confirming whether a vaccine against the disease-causing pathogen has elicited enough neutralizing antibodies to prevent a later infection, or lessen severity of disease caused by, the disease-causing pathogen. Such devices, systems, methods, and kits are also useful for detecting an infection in the subject.

### CROSS-REFERENCE

This application is a continuation of International Application No. PCT/US21/25409, filed Apr. 1, 2021, which claims the benefit of U.S. Provisional Application No. 63/161,869, filed Mar. 16, 2021, U.S. Provisional Application No. 63/119,854, filed Dec. 1, 2020, U.S. Provisional Application No. 63/052,922, filed Jul. 16, 2020, U.S. Provisional Application No. 63/042,394, filed Jun. 22, 2020, U.S. Provisional Application No. 63/013,422, filed Apr. 21, 2020, U.S. Provisional Application No. 63/009,992, filed Apr. 14, 2020, and U.S. Provisional Application No. 63/005,168, filed Apr. 3, 2020, each of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Aug. 19, 2021, is named 58552-701_301_SL.txt and is 1,204,010 bytes in size.

### BACKGROUND

Viral infections on a cellular level are mediated by viruses binding to receptors expressed on the surface of a target cell. For example, the spike glycoprotein of a coronavirus binds to the angiotensin-converting enzyme 2 (ACE2) receptor, and binding between the receptor-binding domain (RBD) of the spike protein and ACE2 precedes entry of the coronavirus into the cell. Individuals who are exposed to a new virus (e.g., coronavirus) may develop neutralizing antibodies against the virus to block viral infection. This adaptive immune response significantly reduces incidences of a second infection by the same virus.

The coronavirus disease of 2019 (COVID-19) is an ongoing pandemic caused by severe acute respiratory syndrome coronavirus 2 (SARS-CoV-2). There is an unprecedented need for effective and safe vaccines against SARS-CoV-2. Even those who are infected with SARS-CoV-2 and recover face severe side-effects, and even death, post recovery. Post-COVID syndrome, which includes cardiac, neuro, and respiratory complications, can persist indefinitely. One third of recovered COVID-19 hospitalized patients are readmitted, and 1 in 8 die within 5 months. In an effort to fast track the regulatory approval process for new vaccines against SARS-CoV-2, regulatory agencies and vaccine developers may look to surrogate markers of protection against future infections by SARS-CoV-2 until proof of protection is available. This means that new vaccines entering the market, although proven to be safe, may not be effective.

Infectious disease experts believe there is a strong correlation between protection from a future infection by SARS-CoV-2 and the presence of neutralizing antibody titers. Yet, out of the dozens of vaccines under clinical investigation today, a vast majority do not test for presence of neutralizing antibodies against SARS-CoV-2 as a primary clinical endpoint. There exists an urgent need for a confirmatory diagnostic test that measures neutralizing antibody titers to determine whether a legally commercialized vaccine is effective to confer immunity against SARS-CoV-2 (e.g., post-allowance or licensure of the vaccine). With nearly 320 million people in the United States alone in need of vaccination, the neutralizing antibody test needed must be scalable and cost-efficient.

Reliance on surrogate markers of protection may result in a larger portion of subjects that will not respond to the new legally commercialized vaccine as compared with a comparable vaccine developed within the traditional 15-20 year timeframe. There is a need for a scalable test that measures neutralizing antibodies in a population of individuals after administration of the new SARS-CoV-2 vaccine (e.g., allowed or approved for human or other animal use) and a system that enables non-responders in that population to know whether the new vaccine was effective to induce neutralizing antibodies conferring protection, or not. For example, there is minimal trial data for important sub-populations, such as those of the non-white race, and those currently having one or more risk factors related to COVID-19. There is also no data on larger subgroups who are excluded from clinical trials, such as those who are pregnant, breastfeeding, or have priorly been infected by COVID-19. Stratifying non-responders from responders to a new vaccine in this context will prevent the unintentional spread of COVID-19 by vaccinated individuals with a “false” sense of immunity. This is particularly critical for protecting our vulnerable populations, including individuals ages 50 and older who are significantly more likely to die from a disease caused by SARS-CoV-2 as compared to younger individuals. For example, published data show that new vaccines entering the market elicit around 40% fewer neutralizing antibodies in subjects ages 65 and older. Clinical trials show the efficacy of the new vaccines in subjects ages 65 and older could be as low as 65%.

Additionally, the vaccine administration methods taking place pose further problems. With many millions of people waiting several weeks to receive a second COVID-19 vaccine dose in some countries, it is possible that SARS-CoV-2 could evolve vaccine resistance. In addition to the timing of the administered doses being an issue, the durability of the vaccine elicited immune response in patients is not yet known. Further, as millions of people are being vaccinated, health officials are struggling to collect critically important information-such as race, ethnicity and occupation—of every person vaccinated. The data being collected is so scattered that there's little insight into which health care workers, or first responders, have been among the people getting the initial vaccines, as intended—or how many doses instead have gone to people who should be much further down the list.

In addition, the fragile thermostability of vaccines means that any delay during distribution poses a risk that the vaccines will have reduced potency upon arrival to its destination. As one example, delays in distribution due to unpredictable and extreme weather conditions across the United States are a source of major concern. Addressing this challenge for vaccines against SARS-CoV-2 is made even more difficult with the limited number of facilities around the world manufacturing the vaccines requiring longer delivery routes.

However, this challenge is not limited to delays in distribution. For a clinic to receive a vaccine, they must have ultra-low cold storage sufficient to hold the vaccine at low enough temperatures. The vaccine must be kept at low temperatures in order to preserve its potency. For example, even opening freezers repeatedly can harm vaccines stored in the freezer and inadvertently lower their potency. Once a clinic receives a vaccine shipment, health workers thaw out the vials in a refrigerator as they prepare to give injections to patients. But once a vaccine is thawed, it is only viable for a few days and cannot be re-frozen. Thus, there exists a need for a test that can determine whether a vaccine administered to a subject is effective to meet this unprecedented need.

Shelter-in-place mandates all over the world designed to reduce the spread of COVID-19 are having severe and long-lasting negative socioeconomic and economic consequences. In theory, reintegration into society of individuals with enough neutralizing antibody titers conferring immunity to SARS-CoV-2 is possible, provided there is a reliable and scalable test to measure whether a subject has sufficient neutralizing antibody titers against SARS-CoV-2 to confer protection. However, existing SARS-CoV-2 tests are not scalable. Thus, there exists a need for a test that can measure neutralizing antibodies that is scalable to meet this unprecedented need.

### SUMMARY

Provided herein are testing devices and methods of their use that detect one or more neutralizing antibodies in a biological sample from a subject that functionally block pathogen binding to its cognate receptor. In some embodiments, a presence or requisite level of neutralizing antibodies is a surrogate for protection against a future infection by the pathogen. Methods described herein provide for centralizing and automated assay systems to measure neutralizing antibodies for screening large numbers of subjects, such as a population or occupation group (e.g., healthcare workers), in a manner that is low cost and scalable.

The testing devices, systems and methods described herein are useful for confirming whether a vaccine against the pathogen is effective to confer immunity in a subject (or population of subjects) or in a manner expected of the vaccine developer, and therefore, protection against a future infection by the pathogen. In some embodiments, the subject is a human or another animal, such as a household pet or farm animal. The ability to understand whether a portion of the population does not respond to (e.g., is not protected by) a novel vaccine licensed for human use will empower highly informed decision-making by vaccine developers and regulatory entities about how and when a novel vaccine should be utilized post-licensure. If a subject is identified as a non-responder to a vaccine, methods described herein include notifying the subject to prevent the spread of infection. When performed on a large scale, the methods described herein will mitigate risk associated with development and licensure of novel vaccines, thereby encouraging more vaccines to enter the market to meet the unprecedented need.

In some embodiments, detecting neutralizing antibodies that functionally block pathogen binding to its cognate receptor and detecting the presence of total antibodies against the pathogen are performed simultaneously by the testing devices described herein to, for example, identify a subject that was exposed to the pathogen or a different pathogen and who may not be immune (or protected) from a future infection by the pathogen. In some embodiments, the testing devices described herein also detect a presence of total antibodies (e.g., IgG, IgM, IgA) against the pathogen that are unique to the subject. In some embodiments, the testing devices, systems and methods also measure a presence or level of a biomarker, such as a proinflammatory biomarker.

Methods of using the devices described herein, comprise assaying a biological sample from a subject with the testing device and detecting a complex between the neutralizing antibodies and a detectable peptide-conjugate derived from the pathogen. In some embodiments, methods comprise detecting with the naked eye. Also provided are systems comprising the testing device and an imaging device, such as a smartphone. In some embodiments, detecting comprises capturing an image of a detection zone of the testing device, analyzing the data from the image, and providing a result to the subject. In some embodiments, the pathogen is severe acute respiratory syndrome coronavirus 2 (SARS-CoV-2).

Aspects disclosed herein provide methods of measuring a presence, an absence, or a quantity of neutralizing antibodies against a coronavirus that functionally block binding between a spike protein of the coronavirus and Angiotensin-converting enzyme 2 (ACE2), the method comprising: (a) providing a biological sample obtained from one or more finger pricks of a subject that was administered a vaccine against the coronavirus; (b) bringing at least a portion of the biological sample into contact with: (i) a first peptide or protein comprising the ACE2 polypeptide or a portion thereof; and (ii) a second peptide or protein comprising the spike protein or a portion thereof, wherein: (1) the first peptide or protein and the second peptide or protein form a complex in an absence of neutralizing antibodies in the biological sample that block binding between the spike protein and ACE2 under conditions otherwise suitable for binding; and (2) the first peptide or protein, the second peptide or protein, or a combination thereof comprises a detectable moiety; (c) detecting an absence, a presence, or a quantity of the complex formed in (b); and (d) measuring the presence, the absence, or the quantity of the neutralizing antibodies against the coronavirus, which is inversely correlated with the presence, the absence, or the quantity of the complex detected in (c), respectively. In some embodiments, the biological sample is classified based on the measuring in (d) by a mobile application on a personal electronic device comprising a smartphone, a tablet, or a personal computer. In some embodiments, the method does not consist of utilizing an immortalized cell or immortalized cell culture. In some embodiments, the methods further comprise providing a result to a user on an electronic device regarding the presence, the absence, or the quantity of the neutralizing antibodies against the coronavirus in the biological sample to aid in the prognosis of the subject related to an infection by the coronavirus based, at least in part, on the measuring in (d). In some embodiments, the method further comprise: (a) bringing the biological sample into contact with a capture moiety specific to one or more antibodies against the coronavirus; and (b) detecting, in the biological sample, a presence, an absence, or a quantity of the one or more antibodies against the coronavirus, wherein the one or more antibodies against the coronavirus is not a neutralizing antibody. In some embodiments, the one or more antibodies against the coronavirus belong to a class of antibodies comprising immunoglobulin A, immunoglobulin M, immunoglobulin G, or a combination thereof. In some embodiments, the vaccine against the coronavirus is approved by a regulatory agency for prevention of a disease caused by the coronavirus. In some embodiments, methods further comprise administering a second dose of the vaccine against the coronavirus to the subject. In some embodiments, the spike protein of the coronavirus comprises a receptor binding domain specific for binding with ACE2 in vivo. In some embodiments, the coronavirus is a Severe acute respiratory syndrome-related (SARS) virus. In some embodiments, the SARS is SARS-CoV-2. In some embodiments, the biological sample comprises capillary blood. In some embodiments, the human subject is an age that is equal to about 50, 51, 52, 53, 54, 55, 56, 57, 58, 59, 60, 61, 62, 63, 64, 65, 66, 67, 68, 69, 70, 71, 72, 73, 74, or 75 years old. In some embodiments, the methods further comprise, prior to (b): eluting the at least the portion of the biological sample from a dried blood spot card.

Aspects disclosed herein provide systems for point ofneed or point of care comprising: a testing device or module to detect neutralizing antibodies against a coronavirus in a biological sample obtained from a subject that was administered a pharmaceutical formulation comprising a vaccine against the coronavirus that is formulated for human administration, wherein the neutralizing antibodies functionally block binding between a spike protein of the coronavirus and Angiotensin-converting enzyme 2 (ACE2) to aid in the diagnosis of a disease or a condition caused by the coronavirus at the point of need or the point of care, the testing device comprising: (a) a first peptide or protein comprising the ACE2 polypeptide or a portion thereof; and (b) a second peptide or protein comprising the spike protein or a portion thereof, wherein the first peptide or protein and the second peptide or protein form a complex in an absence of neutralizing antibodies in the biological sample that block binding between the spike protein and ACE2 under conditions otherwise suitable for binding, and wherein the first peptide or protein, or the second peptide or protein comprises a detectable moiety. In some embodiments, the systems further comprise a mobile application that runs on a personal electronic device, wherein the mobile application is programmed to generate a classification of a biological sample from the subject as having a presence, an absence, or a quantity of one or more neutralizing antibodies against the coronavirus by analyzing image data from an image of the testing device at the point of need or the point of care to aid in the diagnosis of the disease or the condition caused by the coronavirus in the subject. In some embodiments, the testing device is portable. In some embodiments, the system does not consist of a cell or a cell culture. In some embodiments, the spike protein of the coronavirus comprises a receptor binding domain specific for binding with ACE2 in vivo. In some embodiments, the coronavirus is a Severe acute respiratory syndrome-related (SARS) virus. In some embodiments, the SARS is SARS-CoV-2. In some embodiments, the neutralizing antibodies against a coronavirus are detected using the testing device or module by contacting a biological sample with the first peptide or protein and the second peptide or protein, wherein a presence, an absence, or a quantity of neutralizing antibodies against a coronavirus in the biological sample are inversely correlated with a presence, an absence, or a quantity of a labeled complex formed between the first peptide or protein and the second peptide or protein, respectively, in the presence of the biological sample. In some embodiments, the systems further comprise: (a) a biological sample contacted with the first peptide or protein and the second peptide or protein of the device or module, wherein the first peptide or protein and the second peptide or protein are bound to each other in the presence of the biological sample. In some embodiments, the biological sample comprises capillary blood. In some embodiments, the human subject is an age that is equal to about 50, 51, 52, 53, 54, 55, 56, 57, 58, 59, 60, 61, 62, 63, 64, 65, 66, 67, 68, 69, 70, 71, 72, 73, 74, or 75 years old. In some embodiments, the methods further comprise, prior to (b): eluting the at least the portion of the biological sample from a dried blood spot card.

Aspects provided here are methods for detecting a neutralizing antibody against the spike (S) protein of a coronavirus (CoV) in a biological sample from a human subject that was vaccinated against the CoV, the method comprising: (a) obtaining the biological sample obtained from the human subject that was vaccinated against the CoV; (b) analyzing at least a portion of the biological sample to detect a level of the neutralizing antibody against the coronavirus that blocks binding between the S protein of the CoV (CoV S) and Angiotensin-converting enzyme 2 (ACE2) in the biological sample by: (i) contacting the biological sample with (1) a first protein comprising ACE2, or a fragment thereof, that binds to the CoV S protein, or a fragment thereof, and (2) a second protein comprising the CoV S protein, or the fragment thereof, that binds to the ACE2, or the fragment thereof, under conditions that permit binding between the ACE2 or fragment thereof, and the CoV S protein or fragment thereof, to produce an ACE2-CoV S complex, wherein the first protein or the second protein is labeled directly or indirectly with a detection reagent; and (ii) detecting a signal corresponding to a level of the ACE2-CoV S complex in the biological sample wherein the signal is inversely correlated with the level of the neutralizing antibody in the biological sample; and (c) detecting the level of the neutralizing antibody in the biological sample based, at least in part, on the signal detected in (a)(ii), wherein performing steps (b) to (c) is performed without a washing step. In some embodiments, the methods further comprise administering a second dose of the vaccine to the subject, provided the presence or the high quantity of the complex is detected in (c). In some embodiments, the vaccine is approved by a regulatory agency for prevention of a disease caused by the coronavirus. In some embodiments, (a)-(c) are performed at a point of need or a point of care. In some embodiments, analyzing in (b) is performed without use of a cell or a cell culture. In some embodiments, methods further comprise: (d) detecting, in a biological sample obtained from the subject, a presence, an absence, or a quantity of one or more antibodies against coronavirus, wherein the one or more antibodies is not a neutralizing antibody. In some embodiments, wherein analyzing in (b) and detecting in (c) are performed with a single integrated device. In some embodiments, wherein (c) is performed by an application on a personal electronic device based, at least in part, on the analysis of (b). In some embodiments, the spike protein of the coronavirus comprises a receptor binding domain specific for binding with ACE2 in vivo. In some embodiments, the coronavirus is a Severe acute respiratory syndrome-related (SARS) virus. In some embodiments, the SARS is SARS-CoV-2. In some embodiments, the biological sample comprises capillary blood. In some embodiments, the human subject is an age that is equal to about 50, 51, 52, 53, 54, 55, 56, 57, 58, 59, 60, 61, 62, 63, 64, 65, 66, 67, 68, 69, 70, 71, 72, 73, 74, or 75 years old. In some embodiments, the methods further comprise, prior to (b): eluting the at least the portion of the biological sample from a dried blood spot card.

Aspects provided here are systems for analyzing a biological sample from a subject that was administered a dose of a vaccine against a coronavirus that has a spike protein, the system comprising: (a) a first testing device or module to detect a presence, an absence, or a level of neutralizing antibodies that functionally block binding between the spike protein of the coronavirus and Angiotensin-converting enzyme 2 (ACE2), wherein the first testing device or module comprises: (i) a first peptide or protein comprising an ACE2 polypeptide, or a portion thereof; (ii) a second peptide or protein comprising a spike protein, or portion, wherein the portion of the ACE2 polypeptide and the portion of the spike protein are configured to bind; and (iii) a test zone for visualization of a complex between (i) and (ii); and (b) a second testing device or module to measure a presence, an absence, or a level of one or more non-neutralizing antibodies against the coronavirus, wherein the second testing device or module comprises: (i) a surface; and (ii) a detectable capture molecule coupled thereto, wherein the detectable capture molecule is specific to the one or more non-neutralizing antibodies. In some embodiments, the systems further comprise an imaging device operatively coupled to the first device, wherein the imaging device is configured to capture an image of the test zone. In some embodiments, the systems further comprise an imaging device operatively coupled to the second device, the imaging device configured to capture an image of the surface. In some embodiments, the imaging device comprises one or more processors configured to generate a classification of the biological sample as having the presence, the absence, or the level of the neutralizing antibodies against the coronavirus. In some embodiments, the imaging device comprises one or more processors configured to generate a second classification of the biological sample as having the presence, the absence, or the level of the presence, the absence, or the level of the one or more non-neutralizing antibodies against the coronavirus. In some embodiments, the systems further comprise an application on an electronic device, the application configured to display on a graphical user interface a report comprising a classification of the biological sample, wherein the classification comprises: (a) the presence, the absence, or the level of the neutralizing antibodies against the coronavirus; or (b) the presence, the absence, or the level of the one or more non-neutralizing antibodies against the coronavirus. In some embodiments, the test zone is positioned at the surface and wherein the first peptide or protein or the second peptide or protein is coupled to the surface at the test zone directly or indirectly. In some embodiments, the first testing module and the second testing module are in a single integrated device. In some embodiments, the first testing device and the second testing device are portable. In some embodiments, the first testing device or module does not consist of a cell or a cell culture. In some embodiments, the spike protein of the coronavirus comprises a receptor binding domain specific for binding with ACE2 in vivo. In some embodiments, the coronavirus is a Severe acute respiratory syndrome-related (SARS) virus. In some embodiments, the SARS is SARS-CoV-2. In some embodiments, the biological sample comprises capillary blood. In some embodiments, the human subject is an age that is equal to about 50, 51, 52, 53, 54, 55, 56, 57, 58, 59, 60, 61, 62, 63, 64, 65, 66, 67, 68, 69, 70, 71, 72, 73, 74, or 75 years old. In some embodiments, the systems further comprising a dried blood spot card comprising the biological sample contained therein or thereon.

Aspects disclosed herein provide methods for detecting a presence, an absence, or a quantity of neutralizing antibodies against a coronavirus in biological samples obtained from a population of human subjects vaccinated against the coronavirus, the method comprising: (a) providing the biological samples obtained from the population of human subjects vaccinated against the coronavirus, wherein the biological samples have an average volume of less than 1 milliliter when obtained; (b) introducing at least a portion of the biological samples to a testing device or module to detect neutralizing antibodies against the coronavirus that functionally block binding between a spike protein of the coronavirus and Angiotensin-converting enzyme 2 (ACE2), wherein the testing device or module comprises: (i) a first peptide or protein comprising the ACE2 polypeptide or a portion thereof that binds to the spike protein or a portion thereof; and (ii) a second peptide or protein comprising the spike protein or the portion thereof, wherein (i), (ii), or a combination of (i) and (ii) comprises a detectable label; and (c) detecting, with the testing device or module, a presence, an absence, or a quantity of a complex formed between (i) and (ii) in the presence of the biological samples, wherein the presence, the absence, or the quantity of the complex is inversely correlated with the presence, the absence, or the quantity of the neutralizing antibodies against the coronavirus in the biological samples, respectively. In some embodiments, the methods further comprise administering to the subject a dose of the vaccine against the coronavirus before or after performing (a) and (b). In some embodiments, the testing device is a point of need or a point of care device. In some embodiments, the method does not consist of utilizing a cell or a cell culture. In some embodiments, the vaccine is formulated in a pharmaceutical composition approved for human administration by a regulatory agency for prevention of a disease caused by the coronavirus. In some embodiments, at least one human subject of the population of human subjects resides in a geographical location of interest. In some embodiments, the spike protein of the coronavirus comprises a receptor binding domain specific for binding with ACE2 in vivo. In some embodiments, the coronavirus is a Severe acute respiratory syndrome-related (SARS) virus. In some embodiments, the SARS is SARS-CoV-2. In some embodiments, the first biological sample and the second biological sample are capillary blood samples. In some embodiments, the biological sample comprises capillary blood. In some embodiments, the human subject is an age that is equal to about 50, 51, 52, 53, 54, 55, 56, 57, 58, 59, 60, 61, 62, 63, 64, 65, 66, 67, 68, 69, 70, 71, 72, 73, 74, or 75 years old. In some embodiments, the methods further comprise, prior to (b): eluting the at least the portion of the biological sample from a dried blood spot card.

Aspects disclosed herein provide methods of analyzing a biological sample, the method comprising: (a) bringing a first biological sample obtained from a subject and a second biological obtained from the subject in contact with a system comprising: (i) a first testing device or module to detect in the first biological sample a presence, an absence, or a quantity of neutralizing antibodies against a coronavirus that functionally block binding between a spike protein of the coronavirus and Angiotensin-converting enzyme 2 (ACE2), wherein the first testing device or module comprises: (1) a first peptide or protein comprising the ACE2 polypeptide or a portion thereof that binds to the spike protein or a portion thereof; (2) a second peptide or protein comprising the spike protein or the portion thereof, wherein (1), (2), or a combination of (1) and (2) comprises a detectable label; and (3) a first test zone for visualization of a complex formed between (1) and (2) in a presence of the first biological sample; and (ii) a second testing device or module to detect a presence, an absence, or a quantity of one or more antibodies against the coronavirus in the second biological sample, wherein the second testing device or module comprises one or more labeled capture molecules coupled to a surface at a second test zone that is specific to the one or more antibodies; (b) detecting the presence, the absence, or the quantity of the neutralizing antibodies against the coronavirus in the first biological sample using the first testing device or module, wherein the presence, the absence, or the quantity of the neutralizing antibodies against the coronavirus is inversely correlated with the presence, the absence, or the quantity of a complex formed between the first peptide or protein and the second peptide or protein in the presence of the first biological sample, respectively; and (c) detecting the presence, the absence, or the quantity of at least one antibody of the one or more antibodies against the coronavirus in the second biological sample using the second testing device or module, wherein the at least one antibody is an immunoglobulin G, an immunoglobulin M, or an immunoglobulin A. In some embodiments, the first test zone is positioned at the surface adjacent to the second test zone, and wherein the first peptide or protein is coupled to the surface at the second test zone directly or indirectly. In some embodiments, the first testing module and the second testing module are in a single integrated device. In some embodiments, the system is portable. In some embodiments, the neutralizing antibodies are induced by administration of a vaccine against the coronavirus to a subject from whom the first or the second biological sample is obtained prior to performing (a)-(c). In some embodiments, the vaccine is approved by a regulatory agency for prevention of a disease caused by the coronavirus. In some embodiments, the first peptide or protein, the second peptide or protein, or both comprises detectable moiety comprising a nanoparticle, a fluorescent dye, an enzymatic label, or a colorimetric label. In some embodiments, the subject is a population of human subjects. In some embodiments, the spike protein of the coronavirus comprises a receptor binding domain specific for binding with ACE2 in vivo. In some embodiments, the coronavirus is a Severe acute respiratory syndrome-related (SARS) virus. In some embodiments, the SARS is SARS-CoV-2. In some embodiments, the biological sample comprises capillary blood. In some embodiments, the human subject is an age that is equal to about 50, 51, 52, 53, 54, 55, 56, 57, 58, 59, 60, 61, 62, 63, 64, 65, 66, 67, 68, 69, 70, 71, 72, 73, 74, or 75 years old. In some embodiments, the methods further comprise, prior to (b): eluting the at least the portion of the biological sample from a dried blood spot card.

Aspects disclosed herein provide methods for confirming efficacy of a vaccine in a human subject using a lateral flow assembly, the method comprising: (a) providing a biological sample obtained from the human subject without use of a phlebotomy, wherein the human subject was administered a vaccine against a coronavirus that has a spike protein; (b) bringing at least a portion of the biological sample into contact with: (i) a first peptide or protein comprising an Angiotensin-converting enzyme 2 (ACE2) polypeptide or a portion thereof; and (ii) a second peptide or protein comprising the spike protein or a portion thereof, wherein the first peptide or protein and the second peptide or protein form a complex in an absence of neutralizing antibodies in the biological sample that block binding between the spike protein and ACE2 under conditions otherwise suitable for binding; (c) bringing the at least the portion of the biological sample, the first peptide or protein, and the second peptide or protein into contact with a porous membrane at a test zone of a lateral flow assay assembly that captures the complex formed in (b)(ii) in the absence of the neutralizing antibodies in the at least the portion of the biological sample; and (d) measuring a presence or a high level of the complex formed in (b) at the test zone, thereby confirming that the vaccine is not effective to immunize the human subject against the coronavirus; or (e) measuring an absence or a low level of the complex formed in (b) at the test zone, thereby confirming that the vaccine is effective to immunize the human subject against the coronavirus, wherein the high level and the low level of the complex are relative to an index or a control. In some embodiments, the vaccine is approved by a regulatory agency for prevention of a disease caused by the coronavirus. In some embodiments, (a)-(e) are performed at a point of need or a point of care. In some embodiments, the method further comprising: (a) bringing the biological sample in contact with a capture molecule coupled to the porous membrane at a second test zone, wherein the capture molecule forms a second complex with the one or more antibodies against the coronavirus; and (b) detecting a presence, an absence, or a level of the one or more antibodies against the coronavirus in the biological sample by detecting the second complex at the second test zone. In some embodiments, the one or more antibodies against the coronavirus is immunoglobulin G, immunoglobulin M, immunoglobulin A, or a combination thereof. In some embodiments, the spike protein of the coronavirus comprises a receptor binding domain specific for binding with ACE2 in vivo. In some embodiments, the coronavirus is a Severe acute respiratory syndrome-related (SARS) virus. In some embodiments, the SARS is SARS-CoV-2. In some embodiments, (b) and (c) are performed simultaneously. In some embodiments, the human subject is not enrolled in a clinical trial for the vaccine. In some embodiments, the biological sample is obtained from the human subject by one or more finger pricks. In some embodiments, performing (b) to (e) does not require a wash step. In some embodiments, the human subject is a population of human subjects. In some embodiments, the human subject is an age that is equal to about 50, 51, 52, 53, 54, 55, 56, 57, 58, 59, 60, 61, 62, 63, 64, 65, 66, 67, 68, 69, 70, 71, 72, 73, 74, or 75 years old. In some embodiments, the methods further comprise, prior to (b): eluting the at least the portion of the biological sample from a dried blood spot card.

Aspects disclosed herein provide systems to confirm efficacy of a vaccine against a coronavirus in a human subject, the system comprising: (a) a first peptide or protein comprising an Angiotensin-converting enzyme 2 (ACE2) polypeptide, or a portion thereof; (b) a second peptide or protein comprising a spike protein, or a portion thereof, wherein the first peptide or protein and the second peptide or protein form a complex in an absence of neutralizing antibodies that block binding between the spike protein and ACE2 under conditions otherwise suitable for binding; and (c) a lateral flow assay assembly comprising a porous membrane that, when contacted with the first peptide or protein, the second peptide or protein, and a biological sample obtained from a human subject, comprises: (i) a presence or a high level of the complex coupled thereto when there is an absence or a low level of the neutralizing antibodies in the biological sample, wherein the high level of the complex is relative to an index or a control; or (ii) an absence or a low level of the complex coupled thereto when there is a presence or a high level of the neutralizing antibodies in the biological sample, wherein the low level of the complex is relative to the index or the control. In some embodiments, the vaccine is approved by a regulatory agency for prevention of a disease caused by the coronavirus. In some embodiments, the system is a point of need or a point of care system. In some embodiments, wherein (a) and (b) are in a homogenous mixture. In some embodiments, the system further comprises a sample receptor mechanically coupled to the porous membrane, wherein the sample receptor retains the biological sample from a human subject. In some embodiments, the system is portable. In some embodiments, the system further comprises an application on a personal electronic device, wherein the application is programed to generate a classification of the biological sample as having the presence, the absence, or the level of the neutralizing antibodies against the coronavirus by analyzing image data from an image of the porous membrane of the lateral flow assay assembly. In some embodiments, the spike protein of the coronavirus comprises a receptor binding domain specific for binding with ACE2 in vivo. In some embodiments, the coronavirus is a Severe acute respiratory syndrome-related (SARS) virus. In some embodiments, the SARS is SARS-CoV-2. In some embodiments, (b) and (c) are performed simultaneously. In some embodiments, the human subject is not enrolled in a clinical trial for the vaccine. In some embodiments, the biological sample is obtained from the human subject by one or more finger pricks. In some embodiments, performing (b) to (e) does not require a wash step. In some embodiments, the human subject is a population of human subjects. In some embodiments, the human subject is an age that is equal to about 50, 51, 52, 53, 54, 55, 56, 57, 58, 59, 60, 61, 62, 63, 64, 65, 66, 67, 68, 69, 70, 71, 72, 73, 74, or 75 years old. In some embodiments, the systems further comprising a dried blood spot card comprising the biological sample contained therein or thereon.

Aspects disclosed herein provide kits for confirming efficacy of a vaccine in a human subject, the kit comprising: (a) a first peptide or protein comprising an Angiotensin-converting enzyme 2 (ACE2) polypeptide, or a portion thereof; (b) a second peptide or protein comprising a spike protein, or a portion thereof, wherein the first peptide or protein and the second peptide or protein form a complex in an absence of neutralizing antibodies that block binding between the spike protein and ACE2 under conditions otherwise suitable for binding; (c) a lateral flow assay assembly comprising a porous membrane having coupled thereto: (i) the first peptide or protein; (ii) the second peptide or protein; or (iii) a capture molecule specific to (i), (ii), or a protein tag conjugated thereto; and (d) instructions for confirming efficacy of the vaccine in the human subject by: (i) contacting the biological sample with (c)(i) (c)(ii), or (c)(iii) coupled to the porous membrane of the lateral flow assay assembly; and (ii) detecting a presence, an absence, or a level of the neutralizing antibodies against the coronavirus in the biological sample by detecting a presence, an absence, or a level of the complex coupled to the porous membrane of the lateral flow assembly. In some embodiments, the kit further comprises a sample receptor mechanically coupled to the porous membrane, wherein the sample receptor contains the biological sample. In some embodiments, the kit further comprises instructions for: (a) obtaining the biological sample; (b) detecting a presence, an absence, or a level of a complex formed between the first peptide or protein and the second peptide or protein in a presence of the biological sample using the lateral flow assay assembly; and (c) classifying the biological sample as having the presence, the absence, or the level of the neutralizing antibodies against the coronavirus. In some embodiments, the kit further comprises instructions for downloading an application on a personal electronic device, wherein the application is programmed to analyze image data from an image of the test zone of the lateral flow assay assembly to classify the biological sample as having the presence, the absence, or the level of neutralizing antibodies against the coronavirus. In some embodiments, the kit further comprises: (a) a second capture molecule coupled to the porous membrane that is specific to one or more antibodies against the coronavirus, wherein the one or more antibodies comprises an immunoglobulin G, an immunoglobulin M, an immunoglobulin A, or a combination thereof; and (b) instructions for: (i) detecting a presence, an absence, or a level of a second complex between the second capture molecule and the one or more antibodies against the coronavirus in the presence of the biological sample using the lateral flow assembly; and (ii) classifying the biological sample as having a presence, an absence, or a level of the one or more antibodies against the coronavirus in the biological sample based the second complex that is detected. In some embodiments, the spike protein of the coronavirus comprises a receptor binding domain specific for binding with ACE2 in vivo. In some embodiments, the coronavirus is a Severe acute respiratory syndrome-related (SARS) virus. In some embodiments, the SARS is SARS-CoV-2. In some embodiments, the human subject is not enrolled in a clinical trial for the vaccine. In some embodiments, the biological sample is obtained from the human subject by one or more finger pricks. In some embodiments, the human subject is a population of human subjects. In some embodiments, the human subject is an age that is equal to about 50, 51, 52, 53, 54, 55, 56, 57, 58, 59, 60, 61, 62, 63, 64, 65, 66, 67, 68, 69, 70, 71, 72, 73, 74, or 75 years old. In some embodiments, kits further comprising a dried blood spot card comprising the biological sample contained therein or thereon.

Aspects disclosed herein provide methods of measuring vaccine-induced neutralizing antibodies against a coronavirus, the method comprising: (a) providing a biological sample obtained from a subject that was administered a vaccine against a coronavirus, wherein the coronavirus is encoded by a nucleic acid sequence having greater than or equal to about 80% sequence identity to SEQ ID NO 7-10, 13-20, 43, 67, or 91; (b) detecting, in the biological sample, a presence or a quantity of a labeled complex formed between at least two of: (i) a first peptide or protein comprising an amino acid sequence that is at least 80% identical to any one of SEQ ID NOS: 2-6, 11-12, 24, 47-48, 71-72, 95-96, or 115; (ii) a second peptide or protein comprising an amino acid sequence that is at least 80% identical to SEQ ID NO: 1; and (iii) a neutralizing antibody against the coronavirus in the biological sample; and (c) generating a classification of the biological sample based, at least partially, on (b) as having sufficient or insufficient vaccine-induced neutralizing antibodies to confer immunity against the coronavirus. In some embodiments, the vaccine is allowed by a regulatory agency for the prevention of disease caused by the coronavirus. In some embodiments, the method further comprises administering a second dose of the vaccine to the subject, provided a presence or a quantity of a labeled complex formed between (i) and (iii) is detected in (b). In some embodiments, the method further comprises administering a second dose of the vaccine to the subject, provided a presence or a quantity of a labeled complex formed between (i) and (iii) is detected in (b). In some embodiments, (a)-(c) are performed at the point of need or point of care. In some embodiments, (b) is performed using a lateral flow assay, agglutination assay, an enzyme-linked immunoassay, photonic ring resonance, or a combination thereof. In some embodiments, a multiple doses of the vaccine are administered to the subject at different timepoints, and steps (a)-(c) are repeated following each dose of a multiple doses to measure vaccine-induced neutralizing antibodies against the coronavirus. In some embodiments, steps (a)-(c) are performed within at least 1 month after a first dose of the vaccine. In some embodiments, steps (a)-(c) are performed within at least 2 months after a first dose of the vaccine. In some embodiments, steps (a)-(c) are performed within at least 6 months after a first dose of the vaccine. In some embodiments, steps (a)-(c) are performed within at least 12 months after a first dose of the vaccine. In some embodiments, steps (a)-(c) are performed within at least 2 years after a first dose of the vaccine. In some embodiments, steps (a)-(c) are performed within at least 4 years after a first dose of the vaccine. In some embodiments, the subject is a plurality of subjects. In some embodiments, the plurality of subject is not enrolled in a clinical trial for the vaccine. In some embodiments, the plurality of subject is a population of subjects. In some embodiments, the coronavirus is severe acute respiratory syndrome coronavirus 2 (SARS-CoV-2). In some embodiments, the method further comprises determining the efficacy of the vaccine based, at least partially, on (c). In some embodiments, the method further comprises determining the durability of the vaccine based, at least partially, on (c). In some embodiments, the coronavirus is encoded by a nucleic acid sequence having greater than or equal to about 90% sequence identity to SEQ ID NO: 7-10, 13-20, 43, 67, or 91. In some embodiments, the coronavirus is encoded by a nucleic acid sequence having greater than or equal to about 95% sequence identity to SEQ ID NO: 7-10, 13-20, 43, 67, or 91. In some embodiments, the coronavirus is encoded by a nucleic acid sequence having greater than or equal to about 100% sequence identity to SEQ ID NO: 7-10, 13-20, 43, 67, or 91. In some embodiments, the subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide a method comprising: (a) providing a biological sample from a subject that was administered a vaccine against a coronavirus encoded by a nucleic acid sequence having greater than or equal to about 80% sequence identity to SEQ ID NO: 7-10, 13-20, 43, 67, or 91; (b) measuring a presence or level of a neutralizing antibody against the coronavirus in the biological sample by: (i) contacting the biological sample with a first peptide or protein comprising an amino acid sequence that is at least 80% identical to any one of SEQ ID NOS: 2-6, 11-12, 24, 47-48, 71-72, 95-96, or 115, and a second peptide or protein comprising an amino acid sequence that is at least 80% identical to SEQ ID NO: 1; and (ii) detecting a presence or a level a labeled complex between the at least two of (1) the first peptide or protein; (2) the second peptide or protein; and (3) the neutralizing antibody against the coronavirus; and (c) identifying the subject as having or lacking sufficient immunity against the coronavirus, wherein the presence or the level of the complex between (1) and (2) is indicative of lacking sufficient immunity and the presence or the level of the complex between (1) and (3) is indicative of having sufficient immunity. In some embodiments, the vaccine is allowed by a regulatory agency for the prevention of disease caused by the coronavirus. In some embodiments, the method further comprises administering a second dose of the vaccine to the subject, provided a presence or a quantity of a labeled complex formed between (i) and (iii) is detected in (b). In some embodiments, the method further comprises administering a second dose of the vaccine to the subject, provided a presence or a quantity of a labeled complex formed between (i) and (iii) is detected in (b). In some embodiments, (a)-(c) are performed at the point of need or point of care. In some embodiments, (b) is performed using a lateral flow assay, agglutination assay, an enzyme-linked immunoassay, photonic ring resonance, or a combination thereof. In some embodiments, a multiple doses of the vaccine are administered to the subject at different timepoints, and steps (a)-(c) are repeated following each dose of a multiple doses to measure vaccine-induced neutralizing antibodies against the coronavirus. In some embodiments, steps (a)-(c) are performed within at least 1 month after a first dose of the vaccine. In some embodiments, steps (a)-(c) are performed within at least 2 months after a first dose of the vaccine. In some embodiments, steps (a)-(c) are performed within at least 6 months after a first dose of the vaccine. In some embodiments, steps (a)-(c) are performed within at least 12 months after a first dose of the vaccine. In some embodiments, steps (a)-(c) are performed within at least 2 years after a first dose of the vaccine. In some embodiments, steps (a)-(c) are performed within at least 4 years after a first dose of the vaccine. In some embodiments, the subject is a plurality of subjects. In some embodiments, the plurality of subject is not enrolled in a clinical trial for the vaccine. In some embodiments, the plurality of subject is a population of subjects. In some embodiments, the coronavirus is severe acute respiratory syndrome coronavirus 2 (SARS-CoV-2). In some embodiments, the method further comprises determining the efficacy of the vaccine based, at least partially, on (c). In some embodiments, the method further comprises determining the durability of the vaccine based, at least partially, on (c). In some embodiments, the coronavirus is encoded by a nucleic acid sequence having greater than or equal to about 90% sequence identity to SEQ ID NO: 7-10, 13-20, 43, 67, or 91. In some embodiments, the coronavirus is encoded by a nucleic acid sequence having greater than or equal to about 95% sequence identity to SEQ ID NO: 7-10, 13-20, 43, 67, or 91. In some embodiments, the coronavirus is encoded by a nucleic acid sequence having greater than or equal to about 100% sequence identity to SEQ ID NO: 7-10, 13-20, 43, 67, or 91. In some embodiments, the subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide methods of confirming efficacy of a vaccine using a lateral flow assembly, the method comprising: (a) providing a biological sample obtained from a subject that was administered a vaccine against a coronavirus encoded by a nucleic acid sequence having greater than or equal to about 80% sequence identity to SEQ ID NO: 7-10, 13-20, 43, 67, or 91; (b) bringing the biological sample into contact with a porous membrane at a test zone of a lateral flow assay assembly adapted to detect a presence or a level of a labeled complex coupled thereto, where the labeled complex is formed between at least two of: (i) a first peptide or protein comprising an amino acid sequence that is at least 80% identical to any one of SEQ ID NOS: 2-6, 11-12, 24, 47-48, 71-72, 95-96, or 115; (ii) a second peptide or protein comprising an amino acid sequence that is at least 80% identical to SEQ ID NO: 1; and (iii) a neutralizing antibody against the coronavirus in the biological sample; and (c) measuring the presence or the level of the labeled complex at the test zone, wherein the presence or the level of the labeled complex formed between (i) and (iii) indicates the vaccine if effective to confer immunity in the subject against the coronavirus, and the presence or the level of the labeled complex formed between (i) and (iii) indicates the vaccine is not effective to confer immunity in the subject against the coronavirus. In some embodiments, the vaccine is allowed by a regulatory agency for the prevention of disease caused by the coronavirus. In some embodiments, the method further comprises administering a second dose of the vaccine to the subject, provided the presence or the quantity of a labeled complex formed between (i) and (iii) is measured in (c). In some embodiments, steps (a)-(c) are performed at the point of need or point of care. In some embodiments, a multiple doses of the vaccine are administered to the subject at different timepoints, and steps (a)-(c) are repeated following each dose of a multiple doses to confirming efficacy of a vaccine against the coronavirus. In some embodiments, steps (a)-(c) are performed within at least 1 month after a first dose of the vaccine. In some embodiments, steps (a)-(c) are performed within at least 2 months after a first dose of the vaccine. In some embodiments, steps (a)-(c) are performed within at least 6 months after a first dose of the vaccine. In some embodiments, steps (a)-(c) are performed within at least 12 months after a first dose of the vaccine. In some embodiments, steps (a)-(c) are performed within at least 2 years after a first dose of the vaccine. In some embodiments, steps (a)-(c) are performed within at least 4 years after a first dose of the vaccine. In some embodiments, the subject is a plurality of subjects. In some embodiments, the plurality of subjects are enrolled in a clinical trial for the vaccine. In some embodiments, the plurality of subject is not enrolled in a clinical trial for the vaccine. In some embodiments, the plurality of subject is a population of subjects. In some embodiments, the method further comprises determining the efficacy of the vaccine based, at least partially, on (c). In some embodiments, the method further comprises determining the durability of the vaccine based, at least partially, on (c). In some embodiments, the coronavirus is encoded by a nucleic acid sequence having greater than or equal to about 90% sequence identity to SEQ ID NO: 7-10, 13-20, 43, 67, or 91. In some embodiments, the coronavirus is encoded by a nucleic acid sequence having greater than or equal to about 95% sequence identity to SEQ ID NO: 7-10, 13-20, 43, 67, or 91. In some embodiments, the coronavirus is encoded by a nucleic acid sequence having greater than or equal to about 100% sequence identity to SEQ ID NO: 7-10, 13-20, 43, 67, or 91. In some embodiments, the first peptide or protein comprises an amino acid sequence that is at least 85% identical to any one of SEQ ID NOS: 2-6, 11-12, 24, 47-48, 71-72, 95-96, or 115. In some embodiments, the first peptide or protein comprises an amino acid sequence that is at least 90% identical to any one of SEQ ID NOS: 2-6, 11-12, 24, 47-48, 71-72, 95-96, or 115. In some embodiments, the first peptide or protein comprises an amino acid sequence that is at least 99% identical to any one of SEQ ID NOS: 2-6, 11-12, 24, 47-48, 71-72, 95-96, or 115. In some embodiments, the second peptide or protein comprises an amino acid sequence that is at least 85% identical to SEQ ID NO: 1. In some embodiments, the second peptide or protein comprises an amino acid sequence that is at least 90% identical to SEQ ID NO: 1. In some embodiments, the second peptide or protein comprises an amino acid sequence that is at least 99% identical to SEQ ID NO: 1. In some embodiments the vaccine is an antigenic peptide, a live-attenuated virus, a dead coronavirus, or a nucleic acid molecule encoding the antigenic peptide, or a combination thereof. In some embodiments, the subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide methods of measuring vaccine-induced neutralizing antibodies against a coronavirus, the method comprising: (a) providing a biological sample obtained from a subject that was administered a dose of a vaccine against the coronavirus, wherein the coronavirus has a spike protein that comprises an amino acid sequence that is at least about 80% identical to SEQ ID NO: 2; (b) detecting, in a presence of the biological sample, an absence, a presence, or a quantity of a complex formed between: (i) a first peptide or protein comprising an amino acid sequence that is at least about 80% identical to SEQ ID NO: 1; and a second peptide or protein comprising an amino acid sequence that is at least about 80% identical to any one of SEQ ID NOS: 2-6, 11-12, 24, 47-48, 71-72, 95-96, or 115; and (c) generating a classification of the biological sample as having a presence, an absence, or a quantity of vaccine-induced neutralizing antibodies against the coronavirus based on detecting in (b). In some embodiments, the method of generating in (c) is performed by a mobile application on a personal electronic device comprising a smartphone, a tablet, or a personal computer. In some embodiments, the method further comprises: generating a prognosis of the subject based on the classification of the biological sample, wherein the prognosis is related to immunity of the subject to an infection by the coronavirus, or a disease or a condition caused by the coronavirus; or generating a measure of susceptibility of the subject to an infection by the coronavirus. In some embodiments, the method does not consist of utilizing an immortalized cell or immortalized cell culture. In some embodiments, the method further comprises confirming that the vaccine was effective to induce the vaccine-induced neutralizing antibodies against the coronavirus in the subject, provided the absence or a low quantity of the complex is detected in (b), wherein the low quantity is relative to an index or a control. In some embodiments, the method further comprises detecting, in a biological sample obtained from the subject, a presence, an absence, or a quantity of one or more antibodies against the coronavirus, wherein the one or more antibodies is not a neutralizing antibody. In some embodiments, the one or more antibodies against the coronavirus belong to a class of antibodies comprising immunoglobulin A, immunoglobulin M, or immunoglobulin G, or a combination thereof. In some embodiments, the vaccine is approved by a regulatory agency for prevention of a disease caused by the coronavirus. In some embodiments, the method further comprises administering a second dose of the vaccine to the subject, provided the presence or a high quantity of the complex is detected in (b), wherein the high quantity is relative to an index or a control. In some embodiments, the subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide method of aiding in a diagnosis of a disease or condition caused by a coronavirus infection, the method comprising: (a) providing a biological sample obtained from a subject in need thereof; (b) bringing the biological sample into contact with a first peptide or protein comprising an amino acid sequence that is at least about 80% identical to SEQ ID NO: 1 and a second peptide or protein comprising an amino acid sequence that is at least about 80% identical to any one of SEQ ID NOS: 2-6, 11-12, 24, 47-48, 71-72, 95-96, or 115; (c) detecting a presence, an absence, or a quantity of a complex formed between the first peptide or protein and the second peptide or protein in a presence of the biological sample; and (d) classifying the biological sample as having a presence, an absence, or a quantity of neutralizing antibodies against a coronavirus based on detecting in (c), thereby aiding in the diagnosis of a disease or a condition caused by the coronavirus, wherein the coronavirus has a spike protein that comprises an amino acid sequence that is at least about 80% identical to SEQ ID NO: 2. In some embodiments, the classifying in (d) is performed by a mobile application on a personal electronic device comprising a smartphone, a tablet, or a personal computer. In some embodiments, the method does not consist of utilizing a cell or a cell culture. In some embodiments, the classifying in (d) comprises classifying the biological sample as having the presence or a high quantity of the neutralizing antibodies, provided the absence or a low quantity of the complex detected in (c), and wherein the low quantity is relative to an index or a control. In some embodiments, the classifying in (d) comprises classifying the biological sample as having the absence or a low quantity of the neutralizing antibodies, provided the presence or a high quantity of the complex is detected in (c), and wherein the high quantity is relative to an index or a control. In some embodiments, the method further comprises: (a) bringing a biological sample from the subject into contact with one or more capture molecules specific to an IgG antibody, an IgM antibody, or an IgA antibody against the coronavirus; and (b) detecting binding between the IgG antibody, an IgM antibody, or an IgA antibody and the one or more capture molecules. In some embodiments, the method further comprises: (a) generating a prognosis of the subject based on classifying in (d), wherein the prognosis is related to immunity of the subject against an infection by the coronavirus, or the disease or the condition caused by the coronavirus; or (b) generating a measure of susceptibility of the subject to an infection by the coronavirus. In some embodiments, the subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide a system for point of need or point of care comprising: a testing device or module to detect neutralizing antibodies against a coronavirus has a spike protein that comprises an amino acid sequence that is at least about 80% identical to SEQ ID NO: 2 to aid in the diagnosis of a disease or a condition caused by the coronavirus at the point of need or the point of care, the testing device comprising: (a) a first peptide or protein comprising an amino acid sequence that is at least about 80% identical to SEQ ID NO: 1; and (b) a second peptide or protein comprising an amino acid sequence that is at least about 80% identical to any one of SEQ ID NOS: 2-6, 11-12, 24, 47-48, 71-72, 95-96, or 115, wherein the first peptide or protein or the second peptide or protein comprises a detectable moiety. In some embodiments, the system further comprises a mobile application configured to run on a personal electronic device, wherein the mobile application is configured to generate a classification of the biological sample as having a presence, an absence, or a quantity of one or more neutralizing antibodies against the coronavirus by analyzing image data from an image of the testing device at the point of need or the point of care to aid in the diagnosis of the disease or the condition caused by the coronavirus in the subject. In some embodiments, the testing device is portable. In some embodiments, the system does not consist of a cell or a cell culture. In some embodiments, the subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide methods for confirming efficacy of a vaccine using a lateral flow assembly, the method comprising: (a) providing a biological sample obtained from a subject that was administered a vaccine against a coronavirus that has a spike protein comprising an amino acid sequence that is at least about 80% identical to SEQ ID NO: 2; (b) bringing the biological sample into contact with a porous membrane at a test zone of a lateral flow assay assembly adapted to detect a presence, absence, or a level of a complex coupled thereto, where the complex is formed between: (i) a first peptide or protein comprising an amino acid sequence that is at least about 80% identical to SEQ ID NO: 1; and (ii) a second peptide or protein comprising an amino acid sequence that is at least about 80% identical to any one of SEQ ID NOS: 2-6, 11-12, 24, 47-48, 71-72, 95-96, or 115; and (c) measuring the presence or a high level of the complex formed between (i) and (ii) in the presence of the biological sample at the test zone, thereby confirming that the vaccine is not effective to immunize the subject against the coronavirus; or (d) measuring the absence or a low level of the complex formed between (i) and (ii) in the presence of the biological sample at the test zone, thereby confirming that the vaccine is effective to immunize the subject against the coronavirus, wherein the high level and the low level of the complex is relative to an index or a control. In some embodiments, the vaccine is approved by a regulatory agency for prevention of a disease caused by the coronavirus. In some embodiments, steps (a)-(d) of the method are performed at a point of need or a point of care. In some embodiments, the amino acid sequence of the spike protein is at least about 95% identical to SEQ ID NO: 2. In some embodiments, the amino acid sequence of the second peptide or protein is at least about 95% identical to any one of SEQ ID NOS: 2-6, 11-12, 24, 47-48, 71-72, 95-96, or 115. In some embodiments, the method further comprises detecting, in the biological sample obtained from the subject, a presence, an absence, or a level of a second complex coupled to the porous membrane at a second test zone between a capture molecule and one or more antibodies against the coronavirus in the biological sample. In some embodiments, the one or more antibodies against the coronavirus is immunoglobulin G, immunoglobulin M, immunoglobulin A, or a combination thereof. In some embodiments, the subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide systems comprising: a lateral flow assay assembly comprising: (a) a first peptide or protein comprising an amino acid sequence that is at least 80% identical to SEQ ID NO 1; (b) a second peptide or protein comprising an amino acid sequence that is at least about 80% identical to any one of SEQ ID NOS: 2-6, 11-12, 24, 47-48, 71-72, 95-96, or 115; and (c) a porous membrane having a presence or a high level of a complex coupled thereto formed between (a) and (b) in a presence of a biological sample obtained from a subject in need thereof, wherein the presence or the high level of the complex indicates an absence or a level of neutralizing antibodies against a coronavirus that has a spike protein comprising an amino acid sequence that is at least about 80% identical to SEQ ID NO: 2 in the biological sample; or (d) the porous membrane having an absence or a low level of the complex coupled thereto in the presence of the biological sample obtained from the subject in need thereof, wherein the absence or the low level of the complex indicates a presence or a level of the neutralizing antibodies against the coronavirus, wherein the low level and the high level of the complex is relative to an index or a control. In some embodiments, (a) and (b) are in a homogenous mixture that is configured to be applied to the porous membrane. In some embodiments, the system further comprises a sample receptor mechanically coupled to the porous membrane, wherein the sample receptor is configured to retain the biological sample from a subject. In some embodiments, the system is portable. In some embodiments, the system further comprises an application configured to run on a personal electronic device, wherein the application is configured to generate a classification of the biological sample as having the presence, the absence, or the level of the neutralizing antibodies against the coronavirus by analyzing image data from an image of the porous membrane of the lateral flow assay assembly. In some embodiments, the spike protein of the coronavirus comprises an amino acid sequence that is at least about 95% identical to SEQ ID NO: 2. In some embodiments, the amino acid sequence of the second peptide or protein is at least about 95% identical to any one of SEQ ID NOS: 2-6, 11-12, 24, 47-48, 71-72, 95-96, or 115. In some embodiments, the system further comprises a capture molecule coupled to the porous membrane that is specific to one or more antibodies against the coronavirus, wherein the one or more antibodies comprise an immunoglobulin G, immunoglobulin M, an immunoglobulin A, or a combination thereof. In some embodiments, the subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide kits comprising: (a) a lateral flow assay assembly comprising: (i) a first peptide or protein comprising an amino acid sequence that is at least 80% identical to SEQ ID NO: 1; (ii) a second peptide or protein comprising an amino acid sequence that is at least about 80% identical to any one of SEQ ID NOS: 2-6, 11-12, 24, 47-48, 71-72, 95-96, or 115; and (iii) a porous membrane comprising a test zone having coupled thereto: (1) the first peptide or protein; (2) the second peptide or protein; or (3) a capture molecule specific to (1), (2), or a protein tag conjugated thereto; and (b) instructions for assaying a biological sample with the lateral flow assay assembly to detect a presence, an absence, or a level of neutralizing antibodies against a coronavirus that has a spike protein comprising an amino acid sequence that is at least 80% identical to SEQ ID NO: 2 in the biological sample. In some embodiments, the kit further comprises a sample receptor mechanically coupled to the porous membrane, wherein the sample receptor is configured to contain the biological sample. In some embodiments, the kit of further comprises instructions for: (a) obtaining the biological sample; (b) detecting a presence, an absence, or a level of a complex formed between the first peptide or protein and the second peptide or protein in a presence of the biological sample using the lateral flow assay assembly; and (c) classifying the biological sample as having the presence, the absence, or the level of the neutralizing antibodies against the coronavirus. In some embodiments, the kit further comprises instructions for downloading an application on a personal electronic device, wherein the application is configured to analyze image data from an image of the test zone of the lateral flow assay assembly to classify the biological sample as having the presence, the absence, or the level of neutralizing antibodies against the coronavirus. In some embodiments, the kit further comprises: (a) a second capture molecule coupled to the porous membrane that is specific to one or more antibodies against the coronavirus, wherein the one or more antibodies comprises an immunoglobulin G, an immunoglobulin M, an immunoglobulin A, or a combination thereof; and (b) instructions for: (i) detecting a presence, an absence, or a level of a second complex between the second capture molecule and the one or more antibodies against the coronavirus in the presence of the biological sample using the lateral flow assembly; and (ii) classifying the biological sample as having a presence, an absence, or a level of the one or more antibodies against the coronavirus in the biological sample based the second complex that is detected. In some embodiments, the subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide methods comprising: (a) providing a biological sample from a subject that was administered a dose of a vaccine against a coronavirus that has a spike protein comprising an amino acid sequence that is at least about 80% identical to SEQ ID NO: 2, 5, 11, 48, 72, 96, or 115; (b) measuring a presence, an absence, or a level of a vaccine-induced neutralizing antibody against the coronavirus in the biological sample by: (i) contacting the biological sample with a first peptide or protein comprising an amino acid sequence that is at least about 80% identical to SEQ ID NO: 1, and a second peptide or protein comprising an amino acid sequence that is at least about 80% identical to any one of SEQ ID NOS: 2-6, 11-12, 24, 47-48, 71-72, 95-96, or 115; and (ii) detecting a presence or a level of a complex between the first peptide or protein and the second peptide or protein in a presence of the biological sample; and (c) identifying the subject as: (i) lacking sufficient immunity against the coronavirus, provided the presence or a high level of the complex is detected in (b)(ii); or (ii) having sufficient immunity against the coronavirus, provided the absence or a low level of the complex is detected in (b)(ii), wherein the high level and the low level are relative to an index or a control. In some embodiments, the method further comprises administering a second dose of the vaccine to the subject, provided the presence or the high quantity of the complex is detected in (b)(ii). In some embodiments, the vaccine is approved by a regulatory agency for prevention of a disease caused by the coronavirus. In some embodiments, the amino acid sequence of the coronavirus is at least about 95% identical to SEQ ID NO: 2. In some embodiments, the amino acid sequence of the second peptide or protein is at least about 95% identical to any one of SEQ ID NOS: 2-6, 11-12, 24, 47-48, 71-72, 95-96, or 115. In some embodiments, (a)-(c) are performed at a point of need or a point of care. In some embodiments, the method is performed without use of a cell or a cell culture. In some embodiments, the method further comprises detecting, in a biological sample obtained from the subject, a presence, an absence, or a quantity of one or more antibodies against coronavirus, wherein the one or more antibodies is not a neutralizing antibody. In some embodiments, the method is performed with a single integrated device. In some embodiments, (c) is performed by an application on a personal electronic device. In some embodiments, the subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide a system comprising: (a) a first testing device or module to detect a presence, an absence, or a level of neutralizing antibodies against a coronavirus that has a spike protein comprising an amino acid sequence that is at least about 80% identical to SEQ ID NO: 2, 5, 11, 48, 72, 96, or 115, wherein the first testing device or module comprises: (i) a first peptide or protein comprising an amino acid sequence that is at least about 80% identical to SEQ ID NO: 1; (ii) a second peptide or protein comprising an amino acid sequence that is at least about 80% identical to any one of SEQ ID NOS: 2-6, 11-12, 24, 47-48, 71-72, 95-96, or 115; and (iii) a test zone for visualization of a complex between (i) and (ii); and (b) a second testing device or module to measure a presence, an absence, or a level of one or more non-neutralizing antibodies against the coronavirus, wherein the second testing device or module comprises: (i) a surface; and (ii) a detectable capture molecule coupled thereto, wherein the detectable capture molecule is specific to the one or more non-neutralizing antibodies. In some embodiments, the system further comprises an imaging device operatively coupled to the first device, wherein the imaging device is configured to capture an image of the test zone. In some embodiments, the system further comprises an imaging device operatively coupled to the second device, the imaging device configured to capture an image of the surface. In some embodiments, the imaging device comprises one or more processors configured to generate a classification of the biological sample as having the presence, the absence, or the level of the neutralizing antibodies against the coronavirus. In some embodiments, the imaging device comprises one or more processors configured to generate a second classification of the biological sample as having the presence, the absence, or the level of the presence, the absence, or the level of the one or more non-neutralizing antibodies against the coronavirus. In some embodiments, the system further comprises an application on an electronic device, the application configured to display on a graphical user interface a report comprising a classification of the biological sample, wherein the classification comprises: (a) the presence, the absence, or the level of the neutralizing antibodies against the coronavirus; or (b) the presence, the absence, or the level of the one or more non-neutralizing antibodies against the coronavirus. In some embodiments, the test zone is positioned at the surface and wherein the first peptide or protein or the second peptide or protein is coupled to the surface at the test zone directly or indirectly. In some embodiments, the first testing module and the second testing module are in a single integrated device. In some embodiments, the first testing device and the second testing device are portable. In some embodiments, the first testing device or module does not consist of a cell or a cell culture. In some embodiments, the subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide systems for point of need or point of care comprising: (a) a testing device to detect neutralizing antibodies against a SARS-CoV-2 or variant thereof to aid in the diagnosis of a disease or a condition caused by SARS-CoV-2 or variant thereof, the testing device comprising: (i) a composition comprising: a first peptide or protein derived from an ACE2, or portion thereof; and a second peptide or protein derived from a spike glycoprotein of the SARS-CoV-2 or variant thereof, or portion thereof wherein at least one of the first peptide or protein and the second peptide or protein is labeled with a detectable moiety. In some embodiments, the system further comprises an application configured to run on an electronic device, the electronic device comprising a camera to capture an image of a mixture at the point of need or the point of care, wherein the application is configured to generate a classification of the biological sample as having a presence, an absence, or a quantity of neutralizing antibodies against the SARS-CoV-2 or variant thereof using image data from the image, the image data comprising a presence, an absence, or a quantity of a binding complex between the first peptide or protein and the second peptide or protein detected in the biological sample with the testing device at a point of need or point of care to aid in the diagnosis of the disease or the condition caused by SARS-CoV-2 or variant thereof. In some embodiments, the testing device of the system is portable. In some embodiments, the system does not consist of an immortalized cell or an immortalized cell culture. In some embodiments, the presence, the absence, or the quantity of neutralizing antibodies against SARS-CoV-2 or variant thereof is associated with: a prognosis related to adaptive immunity of the subject to an infection by the SARS-CoV-2 or variant thereof, or the disease or the condition caused by the SARS-CoV-2; or a measure of susceptibility of the subject to an infection by the SARS-CoV-2. In some embodiments, the electronic device of the system is a personal electronic device comprising a smartphone, a tablet, or a personal computer. In some embodiments, the subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide methods for confirming a presence of vaccine-induced neutralizing antibodies against a coronavirus in a biological sample obtained from a vaccinated subject, the method comprising: (a) providing a testing device or module to detect neutralizing antibodies induced by a vaccine against a coronavirus that has a spike protein comprising an amino acid sequence that is at least about 80% identical to SEQ ID NO: 2, 5, 11, 48, 72, 96, or 115, wherein the testing device or module comprises: (i) a first peptide or protein comprising an amino acid sequence that is at least about 80% identical to SEQ ID NO: 1; and (ii) a second peptide or protein comprising an amino acid sequence that is at least about 80% identical to any one of SEQ ID NOS: 2-6, 11-12, 24, 47-48, 71-72, 95-96, or 115; and (b) measuring a presence, absence, or quantity of a complex between (i) and (ii) in the presence of the biological sample obtained from a subject who was administered a vaccine against the coronavirus with the testing device or module. In some embodiments, the method further comprises: (a) confirming of the presence or quantity of the neutralizing antibodies induced by the vaccine in the subject; or (b) identifying a risk of infection of the subject by the coronavirus, or a risk that the subject will develop a disease or a condition caused by the coronavirus. In some embodiments, the method further comprises administering to the subject a dose of the vaccine against the coronavirus before or after performing (a) and (b). In some embodiments, the testing device is a point of need or a point of care device. In some embodiments, the method does not consist of utilizing a cell or a cell culture. In some embodiments, the testing device or module is a lateral flow assay device. In some embodiments, the vaccine is approved by a regulatory agency for prevention of a disease caused by the coronavirus. In some embodiments, the amino acid sequence of the spike protein is at least about 95% identical to SEQ ID NO: 2. In some embodiments, the subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide methods comprising: (a) providing a biological sample obtained from a subject in need thereof to measure a presence, an absence, or a quantity of neutralizing antibodies against a coronavirus that has a spike protein comprising an amino acid sequence that is at least about 80% identical to SEQ ID NO: 2 by: (1) contacting the biological sample with a first peptide or protein comprising an amino acid sequence that is at least about 80% identical to SEQ ID NO: 1, and a second peptide or protein comprising an amino acid sequence that is at least about 80% identical to any one of SEQ ID NOS: 2-6, 11-12, 24, 47-48, 71-72, 95-96, or 115; and

(2) detecting an absence, a presence, or a quantity of a complex formed between the first peptide or protein and the second peptide or protein in the presence of the biological sample; and (b) generating a classification of the biological sample as having a presence or a high quantity of the neutralizing antibodies against the coronavirus, provided an absence or a low quantity of the complex is detected in (2). In some embodiments, the neutralizing antibodies are induced by administration of a vaccine against the coronavirus to the subject prior to performing (a)-(b). In some embodiments, the method further comprises identifying a risk of infection of the subject by the coronavirus, or a risk that the subject will develop a disease or a condition caused by the coronavirus. In some embodiments, the vaccine is approved by a regulatory agency for prevention of a disease caused by the coronavirus. In some embodiments, the method further comprising detecting, in a biological sample obtained from the subject, a presence, an absence, or a quantity of one or more antibodies against the coronavirus comprising an immunoglobulin G, immunoglobulin M, an immunoglobulin A, or a combination thereof. In some embodiments, the subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide methods of analyzing a biological sample, the method comprising: (a) providing a system comprising: (i) a first testing device or module to detect a presence, an absence, or a quantity of neutralizing antibodies against a coronavirus that has a spike protein comprising an amino acid sequence that is at least about 80% identical to SEQ ID NO: 2, wherein the first testing device or module comprises: (a) a first peptide or protein comprising an amino acid sequence that is at least about 80% identical to SEQ ID NO: 1; (b) a second peptide or protein comprising an amino acid sequence that is at least about 80% identical to any one of SEQ ID NOS: 2-6, 11-12, 24, 47-48, 71-72, 95-96, or 115; and (c) a first test zone for visualization of a complex formed between (a) and (b) in a presence of a biological sample obtained from a subject in need thereof; and (ii) a second testing device or module to detect a presence, an absence, or a quantity of one or more antibodies against the coronavirus in a biological sample obtained from the subject, wherein the second testing device or module comprises one or more capture molecules coupled to a surface at a second test zone, and wherein the one or more capture molecules is specific to the one or more antibodies; (b) detecting the presence, the absence, or the quantity of the neutralizing antibodies against the coronavirus using the first testing device or module; and (c) detecting the presence, the absence, or the quantity of at least one antibody of the one or more antibodies against the coronavirus using the second testing device or module, wherein the at least one antibody is an immunoglobulin G, an immunoglobulin M, or an immunoglobulin A. In some embodiments, the first test zone is positioned at the surface adjacent to the second test zone, and wherein the first peptide or protein is coupled to the surface at the second test zone directly or indirectly. In some embodiments, the first testing module and the second testing module are in a single integrated device. In some embodiments, wherein system is portable. In some embodiments, the neutralizing antibodies are induced by administration of a vaccine against the coronavirus to the subject prior to performing (a)-(c). In some embodiments, the vaccine is approved by a regulatory agency for prevention of a disease caused by the coronavirus. In some embodiments, the method further comprises (a) confirming the presence or the quantity of neutralizing antibodies induced by a vaccine against the coronavirus that was administered to the subject; or (b) identifying a risk of infection of the subject by the coronavirus, or a risk that the subject will develop a disease or a condition caused by the coronavirus. In some embodiments, the subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide methods of measuring vaccine-induced neutralizing antibodies against a coronavirus, the method comprising: (a) providing a biological sample obtained from an animal subject that was administered a vaccine against a coronavirus, wherein the coronavirus is encoded by a nucleic acid sequence having greater than or equal to about 80% sequence identity to SEQ ID NO: 7-10, 13-20, 43, 67, or 91; (b) detecting, in the biological sample, a presence or a quantity of a labeled complex formed between at least two of: (i) a first peptide or protein comprising an amino acid sequence that is at least 80% identical to any one of SEQ ID NOS: 2-6, 11-12, 24, 47-48, 71-72, 95-96, or 115; (ii) a second peptide or protein comprising an amino acid sequence that is at least 80% identical to SEQ ID NO: 1; and (iii) a neutralizing antibody against the coronavirus in the biological sample; and (c) generating a classification of the biological sample based, at least partially, on (b) as having sufficient or insufficient vaccine-induced neutralizing antibodies to confer immunity against the coronavirus. In some embodiments, the vaccine is allowed by a regulatory agency for the prevention of disease caused by the coronavirus. In some embodiments, the method further comprises administering a second dose of the vaccine to the animal subject, provided a presence or a quantity of a labeled complex formed between (i) and (iii) is detected in (b). In some embodiments, the method further comprises administering a second dose of the vaccine to the animal subject, provided a presence or a quantity of a labeled complex formed between (i) and (iii) is detected in (b). In some embodiments, (a)-(c) are performed at the point of need or point of care. In some embodiments, (b) is performed using a lateral flow assay, agglutination assay, an enzyme-linked immunoassay, photonic ring resonance, or a combination thereof. In some embodiments, a multiple doses of the vaccine are administered to the animal subject at different timepoints, and steps (a)-(c) are repeated following each dose of a multiple doses to measure vaccine-induced neutralizing antibodies against the coronavirus. In some embodiments, steps (a)-(c) are performed within at least 1 month after a first dose of the vaccine. In some embodiments, steps (a)-(c) are performed within at least 2 months after a first dose of the vaccine. In some embodiments, steps (a)-(c) are performed within at least 6 months after a first dose of the vaccine. In some embodiments, steps (a)-(c) are performed within at least 12 months after a first dose of the vaccine. In some embodiments, steps (a)-(c) are performed within at least 2 years after a first dose of the vaccine. In some embodiments, steps (a)-(c) are performed within at least 4 years after a first dose of the vaccine. In some embodiments, the animal subject is a plurality of subjects. In some embodiments, the plurality of subject is not enrolled in a clinical trial for the vaccine. In some embodiments, the plurality of subject is a population of subjects. In some embodiments, the coronavirus is severe acute respiratory syndrome coronavirus 2 (SARS-CoV-2). In some embodiments, the method further comprises determining the efficacy of the vaccine based, at least partially, on (c). In some embodiments, the method further comprises determining the durability of the vaccine based, at least partially, on (c). In some embodiments, the coronavirus is encoded by a nucleic acid sequence having greater than or equal to about 90% sequence identity to SEQ ID NO: 7-10, 13-20, 43, 67, or 91. In some embodiments, the coronavirus is encoded by a nucleic acid sequence having greater than or equal to about 95% sequence identity to SEQ ID NO: 7-10, 13-20, 43, 67, or 91. In some embodiments, the coronavirus is encoded by a nucleic acid sequence having greater than or equal to about 100% sequence identity to SEQ ID NO: 7-10, 13-20, 43, 67, or 91. In some embodiments, the animal subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide a method comprising: (a) providing a biological sample from an animal subject that was administered a vaccine against a coronavirus encoded by a nucleic acid sequence having greater than or equal to about 80% sequence identity to SEQ ID NO: 7-10, 13-20, 43, 67, or 91; (b) measuring a presence or level of a neutralizing antibody against the coronavirus in the biological sample by: (i) contacting the biological sample with a first peptide or protein comprising an amino acid sequence that is at least 80% identical to any one of SEQ ID NOS: 2-6, 11-12, 24, 47-48, 71-72, 95-96, or 115, and a second peptide or protein comprising an amino acid sequence that is at least 80% identical to SEQ ID NO: 1; and (ii) detecting a presence or a level a labeled complex between the at least two of (1) the first peptide or protein; (2) the second peptide or protein; and (3) the neutralizing antibody against the coronavirus; and (c) identifying the animal subject as having or lacking sufficient immunity against the coronavirus, wherein the presence or the level of the complex between (1) and (2) is indicative of lacking sufficient immunity and the presence or the level of the complex between (1) and (3) is indicative of having sufficient immunity. In some embodiments, the vaccine is allowed by a regulatory agency for the prevention of disease caused by the coronavirus. In some embodiments, the method further comprises administering a second dose of the vaccine to the animal subject, provided a presence or a quantity of a labeled complex formed between (i) and (iii) is detected in (b). In some embodiments, the method further comprises administering a second dose of the vaccine to the animal subject, provided a presence or a quantity of a labeled complex formed between (i) and (iii) is detected in (b). In some embodiments, (a)-(c) are performed at the point of need or point of care. In some embodiments, (b) is performed using a lateral flow assay, agglutination assay, an enzyme-linked immunoassay, photonic ring resonance, or a combination thereof. In some embodiments, a multiple doses of the vaccine are administered to the animal subject at different timepoints, and steps (a)-(c) are repeated following each dose of a multiple doses to measure vaccine-induced neutralizing antibodies against the coronavirus. In some embodiments, steps (a)-(c) are performed within at least 1 month after a first dose of the vaccine. In some embodiments, steps (a)-(c) are performed within at least 2 months after a first dose of the vaccine. In some embodiments, steps (a)-(c) are performed within at least 6 months after a first dose of the vaccine. In some embodiments, steps (a)-(c) are performed within at least 12 months after a first dose of the vaccine. In some embodiments, steps (a)-(c) are performed within at least 2 years after a first dose of the vaccine. In some embodiments, steps (a)-(c) are performed within at least 4 years after a first dose of the vaccine. In some embodiments, the animal subject is a plurality of subjects. In some embodiments, the plurality of subject is not enrolled in a clinical trial for the vaccine. In some embodiments, the plurality of subject is a population of subjects. In some embodiments, the coronavirus is severe acute respiratory syndrome coronavirus 2 (SARS-CoV-2). In some embodiments, the method further comprises determining the efficacy of the vaccine based, at least partially, on (c). In some embodiments, the method further comprises determining the durability of the vaccine based, at least partially, on (c). In some embodiments, the coronavirus is encoded by a nucleic acid sequence having greater than or equal to about 90% sequence identity to SEQ ID NO: 7-10, 13-20, 43, 67, or 91. In some embodiments, the coronavirus is encoded by a nucleic acid sequence having greater than or equal to about 95% sequence identity to SEQ ID NO: 7-10, 13-20, 43, 67, or 91. In some embodiments, the coronavirus is encoded by a nucleic acid sequence having greater than or equal to about 100% sequence identity to SEQ ID NO: 7-10, 13-20, 43, 67, or 91. In some embodiments, the animal subject is a mammal. In some embodiments, the mammal is a human, dog, cat, monkey, non-human primate, rodent, or a farm animal. In some embodiments, the age of the subject is at least 50 years old. In some embodiments, the biological sample is deposited on a biological sample medium. In some embodiments, the biological sample medium is a dried blood spot card.

Aspects disclosed herein provide systems for analyzing a biological sample, the system comprising: (a) a biological sample processor comprising: (i) a biological sample receiver configured to receive biological sample medium comprising a biological sample; and (ii) a biological sample chemical extractor configured to elute the biological sample from the biological sample medium; and (b) an assay assembly block comprises a biological sample assay conductor configured to analyze the biological sample to detect a level of neutralizing antibodies against a coronavirus that blocks binding between an S protein of a coronavirus (CoV) and Angiotensin-converting enzyme 2 (ACE2) in the biological by: (i) contacting the biological sample with (1) a first protein comprising the ACE2, or a fragment thereof, that binds to the CoV S protein, or a fragment thereof, and (2) a second protein comprising the CoV S protein, or the fragment thereof, that binds to the ACE2, or the fragment thereof, under conditions that permit binding between the ACE2 or fragment thereof, and the COV S protein or fragment thereof, to produce an ACE2-CoV S complex, wherein the first protein or the second protein is labeled directly or indirectly with a detectable moiety; (ii) detecting a signal corresponding to a level of the ACE2-CoV S complex in the biological sample wherein the signal is inversely correlated with the level of the neutralizing antibody in the biological sample; and (iii) detect the level of the neutralizing antibody in the biological sample based, at least in part, on the signal detected. In some embodiments, the system is automated or semi-automated. In some embodiments, the systems are robotized. In some embodiments, the biological sample is more than or equal to about 1000, 1500, 2000, 25000, 3000, 3500, 4000, 4500, 5000, 5500, 6000, 6500, 7000, 7500, 8000, 8500, 9000, 9500, 1000, 1500, or 20000 biological samples, wherein each of the biological samples is obtained from a different subject. In some embodiments, the system is configured to perform (a) to (b) under 1, 2, 3, 4, 5, 6, 7, 8, 9, 10, 11, or 12 hours. In some embodiments, the system is configured to perform (a) to (b) on more than or equal to about 5000 biological samples in about 8 hours. In some embodiments, the system is configured to perform (a) to (b) on more than or equal to about 20000 biological samples in about 8 hours. In some embodiments, the biological sample medium is a dried blood spot card. In some embodiments, the assay assembly block comprises a fluorescence Resonance Energy Transfer (FRET) assay assembly, or an enzyme-linked immunosorbent assay (ELISA) assembly. In some embodiments, the biological sample receiver is further configured to: (a) orient the biological sample medium; (b) optionally, scan the biological sample medium to obtain patient information; (c) optionally, store the patient information in a database; (d) separate a segment of the biological sample from the biological sample medium; (e) place segment of the biological sample in a assay chamber; or (f) optionally, scan assay chamber to ensure the segment is properly positioned in the assay chamber; or (g) a combination of (a) to (f). In some embodiments, (a) to (f) is performed by the biological sample receiver. In some embodiments, the biological sample medium is a dried blood spot card and the segment of the biological sample is punch from the dried blood spot card having diameter of about 5 millimeters. In some embodiments, the assay chamber comprises a well in a 96 well plate. In some embodiments, the biological sample chemical extractor is further configured to purify the biological sample from an elution containing the biological sample eluted from the biological sample medium. In some embodiments, the assay assembly block further comprises a biological sample diluter that is configured to serially dilute the purified biological sample into multiple dilutions for analysis. In some embodiments, the level of neutralizing antibodies against a coronavirus that blocks binding between an S protein of a coronavirus (CoV) and Angiotensin-converting enzyme 2 (ACE2) is associated with: a prognosis related to adaptive immunity of the subject to an infection by the coronavirus or variant thereof, or the disease or the condition caused by the coronavirus; or a measure of susceptibility of the subject to an infection by the coronavirus. In some embodiments, the biological sample is obtained from a subject is a mammal. In some embodiments, the mammal is a human, dog, cat, monkey, non-human primate, rodent, or a farm animal. In some embodiments, the age of the biological sample is obtained from a subject that is at least or equal to about 50, 51, 52, 53, 54, 55, 56, 57, 58, 59, 60, 61, 62, 63, 64, 65, 66, 67, 68, 69, 70, 71, 72, 73, 74, or 75 years old. In some embodiments, the CoV is Severe acute respiratory syndrome-related virus (SARS-CoV). In some embodiments, the SARS-CoV is SARS-CoV-2. In some embodiments, the biological sample assay conductor is further configured to analyze the biological sample by comparing the level of neutralizing antibodies to a control or an index, wherein the control or index is a known level of neutralizing antibodies determined by a cell-based assay. In some embodiments, the first protein and/or the second protein are in a solution, wherein in the solution having a volume of no greater than or equal to about 5, 10, 15, 20, 25, 30, 35, 40, 45, or 50 μl.

Aspects disclosed herein provide methods for analyzing a biologicals sample, the method comprising: (a) receiving biological sample medium comprising a biological sample; (b) eluting the biological sample from the biological sample medium; (c) analyzing the biological sample to detect a level of neutralizing antibodies against a coronavirus that blocks binding between an S protein of a coronavirus (CoV) and Angiotensin-converting enzyme 2 (ACE2) in the biological by: (i) contacting the biological sample with (1) a first protein comprising the ACE2, or a fragment thereof, that binds to the CoV S protein, or a fragment thereof, and (2) a second protein comprising the CoV S protein, or the fragment thereof, that binds to the ACE2, or the fragment thereof, under conditions that permit binding between the ACE2 or fragment thereof, and the COV S protein or fragment thereof, to produce an ACE2-CoV S complex, wherein the first protein or the second protein is labeled directly or indirectly with a detectable moiety; and (ii) detecting a signal corresponding to a level of the ACE2-CoV S complex in the biological sample wherein the signal is inversely correlated with the level of the neutralizing antibody in the biological sample; and (d) detect the level of the neutralizing antibody in the biological sample based, at least in part, on the signal detected. In some embodiments, steps (a) to (d) are automated or semi-automated. In some embodiments, steps (a) to (d) are performed by robots. The system of claim 53, the biological sample is more than or equal to about 1000, 1500, 2000, 25000, 3000, 3500, 4000, 4500, 5000, 5500, 6000, 6500, 7000, 7500, 8000, 8500, 9000, 9500, 1000, 1500, or 20000 biological samples, wherein each of the biological samples is obtained from a different subject. In some embodiments, (a) to (d) are performed under 1, 2, 3, 4, 5, 6, 7, 8, 9, 10, 11, or 12 hours. The method of claim 75, wherein (a) to (d) are performed on more than or equal to about 5000 biological samples in about 8 hours. In some embodiments, (a) to (d) are performed on more than or equal to about 20000 biological samples in about 8 hours. In some embodiments, the biological sample medium is a dried blood spot card. In some embodiments, the assay assembly block comprises a fluorescence Resonance Energy Transfer (FRET) assay assembly, or an enzyme-linked immunosorbent assay (ELISA) assembly. In some embodiments, the methods further comprise: (e) orienting the biological sample medium; (f) optionally, scanning the biological sample medium to obtain patient information; (g) optionally, storing the patient information in a database; (h) separating a segment of the biological sample from the biological sample medium; (i) placing the segment of the biological sample in a assay chamber; (j) optionally, scanning the assay chamber to ensure the segment is properly positioned in the assay chamber; (g) a combination of (e) to (j). In some embodiments, the methods comprise performing (e) to (k). In some embodiments, the biological sample medium is a dried blood spot card and the segment of the biological sample is punch from the dried blood spot card having diameter of about 5 millimeters. In some embodiments, the assay chamber comprises a well in a 96 well plate. In some embodiments, the methods further comprise purifying the biological sample from an elution containing the biological sample eluted from the biological sample medium. In some embodiments, the methods further comprise serially dilute the purified biological sample into multiple dilutions for analysis. In some embodiments, the level of neutralizing antibodies against a coronavirus that blocks binding between an S protein of a coronavirus (CoV) and Angiotensin-converting enzyme 2 (ACE2) is associated with: a prognosis related to adaptive immunity of the subject to an infection by the coronavirus or variant thereof, or the disease or the condition caused by the coronavirus; or a measure of susceptibility of the subject to an infection by the coronavirus. In some embodiments, the biological sample is obtained from subject that is a mammal. In some embodiments, the mammal is a human, dog, cat, monkey, non-human primate, rodent, or a farm animal. In some embodiments, the age of the subject is at least or equal to about 50, 51, 52, 53, 54, 55, 56, 57, 58, 59, 60, 61, 62, 63, 64, 65, 66, 67, 68, 69, 70, 71, 72, 73, 74, or 75 years old. In some embodiments, the CoV is Severe acute respiratory syndrome-related virus (SARS-CoV). In some embodiments, the SARS-CoV is SARS-CoV-2. In some embodiments, In some embodiments, analyzing the biological sample further comprises comparing the level of neutralizing antibodies to a control or an index, wherein the control or index is a known level of neutralizing antibodies determined by a cell-based assay. In some embodiments, the first protein and/or the second protein are in a solution, wherein in the solution having a volume of no greater than or equal to about 5, 10, 15, 20, 25, 30, 35, 40, 45, or 50 μl.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary assay measuring binding between an immobilized human angiotensin-converting enzyme 2 (ACE2) receptor and a peptide-conjugate derived from a spike glycoprotein of a SARS-CoV-2 in a biological sample obtained from a patient that has not been exposed to SARS-CoV-2.

FIG. 2 shows an exemplary assay measuring binding between an immobilized human ACE2 receptor and a peptide-conjugate derived from the spike glycoprotein of SARS-CoV-2 in a biological sample obtained from a patient exposed to SARS-CoV-2.

FIG. 3 shows an exemplary lateral flow assay to measure binding between human ACE2 receptor and a peptide-conjugate derived from the spike glycoprotein of SARS-CoV-2 in a biological sample obtained from a patient that has not been exposed to SARS-CoV-2.

FIG. 4 shows an exemplary lateral flow assay to measure binding between human ACE2 receptor and a peptide-conjugate derived from the spike glycoprotein of SARS-CoV-2 in a biological sample obtained from a patient that was exposed to SARS-CoV-2.

FIG. 5 shows an exemplary system according to some embodiments.

FIG. 6 shows a computing device; in this case, a device with one or more processors, memory, storage, and a network interface, in accordance with some embodiments.

FIG. 7 shows an exemplary assay in FIG. 2 in an image-free system; results from the exemplary assay is visible and can be interpreted by the naked eye.

FIG. 8A-8D shows an exemplary assay assembly to measure inhibition of binding between the receptor binding domain (RBD) of SARS-CoV-2 and its cognate receptor, ACE2. FIG. 8A illustrates assay with a first RBD (RBD1) with a patient sample that does not contain neutralizing antibodies that functionally inhibit RBD1-ACE2 binding. FIG. 8B shows the same assay from FIG. 8A with a patient sample that contains neutralizing antibodies that functionally inhibit RBD1-ACE2 binding. FIG. 8C shows a neutralization assay that measures a presence of neutralizing antibodies against at least two different RBD peptides (RBD1 and RBD2) in the absence of neutralizing antibodies (FIG. 8C) and in the presence of neutralizing antibodies (FIG. 8D).

FIG. 9A-9D illustrate that the assay assembly from FIG. 8A-8D may be performed in solution (e.g., not on a solid surface) using fluorescence resonance energy transfer (FRET). FIG. 9A shows electron transfer between the donor fluorophore and the acceptor fluorophore, indicating an absence of neutralizing antibodies that block binding between RBD and ACE2.

FIG. 9B shows no electron transfer between the donor fluorophore and the acceptor fluorophore, indicating a presence of neutralizing antibodies that block binding between RBD.

FIG. 9C shows a neutralization assay in solution in which electron transfer occurs between two different donor fluorophore and the acceptor fluorophore pairs, indicating an absence of neutralizing antibodies that block binding between first RBD, RBD1, and second RBD, RBD2.

FIG. 9D shows a neutralization assay in solution in which no electron transfer occurs between two different donor fluorophore and the acceptor fluorophore pairs, indicating a presence of neutralizing antibodies that block binding between RBD1 and RBD2.

FIG. 10A-10E illustrate homogenous solution with reaction components, where the homogenous solution is applied to a surface with surface-bound capture molecules. FIG. 10A illustrates a homogenous solution comprising the reaction components of FIG. 8A being applied to surface-bound capture molecules of FIG. 8A. The capture molecule captures an epitope tag conjugated to RBD, which forms a complex with ACE2 conjugated to the detective moiety. FIG. 10B illustrates a homogenous solution comprising reaction components being applied to surface-bound capture molecules of FIG. 8A. The capture molecule captures an epitope tag conjugated to ACE2, which forms a complex with RBD conjugated to the detective moiety. FIG. 10C illustrates a homogenous solution comprising the reaction components of FIG. 8B being applied to surface-bound capture molecules of FIG. 8B. FIG. 10D illustrates a homogenous solution comprising the reaction components of FIG. 8C being applied to surface-bound capture molecules of FIG. 8C. FIG. 10E illustrates a homogenous solution comprising the reaction components of FIG. 8D being applied to surface-bound capture molecules of FIG. 8D.

FIG. 11A illustrates two vaccines having different levels of efficacy in a population of individuals, and the risks and benefits associated therewith. FIG. 11B illustrates that the methods provided herein, in some embodiments, can reduce or remove the risk associated with usage of a commercialized vaccine.

FIG. 12A-12C illustrate a biological sample medium (BSM) capable of storing one or more biological samples in both a closed (FIG. 12A) and open (FIG. 12B) configuration. FIG. 12C illustrates a survey printed on the BSM.

FIG. 13 illustrates a system according to some embodiments disclosed herein.

FIG. 14 illustrates a workflow of the automated systems disclosed herein according to some embodiments.

FIG. 15 shows results neutralizing antibody inhibition of SARS-CoV-2 S1 binding to ACE2 from a time-resolved fluorescence resonance energy transfer (TR-FRET) assay run on 384 well plate (left) and a 1536 well plate (right).

### DETAILED DESCRIPTION

The inventors of the instant disclosure discovered an urgent and unmet need for a confirmatory diagnostic test that determines whether an allowed or approved vaccine for the treatment of a disease caused by SARS-CoV-2 is effective to confer adaptive immunity against SARS-CoV-2 outside the clinical trial context. The inventors also discovered that existing tests for antibodies against SARS-CoV-2 fail to measure the functional inhibition (e.g., blocking) of binding between the spike protein and its cognate receptor (ACE2), which mediates infection by SARS-CoV-2. This means that existing tests are not able to determine whether a subject has sufficient adaptive immunity against SARS-CoV-2 induced by the vaccine following vaccine administration.

To address this urgent need, the inventors of the instant disclosure developed a neutralizing antibody test that measures the functional inhibition between the spike protein and ACE2, which can be used to determine whether a vaccine is effective to confer an adaptive immune response sufficient to protect a subject from a future infection. The neutralizing antibody tests described herein can also be used to measure the durability of a vaccine over time following a prior administration of the vaccine, which indicates whether a booster of the vaccine may be needed. The neutralizing antibody tests described herein can be used to screen a population of individuals. The neutralizing antibody tests described herein can be used to identify individuals in a population that respond to a vaccine and individuals in a population that do not respond to a vaccine. The neutralizing antibody tests described herein can be used to minimize or reduce the risk associated with vaccine that has been commercialized for usage in a population (e.g., by identifying individuals that do not respond to the vaccine). The neutralizing antibody tests described herein can provide a surrogate marker of protection from a vaccine. The neutralizing antibody tests described herein can be used to classify vaccines by % efficacy in a population. With over 300 million people in the United States alone in need of vaccination, the neutralizing antibody tests described herein are both scalable and cost-efficient.

Patients recovered from COVID-19 cannot be assumed to be at low risk for rebound or re-infection, because many patients recovered from COVID-19 in several recent studies were found to never produce neutralizing antibodies against the spike protein. This is true even if the patient is given a vaccine against SARS-CoV-2. Thus, there exists a need for at testing device and systems for measuring sufficient neutralizing antibodies against SARS-CoV-2 in a patient sample conferring adaptive immunity in the patient. With over 300 million individuals in need of vaccination in the United States alone, these testing devices and systems must be scalable, cost-effective, and accessible at the point of need or point of care.

Existing SARS-CoV-2 tests marketed to aid in the diagnosis of COVID-19 at best measure exposure to SARS-CoV-2. Further, existing SARS-CoV-2 tests cannot predict severity of COVID-19 in a subject upon reinfection by SARS-CoV-2. For example, existing tests that detect a presence of antibodies against SARS-CoV-2 in a patient sample cannot discriminate neutralizing antibodies against SARS-CoV-2 and non-neutralizing antibodies. A “neutralizing antibody to SARS-CoV-2” as used herein, refers to an antibody that, when bound to the spike protein of SARS-CoV-2, prevents binding between the spike protein and its cognate receptor, ACE2. Further, existing SARS-CoV-2 tests that detect a presence of antibodies against SARS-CoV-2 lack specificity because they cannot assess the amount of cross reactivity between the antibodies in a patient sample and SARS-CoV-2 versus more benign (e.g., seasonal) coronaviruses. Although SARS-CoV-2 tests that detect binding between an antibody in a patient sample and the spike protein exist, they do not necessarily indicate when a sufficient adaptive immune response to SARS-CoV-2 has occurred. Even direct antibody binding to the receptor binding domain of the spike protein does not always inhibit viral entry into a cell initiating an infection.

The gold standard test for assessing clinical immunity to a virus utilizes a cell-based assay. The cell-based assay quantitatively measures neutralizing antibody titers in patient sera by adding patient serum and a solution containing the virus to viral-susceptible cells, and analyzing the cells to determine if the virus can no longer infect the cells. In this cell-based assay, a reduction in viral induced cytotoxicity is a measure of neutralization activity in the sera. The strength of neutralization is reported in two ways: (i) the IC50 (e.g., half the cells are killed) or (ii) highest dilution at which neutralization activity disappears. This cell-based assay has limited clinical utility, because it is difficult to scale up, is cost and time intensive, and require technical training to perform. The inability to scale these cell-based assays means clinical utility for larger populations of individuals is limited. The time, cost, and technical expertise required means clinical utility of cell-based assay at the point of need is limited. Accordingly, there is an urgent need for a scalable assay that can also be used at the point of need to measure clinical immunity.

By contrast, the testing devices and systems described herein directly detect the functional inhibition of binding between the receptor binding domain of the spike protein and its cognate receptor (e.g., ACE2) by neutralizing antibodies in a patient sample. In this manner, the devices and systems described herein have increased specificity to neutralizing antibodies to SARS-CoV-2, because they can (i) discriminate neutralizing antibodies from non-neutralizing antibodies, and (ii) assess cross reactivity between antibodies in the patient sample and benign coronaviruses. The testing devices and systems described herein are designed to indicate when a sufficient adaptive immune response to SARS-CoV-2 has occurred, by detecting the functional inhibition of binding between ACE2 and the spike protein when in a presence of antibodies in a patient sample. Testing devices and systems described herein are portable, do not require technical expertise to use or perform, and are far less costly than the gold standard test (e.g., cell-based assay) for assessing clinical immunity. For at least these reasons, the devices and systems described herein are an ideal test at the point of need.

The testing devices and systems described herein are easily scalable, at least because the testing device (i) has an assay assembly that is simple (e.g., lateral flow, solution, or solid phase capture), (ii) does not require complex imaging devices, like for e.g., BioTek® Cytation plate reader, and (iii) does not require use of a cell (or cell line). In some embodiments, the testing device is manufactured before the discovery of a new strain of virus. Upon discovery of the new strain, the assay assembly of the testing device is modified by replacing one component of the assay assembly (e.g., the recombinant RBD peptide unique to the new strain of SARS-CoV). In some embodiments, the new strain may comprise one or more mutations to SEQ ID NO: 5. In some embodiments, the new strain may comprise 1, 2, 3, 4, 5, 6, 7, 8, 9, 10, 11, 12, 13, 14, 15, 16, 17, 18, 19, 20, 21, 22, 23, 24, or 25 or more of the mutations to SEQ ID NO: 5 listed in Table 1. In some embodiments, the assay assembly is configured for centralized and automated processing, enabling high throughput performance with significantly lower reagent amounts. Methods of utilizing the testing devices and systems described herein, in some cases, leverage robotics or other means of automation that can perform upwards of 60,000 tests per day using less than one-quarter of the reagents required for existing tests.

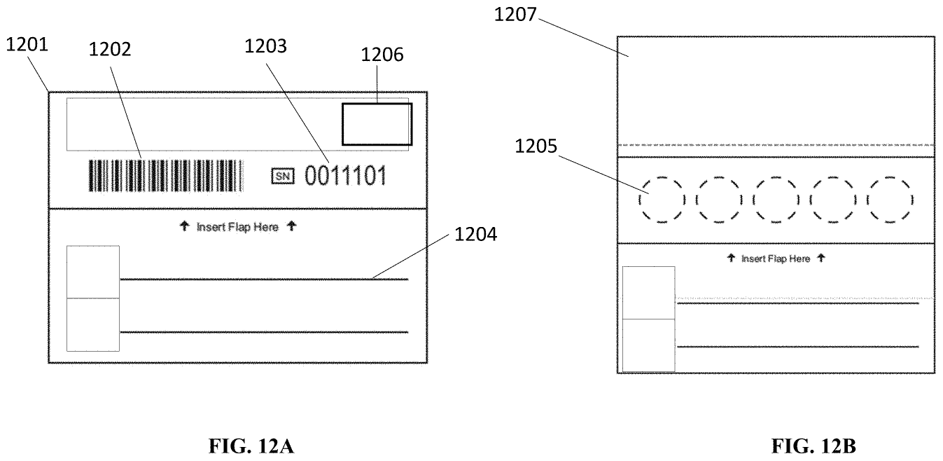

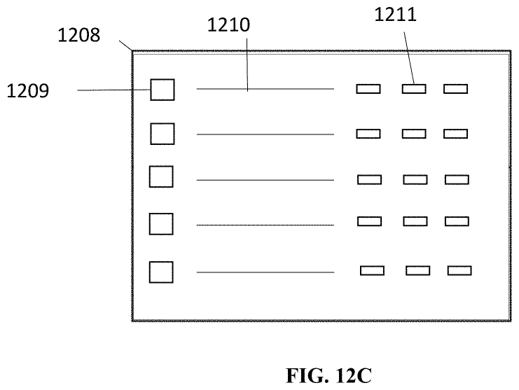

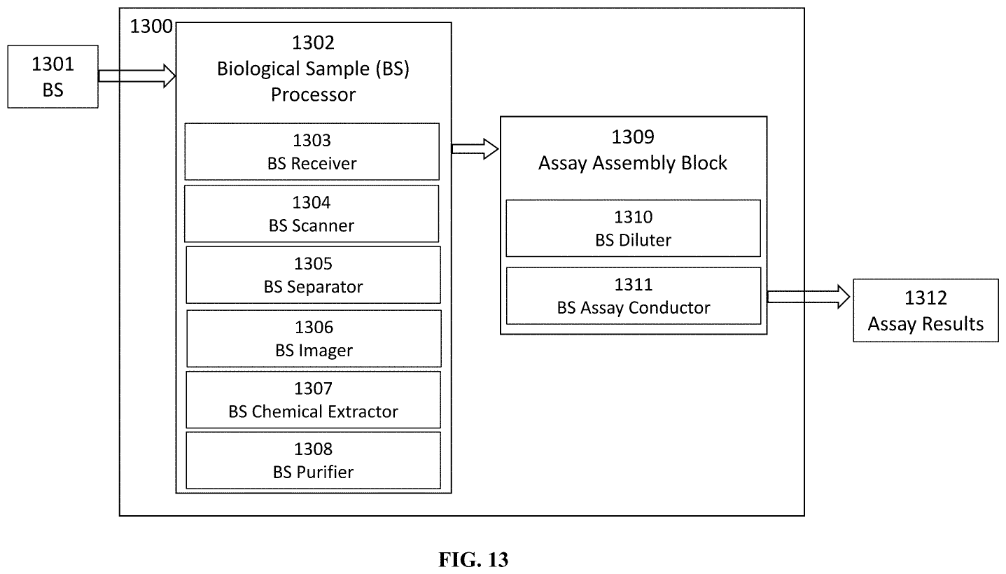

The high throughput systems and methods for analyzing a biological sample described herein may analyze more than one biological sample (e.g., hundreds or thousands of biological samples) in a single day. In some embodiments, the high throughput performance may include between about 5,000 to about 20,000 unique processed biological samples every 8 hours. In some embodiments, the high throughput performance may be achieved by utilizing a biological sample medium (BSM) 1201, as shown in FIG. 12A-12B, which contains a biological sample obtained from a subject (e.g., a finger prick) and, in some embodiments, integrates the biological sample into an automated or semi-automated system for sample processing and assaying the biological sample. In some embodiments, the BSM may be a dried blood spot card (DBS). The automated or semi-automated systems described herein, automate sample processing, sample elution, sample preparation for assaying, and/or assaying the biological sample (e.g., without a need for an operator).

In some embodiments, as shown in FIG. 13, the automated or semi-automated systems 1300 described herein comprise a biological sample processor 1302 and an assay assembly block 1309. In some embodiments, the biological sample processor 1302 of the automated or semi-automated systems 1300 provided in FIG. 13 are configured to receive the biological sample, scan the biological sample (e.g., barcode on BSM) to register information regarding the origin of the biological sample (e.g., patient identifying information), separate a segment of the biological sample (e.g., a hole punch segment of the BSM containing the biological sample), place the segment of the biological sample into a vessel (e.g., a well), image the vessel to ensure that the segment of the biological sample is correctly placed into the vessel, extract the biological sample from the BSM, and/or purify the biological sample. In some embodiments, the assay assembly block 1309 of the automated or semi-automated systems 1300 provided in FIG. 13 are configured to dilute the optionally purified sample into serial dilutions (e.g., dilutions of 1:20, 1:160, 1:320, 1:640 sample to solution), and/or conduct the assay (e.g., FRET, ELISA) disclosed herein on the serial dilutions, to yield the assay results 1312.

In addition to directly detecting the functional inhibition of binding between the receptor binding domain of the spike protein and its cognate receptor (e.g., ACE2) by neutralizing antibodies in a patient sample, the testing devices described herein measure a presence or quantity of all antibodies (e.g., neutralizing or otherwise) against the pathogen (e.g., SARS-CoV-2) that are unique to a subject. In some embodiments, the testing devices differentiate between antibody classes, such as immunoglobulin G (IgG) and immunoglobulin M (IgM). In some embodiments, a presence or a quantity of IgG, IgM, or a combination thereof, in a patient sample is indicative of an acute infection. Thus, the testing device described herein can determine whether a subject is suffering from an acute infection by the pathogen (e.g., SARS-CoV-2), and whether the subject has developed a sufficient adaptive immunity to the pathogen such that they are immune from a future infection. The testing devices and systems described here are capable of adapting to rapidly evolving viral outbreaks in a manner that is considerably faster than existing technologies. In some embodiments, the testing devices test for more than one strain of a virus. For example, the testing devices and systems described herein are capable of identifying whether an individual is immune to human 2002-2003 and 2003-2004 SARS-CoV, with S proteins corresponding to GenBank accession no. AY274119 and GenBank accession no. AY525636, respectively. The testing devices and systems, in some embodiments, utilize recombinant RBD peptides that each have a different epitope region unique to the strain of SARS-CoV. Proper epitope presentation is accomplished by engineering the recombinant peptides to exhibit the same tertiary and quatemary structures as the corresponding pathogenic virus.

Provided herein are testing devices and systems for measuring adaptive immunity to a pathogen in a subject. In some embodiments, the testing devices are point of need or point of care devices. In some embodiments, the testing devices are configured to perform an assay on a biological sample of the subject to detect sufficient antibody titers to confer adaptive immunity to a pathogen of interest in the subject.

The biological sample includes cell, tissue, or bodily fluid obtained from the subject. Non-limiting examples of biological samples include aqueous humour, vitreous humour, bile, breast milk, cerebrospinal fluid, endolymph, perilymph, female ejaculate, amniotic fluid, gastric juice, menses, mucus, peritoneal fluid, pleural fluid, saliva, sebum, semen, sweat, tears, vaginal secretion, vomit, urine, feces, whole blood, blood serum, blood plasma, sputum, cerebrospinal fluid, synovial fluid, lymphatic fluid, nasal swab, or cheek swab. In some embodiments, the biological sample comprises blood, urine, saliva, or feces. In some embodiments, the blood is capillary blood. In some embodiments, the capillary blood is obtained by a finger prick. In some embodiments, the capillary blood sample comprises 1, 2, 3, 4, 5, 6, 7, 8, 9, 10, 11, 12, 13, 14, 15, 16, 17, 18, 19, or 20 drops of blood. In some embodiments, the capillary blood sample comprises a volume of blood less than 100, 95, 90, 85, 80, 75, 70, 65, 60, 55, 50, 45, 40, 35, 30, 25, 20, 15, 10, 9, 8, 7, 6, 5, 4, 3, 2, or 1 μL. In a non-limiting example, the subject pricks a finger, obtains a drop of blood from the finger prick, and places the drop of blood on a blood card to be stored. The subject may do this 3-4 times for the same blood card. The subject mails the blood card to the laboratory for processing.

In some embodiments, the assay is a competition assay comprising one or more capture molecules, optionally coupled to a surface, and a detectable peptide-conjugate. In some embodiments, a signal from the detectable peptide-conjugate is detected by capturing an image of a detection zone of the device by an imaging device. In some embodiments, the imaging device is a smartphone. In some embodiments, the assay is a lateral flow assay (LFA). In some embodiments, the pathogen of interest is severe acute respiratory syndrome coronavirus 2 (SARS-CoV-2).

In some embodiments, the detectable peptide-conjugate comprises a peptide derived from a spike glycoprotein of a coronavirus, and the one or more capture molecules is derived from an angiotensin-converting enzyme 2 (ACE2) receptor, or a surrogate thereof (e.g., heparin or a fragment thereof). In some cases, the detectable peptide-conjugate comprises a peptide derived from the ACE2 receptor, and the one or more capture molecules is derived from the spike glycoprotein of a coronavirus. In some embodiments, the coronavirus is a severe acute respiratory syndrome coronavirus (SARS-CoV). In some embodiments, the SARS-CoV is SARS-CoV-2. In some embodiments, the ACE2 receptor is derived from a human ACE2 receptor. In some embodiments, more than one detectable peptide-conjugate is utilized to measure neutralizing antibodies against more than one strain of SARS-CoV.

Systems described herein comprise the testing device and the imaging device. In some embodiments, systems further comprise a computing device with an application (e.g., web or mobile) comprising a data analytics module for receiving an analyzing data from the imaging device to provide a result. In some embodiments, the imaging device is the computing device (e.g., smartphone). In some embodiments, the imaging device is not the computing device. In some embodiments, the result is a positive result indicating that a subject is immune to an infection by the pathogen. In some embodiments, the result is a negative result indicating that the subject is not immune to an infection by the pathogen. In some embodiments, the application further comprises a communication module configured to display via a graphical user interface (GUI) one or more results to a user. In some embodiments, systems comprise a data store configured to store and retrieve data from the imaging device, the external device, or both.

In some embodiments, systems comprise an external device, such as a wearable tracking device (e.g., Aurora®, Fitbit®, Apple Watch). In some embodiments, the data analytics module receives and analyzes external data from the external device. In some embodiments, external data comprise body temperature, heart rate, heart rate variability, sleep quality, or sleep quantify of the subject. In some embodiments, data analytics module is configured to analyze the external data in combination with the data received from the imaging device to identify the subject as being infected with a specific pathogen.

In some embodiments, systems described herein comprise multiple users with multiple computing devices. Within a geographical location of interest, systems described herein are designed to determine whether a population of users has become immune to an infection by the pathogen (e.g., SARS-CoV-2). In some instances, global position system (GPS) tracking of the personal computing device, the imaging devices, or both, can be used to produce a geofence surrounding the geographical location of interest.

### Testing Devices and Systems

Disclosed herein, in some embodiments, are testing devices that can be deployed at the point of need to determine whether a patient is immune to an infection by the pathogen. In some embodiments, devices comprise an assay assembly capable of assaying a biological sample obtained from the patient. In some embodiments, the testing devices comprise one or more components, such as a housing, a sample receiver, a sample processor, a sample purifier, or a detection zone. In some embodiments, the testing systems described herein comprise one or more testing devices described herein and a biological sample medium (BSM) that stores the biological sample. In some embodiments, the one or more components of the testing devices or systems described herein elutes the biological sample from the BSM prior to assaying the sample using the assay assembly of the testing device or system.

### Assay Assembly

Disclosed herein, in some embodiments are devices comprising an assay assembly that is capable of detecting a target analyte. In some embodiments, the target analyte is an antibody specific to a pathogen of interest. In some embodiments, the antibody specific to the pathogen of interest functionally blocks binding between the pathogen to its cognate receptor (e.g., a “neutralizing antibody”). In some embodiments, the neutralizing antibody is elicited in a subject by a vaccine or an infection by the pathogen. In some embodiments, the testing devices comprise two assay assemblies, the first assay assembly capable of detecting an antibody specific to a pathogen of interest, and the second assay assembly capable of detecting a neutralizing antibody that functionally blocks binding between the pathogen and its cognate (host) receptor. In some embodiments, the target analyte is a biomarker. It is contemplated that any combination of assay assemblies described herein may be used in combination, depending on the desired result. In some embodiments, the combination is housed in a single testing device. In some embodiments, the combination is housed in more than one testing device.

In some embodiments, the pathogen comprises a virus, a bacteria, a parasite, a fungus, or a combination thereof. In some embodiments, the virus is a coronavirus. In some embodiments, the coronavirus is a severe acute respiratory syndrome coronavirus (SARS-CoV). In some embodiments, the SARS-CoV is SARS-CoV-2. In some embodiments, the coronavirus is Middle East Respiratory Syndrome coronavirus (MERS-CoV). In some embodiments, the coronavirus is an alpha coronavirus (e.g., 229E, NL63). In some embodiments, the coronavirus is a beta coronavirus (e.g., OC43, HKU1). In some embodiments, the coronavirus comprises a protein having an amino acid sequence with greater than or equal to about 70%, 75%, 80%, 85%, 86%, 87%, 88%, 89%, 90%, 91%, 92%, 93%, 94%, 95%, 96%, 97%, 98%, 99%, or 100% sequence identity to one or more sequences, or portions thereof, provided in SEQ ID NOS: 1-116. In some embodiments, the coronavirus comprises a protein having an amino acid sequence with greater than or equal to about 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 2, or a portion thereof. In some embodiments, the coronavirus comprises a protein having an amino acid sequence with greater than or equal to about 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 3, or a portion thereof. In some embodiments, the coronavirus comprises a protein having an amino acid sequence with greater than or equal to about 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 4, or a portion thereof. In some embodiments, the coronavirus comprises a protein having an amino acid sequence with greater than or equal to about 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 5, or a portion thereof. In some embodiments, the coronavirus comprises a protein having an amino acid sequence with 1, 2, 3, 4, 5, 6, 7, 8, 9, 10, 11, 12, 13, 14, 15, 16, 17, 18, 19, 20, 21, 22, 23, 24, or 25 or more of the mutations to SEQ ID NO: 5 listed in Table 1. In some embodiments, the coronavirus comprises a protein having an amino acid sequence with greater than or equal to about 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 6, or a portion thereof. In some embodiments, the coronavirus comprises a protein having an amino acid sequence with greater than or equal to about 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 11, or a portion thereof. In some embodiments, the coronavirus comprises a protein having an amino acid sequence with greater than or equal to about 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 12, or a portion thereof. In some embodiments, the coronavirus comprises a protein having an amino acid sequence with greater than or equal to about 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 24, 47-48, 71-72, 95-96, or 115, or a portion thereof. In some embodiments, the coronavirus comprises a viral genome comprising a sequence that is greater than or equal to about 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 7-10, 13-20, 43, 67, or 91, or a portion thereof.

In some embodiments, the pathogen of interest is a variant of the pathogen, for example, a pathogen that has evolved through mutations in the genes encoding the pathogen. In the case of a virus, the variant virus may have a mutation in an amino acid sequence of a viral protein, such as an envelope protein, nucleocapsid protein, membrane protein, hemaggluitinin, neuraminidase, or spike protein. In some embodiments, the virus (e.g., coronavirus) comprises a mutation in one or more of the sequences provided in SEQ ID NOS: 1-116. In some embodiments, the mutation does not affect, or minimally affects, host receptor binding by the spike protein of the coronavirus. In some embodiments, the mutation is an insertion, a deletion, or a substitution at an amino acid (or a plurality of amino acids) of one or more sequences provided in SEQ ID NOS: 1-116 for coronaviruses (e.g., SARS, SARS-CoV, and NL63). In some embodiments, the Non-limiting examples of mutations in an amino acid sequence of the spike protein of SARS-CoV-2 (SEQ ID NO: 5) are provided in Long et al., Molecular Architecture of Early Dissemination and Massive Second Wave of the SARS-CoV-2 Virus in a Major Metropolitan Area, Am. Soc. M. Bio., 11:6 (November/December 2020) e02707-20, which is hereby incorporated by reference in its entirety. In some embodiments, the variant of SARS-CoV-2 comprises a spike protein having an amino acid sequence comprising a substitution of an aspartic acid (Asp) to a glycine (Gly) at amino acid position 614 (Asp614Gly) with reference to SEQ ID NO: 5 (SARS-CoV-2). In some embodiments, the variant of SARS-CoV-2 comprises a spike protein having an amino acid sequence comprising one or more substitution selected from Ala442Val, Ala448Val, Ala553Pro or Val, Gly682Arg, Ser758Pro, and Cys812Phe, with reference to SEQ ID NO: 5 (SARS-CoV-2).

In some embodiments, the antibody specific to coronavirus is specific to the receptor binding domain of the spike protein of the coronavirus, such that the antibody blocks binding of the spike protein to its cognate receptor (e.g., ACE2). In some embodiments, the analyte is a complex comprising the spike protein bound to the antibody at the receptor binding region of the spike protein. In some embodiments, the antibody specific to the pathogen of interest belongs to an immunoglobulin class comprising immunoglobulin M, immunoglobulin G, immunoglobulin A, immunoglobulin E, or immunoglobulin D.

In some embodiments, the target analyte (or second target analyte) is a biomarker. In some embodiments, the assay assembly is configured to detect at least one, two, three, four, five, 10, 20, 30, 40, 50, 60, 70, 80, 90, 100, 200, 500, 1,000 or more biomarkers in the biological sample. In some embodiments, the biomarker comprises a non-peptide coding nucleic acid sequence. In some embodiments, the biomarker comprise a peptide coding nucleic acid sequence such as mRNA or cDNA. In some embodiments, the biomarker comprises a peptide or a protein.

In some embodiments, the biomarker is derived from the pathogen of interest (e.g., coronavirus). In some embodiments, the assay assembly is adapted to detect a region of the pathogen of interest. In some embodiments, the biomarker comprises orf1a, orf1ab, spike protein (S protein), 3a, 3b, envelope protein (E protein), matrix protein (M protein), p6, 7a, 7b, 8b, 9b, nucleocapsid protein (N protein), orf14, nsp1 (leader protein), nsp2, nsp3, nsp4, nsp5 (3C-like proteinase), nsp6, nsp7, nsp8, nsp9, nsp10 (growth-factor-like protein), nsp12 (RNA-dependent RNA polymerase, or RdRp), nsp13 (RNA 5′-triphosphatase), nsp14 (3′-to-5′ exonuclease), nsp15 (endoRNAse), and nsp16 (2′-O-ribose methyltransferase), a portion thereof, or combinations thereof.

In some embodiments, the biomarker is a proinflammatory marker. In some embodiments, the proinflammatory marker comprises 4-1BBL, acylation stimulating protein, adipokine, albinterferon, APRIL, Arh, BAFF, Bcl-6, CCL1, CCL1/TCA3, CCL1, CCL12/MCP-5, CCL13/MCP-4, CCL14, CCL15, CCL16, CCL17/TARC, CCL18, CCL19, CCL2, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CCR10, CCR3, CCR4, CCR5, CCR6, CCR7, CCR8, CD153, CD154, CD178, CD40LG, CD70, CD95L/CD178, Cerberus (protein), chemokines, CLCF1, CNTF, colony-stimulating factor, common b chain (CD131), common g chain (CD132), CX3CL1, CX3CR1, CXCL1, CXCL10, CXCL11, CXCL12, CXCL13, CXCL14, CXCL15, CXCL16, CXCL17, CXCL2, CXCL2/MIP-2, CXCL3, CXCL4, CXCL5, CXCL6, CXCL7, CXCL9, CXCR3, CXCR4, CXCR5, EDA-A1, Epo, erythropoietin, FAM19A1, FAM19A2, FAM19A3, FAM19A4, FAM19A5, Flt-3L, FMS-like tyrosine kinase 3 ligand, Foxp3, GATA-3, GcMAF, G-CSF, GITRL, GM-CSF, granulocyte colony-stimulating factor, granulocyte-macrophage colony-stimulating factor, hepatocyte growth factor, IFNA1, IFNA10, IFNA13, IFNA14, IFNA2, IFNA4, IFNA5/IFNaG, IFNA7, IFNA8, IFNB1, IFNE, IFNG, IFNZ, IFN-α, IFN-β, IFN-γ, IFNω/IFNW1, IL-1, IL-10, IL-10 family, IL-10-like, IL-11, IL-12, IL-13, IL-14, IL-15, IL-16, IL-17, IL-17A-F, IL-18, IL-18BP, IL-19, IL-IA, IL-1B, IL-1F10, IL-1F3/IL-1RA, IL-1F5, IL-1F6, IL-1F7, IL-1F8, IL-1F9, IL-1-like, IL-1RA, IL-1RL2, IL-1α, IL-1β, IL-2, IL-20, IL-21, IL-22, IL-23, IL-24, IL-28A, IL-28B, IL-29, IL-3, IL-31, IL-33, IL-35, IL-4, IL-5, IL-6, IL-6-like, IL-7, IL-8/CXCL8, IL-9, inflammasome, interferome, interferon, interferon beta-1a, interferon beta-1b, interferon gamma, interferon type I, interferon type II, interferon type III, interferons, interleukin, interleukin 1 receptor antagonist, Interleukin 8, IRF4, Leptin, leukemia inhibitory factor (LIF), leukocyte-promoting factor, LIGHT, LTA/TNFB, LT-β, lymphokine, lymphotoxin, lymphotoxin alpha, lymphotoxin beta, macrophage colony-stimulating factor, macrophage inflammatory protein, macrophage-activating factor, M-CSF, MHC class III, miscellaneous hematopoietins, monokine, MSP, myokine, myonectin, nicotinamide phosphoribosyltransferase, oncostatin M (OSM), oprelvekin, OX40L, platelet factor 4, promegapoietin, RANKL, SCF, STAT3, STAT4, STAT6, stromal cell-derived factor 1, TALL-1, TBX21, TGF-α, TGF-β, TGF-β1, TGF-β2, TGF-β3, TNF, TNFSF10, TNFSF11, TNFSF12, TNFSF13, TNFSF14, TNFSF15, TNFSF4, TNFSF8, TNF-α, TNF-β, Tpo, TRAIL, TRANCE, TWEAK, vascular endothelial growth inhibitor, XCL1, or XCL2. In some embodiments, the biomarkers includes GM-CSF, IL-1α, IL-5, IL-7, IL-12/23 p40, IL-15, IL-16, IL-17A, TNF-β, VEGF, INF-γ, IL-1β, IL-2, IL-4, IL-6, IL-8, IL-10, IL-12 p70, IL-13, TNF-α, Eotaxin, MIP-10, Eotaxin-3, TARC, IP-10, MIP-1α, IL-8, MCP-1, MDC, MCP-4, IFN-g, or VEGF-A, or a combination thereof.

In some embodiments, the assay assembly comprises one or more capture molecules. Non-limiting examples of capture molecules include a nucleic acid molecule, peptide, protein, or fragments thereof. In some embodiments, the nucleic acid molecule is a deoxyribonucleic acid (DNA) molecule or a ribonucleic acid (RNA) molecule. In some embodiments, the nucleic acid molecule or peptide is an aptamer that binds to an analyte described herein. In some embodiments, the peptide or protein is derived from the receptor binding domain (RBD) or the spike protein from a coronavirus. In some embodiments, the peptide or protein is derived from the ACE2 receptor surfaces for biomolecular detection (e.g., human ACE2). In some embodiments, the peptide or protein is an antibody or antigen-binding fragment that directly or indirectly binds the RBD of the spike protein or ACE2. In some embodiments, the capture molecule may include heparin, or a fragment thereof.

In some embodiments, the one or more capture molecules is coupled to a solid surface. In some embodiments, the one or more capture molecules is not coupled to a solid surface. In some embodiments, the solid surface is made of silicon, glass (silicon dioxide), nitrocellulose, gold, silver, polystyrene, graphene, or crystal. In some embodiments, the solid surface is a bead or a plate. In some embodiments, the plate comprises reaction wells, such as in a 96-well plate, a 384-well plate, or a 1536-well plate. In some embodiments, the bead is substantially spherical in shape.

In some embodiments, the surface is passivated. In some embodiments, the surface comprises a polymer coating comprising a polymer selected from polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran.

In some embodiments, the one or more capture molecules is coupled to the surface directly or indirectly. Capture molecules coupled indirectly to the surface may, for example, be coupled to the surface by a linker. In some embodiments, the linker is a chemical linker, a peptide link, a polymer linker, or a combination thereof. In some embodiments, an epitope tag is conjugated to the one or more capture molecules. Non-limiting examples of an epitope tag include MBP, His-tag, V5 tag, c-my, HA, S-tag, strep-tag, strep-MAB, VSV-G, GST, GFP, mCherry, CFP, BFP, and DYKDDDDK (SEQ ID NO: 116). In some embodiments, the one or more capture molecules is bound by a primary capture antibody that is bound to the surface covalently or non-covalently. In some embodiments, the one or more capture molecules is conjugated to an epitope tag and the epitope tag is coupled to a primary capture antibody that is bound to the surface. Capture molecules coupled directly to the surface may, for example, be covalently or non-covalently bound to the surface.

The one or more capture molecules described herein may be immobilized to the surface directly or indirectly using a suitable surface modification chemistry depending on the intended use. Non-limiting examples of suitable surface modification chemistries are provided in Sonawane et al., Surface Modification Chemistries of Materials used in Diagnostic Platforms with Biomolecules, Journal of Chemistry (2016), which is hereby incorporated by references in its entirety.

In some embodiments, the one or more capture molecules is derived from an angiotensin-converting enzyme 2 (ACE2) receptor or a fragment thereof, or a surrogate of ACE2 (e.g., heparin) or a fragment thereof. In some embodiments, the fragment of ACE2 comprises an extracellular portion of ACE2 that binds to the spike protein. In some embodiments, the extracellular portion of ACE2 comprises the peptidase domain. In some embodiments, the fragment of ACE2 is a fragment of the extracellular portion of ACE2 that is sufficient for binding to the spike protein of a coronavirus, which are provided in Li et al., Structure of SARS coronavirus spike receptor-binding domain complexed with receptor. Science 309, 1864-1868 (2005); and Yan et al., Structural basis for the recognition of SARS-CoV-2 by full-length human ACE2, Science. 2020 Mar. 27; 367(6485): 1444-1448, which are incorporated by reference herein in their entireties. In some embodiments, ACE2 is human ACE2 (Entrez ID 59272). In some embodiments, the one or more capture molecules comprises a portion of the ACE2 polypeptide provided in SEQ ID NO: 1. In some embodiments, the one or more capture molecules comprises an amino acid sequence at least 70%, 75%, 80%, 85%, 86%, 87%, 88%, 89%, 90%, 91%, 92%, 93%, 94%, 95%, 96%, 97%, 98%, 99%, or 100% identical to the human ACE2 polypeptide (SEQ ID NO: 1). In some embodiments, the one or more capture molecules comprises an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence from amino acid 19 to amino acid 615 of SEQ ID NO: 1. In some embodiments, the one or more capture molecules is a peptide or protein comprising an amino acid sequence encoding a variant of the human ACE2 polypeptide (SEQ ID NO: 1), or a fragment thereof. In some embodiments, the one or more capture molecules comprises an amino acid sequence comprising a Ser19Pro or a Asp329Gly substitution with reference to SEQ ID NO: 1. Variants of ACE2 are provided in Hussain et al., Structural variants in human ACE2 may influence its binding with SARS-CoV-2 spike protein, J. Med. Virol. 2020; 92:1580-1586, which is incorporated by reference herein in its entirety. In some embodiments, the one or more capture molecules comprises at least 2, 3, 4, 5, 6, 7, 8, 9, or 10 capture molecules.

The one or more capture molecules, in some embodiments, is a surrogate for ACE2. A non-limiting example of a surrogate for ACE2 is heparin. In some embodiments, the one or more capture molecules comprises heparin, or a fragment thereof that is sufficient for binding to the spike protein. Heparin may competitively bind to the spike protein of a SARS-CoV with an affinity that is similar to that of ACE2. In some embodiments, the affinity of binding between heparin and the spike protein is less than about 1E⁻⁷, 1E⁻⁸, 1E⁻⁹, or 1E⁻¹⁰ Kd. In some cases, the binding affinity is from about 1E⁻⁹ to about 1E⁻¹⁰ Kd. In some embodiments, a surrogate of ACE2 (e.g., heparin) provided herein has a binding affinity to a spike protein of less than about 1E⁻⁷, 1E⁻⁸, 1E⁻⁹, 1E⁻¹⁰, or 1E⁻¹¹ Kd In some embodiments, the affinity of binding between heparin (or other surrogate of ACE2) and the spike protein is 1.20E⁻⁸. In some embodiments, the affinity of binding between heparin (or other surrogate of ACE2) and the spike protein is 8.30E⁻¹⁰. In some embodiments, the one or more capture molecules is a surrogate for ACE2 with a binding affinity or avidity for the spike protein of less than about 1E⁻⁷, 1E⁻⁸, 1E⁻⁹, 1E⁻¹⁰, or 1E⁻¹¹ Kd. Suitable methods of identifying surrogates for ACE2 to comparable binding affinities to the spike protein are provided at least in Guan-Yu et al., The discovery of potential natural products for targeting SARS-CoV-2 spike protein by virtual screening, bioRxiv 2020.06.25.170639. Suitable methods of calculating the affinity of binding are provided in Vangone A, Bonvin A. PRODIGY: a contact-based predictor of binding affinity in protein-protein complexes. Bio-Protocol. (2017) 7:2124.

In some embodiments, the one or more capture molecules is derived from a spike glycoprotein of a coronavirus. In some embodiments, the coronavirus is a severe acute respiratory syndrome coronavirus (SARS-CoV). In some embodiments, the SARS-CoV is SARS-CoV-2. In some embodiments, the coronavirus is Middle East Respiratory Syndrome coronavirus (MERS-CoV). In some embodiments, the coronavirus is an alpha coronavirus (e.g., 229E, NL63). In some embodiments, the coronavirus is a beta coronavirus (e.g., OC43, HKU1). In some embodiments, the one or more capture molecules comprises a portion of the spike protein. In some embodiments, the portion comprises subunit 1 of the spike protein. In some embodiments, the portion comprises the receptor binding domain (RBD) of subunit 1 of the spike protein. In some embodiments, the one or more capture molecules comprises an amino acid sequence at least 70%, 75%, 80%, 85%, 86%, 87%, 88%, 89%, 90%, 91%, 92%, 93%, 94%, 95%, 96%, 97%, 98%, 99%, or 100% identical to the spike protein, the subunit 1 of the spike protein, or the RBD of the subunit 1 of the spike protein, or a combination thereof. In some embodiments, the one or more capture molecules comprises at least 2, 3, 4, 5, 6, 7, 8, 9, or 10 capture molecules.

In some embodiments, the one or more capture molecules comprises a peptide or protein having an amino acid sequence at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 2. In some embodiments, the one or more capture molecules comprises a peptide or protein having an amino acid sequence at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 3. In some embodiments, the one or more capture molecules comprises a peptide or protein having an amino acid sequence least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 4.

In some embodiments, the one or more capture molecules comprises a peptide or protein having an amino acid sequence least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 5. In some embodiments, the one or more capture molecules comprises a peptide or protein having an amino acid sequence with 1, 2, 3, 4, 5, 6, 7, 8, 9, 10, 11, 12, 13, 14, 15, 16, 17, 18, 19, 20, 21, 22, 23, 24, or 25 or more of the mutations to SEQ ID NO: 5 listed in Table 1. In some embodiments, the one or more capture molecules comprises a peptide or protein having an amino acid sequence at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 6.

In some embodiments, the one or more capture molecules comprises a peptide or protein having an amino acid sequence at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 11. In some embodiments, the one or more capture molecules comprises a peptide or protein having an amino acid sequence at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 12. In some embodiments, the one or more capture molecules comprises a peptide or protein having an amino acid sequence at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 24, 47-48, 71-72, 95-96, or 115.

In some embodiments, the one or more capture molecules comprises an amino acid sequence comprising asparagine at amino acid positions 90, 322, 546, and 343 of the amino acid sequence of the spike protein, such as SEQ ID NO: 2, 5, or 11. In some embodiments, a substitution of an amino acid residue corresponding to an amino acid position selected from 344, 360, 472, 479, 480, and 487 in SEQ ID NO: 2, 5, or 11. In some embodiments, the spike protein has an amino acid sequence comprising one or more of 344K, 360F, 472L, 479N, 480D, and 487T with reference to SEQ ID NO: 2, 5, or 11. In some embodiments, the spike protein has an amino acid sequence comprising one or more of 344R, 360S, 472P, 479N, 480G, and 487S with reference to SEQ ID NO: 2, 5, or 11. In some embodiments, the spike protein has an amino acid sequence comprising 344R, 360S, 472L, 479K, 480D, 487S with reference to SEQ ID NO: 2, 5, or 11.

In some embodiments, the spike protein is derived from a mammal. In some embodiments, the spike protein is derived from a human. In some embodiments, the spike protein is derived from a non-human mammal, such as a palm civet or a bat.

In some embodiments, the one or more capture molecules is an antibody, or antibody fragment, specific to one or more antibodies against the pathogen described herein (e.g., IgM, IgG, IgA, IgE, IgD). In some embodiments, the one or more capture molecules is a monoclonal antibody or a polyclonal antibody. In some embodiments, the one or more capture molecules comprises at least 2, 3, 4, 5, 6, 7, 8, 9, or 10 capture molecules.

In some embodiments, the one or more capture molecules is a fusion protein comprising a peptide directly or indirectly bound to the surface. In some embodiments, the peptide comprises a fragment crystallizable (Fc) region of a monoclonal antibody. In some embodiments, the Fc region is derived from an antibody belonging to an immunoglobulin class comprising immunoglobulin M, immunoglobulin G, immunoglobulin A, immunoglobulin E, or immunoglobulin D.

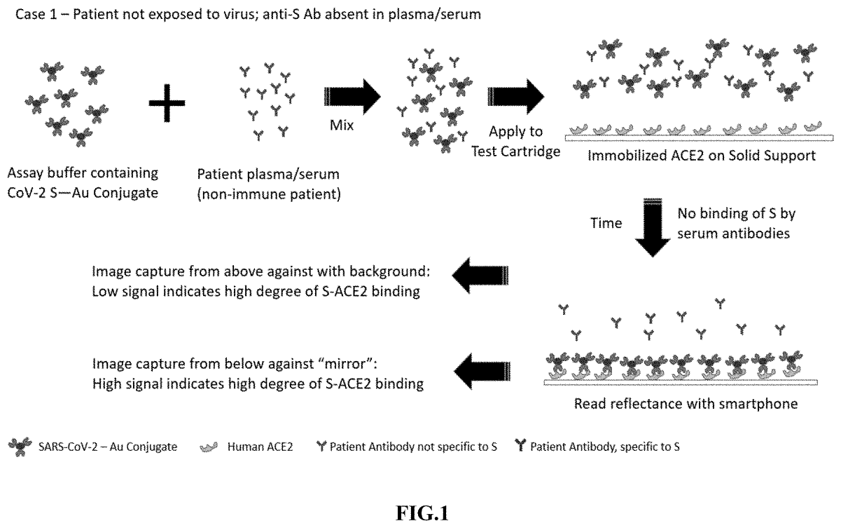

Referring to FIG. 1, a biological sample obtained from a subject that has not been exposed to a coronavirus is assayed using the assay assembly described herein. In some embodiments, a labeled capture molecule (“CoV-2 S—Au”) comprises a peptide-conjugate comprising a detection agent (e.g., gold nanoparticle) and a peptide derived from the spike protein of SARS-CoV-2. A fluid formulation comprising the peptide-conjugate is contacted with a biological sample from the subject. In this example, the target analyte is an activity of one or more antibodies comprising blocking the binding between the spike protein of SARS-CoV-2 and ACE2. In this example the patient has not been exposed to the coronavirus, so a presence of the analyte is not expected to be detected.

In some embodiments, an immobilized capture molecule is coupled to a solid surface. In some embodiments, the immobilized capture molecule is human ACE2 receptor, surrogate thereof (e.g., heparin), or a fragment thereof. The liquid formulation is applied to the solid surface. A high number of complexes between the immobilized human ACE2 and the detectable peptide-conjugate is detected, correlating with an absence or a low amount of analyte in the sample. In some embodiments, an image of the surface is captured with an imaging device. In some embodiments, the image is a video or still image. In some embodiments, the imaging device comprises a reflectance reader. In some embodiments, the imaging device is a personal electronic device, such as a smartphone. When imaged from above the surface, a low signal indicates a high degree of binding between human ACE2 and the peptide-conjugate (therefore, a low amount of the analyte). When imaged from below the surface, a high signal indicates a high degree of binding between the human ACE2 and the peptide-conjugate.

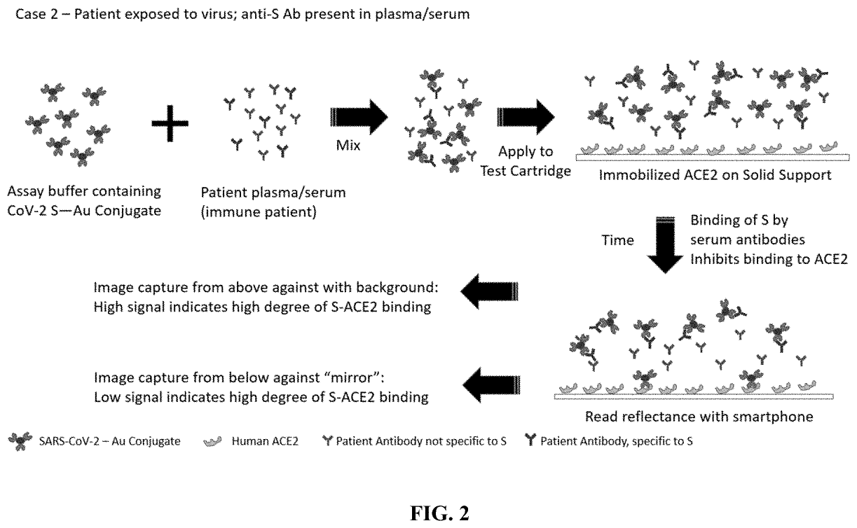

In contrast, a biological sample obtained from a subject that was exposed to a coronavirus assayed using the assay assembly described herein, is provided in FIG. 2. A low number of complexes between the immobilized human ACE2 and the detectable peptide-conjugate is detected, which correlates with a presence absence or a high amount of analyte in the sample.

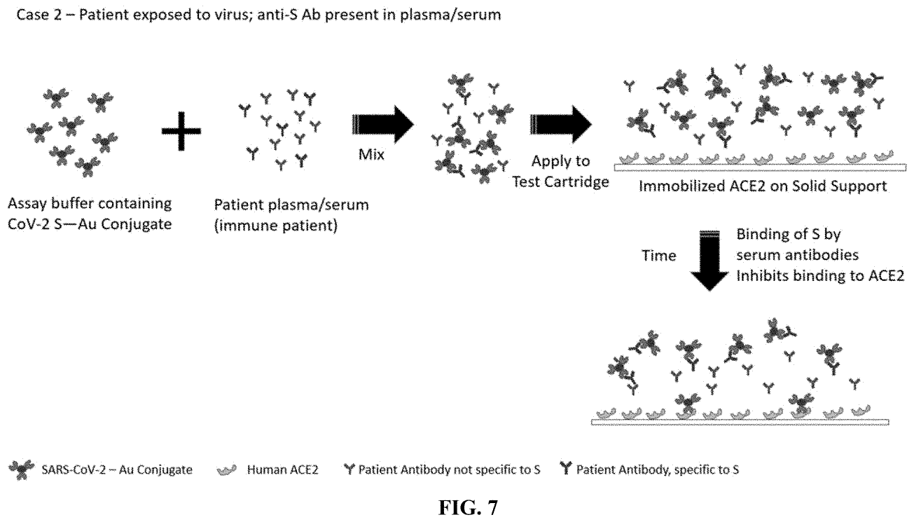

In some embodiments, the surface of the image is not captured. Referring to FIG. 7, a biological sample obtained from the subject exposed to a coronavirus is assayed using the assay assembly described here. A low number of complexes between the immobilized human ACE2 and the detectable peptide-conjugate is detectable by the human eye in the detection zone of the device.

In some embodiments, the assay assembly is adapted for nucleic acid molecule detection, such as to detect a nucleic acid molecule encoding a biomarker or an antibody described herein. In a non-limiting example, the assay assembly is adapted to detect RNA (or cDNA) encoding a region of the pathogen of interest or a marker of an adaptive immune response, such as a cytokine.

In some embodiments, the assay assembly performs reverse transcription polymerase chain reaction (RT-PCR). In some embodiments, the assay assembly is a singleplex (e.g., individual assays) or multiplexed (e.g., detection of more than one biomarker in a single reaction). The assay assembly comprises a primer pair and a probe set to amplify and detect the biomarker. In some embodiments, the assay assembly comprises at least 2, 3, 4, 5, 6, 7, 8, 9, 10, 11, 12, 13, 14, 15, 16, 17, 18, 19, or 20 primer pairs. In some embodiments, the assay assembly comprises at least 2, 3, 4, 5, 6, 7, 8, 9, 10, 11, 12, 13, 14, 15, 16, 17, 18, 19, or 20 probes. A “probe” is a nucleic acid molecule comprising greater than or equal to about 30 contiguous nucleobases adapted to hybridize to the nucleic acid sequence of the target region of the biomarker. In some embodiments, the RT-PCR is qualitative PCR (qPCR), and the probe comprises a detectable moiety. In some embodiments, the detectable moiety comprises TaqMan™, SYBR green, SYBR green I, SYBR green II, SYBR gold, ethidium bromide, methylene blue, Pyronin Y, DAPI, acridine orange, Blue View or phycoerythrin, or a combination thereof. In some embodiments, the probe may be a hydrolysable probe comprising a fluorophore and quencher that is hydrolyzed by DNA polymerase when hybridized to a target nucleic acid, such as for a TaqMan™ assay assembly.

In some embodiments, the assay assembly is an array comprising probes conjugated or otherwise immobilized on a surface described herein (e.g., a bead, multi-well plate), wherein the probes are configured to hybridize with the nucleic acid sequence of the target region. In some embodiments, the target region of the biomarker comprises a mutation, such as a single nucleotide variation or an indel. In some embodiments, the surface comprises an Affymetrix gene chip array, and the like.

### Lateral Flow Assay

In some embodiments, the assay assembly is a lateral flow assay (LFA). In some embodiments, the surface is a membrane comprising porous paper, a polymer structure, a sintered polymer, or a combination thereof. In some embodiments, the LFA assembly has one or more zones situated laterally, including a detectable zone. The detectable zone comprise at least a control region and a test region.

In some embodiments, the LFA assembly further comprises a sample receptor (e.g., a sample pad) configured to receive a biological sample. In some embodiments, the sample receptor comprises a filter designed to separate a component of the biological sample to be tested. For instance, if a biological sample were blood, the sample component would be blood serum. In some embodiments, the LFA assembly further comprises an absorbent pad.

In some instances, the target analyte moves without the assistance of external forces, e.g., by capillary action. In some instances, the target analyte moves with assistance of extemal forces, e.g., by facilitation of capillary action by movement of the lateral flow assembly (e.g., shaking, turning, centrifuging, applying an electrical field or magnetic field, applying a pump, applying a vacuum, or rocking).

Any suitable lateral flow test strip detection format known to those of skill in the art is contemplated for use in an assay assembly of the present disclosure. Lateral flow test strip detection formats are well known and have been described in the literature. Lateral flow test strip assay formats are generally described by, e.g., Sharma et al., (2015) Biosensors 5:577-601, incorporated by reference herein in its entirety. Detection of nucleic acids using lateral flow test strip sandwich assay formats is described by, e.g., U.S. Pat. No. 9,121,849, “Lateral Flow Assays,” incorporated by reference herein in its entirety. Detection of nucleic acids using lateral flow test strip competitive assay formats is described by, e.g., U.S. Pat. No. 9,423,399, “Lateral Flow Assays for Tagged Analytes,” incorporated by reference herein in its entirety.

Disclosed herein, in some embodiments, are signal detection devices, such as an imaging device. In some embodiments, the imaging device is a personal electronic device (e.g., smartphone or tablet). In some embodiments, the imaging device comprises a fluorescence reader, a colorimeter, or a sensor.

In some embodiments, the peptide-conjugate described herein comprises detection reagent or a label. Non-limiting examples of a detection reagent include a fluorophore, a chemical, a nanoparticle, an antibody, a peptide, and a nucleic acid probe. In some embodiments, the nanoparticle comprises a material selected from agarose, plastic, acrylic, or metal. In some embodiments, the nanoparticle is a microsphere. In some embodiments, the nanoparticle is magnetic. In some embodiments, the imaging device detects color, reflectance, fluorescence, bioluminescence, chemiluminescence, light, or an electrical signal.

In some embodiments, the LFA assembly is in a sandwich format, a competitive format. In a sandwich assay format, the detected signal is directly proportional to the amount of the target analyte present in the sample, so that increasing amounts of the target analyte lead to increasing signal intensity. In a competitive assay format, the detected signal has an inverse relationship with the amount of analyte present, and increasing amounts of analyte lead to decreasing signal intensity.

In a lateral flow sandwich format, also referred to as a “sandwich assay,” the biological sample (test sample) is applied to a sample receptor (“sample pad”) at a distal end of the LFA test strip. The biological sample flows through the test strip, from the sample pad to a conjugate pad located adjacent to, and downstream from, the sample pad. In some embodiments, the conjugate pad comprises a labeled capture molecule, e.g., an antibody or aptamer labeled with e.g., a dye, enzyme, or nanoparticle. A complex between the capture molecule and the target analyte is formed if the target analyte is present in the test sample. This complex then flows to a first test zone or sector (e.g., a test line) comprising an immobilized second capture molecule which is specific to the target analyte, thereby trapping any labeled capture molecule-target analyte complexes. In some embodiments, the intensity or magnitude of signal, e.g., color, fluorescence, reflectance, at the first test zone or sector is used to indicate the presence or absence, quantity, or presence and quantity of target analyte in the test sample. In some embodiments, the assay assembly comprises a second test zone or sector comprising a third capture molecule that binds to excess labeled capture molecule. If the applied test sample comprises the target analyte, little or no excess labeled capture molecule will be present on the test strip following capture of the target analyte by the labeled capture molecule on the conjugate pad. Consequently, the second test zone or sector will not bind any labeled capture molecule, and little or no signal (e.g., color, fluorescence, reflectance) at the second test zone or sector is expected to be observed. The absence of signal at the second test zone or sector thus provides assurance that signal observed in the first test zone or sector is due to the presence of the target analyte.

In some embodiments, the sandwich assay is configured to receive a biological sample disclosed herein and retain sample components (e.g., target analyte). In some embodiments, the sandwich assay is configured to receive a flow solution that flushes unwanted cellular components (other than the analyte) of the biological sample, leaving the target analyte behind. In some embodiments, the sandwich assay comprises a membrane that binds the target analyte to help retain the target analyte when the flow solution is applied. Non-limiting examples of a membrane that binds a target analyte includes chitosan modified nitrocellulose.

In some embodiments, the assay assembly comprises a sandwich assay. In some embodiments, the target analyte is an antibody specific to a pathogen of interest (e.g., SARS-CoV-2). In some cases, capture molecule is a peptide derived from a spike protein from a coronavirus (e.g., SARS-CoV-2). In some embodiments, immobilized capture molecule is an antibody specific to the target analyte, such as an antibody or antigen-binding fragment. In some embodiments, the capture molecule is labeled, and a signal corresponding to the presence of the target analyte is visualized. In some embodiments, a labeled secondary molecule (e.g., antibody or antibody fragment) specific to the capture molecule added, and a signal corresponding to the presence of a complex between the target analyte, the capture molecule, and the secondary molecule is visualized. In some embodiments, signal is observed at the first test zone comprising color, fluorescence, or reflectance emitted from the labeled capture molecule. In some embodiments, the labeled capture molecule is a peptide-conjugate comprising a peptide derived from a spike protein from a coronavirus, and a label comprising a nanoparticle (e.g., microsphere), an enzymatic label, or a fluorescent dye.

In some embodiments, the assay assembly detects more than one target analyte. In some embodiments, the assay assembly provides two or more capture molecules that are specific to two or more antibodies specific to a pathogen of interest, such as, for example IgG, IgM, or IgA antibodies against SARS-CoV-2. In some embodiments, the two or more capture molecules are labeled. In some embodiments, the two or more capture molecules are not labeled. In some embodiments, two or more labeled secondary molecules specific to the two or more capture molecules are added to the assay assembly, and visualized as described herein.

In a lateral flow competitive format, also referred to as a “competitive assay,” the test sample is applied to a sample pad at one end of the test strip, and the target analyte binds to a labeled capture molecule to form a complex between the target analyte and the labeled capture molecule in a conjugate pad downstream of the sample application pad. In the competitive format, the first test zone comprises an immobilized capture molecule specific to second capture molecule that is labeled. In the absence of the target analyte, the immobilized capture molecule and the labeled second capture molecule form a detectable complex. In the presence of the target analyte, fewer complexes between the capture molecule and the labeled second capture molecule form due to competition for binding to the labeled second capture molecule. In some embodiments, the intensity or magnitude of signal, e.g., color, fluorescence, reflectance, at the first test zone or sector is inversely proportionate to the presence or absence, quantity, or presence and quantity of the target analyte in the test sample.

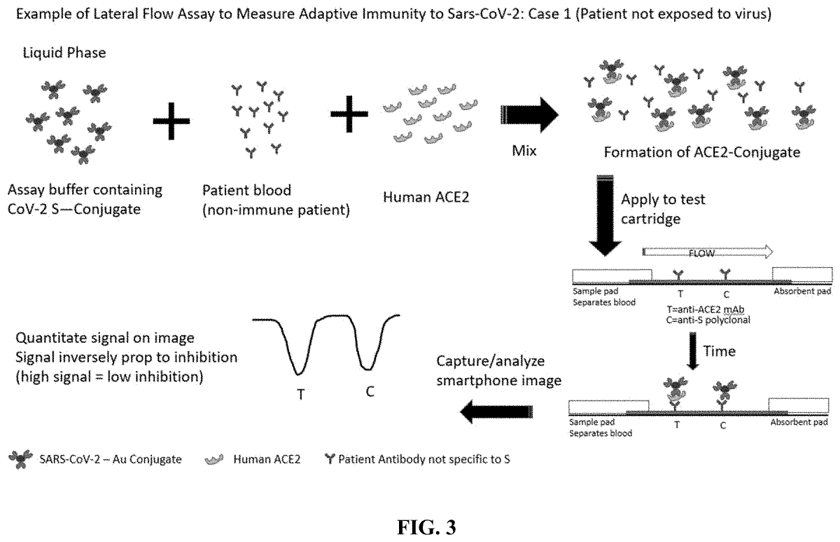

In some embodiments, the assay assembly comprises a competitive assay. Referring to FIG. 3, the labeled capture molecule comprises a peptide-conjugate comprising a detection agent (e.g., gold nanoparticle) and a peptide derived from the spike protein of a coronavirus. A fluid formulation comprising the peptide-conjugate is contacted with a biological sample from a patient and human ACE2, wherein the peptide derived from the spike protein of the coronavirus comprises a receptor binding domain (RBD) specific to the human ACE2. In this example, the target analyte is one or more antibodies against the spike protein of the coronavirus. In this example the patient has not been exposed to the coronavirus, so a presence of the analyte is not expected to be detected.

The liquid formulation comprises at least one detectable complex comprising the peptide-conjugate RBD and the human ACE2, because the biological sample does not consist of antibodies against the RBD of the peptide. The liquid formulation comprising the peptide-conjugate, the biological sample, and the human ACE2 is applied to a solid surface at least partially enclosed in a housing (“test cartridge”). The liquid formulation is applied to the distal end of the solid surface at a sample pad, and flows unidirectionally over the solid surface towards the opposite distal end of the solid surface. In some embodiments, the biological sample is blood, and the sample pad separates serum from blood, permitting only the serum to flow across the solid surface. In some embodiments, the solid surface is a nitrocellulose membrane. In some embodiments, the first test zone comprises an immobilized antibody specific to the human ACE2. In some embodiments, the second test zone comprises an immobilized antibody specific to the RBD of the spike protein. In some embodiments, a high signal is observed at the first test zone, because there is an absence of target analyte in the biological sample. In some embodiments, a high signal is observed at the second test zone (positive control), for the same reason.

In alternative embodiments, the immobilized capture molecule in the first test zone is human ACE2 receptor, and the fluid formulation does not contain human ACE2. In this example, the liquid formulation comprises the peptide-conjugate and the biological sample. No complexes form between the peptide-conjugate in the absence of the target analyte (antibodies against the RBD of the spike protein). The liquid formulation is applied to the distal end of the solid surface at a sample pad, and flows unidirectionally over the solid surface towards the opposite distal end of the solid surface. Like above, in the first and second test zones, a high signal is observed in the absence of the test analyte.

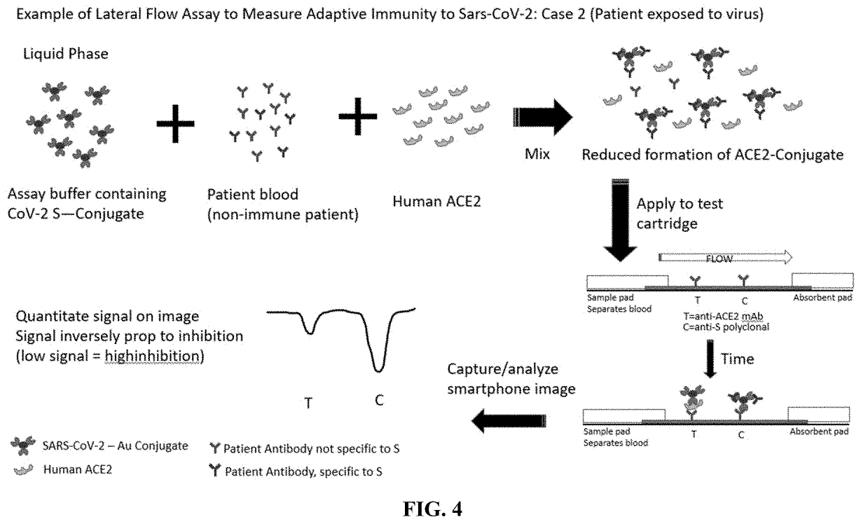

Referring to FIG. 4, the same competition assay is performed on biological sample obtained from a subject that has been exposed to the coronavirus. In some embodiments, there is a presence of the target analyte in the biological sample. Therefore, a low signal is observed at the first and second test zones, which indicates a high amount of competitive binding between the target analyte and the peptide-conjugate.

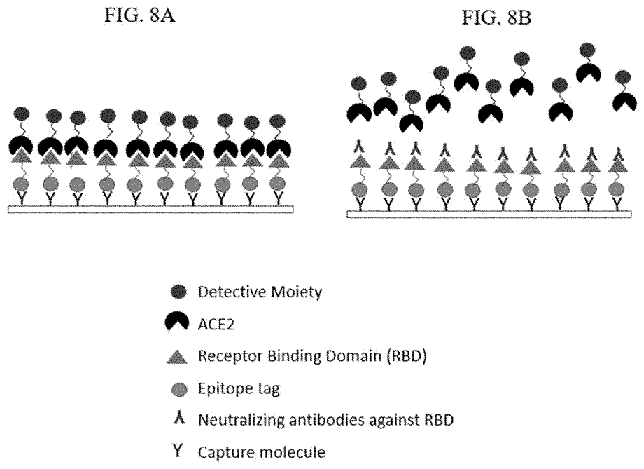

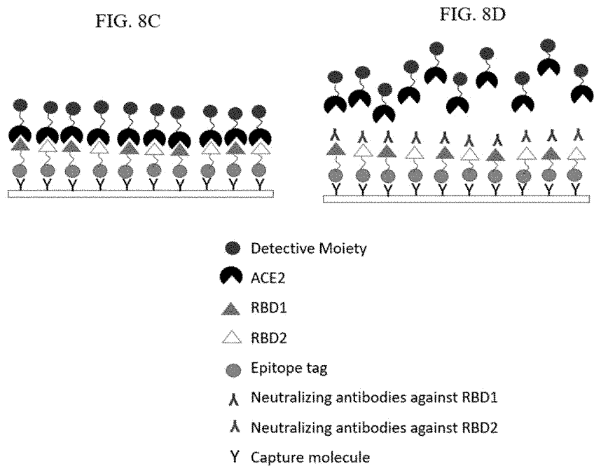

Referring to FIG. 8A-8D, the assay assembly measures analyte in the biological sample. In some embodiments, the assay assembly measures one analyte (e.g. an neutralizing antibody) in the biological sample. Referring to FIG. 8A-8B, one capture molecule is used, for example RBD. FIG. 8A exemplifies an assay assembly after it is performed with a biological sample that is obtained from an individual that does not have neutralizing antibodies against RBS. The peptide-conjugate (e.g., ACE2) conjugated to a detectable moiety binds to the RBD in the absence of neutralizing antibodies. The detectable moiety is visualized using the naked eye, or an imaging device described herein. FIG. 8B exemplifies an assay assembly after it is performed with a biological sample that is obtained from an individual that has neutralizing antibody against RBD. The peptide-conjugate (e.g., ACE2) conjugated to a detectable moiety does not bind to the RBD in the presence of neutralizing antibody in the patient sample.

In some embodiments, the assay assembly measures more than one analyte (e.g., neutralizing antibody) in the biological sample. Referring to FIG. 8C-8D, more than one capture molecule is used, for example RBD1 and RBD2. In some embodiments, two or more capture molecules are used. In some embodiments, the two or more capture molecules comprise a first capture molecule and a second capture molecule, wherein the first capture molecule comprises a first variant RBD and the second capture molecules comprises a second variant RBD. In some embodiments, at least 3, 4, 5, 6, 7, 8, 9, or 10 capture molecules are used. In some embodiments, one of the capture molecules are the same. FIG. 8C exemplifies an assay assembly after it is performed with a biological sample that is obtained from an individual that does not have neutralizing antibodies against either of RBD1 and RBD2. The peptide-conjugate (e.g., ACE2) conjugated to a detectable moiety binds to the RBD1 and RBD2 in the absence of neutralizing antibodies. The detectable moiety is visualized using the naked eye, or an imaging device described herein. FIG. 8D exemplifies an assay assembly after it is performed with a biological sample that is obtained from an individual that has neutralizing antibodies against both RBD1 and RBD2. The peptide-conjugate (e.g., ACE2) conjugated to a detectable moiety does not bind to the RBD1 and RBD2 in the presence of neutralizing antibodies in the patient sample.

In a lateral flow test strip detection format, more than one target analyte is detected using the test strip through the use of additional test zones or sectors comprising, e.g., probes specific for each of the target analytes.

In some instances, the lateral flow device is a layered lateral flow device, comprising zones or sectors that are present in layers situated medially, e.g., above or below each other. In some instances, one or more zones or sectors are present in a given layer. In some instances, each zone or sector is present in an individual layer. In some instances, a layer comprises multiple zones or sectors. In some instances, the layers are laminated. In a layered lateral flow device, processes controlled by diffusion and directed by the concentration gradient are possible driving forces. For example, multilayer analytical elements for fluorometric assay or fluorometric quantitative analysis of an analyte contained in a sample liquid are described in EP0097952, “Multilayer analytical element,” incorporated by reference herein.

A lateral flow device comprises one or more functional zones or sectors. In some embodiments, the test assembly comprises 1 to 20 functional zones or sectors. In some instances, the functional zones or sectors comprise at least one sample purification zone or sector, at least one target analyte amplification zone or sector, at least one target analyte detection zone or sector, and at least one target analyte detection zone or sector.

In some embodiments, the assay assembly comprises a detection zone made up of at least the first and the second test zones (test, and control). In some embodiments, the assay assembly comprises more than one test zone. In some embodiments, the assay assembly comprises a first test zone and a second test zone, wherein the first test zone indicates a presence or a quantity of neutralizing antibodies that competitively bind to the peptide-conjugate; and the second test zone indicates a presence or a quantity of antibodies against the pathogen (neutralizing or otherwise). In some embodiments, the assay assembly comprises a third test zone. In some embodiments, the second test zone detects IgG antibodies against the pathogen. In some embodiments, the third test zone detects IgM antibodies against the pathogen. In some embodiments, the assay assembly comprises a plurality of test zones, wherein each test zone of the plurality measures a different analyte. In some embodiments, an image of the detection zone is captured with an imaging device. In some embodiments, the image is a video or still image. In some embodiments, the imaging device comprises a fluorescence reader, a colorimeter, or a sensor. In some embodiments, the imaging device is a personal electronic device, such as a smartphone.

In some embodiments, the assay assembly is a lab-on-chip system (e.g., Maverik@). In some embodiments, the solid surface comprises a silicon chip. In some embodiments, the imaging device comprises photonic biosensors that measure changes in refractive index caused by binding between the analyte and the peptide-conjugate to form complexes, as described in Iqbal M., et al. (2010) Label-Free Biosensor Arrays Based on Silicon Ring Resonators and High-Speed Optical Scanning Instrumentation. IEEE J Sel Quantum Elec 16.

In some embodiments, the lateral flow assay assembly confirms presence, absence, or quantity of the neutralizing antibody described herein from a subject's biological sample. In some embodiments, a subject may be a member of a variety of races. For example, the subject may be of the White, Black, Hispanic, or Asian race. In some embodiments, a subject may be of a variety of ages. For example, the subject may be age 0 to age 18, age 18 to age 65, or age 65 and above. In some embodiments, a subject may be a male or a female. In some embodiments, a subject may be pregnant, breastfeeding, or may have priorly been infected with a coronavirus. In some embodiments, a subject may be a high risk subject and have one or more risk factors related to a coronavirus. In some embodiments, a subject may be obese. In some instances, the lateral flow assay assembly prognoses a subject's immunity to any one of the pathogens described herein. In some embodiments, the lateral flow assay assembly prognoses a subject's immunity to coronavirus. In some embodiments, the lateral flow assay assembly prognoses a subject's immunity to SARS-CoV-2. In some embodiments, the lateral flow assay assembly measures the subject's susceptibility to an infection by any one of the pathogens described herein. In some embodiments, the lateral flow assay assembly measures the subject's susceptibility to an infection by coronavirus. In some embodiments, the lateral flow assay assembly measures the subject's susceptibility to an infection by SARS-CoV-2. In some embodiments, the lateral flow assay assembly identifies individuals in a population that respond to a vaccine and individuals in a population that do not respond to a vaccine. In some embodiments, the lateral flow assay assembly minimizes or reduces the risk associated with usage of a commercialized vaccine in a population (e.g., by identifying individuals that do not respond to the vaccine). In some embodiments, the lateral flow assay assembly identifies neutralizing antibodies as a surrogate marker of protection from a vaccine (e.g., in place of traditional, randomized, placebo-controlled trials). In some embodiments, the lateral flow assay assembly classifies vaccines by % efficacy in a population.

In some embodiments, the lateral flow assays provided herein have an accuracy of at least 80%, for example, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or greater. In some embodiments, the lateral flow assays provided herein have a sensitivity of at least 80%, for example, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or greater. In some embodiments, the lateral flow assays provided herein have a specificity of at least 80%, for example, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or greater.

### Fluorescence Resonance Energy Transfer (FRET) Assay

In some embodiments, the assay assembly is a solution-based assay and does not consist of a solid surface. In some embodiments, the solution-based assay comprises labeling any one of the capture molecules described herein by conjugating the capture molecule to a detectable moiety of any one of the moieties described herein. In some embodiments, the signal is detected when the capture molecule forms a complex between the capture molecule and the target analyte. In some embodiments, the signal is not detected when the capture molecule forms a complex between the capture molecule and the target analyte. Exemplary solution-based assay comprising the labeled capture molecules includes Förster resonance energy transfer (FRET), fluorescence polarization (FP), radioligand binding assay, bioluminescent binding assay, and immunoprecipitation (e. g. ELISA and western blotting). In some embodiments, the solution-based assay comprises unlabeled capture molecules, where the detection of the complex formed between the capture molecule and the target analyte is detected by a separate mechanism. Exemplary solution-based assay comprising unlabeled capture molecules includes surface plasmon resonance (SPR), plasmon-waveguide resonance, whispering gallery microresonator (WGM), resonant waveguide grating (RWG), mass spectrometry, nuclear magnetic resonance, X-ray crystallography, thermal denaturation assays (TDA), and isothermal titration calorimetry (ITC).

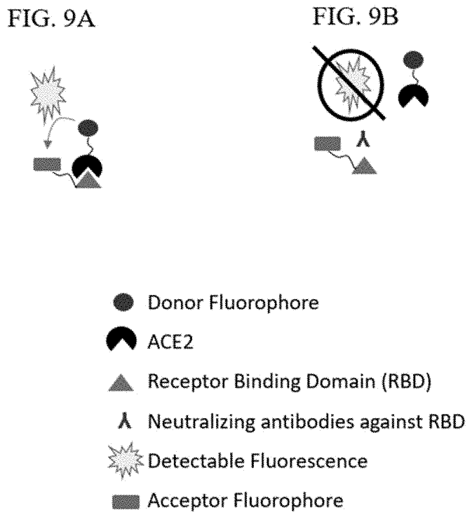

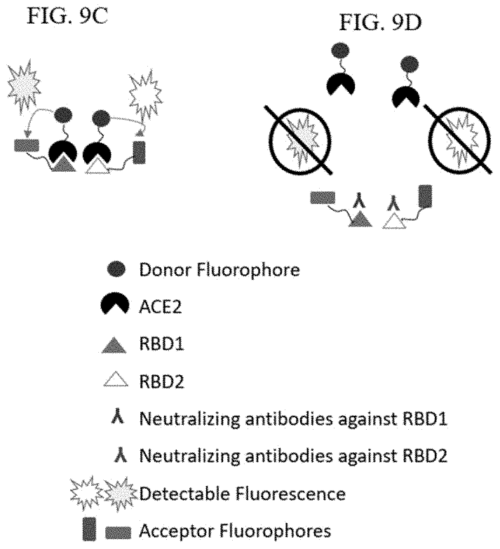

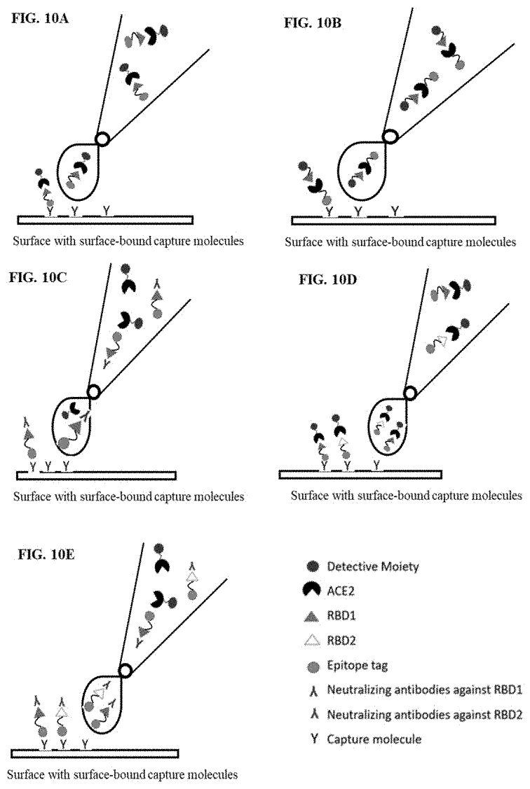

In some embodiments, the assay assembly is a (FRET) system. In some embodiments, the FRET system comprises conjugating donor and acceptor fluorophores to any one of the capture molecules described herein. In some embodiments, a complex is formed between the capture molecule and the target analyte, which prevents the donor fluorophore to be in close proximity to acceptor fluorophore to generate a detectable fluorescence. In some embodiments, referring to FIG. 9A-9D, fluorescence resonance transfer (FRET) is utilized in solution to identify a presence or a quantity of neutralizing antibodies that functionally block binding between the RBD of the spike protein derived from SARS-CoV and the cognate receptor, ACE2. FRET relies on the excitation energy of a donor fluorophore to a nearby acceptor fluorophore. In some embodiments, the peptide-conjugate (e.g., RBD peptide, or portion thereof) is coupled to a donor fluorophore, and the cognate receptor (e.g., ACE2, or portion thereof) is coupled to an acceptor fluorophore. In some embodiments, the donor or the acceptor fluorophore may be any suitable fluorescent protein. In some embodiments, the fluorescent proteins comprise a color selected from, blue, cyan, green, and yellow.

Referring to FIG. 9A, when a biological sample that is obtained from an individual that does not have neutralizing antibodies against the RBD, excitation of the acceptor fluorophore is observed. Whereas, no excitation of the acceptor fluorophore is observed if the biological sample contains neutralizing antibodies against RBD, as shown in FIG. 9B.

Operating in a similar manner to FIG. 8A-8B, FIG. 9C-9D provides an assay assembly in solution (e.g., not involving a solid surface) using FRET.

The FRET assay may be performed in a vessel, such as a container or a test tube. In some embodiments, the FRET assay is performed in an assay plate, such as, a 96-well plate, a 384-well plate, or a 1536-well plate. In some embodiments, the FRET assay is performed in a high-throughput method to decrease the amount of reagents and the cost of performing the assay. In some embodiments, the FRET assay utilizes small volumes of reagents, for example, less than 100 nL, less than 90 nL, less than 80 nL, less than 70 nL, less than 60 nL, or less than 50 nL of reagents.

In some embodiments, the biological sample (e.g., blood from a finger prick) is stored in or on a biological sample medium (BSM), such as a dried blood spot card (DBS), described herein. The biological sample is removed from the BSM and placed into the vessel (e.g., a well of a 96-well plate) to be eluted from the BSM. Next, the eluted biological sample is assayed using the FRET system deposited on a solid surface described herein.

In some embodiments, the FRET assay confirms presence, absence, or quantity of the neutralizing antibody described herein from a subject's biological sample. In some instances, the FRET assay prognoses a subject's immunity to any one of the pathogen described herein. In some embodiments, the FRET assay prognoses a subject's immunity to coronavirus. In some embodiments, the FRET assay prognoses a subject's immunity to SARS-CoV-2. In some embodiments, the FRET assay measures the subject's susceptibility to an infection by any one of the pathogens described herein. In some embodiments, the FRET assay measures the subject's susceptibility to an infection by coronavirus. In some embodiments, the FRET assay measures the subject's susceptibility to an infection by SARS-CoV-2. In some embodiments, the FRET assay identifies individuals in a population that respond to a vaccine and individuals in a population that do not respond to a vaccine. In some embodiments, the FRET assay minimizes or reduces the risk associated with usage of a commercialized vaccine in a population (e.g., by identifying individuals that do not respond to the vaccine). In some embodiments, the FRET assay identifies neutralizing antibodies as a surrogate marker of protection from a vaccine (e.g., in place of traditional, randomized, placebo-controlled trials). In some embodiments, the FRET assay classifies vaccines by % efficacy in a population.

In some embodiments, the FRET assays provided herein have an accuracy of at least 80%, for example, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or greater. In some embodiments, the FRET assays provided herein have a sensitivity of at least 80%, for example, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or greater. In some embodiments, the FRET assays provided herein have a specificity of at least 80%, for example, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or greater.

### Agglutination Assay

In some embodiments, the assay assembly comprises an assembly for agglutination assay. Agglutination assay relies on agglutination of particles or nanoparticles due to a capture molecule or peptide-conjugate forming a complex with the target analyte in the sample, thus indicating the presence of the target analyte in the sample. In some embodiments, the agglutination assay comprises separating free and bound label by chromatographically separating agglutinated and non-agglutinated antibody-coated colored particles along a membrane. In some embodiments, the agglutination assay comprises a colored latex agglutination reaction, where agglutinated and non-agglutinated particles are separated by a capillary which allows non-agglutinated latex beads through but traps the aggregated latex beads. In some embodiments, the agglutination assay comprises multivalent analytes whereby in the absence of analyte label passes through a filter, but in the presence of analyte an agglutinate is formed which is trapped. In some embodiments, the agglutination assay comprises a system, where a test mixture is formed by contacting the sample with colored particles having analyte-specific receptors on the surface of the particles. The test mixture is passed through a filter having pores which are larger than the colored particles but smaller than the particle-analyte aggregates, thus causing trapping of the aggregates. Presence of aggregates from the mixture is determined by checking the color of the filtrate. In some embodiments, the agglutination assay comprises a lateral flow assay without immobilizing antibody for very large analytes. In some embodiments, the agglutination assay comprises a 2-zone system, where one zone having large pores and one zone having small pores, such that analyte passes through the large pores but becomes trapped by the zone of small pores. Such arrangement is used in conjunction with a small label (e.g. gold particle) which passes through both zones. In the presence of target analyte, a fraction of the gold particle becomes bound to the target analyte and becomes trapped at the zone with small pores. In some embodiments, the agglutination assay is a non-capillary agglutination assay.

In some instances, the agglutination assay comprises covalently or non-covalently linking a particle or nanoparticle with at least one capture molecule, at least one detectable moiety, and/or at least one peptide-conjugate. In some embodiments, the particle or nanoparticle comprises macromolecule, colloidal metal (such as gold or silver) particle, bead (e.g. latex bead), charcoal, kaolinite, or bentonite. In some embodiments, the particle or nanoparticle also function as detectable moiety. In some embodiments, the particle or nanoparticle comprises of the same material as the detectable moiety. In some embodiments, the particle, nanoparticle, or detectable moiety comprises material that is agglutinable, with other (preferably similar or identical) particle, nanoparticle, or detectable moiety. The ability of the particle, nanoparticle, and detectable moiety to agglutinate enables the formation of larger agglutinates, resulting in increased stability or the detectable signal generated from the agglutinated particle, nanoparticle, or detectable moiety.

In some embodiments, the detectable moiety comprises detectable material such as a fluorescent dye, an enzymatic label, or a colorimetric label. Detectable moiety includes enzymatic label (e.g., horseradish peroxidase (HRP), beta-galactosidase, alkaline phosphatase, etc), fluorescent dye, luminescent moiety, radioactive moiety, colorimetric label, colored latex particle or nanoparticle, and metal-conjugated moiety such as metallic nanolayer, metallic nanoparticle, or metallic nanoshell-conjugated moiety. Suitable metallic nanoparticle or metallic nanoshell moiety includes gold particle, silver particle, copper particle, platinum particle, cadmium particle, composite particle, gold hollow sphere, gold-coated silica nanoshell, and silica-coated gold shell. Metallic nanolayer suitable for detectable moiety includes nanolayer comprising cadmium, zinc, mercury, gold, silver, copper, and platinum.

In some embodiments, the detectable moiety is directly or indirectly tagged for a colorimetric assay (e.g., for detection of HRP or beta-galactosidase activity), visual inspection using light microscopy, immunofluorescence microscopy, confocal microscopy, by flow cytometry (FACS), autoradiography electron microscopy, immunostaining, or subcellular fractionation. In some embodiments, the detectable moiety is directly incorporated into the capture molecule. For example, a radioactive amino acid is inserted into the capture molecule comprising a peptide. In some embodiments, the detectable moiety is part of the peptide-conjugate. In some embodiments, the detection method comprises visually examining for signs of agglutination. In some embodiments, the detection method comprises visibly examining the agglutination for a color change or a physical-chemical change. Physical-chemical changes can occur with oxidation reactions or other chemical reactions, which is detected both visually or via a spectrophotometer. In some embodiments, the agglutination of the particles or nanoparticles due to formation of a complex formed between: capture molecule and target analyte; capture molecule and peptide-conjugate; and/or target analyte and peptide-conjugate is visually examined. In some embodiments, the complex formed is examined using color, reflectance, fluorescence, bioluminescence, or chemiluminescence. In some embodiments, the target analyte is a monoclonal or polyclonal antibody against a protein of any one of the pathogen described herein. In some embodiments, the target comprises a neutralizing antibody. In some embodiments, the target analyte is a monoclonal or polyclonal neutralizing antibody recognizing a spike glycoprotein of a coronavirus. In some embodiments, the target analyte is a monoclonal or polyclonal neutralizing antibody recognizing a spike glycoprotein of SARS-CoV-2. In some embodiments, the target analyte is targeted and captured by the capture molecules described herein.

In some embodiments, agglutination assay assembly comprises particle or nanoparticle linked to capture molecules comprising both ACE2 and spike glycoprotein. If the target analyte comprising the neutralizing antibody recognizing spike glycoprotein is absent in a biological sample, agglutination is observed due to the complex formed by the spike glycoprotein binding to ACE2. In such case, the observed agglutination or signal of the agglutination is indicative of the absence of the target analyte in the biological sample. Conversely, if the target analyte comprising the neutralizing antibody recognizing the spike glycoprotein is present in the biological sample, the binding of the target analyte to the spike glycoprotein prevents the complex formation between spike glycoprotein and ACE2. In this case, the absence of agglutination or signal of the agglutination is indicative of the presence of the target analyte in the biological sample. In some embodiments, the agglutination assay assembly confirms presence, absence, or quantity of the neutralizing antibody described herein from a subject's biological sample. In some instances, the agglutination assay assembly prognoses a subject's immunity to any one of the pathogen described herein. In some embodiments, the agglutination assay assembly measures the subject's susceptibility to an infection by any one of the pathogen described herein.

### ELISA

In some embodiments, the assay assembly comprises an assembly for enzyme-linked immunosorbent assay (ELISA). In certain embodiments, one or more capture molecules are attached or immobilized on a solid support. In some cases, the peptide-conjugate is attached or immobilized on the solid support. In some embodiments, the target analyte is attached or immobilized on the solid support. The solid support is a bead such as colloidal particle, metallic nanoparticle, nanoshell, or latex bead; a flow path in a lateral flow immunoassay device (e.g., a porous membrane); a blot (e.g., Western blot, a slot blot, or dot blot); a flow path in an analytical or centrifugal rotor, or a tube or well (e.g., in a plate suitable for an ELISA or microarray). In some embodiments, the solid support is a plate such as a microtiter plate with multiple wells. In some embodiments, the one or more capture molecules or one or more peptide conjugates is biotinylated, or conjugated to streptavidin, avidin, or neutravidin. In some embodiments, the target analyte is attached to the surface of the well of the microtiter plate via direct binding to the well of the microtiter plate. In some embodiments, the target analyte is attached to the well via binding to one or more capture molecules that are attached to the well of the microtiter plate. In some embodiments, the target analyte is attached or immobilized to the well via binding to one or more peptide-conjugates that are attached or immobilized to the well of the microtiter plate. In some embodiments, the target analyte is attached to the well via binding to one or more peptide-conjugates that form a complex with the one or more capture molecules that are attached or immobilized to the well of the microtiter plate. Residual or nonspecific protein-binding on the solid support can then blocked with an blocking agent, such as bovine serum albumin (BSA) or heat-inactivated normal goat serum (NGS).

In some embodiments, the solid support is incubated with a biological sample suspected of containing the target analyte. The sample is applied and/or attached to the solid support undiluted or diluted. After incubation, the solid support is washed to remove unbound protein and then incubated with an optimal concentration of the one or more capture molecules conjugated to a detectable moiety or to a substrate of the detectable moiety. In some cases, one or more capture molecules conjugated to the detectable moiety comprises secondary antibody, which binds to the neutralizing antibody in the target analyte. In some embodiments, the target analyte comprises any one of the antibody or any one of the neutralizing antibody described herein.

The detectable moiety comprises an enzyme, including HRP, beta-galactosidase, alkaline phosphatase (ALP), and glucose oxidase. Sufficient time is allowed for specific binding between the one or more capture molecules and the target analyte to occur. Color is allowed to develop and the optical density of the contents of the well is determined visually or instrumentally (measured at an appropriate wave length) via the use of a spectrophotometer. In some embodiments, the ELISA assay assembly confirms presence, absence, or quantity of the neutralizing antibody described herein from a subject's biological sample. In some instances, the ELISA assay assembly prognoses a subject's immunity to any one of the pathogen described herein. In some embodiments, the ELISA assay assembly measures the subject's susceptibility to an infection by any one of the pathogen described herein.

### Testing Device and System Components

Disclosed herein, in some embodiments, are testing devices comprising a housing. In some embodiments, a testing device is at least partially enclosed by the housing. In some embodiments, the testing device is fully enclosed by the housing. In some embodiments, the housing comprises a synthetic polymer material, such as plastic.

In some embodiments, the housing is configured to provide information to an imaging device described herein. Information can include, but is not limited to, information to normalize an image of the testing device and identifying information (e.g., barcode, RFID chip). In some embodiments, the identifying information comprises test parameters, test result interpretation instructions, expected values for imaging controls, and the like.

Disclosed herein, in some embodiments, are testing devices comprising a sample receptor. In some embodiments, the sample receptor is a sample receiver, a sample processor, a sample purifier, or a combination thereof. In some embodiments, the sample receptor is a sample receiver configured to receive and retain a biological sample obtained from a subject. In some embodiments, the sample receptor is a sample processor configured to remove a component of the sample or separate the sample into multiple fractions (e.g., blood cell fraction and plasma or serum).

Useful separation materials may include specific binding moieties that bind to or associate with the substance. Binding is covalent or noncovalent. Any suitable binding moiety known in the art for removing a particular substance is used. For example, antibodies and fragments thereof are commonly used for protein removal from samples. In some instances, a sample purifier disclosed herein comprises a binding moiety that binds a nucleic acid, protein, cell surface marker, or microvesicle surface marker in the biological sample. In some instances, the binding moiety comprises an antibody, antigen binding antibody fragment, a ligand, a receptor, a peptide, a small molecule, or a combination thereof.

The sample receptor is a sample purifier, configured to remove an unwanted substance or non-target component of a biological sample. Depending on the source of the biological sample, unwanted substances can include, but are not limited to, proteins (e.g., antibodies, hormones, enzymes, serum albumin, lipoproteins), free amino acids and other metabolites, microvesicles, nucleic acids, lipids, electrolytes, urea, urobilin, pharmaceutical drugs, mucous, bacteria, and other microorganisms, and combinations thereof. In some embodiments, the sample purifier separates components of a biological sample disclosed herein. In some embodiments, the sample purifier disclosed herein removes one or more components of a sample that would inhibit, interfere with or otherwise be detrimental to the analyses of the target analyte. In some embodiments, the resulting modified sample is enriched for the target analyte. This is considered indirect enrichment of target analytes. Alternatively or additionally, target analytes may be captured directly, which is considered direct enrichment of target analytes.

In some embodiments, sample purifiers disclosed herein comprise a filter. In some embodiments, sample purifiers disclosed herein comprise a membrane. Generally the filter or membrane is capable of separating or removing cells, cell particles, cell fragments, blood components other than cell-free nucleic acids, or a combination thereof, from the biological samples disclosed herein.

In some embodiments, the sample purifier facilitates separation of plasma or serum from cellular components of a blood sample. In some embodiments, the sample purifier facilitates separation of plasma or serum from cellular components of a blood sample before starting a molecular amplification reaction or a sequencing reaction. Plasma or serum separation is achieved by several different methods such as centrifugation, sedimentation or filtration. In some embodiments, the sample purifier comprises a filter matrix for receiving whole blood, the filter matrix having a pore size that is prohibitive for cells to pass through, while plasma or serum can pass through the filter matrix uninhibited. In some embodiments, the filter matrix combines a large pore size at the top with a small pore size at the bottom of the filter, which leads to very gentle treatment of the cells preventing cell degradation or lysis, during the filtration process. This is advantageous because cell degradation or lysis would result in release of nucleic acids from blood cells or maternal cells that would contaminate target cell-free nucleic acids. Non-limiting examples of such filters include Pall Vivid™ GR membrane, Munktell Ahlstrom filter paper, and TeraPore filters.

In some embodiments a vertical filtration system is used to facilitate separation of plasma or serum from a cellular component of a blood sample. In this instance, the filtration is driven by capillary force to separate a component or fraction from a sample (e.g., plasma from blood). By way of non-limiting example, vertical filtration may comprise gravitation assisted plasma separation. A high-efficiency superhydrophobic plasma separator is described, e.g., by Liu et al., A High Efficiency Superhydrophobic Plasma Separation, Lab Chip 2015.

In some embodiments, the sample purifier comprises a lateral filter (e.g., sample does not move in a gravitational direction or the sample moves perpendicular to a gravitational direction). The sample purifier may comprise a vertical filter (e.g., sample moves in a gravitational direction). The sample purifier may comprise vertical filter and a lateral filter. The sample purifier may be configured to receive a sample or portion thereof with a vertical filter, followed by a lateral filter. The sample purifier may be configured to receive a sample or portion thereof with a lateral filter, followed by a vertical filter. In some embodiments, a vertical filter comprises a filter matrix. In some embodiments, the filter matrix of the vertical filter comprises a pore with a pore size that is prohibitive for cells to pass through, while plasma can pass the filter matrix uninhibited. In some embodiments, the filter matrix comprises a membrane that is especially suited for this application because it combines a large pore size at the top with a small pore size at the bottom of the filter, which leads to very gentle treatment of the cells preventing cell degradation during the filtration process.

In some embodiments, the filter comprises a material that moves, draws, pushes, or pulls the biological sample through the filter. In some embodiments, the material is a wicking material. Examples of appropriate materials used in the sample purifier to remove cells include, but are not limited to, polyvinylidene difluoride, polytetrafluoroethylene, acetylcellulose, nitrocellulose, polycarbonate, polyethylene terephthalate, polyethylene, polypropylene, glass fiber, borosilicate, vinyl chloride, or silver. In some embodiments, the separation material is a hydrophobic filter, for example a glass fiber filter, a composite filter, for example Cytosep (e.g., Ahlstrom Filtration or Pall Specialty Materials, Port Washington, N.Y.), or a hydrophilic filter, for example cellulose (e.g., Pall Specialty Materials). In some embodiments, whole blood is fractionated into red blood cells, white blood cells and serum components for further processing according to the methods of the present disclosure using a commercially available kit (e.g., Arrayit Blood Card Serum Isolation Kit, Cat. ABCS, Arrayit Corporation, Sunnyvale, Calif.).

In some embodiments, the sample purifier comprises at least one filter or at least one membrane characterized by at least one pore size. In some embodiments, at least one pore size of at least one filter is about 0.05 microns to about 10 microns. In some embodiments, the pore size is about 0.05 microns to about 8 microns. In some embodiments, the pore size is about 0.05 microns to about 6 microns. In some embodiments, the pore size is about 0.05 microns to about 4 microns. In some embodiments, the pore size is about 0.05 microns to about 2 microns. In some embodiments, the pore size is about 0.05 microns to about 1 micron. In some embodiments, at least one pore size of at least one filter is about 0.1 microns to about 10 microns. In some embodiments, the pore size is about 0.1 microns to about 8 microns. In some embodiments, the pore size is about 0.1 microns to about 6 microns. In some embodiments, the pore size is about 0.1 microns to about 4 microns. In some embodiments, the pore size is about 0.1 microns to about 2 microns. In some embodiments, the pore size is about 0.1 microns to about 1 micron.

In some embodiments, the sample processor is configured to separate blood cells from whole blood. In some embodiments, the sample processor is configured to isolate plasma from whole blood. In some embodiments, the sample processor is configured to isolate serum from whole blood. In some embodiments, the sample processor is configured to isolate plasma or serum from less than 1 milliliter of whole blood. In some embodiments, the sample processor is configured to isolate plasma or serum from less than 1 milliliter of whole blood. In some embodiments, the sample processor is configured to isolate plasma or serum from less than 500 microliters (μL) of whole blood. In some embodiments, the sample processor is configured to isolate plasma or serum from less than 400 μL of whole blood. In some embodiments, the sample processor is configured to isolate plasma or serum from less than 300 μL of whole blood. In some embodiments, the sample processor is configured to isolate plasma or serum from less than 200 μL of whole blood. In some embodiments, the sample processor is configured to isolate plasma or serum from less than 150 μL of whole blood. In some embodiments, the sample processor is configured to isolate plasma or serum from less than 100 μL of whole blood.

Disclosed herein, in some embodiments, are devices comprising a detection zone. In some embodiments, the detection zone comprises a test region and a control region. In some embodiments, the imaging device captures an image of the detection zone. In some embodiments, the control region is a positive control. In some embodiments, the control region is a negative control. In some embodiments, the control region comprises a positive and a negative control.

In some embodiments, multiple test regions are provided in the detection zone. In some embodiments, the detection zone comprises a first test region comprising a competition assay assembly to measure a presence of neutralizing antibodies in a patient sample against a first SARS-CoV-2 spike protein receptor binding domain (RBD). The first test region, in some embodiments, comprises a first capture molecule described herein. In some embodiments, the first capture molecule comprises an amino acid sequence that is at least 95% identical to SEQ ID NO: 3. In some embodiments, the competition assay assembly further comprises a peptide-conjugate comprising a peptide derived from a cognate receptor of the spike protein (e.g., human ACE2) conjugated to a detectable moiety.

In some embodiments, the detection zone comprises a second test region comprising a second competition assay assembly to measure a presence of neutralizing antibodies in a patient sample against a second SARS-CoV-2 spike protein RBD that differs from the first SARS-CoV-2 spike protein RBD by at least 1 amino acid. In some embodiments, the first SARS-CoV-2 spike protein RBD and the second SARS-CoV-2 spike protein RBD differ by at least 2 amino acids. In some embodiments, the first SARS-CoV-2 spike protein RBD and the second SARS-CoV-2 spike protein RBD differ by at least 3, 4, 5, 6, 7, 8, 9, or 10 amino acids.

In some embodiments, the second test region comprises a second assay assembly configured to measure total antibodies (e.g., neutralizing or otherwise) against SARS-CoV-2 or another virus. In some embodiments, the second assay assembly comprises a capture molecule that binds to anti-nucleocapsid IgG or IgM, anti-spike RBD IgG or IgM, anti-Spike SiS2 IgG or IgM, anti-spike S2 IgG or IgM, anti-spike S1 IgG or IgM, or a combination thereof. In some embodiments, the capture molecule binds to anti-nucleocapsid IgG or IgM for SARS-CoV, anti-S1 IgG or IgM for MERS, anti-H1 IgG or IgM for influenza and anti-H3 IgG or IgM for influenza.

In some embodiments, the testing device comprises multiple detection zones. In some embodiments, the testing device comprises a first detection zone and a second detection zone. In some embodiments, the first detection zone comprises the first and second testing regions described herein. In some embodiments, the first and second detection zones are partially enclosed in a single housing. In some embodiments, the first and second detection zones are not enclosed in a single housing. The testing device of the present disclosure may comprise any number of detection zones or testing regions, depending on the desired output. In some embodiments, the testing device comprises at least 2, 3, 4, 5, 6, 7, 8, 9, or 10 detection zones. In some embodiments, at least one of the detection zones comprises at least 2, 3, 4, 5, 6, 7, 8, 9, or 10 testing regions. In some embodiments, the detection zones are partially enclosed in a single housing. In some embodiments, the detection zones are not enclosed in a single housing.

### Biological Sample Medium

A biological sample medium (BSM) may be used to store a biological sample to be assayed using the testing devices, systems or methods described herein. In some embodiments, the BSM is a dried blood spot card (DBS). Referring to FIG. 12B, in some embodiments, the BSM 1201 may comprise one or more regions 1205 for containing the one or more biological sample(s). In some embodiments, the one or more regions may comprise at least 2, 3, 4, 5, 6, 7, 8, 9, 10, or more regions. In some embodiments, the one or more regions may comprise no more than 2, 3, 4, 5, 6, 7, 8, 9, or 10 regions. In some embodiments, the one or more regions may comprise 2 to 5 regions (e.g., 2, 3, 4, or 5). In some embodiments, the one or more regions 1205 may comprise spatially distinct adjacent regions. In some embodiments, the biological sample(s) stored on the BSM may comprise two or more biological samples. In some embodiments, the one or more regions may comprise at least 2, 3, 4, 5, 6, 7, 8, 9, 10, or more samples. In some embodiments, the one or more regions may comprise no more than 2, 3, 4, 5, 6, 7, 8, 9, or 10 samples. In some embodiments, the one or more regions may comprise 2 to 5 samples (e.g., 2, 3, 4, or 5).

Still referring to FIG. 12B, in some embodiments, the BSM may comprise a sealable member 1207 to protect the one or more regions of one or more deposited biological sample 1205 during transport of the BSM. As shown in FIG. 12A-12B, in some embodiments, the BSM may comprise subject identifying information associated with a barcode 1202, numeric 1203, text-based characters 1204, RFID 1206, or any combination thereof to identify the subject from whom the one or more biological samples originated. As shown in FIG. 12C, in some embodiments, the BSM may comprise a fillable region 1208 for a subject to provide answers to a survey related to the health of the subject, including for example symptoms related to an infection by a pathogen of interest (e.g., SARS-CoV-2).

In some embodiments, the fillable region is positions on a dried spot card (DSC). In some embodiments, the survey may include one or more clinical questions 1210 pertaining to a symptom associated with an infection by a pathogen of interest. In some embodiments, the BSM may comprise a survey specific to coronavirus symptoms. In some embodiments, the associated symptoms may include cough, nasal congestion, chest pain, headache, diarrhea, vomiting, difficulty breathing or any combination thereof. In some embodiments, the survey may comprise a response severity scale 1211 of associated symptoms. In some embodiments, said severity scale may include terminology to indicate symptoms never present, sometimes present, often present, or present every day. In some embodiments, clinical questions may include indicating whether or not the subject has been exposed to a second subject whom has confirmed contracting one or more viruses or microbes. In some embodiments, clinical questions may further comprise a unique computer recognizable code 1209 (e.g. QR code, alpha numeric character, RFID or any combination thereof) that a computer software image processing algorithm may recognize, and associate said medical differential diagnosis survey response with. In some embodiments, a computer software image processing algorithm may further provide a combined interpretation of a subject's assay results in view of survey severity scale responses.

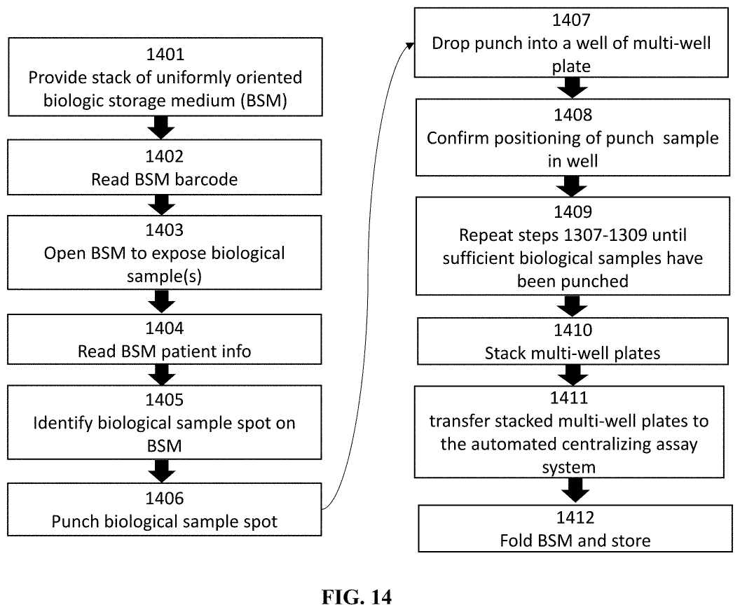

As referred to in FIG. 14, the systems and methods described herein, in some embodiments may perform steps on a BSM including: (a) providing a stack of uniformly oriented BSMs 1401; (b) reading the BSM barcode 1402, (c) opening the BSM to expose biological sample(s) 1403, (d) reading BSM patient info 1404, (e) identifying a region on the BSM 1405, (f) punching the biological sample spot 1406, (g) dropping the punch into a well of a multi-well microtiter plate 1407, (h) confirming the position of punch sample in the well 1408, (i) repeating steps (a)-(h) until sufficient biological samples have been punched 1409, (j) stacking the multi-well microtiter plates 1410, (k) transferring the stacked multi-well microtiter plates to the automated centralizing assay system 1411, (l) folding the BSM for storage 1412 or any combination thereof. One or more of the steps performed by the system may be automated.

### Automated and Centralizing Systems

In some embodiments, the systems described herein are automated high throughput systems. In some embodiments, the systems described may comprise an automated system capable of: (a) receiving and processing a biological sample in preparation for assaying, and (b) conducting the assay. In some embodiments, one or more of (a) or (b) is automated. In some embodiments, (a) and (b) are automated. For example, an automated system may comprise system components operated by one or more robots. In some embodiments, the system components are otherwise in mechanical, electrical, fluidic or optical communication with one another such that human intervention is not necessary to perform the disclosed functions. Such automated systems may be capable of completing the methods disclosed herein with or without the intervention of a human user. In some embodiments, the systems utilize a BSM comprising the biological sample to be assayed with the system.

Biological Sample Processor. As shown in FIG. 13, in some embodiments, the systems disclosed herein comprise a biological sample (BS) processor 1302 and an assay assembly block 1309. In some embodiments, the BS processor comprises: (a) a BS receiver 1303; (b) a BS scanner 1304; (c) a BS separator 1305; (d) BS imager 1306; (e) BS chemical extractor 1307; and (f) a BS purifier 1308. In some embodiments, the biological sample may be contained within a BSM and further processed by the high throughput system.

In some embodiments, the BS receiver 1303 may mechanically manipulate the BS through linear translation or rotation of the BS to properly position the BS for further processing. In some embodiments, the BS receiver comprises a receptacle for a container (e.g., a vial, 96 well plate). In some embodiments, BS receiver is a crate with multiple receptacles for containers. In some embodiments, the BS receiver comprises a mechanical arm with a distal hand-like member configured to hold the container and transfer the container to the BS scanner to be scanned.

In some embodiments, the BS processor 1302 may comprise a BS scanner 1304 in electrical communication with the BS processor 1302. In some embodiments, the BS scanner 1304 may interpret and record subject identifying information on the BS. In some embodiments, the BS scanner 1304 may scan identifying information provided in, for example, barcode 1202, numeric 1203, text-based characters 1204, RFID 1206 or any combination thereof, as shown in FIG. 12A.

In some embodiments, the BS processor 1302 may further comprise a BS separator 1305 capable of mechanically separating a segment of the BS. In the case where the BS is stored on a BSM, such as a dried blood spot card, the BS separator 1305 may comprise a cutting tool that isolates a segment of the BSM containing the BS. In some embodiments, the cutting tool may comprise a knife or a punch. In some embodiments, the punch comprises a plurality of diameters. In some embodiments, the punch outer-diameter may be at least 2, 3, 4, 5, 6, 7, 8, 9, 10 or more millimeters. In some embodiments, the punch outer-diameter may comprise no more than 2, 3, 4, 5, 6, 7, 8, 9, or 10 millimeters. In some embodiments, the one or more punched segments of BS may be placed into an assay chamber for further processing. In some embodiments, such as when the BS is stored in a container (e.g., a vial), the BS separator may be a syringe or a needle for isolating a portion of the BS to be assayed. In some embodiments, the BS separator 1305 may aspirate a segment of the BS using a needle. In some embodiments, the needle may be 18 AWG, 19AWG, 20AWG, 21AWG, 22AWG, 23AWG, 24AWG, 25AWG, 26AWG, 27AWG, or 28 AWG. In some embodiments, the one or more aspirated segment of BS may be placed into an assay chamber for further processing the BS. In some embodiments, the BS processor 1302 may further comprise a BS imager 1306 that may provide quality control feedback position of the BS in the assay chamber to ensure proper processing. After proper placement of one or more segments of BS in the assay chamber, the BS may then be catalogued and stored to be referenced in the future.

In some embodiments, the assay chamber may comprise a well plate. In some embodiments, the well plate may be a 96-well, 384-well, or a 1536-well plate. In some embodiments, the well plate may be a microtiter well plate. In some embodiments, the assay chamber comprises a unique identifier that may be recognized as it moves between the biological sample processor 1302 and the assay assembly block 1309 described herein. In some embodiments, after successful placement of the one or more BS segments in the assay chamber, the BS may be further processed by the biological sample processor 1302 in preparation for assaying

Biological Sample Preparation. Biological sample preparation, in some embodiments, is accomplished by a (a) BS chemical extractor 1307 and (b) a BS purifier 1308. Suitable buffers that may be used to elute the BS from the BSM includes phosphate-buffered saline (PBS). In some embodiments, the BS chemical extractor 1307 may be capable of eluting the BS to isolate the BS (e.g., from a biological sample medium). In some embodiments, the chemical extractor elutes the BS from a biological sample medium in the assay chamber. In some embodiments, the BS chemical extractor 1307 may be in fluidic communication with one or more reservoirs of elution buffers and the assay chamber to dispense elution buffer as needed into the assay chamber. In some embodiments, the assay chamber containing the elution buffer and BS may be agitated by mechanical shaking to facilitate the elution process. In some embodiments, the eluted BS may be purified by the BS purifier 1308 prior to transferring the assay chamber to the assay assembly block 1309. In some embodiments the purification process may comprise centrifugation to isolate BS by density. In some embodiments, upon sufficient BS processing, the biological sample processor 1302 may mechanically transfer the assay chamber to the assay assembly block 1309.

Assaying the Biological Sample. Assaying the biological sample, in some embodiments, may be accomplished by an assay assembly block 1309. In some embodiments, the assay assembly block comprises: (a) BS Diluter 1310 and (b) BS assay conductor 1311. In some embodiments, the BS diluter 1310 may be in fluidic communication with the assay chamber to prepare the serial dilutions of the BS. In some embodiments, assay chamber identification may be interpreted by the assay assembly block to indicate which assay to conduct on which BS. In some embodiments, upon completion of the assay by the BS assay conductor 1311, the assay results 1312 will be saved with respect to the identification of the assay chamber and more broadly the subject providing the biological sample assayed.

In some embodiments, the system comprises the use of a preprogramed and automated robotics to perform one or more of the steps above, including the assays described herein. In some embodiments, the automated centralizing assay system comprises the use of preprogramed and automated robotics to perform the assay comprising a lateral flow assay, agglutination assay, ELISA, or FRET assay as described herein. In some embodiment, the system is capable of performing at least or about 40,000, 50,000, 60,000, 70,000, 80,000, 90,000, or more than 100,000 tests per day. In some embodiments, the system utilizes small volumes of reagents, such as less than 100 nL, less than 90 nL, less than 80 nL, less than 70 nL, less than 60 nL, or less than 50 nL of reagents. In some embodiments, the system performs the assay (e.g., a solution-based assay) in a vessel, such as a container or a test tube. In some embodiments, the system performs the assay (e.g., a solution-based assay) in an assay plate, such as a 96-well plate, a 384-well plate, or a 1536-well plate.

In some embodiments, the biological sample (e.g., blood from a finger prick) is stored in or on a BSM, such as a DSC, described herein. The biological sample is removed from the BSM and placed into the vessel (e.g., a well of a 96-well plate) to be eluted from the BSM. Next, the eluted biological sample is assayed using an assay (e.g., FRET, ELISA) described herein deposited on a solid surface.

In some embodiments, biological samples are collected from the subjects via the use of blood or sample collection cards. The biological samples is tested by the automated centralizing assay system. In some embodiments, the blood or sample collection card comprises multiple slots for the blood or sample collection. In such instances, the biological sample from each slot is tested separately by a different assay method. For example, a blood collection card (i.e. a blood spot card) comprises five slots that is tested by the automated centralizing assay system performing fives assays such as lateral flow assay, an ELISA, a biomarker panel, a qPCR, and a neutralizing antibody titer assay. In some embodiments, the same assay is performed multiple times (e.g., duplicates) to ensure accuracy of the results. In some embodiments, the assay is performed at least 2, 3, 4, 5, 6, 7, 8, 9, or 10 times. In some embodiments, multiple dilutions are tested, for e.g., 1:40, 1:80, 1:160, and 1:320 dilutions are tested at the same time. In some embodiments, the dilutions tested are one or more of 1:5, 1:10, 1:15, 1:20, 1:25, 1:30, 1:35, 1:40, 1:45, 1:50, 1:55, or 1:60, 1:65, 1:70, 1:75, 1:80, 1:85, 1:90, 1:95, 1:100, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, or 1:250. In some embodiments, at least 2, 3, 4, 5, 6, 7, 8, 9, or 10 different dilutions are tested simultaneously.

In some embodiments, the automated centralizing assay system confirms the efficacy or effectiveness of a vaccine in a population. In some embodiments, the biological samples are collected from a population before and after the population is vaccinated. In some embodiments, additional biological samples are collected at any time frame or frequency described herein to confirm presence and sufficient quantity of neutralizing antibody (induced by the vaccine) in the population. In some embodiment, the automated centralizing assay system can prognose immunity or measure susceptibility of the population to any one of the pathogen described herein by confirming the absence or insufficient quantity of neutralizing antibody in the population. In some embodiments, the automated centralizing assay system confirms presence, absence, or quantity of the neutralizing antibody described herein from a subject's biological sample. In some instances, the automated centralizing assay system prognoses a subject's immunity to any one of the pathogen described herein. In some embodiments, the automated centralizing assay system prognoses a subject's immunity to coronavirus. In some embodiments, the automated centralizing assay system prognoses a subject's immunity to SARS-CoV-2. In some embodiments, the automated centralizing assay system measures the subject's susceptibility to an infection by any one of the pathogens described herein. In some embodiments, the automated centralizing assay system measures the subject's susceptibility to an infection by coronavirus. In some embodiments, the automated centralizing assay system measures the subject's susceptibility to an infection by SARS-CoV-2. In some embodiments, the automated centralizing assay system identifies individuals in a population that respond to a vaccine and individuals in a population that do not respond to a vaccine. In some embodiments, the automated centralizing assay system minimizes or reduces the risk associated with usage of a commercialized vaccine in a population (e.g., by identifying individuals that do not respond to the vaccine). In some embodiments, the automated centralizing assay system identifies neutralizing antibodies as a surrogate marker of protection from a vaccine (e.g., in place of traditional, randomized, placebo-controlled trials). In some embodiments, the automated centralizing assay system classifies vaccines by % efficacy in a population.

In some embodiments, the automated centralizing assay systems provided herein have an accuracy of at least 80%, for example, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or greater. In some embodiments, the automated centralizing assay systems provided herein have a sensitivity of at least 80%, for example, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or greater. In some embodiments, the automated centralizing assay systems provided herein have a specificity of at least 80%, for example, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or greater.

### Computer-Implemented Systems

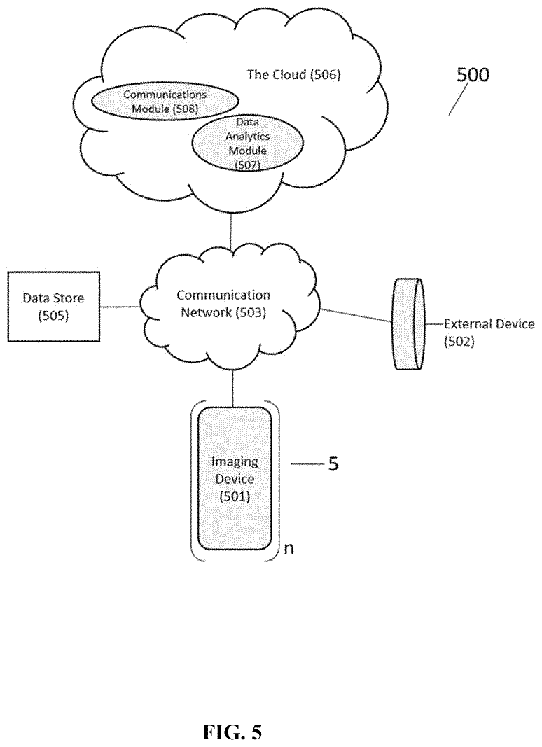

Disclosed herein, in some embodiments, are systems. Referring to FIG. 5, the system 500 comprises, in some embodiments, a testing device, an imaging device 501, and a computing device, for determining whether a subject is immune to an infection by a pathogen of interest (e.g., SARS-CoV-2). The imaging device and/or the computing device are configured to receive and analyze data generated by the testing device and/or one or more external devices, to provide a result to the subject. The result is provided to the subject via a graphical user interface (GUI) by the imaging device and/or computing device using an application (web application or mobile application). Systems further comprise one or more data store for storing and retrieving the data.

Disclosed herein, in some embodiments, are data analyzed by one or more components of the system described herein. In some embodiments, the data are structured or unstructured. In some embodiments, the data are generated by the imaging device when an image is captured of the detection zone of the testing devices described herein. In some embodiments, the data are external data generated from an external device. In some embodiments, the external device comprises a diagnostic device, a prognostic device, or a health or fitness tracking device. In some embodiments, the external data comprises body temperature, heart rate variability, resting heart rate, sleep quality, or sleep quantity, or a combination thereof.

Provided here, in some embodiments, are imaging devices for capturing an image of the detection zone of the testing devices described herein. In some embodiments, the imaging device is a camera. In some embodiments, the imaging device is a computing device with a camera, such as a smartphone, laptop, or tablet. In some embodiments, the imaging device is not a computing device, but is in communication with the computing device via a communication network. In some embodiments, the communication network is wireless, such as wireless Internet or Bluetooth.

Referring to FIG. 5, the system 500 comprises an imaging device 501, an external device 502, a data store 505, all in communication via a communication network 503, equipped with cloud-based computing executed by the data analytics module 507 and communications module 508. In this example, the imaging device transmits data from an image captured of the detection zone of the testing device described herein, via the communication network, to the data analytics module 507 in the cloud 506 to be analyzed. The data analytics module 507 transmits the result to the communications module 508 to be packaged for display to the user. In this example, the result is displayed on the imaging device 501, which is a personal electronic device belonging to the user (e.g., the subject in this example). The data store 505 is a remote server in this example. Alternatively, the data store is a cloud-based data store.

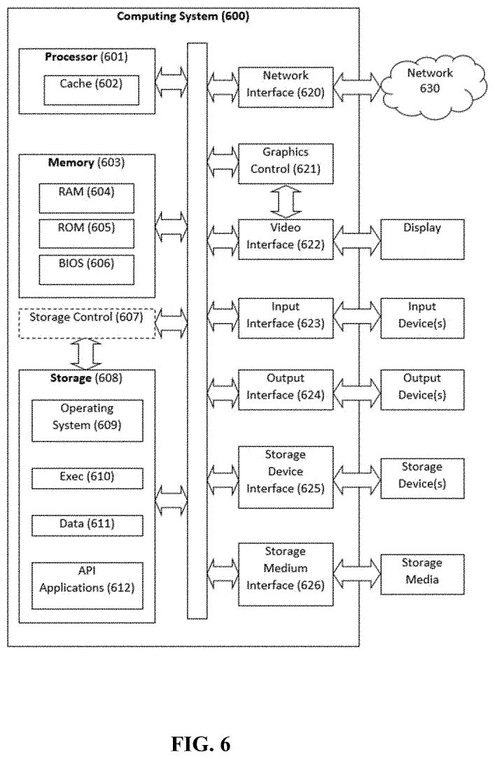

Also provided, in some embodiments, are computing devices comprising a computing system configured to analyze data described herein to provide a result. Referring to FIG. 6, a block diagram is shown depicting an exemplary computing device that includes a computing system 600 (e.g., a processing or computing system) within which a set of instructions can execute for causing a device to perform or execute any one or more of the aspects and/or methodologies for static code scheduling of the present disclosure. The components in FIG. 6 are examples only and do not limit the scope of use or functionality of any hardware, software, embedded logic component, or a combination of two or more such components implementing particular embodiments.

Computer system 600 can include one or more processors 601, a memory 603, and a storage 608 that communicate with each other, and with other components, via a bus 640. The bus 640 can also link a display 632, one or more input devices 633 (which may, for example, include a keypad, a keyboard, a mouse, a stylus, etc.), one or more output devices 634, one or more storage devices 635, and various tangible storage media 636. All of these elements can interface directly or via one or more interfaces or adaptors to the bus 640. For instance, the various tangible storage media 636 can interface with the bus 640 via storage medium interface 626. Computer system 600 can have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 600 includes one or more processor(s) 601 (e.g., central processing units (CPUs) or general purpose graphics processing units (GPGPUs)) that carry out functions. Processor(s) 601 optionally contains a cache memory unit 602 for temporary local storage of instructions, data, or computer addresses. Processor(s) 601 are configured to assist in execution of computer readable instructions. Computer system 600 can provide functionality for the components depicted in FIG. 6 as a result of the processor(s) 601 executing non-transitory, processor-executable instructions embodied in one or more tangible computer-readable storage media, such as memory 603, storage 608, storage devices 635, and/or storage medium 636. The computer-readable media can store software that implements particular embodiments, and processor(s) 601 can execute the software. Memory 603 can read the software from one or more other computer-readable media (such as mass storage device(s) 635, 636) or from one or more other sources through a suitable interface, such as network interface 620. The software can cause processor(s) 601 to carry out one or more processes or one or more steps of one or more processes described or illustrated herein. Carrying out such processes or steps can include defining data structures stored in memory 603 and modifying the data structures as directed by the software.

The memory 603 can include various components (e.g., machine readable media) including, but not limited to, a random access memory component (e.g., RAM 604) (e.g., static RAM (SRAM), dynamic RAM (DRAM), ferroelectric random access memory (FRAM), phase-change random access memory (PRAM), etc.), a read-only memory component (e.g., ROM 605), and any combinations thereof. ROM 605 can act to communicate data and instructions unidirectionally to processor(s) 601, and RAM 604 can act to communicate data and instructions bidirectionally with processor(s) 601. ROM 605 and RAM 604 can include any suitable tangible computer-readable media described below. In one example, a basic input/output system 606 (BIOS), including basic routines that help to transfer information between elements within computer system 600, such as during start-up, is stored in the memory 603.

Fixed storage 608 is connected bidirectionally to processor(s) 601, optionally through storage control unit 607. Fixed storage 608 provides additional data storage capacity and can also include any suitable tangible computer-readable media described herein. Storage 608 is used to store operating system 609, executable(s) 610, data 611, applications 612 (application programs), and the like. Storage 608 can also include an optical disk drive, a solid-state memory device (e.g., flash-based systems), or a combination of any of the above. Information in storage 608 may, in appropriate cases, be incorporated as virtual memory in memory 603.

In one example, storage device(s) 635 is removably interfaced with computer system 600 (e.g., via an external port connector (not shown)) via a storage device interface 625. Particularly, storage device(s) 635 and an associated machine-readable medium can provide non-volatile and/or volatile storage of machine-readable instructions, data structures, program modules, and/or other data for the computer system 600. In one example, software can reside, completely or partially, within a machine-readable medium on storage device(s) 635. In another example, software can reside, completely or partially, within processor(s) 601.

Bus 640 connects a wide variety of subsystems. Herein, reference to a bus can encompass one or more digital signal lines serving a common function, where appropriate. Bus 640 is any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 600 can also include an input device 633. In one example, a user of computer system 600 can enter commands and/or other information into the computer system 600 via input device(s) 633. Examples of an input device(s) 633 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 633 is interfaced to bus 640 via any of a variety of input interfaces 623 (e.g., input interface 623) including, but not limited to, serial, parallel, game port, USB, FIREWIRE, THUNDERBOLT, or any combination of the above.

In particular embodiments, when computer system 600 is connected to network 630, computer system 600 can communicate with other devices, specifically mobile devices and enterprise systems, distributed computing systems, cloud storage systems, cloud computing systems, and the like, connected to network 630. Communications to and from computer system 600 is sent through network interface 620. For example, network interface 620 can receive incoming communications (such as requests or responses from other devices) in the form of one or more packets (such as Internet Protocol (IP) packets) from network 630, and computer system 600 can store the incoming communications in memory 603 for processing. Computer system 600 can similarly store outgoing communications (such as requests or responses to other devices) in the form of one or more packets in memory 603 and communicated to network 630 from network interface 620. Processor(s) 601 can access these communication packets stored in memory 603 for processing.

Examples of the network interface 620 include, but are not limited to, a network interface card, a modem, and any combination thereof. Examples of a network 630 or network segment 630 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 630, can employ a wired and/or a wireless mode of communication. In general, any network topology is used.

Information and data is displayed through a display 632. Examples of a display 632 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 632 can interface to the processor(s) 601, memory 603, and fixed storage 608, as well as other devices, such as input device(s) 633, via the bus 640. The display 632 is linked to the bus 640 via a video interface 622, and transport of data between the display 632 and the bus 640 is controlled via the graphics control 621. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 632, computer system 600 can include one or more other peripheral output devices 634 including, but not limited to, an audio speaker, a printer, a storage device, and any combinations thereof. Such peripheral output devices is connected to the bus 640 via an output interface 624. Examples of an output interface 624 include, but are not limited to, a serial port, a parallel connection, a USB port, a FIREWIRE port, a THUNDERBOLT port, and any combinations thereof.

In addition or as an alternative, computer system 600 can provide functionality as a result of logic hardwired or otherwise embodied in a circuit, which can operate in place of or together with software to execute one or more processes or one or more steps of one or more processes described or illustrated herein. Reference to software in this disclosure can encompass logic, and reference to logic can encompass software. Moreover, reference to a computer-readable medium can encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein is implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein is implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor is a microprocessor, but in the alternative, the processor is any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein is embodied directly in hardware, in a software module executed by one or more processor(s), or in a combination of the two. A software module can reside in RAM memory, flash memory, ROM memory, EPROM memory, EEPROM memory, registers, hard disk, a removable disk, a CD-ROM, or any other form of storage medium known in the art. An exemplary storage medium is coupled to the processor such that the processor can read information from, and write information to, the storage medium. In the alternative, the storage medium is integral to the processor. The processor and the storage medium can reside in an ASIC. The ASIC can reside in a user terminal. In the alternative, the processor and the storage medium can reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, smart phones, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art. In some embodiments, the smart phone is an Apple iPhone or an android device (e.g., Samsung Galaxy).

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360), Microsoft Xbox One, Nintendo® Wii®, Nintendo® Wii U®, and Ouya®.

Disclosed herein are computing systems comprising a data processor. In some embodiments, the data processor is a mobile processor. In some embodiments, the data processor is configured to receive data from an imaging device, an external device, or a data store, or a combination thereof. In some embodiments, data processor analyzes the data to provide a result. In some embodiments, the imaging device and the data processor are housed in the same device, such as a smartphone.

In some embodiments, the data processor is configured to provide a computer program or application, comprising a data analytics module. In some embodiments, the application is a web application. In some embodiments, the application is a mobile application. In some embodiments, the data analytics module is configured to receive data from an imaging device and analyze the data to provide a result. In some embodiments, the data analytics module is configured to receive external data from an external device. In some embodiments, the external data comprises body temperature, heart rate variability, resting heart rate, sleep quality, or sleep quantity, or a combination thereof. In some embodiments, the external device comprises a diagnostic device, a prognostic device, or a health or fitness tracking device. In some embodiments, the health tracking device is the Aurora®, Fitbit®, or Apple Watch. In some embodiments, systems comprise multiple external devices. In some embodiments, the data analytics module is configured to analyze the external data to provide a result. In some embodiments, the data analytics module is configured to analyze the external data and the data received from the imaging device to provide a result.

In some embodiments, the mobile application comprises a communication module. In some embodiments, the communication module is configured to communicate the result to the subject. In some embodiments, the communication module is configured to display the result to the subject via a graphical user interface (GUI) of an electronic device. In some embodiments, the electronic device is the imaging device described herein, the computing device described herein, or a combination thereof. In some embodiments, the electronic device is a smartphone, such as those described herein.

### Computer Program

Disclosed herein, in some embodiments, the data processor is configured to run a computer program. A computer program includes a sequence of instructions, executable by one or more processor(s) of the computing device's CPU, written to perform a specified task. Computer readable instructions is implemented as program modules, such as functions, objects, Application Programming Interfaces (APIs), computing data structures, and the like, that perform particular tasks or implement particular abstract data types. In light of the disclosure provided herein, those of skill in the art will recognize that a computer program is written in various versions of various languages.

The functionality of the computer readable instructions is combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof

In some embodiments, the computer programs described herein are configured to perform any one of the methods described herein. In some embodiments, the computer program is configured to analyze data obtained from an assay described herein (e.g., lateral flow assay, FRET assay) and provide a result based on the analysis of the data.

In some embodiments, the computer program is configured to confirm a the presence, absence, or quantity of the neutralizing antibody described herein from a subject's biological sample. In some instances, the computer program is configured to prognose a subject's immunity to any one of the pathogen described herein. In some embodiments, the computer program is configured to prognose a subject's immunity to coronavirus. In some embodiments, the computer program is configured to prognose a subject's immunity to SARS-CoV-2. In some embodiments, the computer program is configured to measure a subject's susceptibility to an infection by any one of the pathogen described herein. In some embodiments, computer program is configured to measure a subject's susceptibility to an infection by coronavirus. In some embodiments, the computer program is configured to measure a subject's susceptibility to an infection by SARS-CoV-2. In some embodiments, the computer program is configured to identify individuals in a population that respond to a vaccine and individuals in a population that do not respond to a vaccine. In some embodiments, the computer program is configured to identify neutralizing antibodies as a surrogate marker of protection from a vaccine. In some embodiments, the computer program is configured to classify a vaccine by % efficacy in a population. In some embodiments, the computer program is configured to minimize or reduce the risk associated with vaccine usage following market entry (e.g., by identifying individuals that do not respond to the vaccine).

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media

### Web Application

Disclosed here, in some embodiments, are data processors comprising one or more web applications. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Javam, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®

### Mobile Application

Also disclosed herein, in some embodiments, are data processors comprising one or more mobile applications. In some embodiments, the mobile application is provided to a mobile digital processing device at the time it is manufactured. In some embodiments, the mobile application is provided to a mobile digital processing device via the computer network described herein. Mobile applications disclosed herein are configured to locate, encrypt, index, and/or access information. Mobile applications disclosed herein are configured to acquire, encrypt, create, manipulate, index, and peruse data.

A mobile application is created by suitable techniques using hardware, languages, and development environments known to the art. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, and Samsung® Apps.

### Standalone Application

Disclosed here, in some embodiments, are data processors comprising one or more standalone applications. A standalone application is an independent computer process; not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Software Modules

Disclosed herein, in some embodiments, the data processor comprises a computer program configured with one or more software modules. In some embodiments, the one or more software module is a data analytics module. In some embodiments, the one or more software module is a communication module.

In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location

In some embodiments, the computing system described herein comprise a data analytics module. In some embodiments, the data analytics module is configured to run one or more algorithms. In some embodiments, the one or more algorithms comprise a machine learning algorithm. The machine learning algorithm is capable of supervised learning, unsupervised learning, reinforcement learning, semi-supervised learning, self-supervised learning, multi-instance learning, inductive learning, deductive inference, transduction learning, multi-task learning, active learning, online learning, transfer learning, or ensemble learning. In some embodiments, the data analytics module is configured with artificial intelligence (AI), such as a limited memory AI.

In some embodiments, the data analytics module receives data from the testing device, and optionally, external data from one or more external devices, and analyzes the data to provide a result. Referring to analysis of the data received from the testing device, the data analytics module normalizes the result by subtracting background signal intensity of the testing device (e.g., background plasma concentration, non-specific binding to the solid surface). Referring to external data, the data analytics module identifies whether external data indicates symptoms to an acute infection by a pathogen.

In some embodiments, the result is either positive or negative. In some embodiments, the positive result indicates an acute infection and negative result indicates a lack of an acute infection. In some embodiments, the positive result indicates that the subject is immune to a future infection by a pathogen of interest. In some embodiments, the data analytics module transmits the result to a communications module for display to the subject.

In some embodiments, the communication module is configured to display one or more results to the subject via a graphical user interface (GUI).

### Data Store

Disclosed herein, in some embodiments, are systems comprising one or more data stores. In some embodiments, the data store is a database suitable for the storage and retrieval of data. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices, such as a smartphone.

### Graphical User Interface

Also disclosed herein, in some embodiments, are graphical user interfaces (GUI) configured to display a result to a user. In some embodiments, the user is the subject. In some embodiments, the GUI comprises one or more dashboards of an application (e.g., web application or mobile application). In some embodiments, the dashboard comprises relevant health information to a subject. In some embodiments, the GUI is a part of personal electronic device, such as a smartphone or tablet, belonging to the user.

### Web Portal

Disclosed herein, in some embodiments, is a web portal providing a single access point for multiple users to access information about the immune status of a subject. In some embodiments, a web portal provides access to a subject, a subject's doctor, or a healthcare worker responding to an urgent public health crisis. In some embodiments, the portal provides a single access point for a population of individuals, wherein the data is anonymized. In this example, the web portal provides access to policy makers, health care professionals, governmental organization, and non-governmental organizations responding to a public health crisis. Among other information, the portal can indicate one or more geographical locations comprising subjects that are either acutely infected by a pathogen of interest, or immune to the pathogen of interest.

### Methods

Disclosed herein, in some embodiments, are methods of measuring a target analyte disclosed herein in biological sample obtained from subject. In some embodiments, a subject may be a member of a variety of races. For example, the subject may be of the White, Black, Hispanic, or Asian race. In some embodiments, a subject may be of a variety of ages. For example, the subject may be age 0 to age 18, age 18 to age 65, or age 65 and above. In some embodiments, a subject may be a male or a female. In some embodiments, a subject may be pregnant, breastfeeding, or may have priorly been infected with a coronavirus. In some embodiments, a subject may be a high risk subject and have one or more risk factors related to a coronavirus. In some embodiments, a subject may be obese. In some embodiments, methods comprise utilizing the testing devices described herein. In some embodiments, methods further comprise analyzing data generated by the testing device disclosed herein, and providing a result to a user of an electronic device. In some embodiments, providing the result comprises displaying the result on a GUI of the electronic device. In some embodiments, the analyzing and displaying is performed by a single computing device (e.g., smartphone, tablet). In some embodiments, analyzing and displaying is performed at the point of need (e.g., at the time and space that the analyte is detected in the biological sample using the testing device described herein).

Provided herein are methods of processing a biological sample comprising: (a) providing a biological sample from a subject; (b) measuring a presence, an absence, or a level of a labeled complex between (i) a capture molecule and (ii) a peptide-conjugate comprising a peptide derived from a pathogen in the presence of the biological sample; (c) classifying the biological sample as having a presence or a level of the neutralizing antibody.

In some embodiments, methods further comprise determining whether a subject suffers from an acute infection by the pathogen (e.g., SARS-CoV-2). In some embodiments, methods further comprise determining whether the subject is immune to an infection by the pathogen (e.g., SARS-CoV-2). In some embodiments, methods further comprise determining whether a vaccine administered to the subject is effective to immunize the subject against the pathogen. In some embodiments, the methods further comprise determining whether the biological sample is safe for transfusion into another subject (e.g., blood, or blood plasma transfusion). In some embodiments, the methods further comprise determining whether the biological sample is safe as a convalescent plasma therapy for the prevention of an infection by the pathogen or treatment of a disease or condition associated with the pathogen. In some embodiments, the methods further comprise identifying the subject as having an acute infection by the pathogen of interest. In some embodiments, the methods further comprise identifying a subject in need of a vaccination to the pathogen of interest. The methods described herein may be performed at the point of care or point of need.

In some embodiments, the methods provided herein confirm presence, absence, or quantity of the neutralizing antibody described herein from a subject's biological sample. In some instances, the methods described herein prognose a subject's immunity to any one of the pathogens described herein. In some embodiments, the methods described herein prognose a subject's immunity to coronavirus. In some embodiments, the methods described herein prognose a subject's immunity to SARS-CoV-2. In some embodiments, the methods provided herein measure the subject's susceptibility to an infection by any one of the pathogens described herein. In some embodiments, the methods provided herein measure the subject's susceptibility to an infection by coronavirus. In some embodiments, the methods provided herein measure the subject's susceptibility to an infection by SARS-CoV-2. In some embodiments, the methods provided herein identify individuals in a population that respond to a vaccine and individuals in a population that do not respond to a vaccine. In some embodiments, the methods provided herein minimize or reduce the risk associated with usage of a commercialized vaccine in a population (e.g., by identifying individuals that do not respond to the vaccine). In some embodiments, the methods provided herein identify neutralizing antibodies as a surrogate marker of protection from a vaccine. In some embodiments, the methods provided herein classify vaccines by % efficacy in a population.

In some embodiments, the methods provided herein have an accuracy of at least 80%, for example, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or greater. In some embodiments, the methods provided herein have a sensitivity of at least 80%, for example, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or greater. In some embodiments, the methods provided herein have a specificity of at least 80%, for example, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or greater.

In some embodiments, the pathogen is a virus, a bacterium, a fungus, or a parasite. In some embodiments, the virus is a DNA virus or an RNA virus. In some embodiments, the virus is a single stranded virus, or a double stranded virus. In some embodiments, the virus is a plus strand or a minus strand DNA or RNA virus. In some embodiments, the virus replicates through reverse transcription of an RNA intermediate. In some embodiments, the virus is a coronavirus. In some embodiments, the coronavirus is a severe acute respiratory syndrome coronavirus (SARS-CoV). In some embodiments, the SARS-CoV is SARS-CoV-2. In some embodiments, the coronavirus is Middle East Respiratory Syndrome coronavirus (MERS-CoV). In some embodiments, the coronavirus is an alpha coronavirus (e.g., 229E, NL63). In some embodiments, the coronavirus is a beta coronavirus (e.g., OC43, HKU1).

In some embodiments, the coronavirus comprises a protein having an amino acid sequence with greater than or equal to about 70%, 75%, 80%, 85%, 86%, 87%, 88%, 89%, 90%, 91%, 92%, 93%, 94%, 95%, 96%, 97%, 98%, 99%, or 100% sequence identity to one or more sequences, or portions thereof, provided in SEQ ID NOS: 1-116. In some embodiments, the pathogen of interest is a variant of the pathogen, for example, a pathogen that has evolved through mutations in the genes encoding the pathogen. In the case of a virus, the variant virus may have a mutation in an amino acid sequence of a viral protein, such as an envelope protein, nucleocapsid protein, membrane protein, hemagglutinin, neuraminidase, or spike protein. In some embodiments, the virus (e.g., coronavirus) comprises a mutation in one or more of the sequences provided in SEQ ID NOS: 1-116. In some embodiments, the mutation does not affect, or minimally affects, host receptor binding by the spike protein of the coronavirus. In some embodiments, the mutation is an insertion, a deletion, or a substitution at an amino acid (or a plurality of amino acids) of one or more sequences provided in SEQ ID NOS: 1-116.

In some embodiments, the human is an animal, such as a mammal. In some embodiment, the mammal is a dog, cat, monkey, non-human primate, rodent, or a farm animal. In some embodiments, the rodent is a gerbil, hamster, chinchilla, rat, mouse, or guinea pig. In some embodiments, the farm animal is a horse, a pig, a cow, a sheep, an alpaca, or a lama. In some embodiments, the mammal is a human subject. In some embodiments, the subject is pediatric (e.g., age 0-18). In some embodiments, the subject is not pediatric. In some embodiments, the subject is age 50 or older. In some embodiments, the subject is an age between 50-100, 55-95, 60-90, 65-85, or 70-80. In some embodiments, the subject is female or male. In some embodiments, the subject has been exposed to the pathogen. In some embodiments, the subject has not been exposed to the pathogen. In some embodiments, the subject exhibits one or more symptoms comprising a cough, fever, tiredness, or difficulty breathing. In some embodiments, the subject has an underlying health problem comprising high blood pressure, a heart problem, diabetes, immunodeficiency, autoimmune disease. In some embodiments, the subject is immunocompromised.

Described herein are methods for testing a population using the testing device or systems described herein. In some embodiments, the population comprises subjects of any demographics, age, or geographical locations. In some embodiments, all subjects in the population are vaccinated against the pathogen. In some embodiments, some subjects in the population are vaccinated against the pathogen. In some embodiments, none of the subjects in the population are vaccinated against the pathogen. In some embodiments, the number of vaccinated subjects in the population is unknown. In some embodiments, all subjects in the population are previously infected by the pathogen. In some embodiments, some subjects in the population are previously infected by the pathogen. In some embodiments, none of the subjects in the population are previously infected by the pathogen. In some embodiments, the number of subjects previously infected by the pathogen is unknown. In some embodiments, all subjects in the population are currently infected by the pathogen. In some embodiments, some subjects in the population are currently infected by the pathogen. In some embodiments, none of the subjects in the population are currently infected by the pathogen. In some embodiments, the number of subjects currently infected by the pathogen is unknown. In some embodiments, the population is a general population. In some embodiments, the population is a population undergoing clinical trial, where all subjects of the population are vaccinated or unvaccinated. In some embodiments, the population is a population that is at higher risk of the infection. For example, the population that is at higher risk of the infection includes healthcare workers, essential workers, elderly, subjects with pre-existing condition, subjects with respiratory disease or disorder, or subjects with comorbidity or any of the health problems described herein. In some embodiments, the population comprises individuals who are vaccine responders (e.g., develop neutralizing antibodies as described herein in response to the vaccine). In some embodiments, the population comprises individuals who are vaccine non-responders (e.g., do not develop neutralizing antibodies as described herein in response to the vaccine). In some embodiments, the population comprises individuals who have been infected and/or exposed to the natural pathogen.

In some embodiments, obtaining the biological sample from the subject or population of subjects is direct or indirect. Indirectly obtaining a biological sample from a subject may include receiving it from a laboratory or processing/storage facility by mail, or otherwise. In some embodiments, indirectly obtaining the biological sample from the subject or the population comprises the use of blood collection means such as blood card. In some embodiments, the blood card comprises multiple slots for collecting multiple samples of blood droplets for multiple testing. For example, a biological sample medium (BSM) (e.g., a dried blood spot card) comprises multiple slots is tested by the automated centralizing assay system performing assays such as control assay to verify the quality of the biological sample comprising the blood, lateral flow assay, ELISA, biomarker panel, qPCR, western blotting, and a neutralizing antibody titer assay. Non-limiting examples of biological samples include cell, tissue, or bodily fluid obtained from the subject. Non-limiting examples of biological samples include aqueous humour, vitreous humour, bile, breast milk, cerebrospinal fluid, endolymph, perilymph, female ejaculate, amniotic fluid, gastric juice, menses, mucus, peritoneal fluid, pleural fluid, saliva, sebum, semen, sweat, tears, vaginal secretion, vomit, urine, feces, whole blood, blood serum, blood plasma, sputum, cerebrospinal fluid, synovial fluid, lymphatic fluid, nasal swab, or cheek swab. In some embodiments, the biological sample comprises blood, urine, saliva, or feces. In some embodiments, the blood is capillary blood. In some embodiments, the blood is not venous blood (e.g., from a phlebotomy). In some instances, methods disclosed herein comprise obtaining a blood sample by administering a finger prick. In some embodiments, the biological sample is a swab sample (e.g., buccal swab, nasopharyngeal swab). Directly obtaining the biological sample from the subject, in some embodiments, is performed by a healthcare professional (e.g., nurse or doctor), laboratory technician, or the subject at the point of need or the point of care.

In some embodiment, the target analyte comprises macromolecules such as peptides, proteins, nucleic acids, pathogens, or pathogen particles that can be detected for presence or quantity (i.e. concentration) in a biological sample. In some embodiments, the target analyte is targeted and formed complex with an antigen or with an antibody. In some embodiments, the target analyte comprises an antibody against a pathogen. In some embodiments, the antibody against the pathogen is an antibody against an antigenic peptide derived from the pathogen. In some cases, the antibody is neutralizing antibody induced by an infection. In some instances, the antibody is neutralizing antibody induced by vaccination. As a non-limiting example, the analyte may be an antibody against a portion of the spike protein derived from a coronavirus (e.g., SARS-CoV-2). In some embodiments, the analyte is the activity of the antibody. In some embodiments, the activity is blocking binding between the spike protein of a coronavirus and its cognate receptor (e.g., ACE2). In some embodiments, the analyte is a complex comprising the spike protein bound to the antibody at the receptor binding region of the spike protein. In some embodiments, the antibody belongs to an immunoglobulin class comprising immunoglobulin M, immunoglobulin G, immunoglobulin A, immunoglobulin E, or immunoglobulin D.

In some embodiments, the detectable peptide-conjugate is the peptide-conjugate described herein comprising a detection agent and a peptide. In some embodiments, the peptide is an antigenic peptide. In some embodiments, the analyte (e.g., antibody) is specific to at least a portion of the peptide. In some embodiments, the peptide comprises at last a portion of the spike glycoprotein of a coronavirus (e.g., SARS-CoV-2) described herein. In some embodiments, the peptide is a receptor to the antigenic peptide. In some embodiments, the receptor comprises ACE2 receptor described herein.

In some embodiments, measuring comprises performing an assay on the biological sample to detect a number of complexes formed between the analyte and the peptide-conjugate, or the peptide-conjugate and the capture molecule, or both. In some embodiments, the assay comprises an assay assembly described herein. In some embodiments, the assay is a lateral flow assay. In some embodiments, the lateral flow assay is a competition assay. In some embodiments, measuring a number of complexes formed between the analyte and the peptide-conjugate, or the peptide-conjugate and the capture molecule, or both can be performed in one step without the need for washing any portion of the assay assembly.

In some embodiments, the index or the control is derived from a subject that has not been exposed to the pathogen (negative control). In some embodiments, the index or control is from a subject that has been exposed to the pathogen (positive control). In some embodiments, the index or control is obtained from a convalescent plasma donor. In some embodiments, the index or control is synthetically derived. In a non-limiting example of a control that is synthetically derived, the control can be sample with cells engineered to express a peptide or protein that is known to induce production of neutralizing antibodies against the pathogen to a known degree. In some embodiments, the control or index is a maximum absolute dilution of a reference sample in which neutralizing activity is observed in the reference sample. The higher the dilution, the stronger the neutralizing activity. In a non-limiting example, the dilution of 1:160 may be considered sufficient neutralizing activity and anything above 1:160 is considered to be high neutralizing activity. In another example, the dilution of 1:40 may be considered to be low or weak neutralizing activity. In this example, neutralizing activity in the diluted reference sample is a correlate of the level of neutralizing antibodies titers in the reference sample.

In some embodiments, if the level of neutralizing antibodies detected in the biological sample using the testing devices, systems and methods described herein correlates with a dilution that is above a threshold dilution (the control), then the biological sample is classified as having a sufficient presence or level of neutralizing antibodies to confer adaptive immunity against a pathogen. In some embodiments, the biological sample is classified as having a sufficient presence or high level of neutralizing antibodies when the level of neutralizing antibodies correlates with a dilution that is above 1:60, or above or equal to about 1:65, 1:70, 1:75, 1:80, 1:85, 1:90, 1:95, 1:100, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, :375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, or 1:640. In some embodiments, the dilution comprises four (4) dilutions equal to about 1:80, 1:160, 1:320, and 1:640.

Conversely, if the level of neutralizing antibodies detected in the biological sample correlates with a dilution that is below a threshold dilution, then the biological sample is classified as having a low level or insufficient presence of neutralizing antibodies to confer adaptive immunity. In some embodiments, the biological sample is classified as having an insufficient presence or a low level of neutralizing antibodies when the level of the neutralizing antibodies correlates with a dilution that is below or equal to about 1:5, 1:10, 1:15, 1:20, 1:25, 1:30, 1:35, 1:40, 1:45, 1:50, 1:55, or 1:60.

Methods described herein, in some embodiments, do not consist of utilizing a cell culture, such as an immortalized cell line expressing human ACE2. In some embodiments, methods do not consist of handling or administering to a cell or cell line a purified or isolated pathogen, such as a live virus of pseudovirus.

In some embodiments, the methods comprise measuring the target analyte in a biological sample from a subject or in a population of subjects multiple times. In some embodiments, the analyte is measured at least once every 12 hours, 24 hours, 36 hours, two days, three days, four days, five days, six days, one week, two weeks, three weeks, one month, two months, three months, four months, five months, six months, seven months, eight months, nine months, ten months, 11 months, one year, 18 months, two years, three years, four years, five years, six years, seven years, eight years, nine years, decade, or a combination thereof. In some embodiments, the analyte is measured at least 1, 2, 3, 4, 5, 6, 7, 8, 9, 10, 11, 12, 13, 14, 15, 16, 17, 18, 19, 20 or more times. In some embodiments, the analyte is measured at least 1, 2, 3, 4, 5, 6, 7, 8, 9, 10, 11, or 12 months following an event. In some embodiments, the event is an exposure to the pathogen, a first symptom of a disease or condition caused by the pathogen, a first dose of a vaccine against the pathogen, or a first dose of an anti-viral therapy to treat an acute infection by the pathogen. In some embodiments, the analyte is measured at least 1, 2, 3, 4, 5, 6, 7, 8, 9, or 10 years following the event. In some embodiments, the analyte is measured before the event, such as, for example before a first exposure to the pathogen to identify a subject or population of subjects who do not have sufficient immunity against the pathogen and who may be in need of a vaccine.

### Convalescent Plasma Therapy

The use of convalescent plasma for therapy to prevent SARS-CoV-2 infection requires demonstration that the plasma contains sufficient titers of neutralizing antibodies to confer immunity to SARS-CoV-2. Existing methods of detecting sufficient neutralizing antibody titers for this purpose are limited at the point of need because they require use of an immortalized cell line expressing human angiotensin converting enzyme II (ACE2), e.g., 293T cells. By contrast, the testing devices described herein are portable and methods of using the testing devices described herein are cell-free (e.g., do not require use of a cell or cell line). In this manner, the testing devices are more cost effective, do not require a laboratory technician, and is scaled quickly to meet the growing demand of individuals in need of convalescent plasma therapeutic interventions.

Disclosed herein, in some embodiments are methods of screening a biological sample for use as a convalescent plasma therapy, the method comprising: (a) providing a biological sample from a donor subject; (b) measuring a presence, an absence, or a level of a labeled complex between at least two of (i) a capture molecule, (ii) a peptide-conjugate comprising a peptide derived from a pathogen, and (ii) a neutralizing antibody against the pathogen; (c) classifying the biological sample as having a presence or a level of the neutralizing antibody; and (d) identifying the biological sample as safe or not safe as a convalescent therapy based at least in part on the classifying in (c). In some embodiments, the level is high relative to an index or a control. In some embodiments, the biological sample is identified as safe for convalescent therapy provided the presence or the level of the labeled complex between the peptide-conjugate and the neutralizing antibody against the pathogen is measured. In some embodiments, the biological sample is identified as unsafe for convalescent therapy provided the presence or the level of the labeled complex between the peptide-conjugate and the capture molecule is measured.

### Detecting Serological Antibodies and Neutralizing Antibodies

Disclosed herein, in some embodiments, are combined methods of identifying adaptive immunity to a pathogen in a subject and identifying the total antibodies against the pathogen that the subject is producing due to a past exposure. In some embodiments, the method comprises: (a) providing a biological sample from a subject in need thereof; (b) measuring a presence, or a level of a labeled complex between at least two of (i) a capture molecule, (ii) a peptide-conjugate comprising a peptide derived from a pathogen, and (iii) a neutralizing antibody against the pathogen; (c) measuring a presence, an absence or a level of an antibody against the pathogen (e.g., neutralizing or otherwise); (d) classifying the biological sample as having a presence or a level of the neutralizing antibody and/or a presence or a level of a type of antibody (e.g., IgG, IgM, IgA) against the pathogen; and (d) identifying the subject as having sufficient adaptive immunity against the pathogen, based at least in part, on classifying in (c); and/or (e) identifying the subject as having been exposed to the pathogen prior based on classifying in (d). In some embodiments, if the number of the labeled complexes between (ii) and (iii) is high relative to an index or a control, then the subject has a sufficient adaptive immunity to an infection by the pathogen. In some embodiments, if the number of the complexes between (i) and (ii) is high relative to an index or a control, then the subject has a sufficient adaptive immunity to the infection by the pathogen. In some embodiments, the methods described herein are performed using the testing device of the present disclosure. In some embodiments, measuring a presence, an absence or a level of an antibody against the pathogen (e.g., neutralizing or otherwise) can be performed in one step without the need for washing any portion of the testing device. In some embodiments, the capture molecule or peptide-conjugate may comprise a peptide or protein that is encoded by a nucleic acid sequence, or that has an amino acid sequence, that is at least 80% identical to any one of SEQ ID NOS: 21-23, 25-42, 44-46, 49-66, 68-70, 73-90, 92-94, or 97-114.

The presence, absence, or the level of the antibody against the pathogen may be measured by detecting a complex between the antibody (e.g., IgG, IgM, IgA) and a capture molecule that is directly or indirectly labeled. In some embodiments, a level of the antibody that is high, relative to an index or a control is indicative that the subject was exposed to the pathogen. In contrast, if the level of the antibody is low relative to the index or control, the subject was not exposed to the pathogen.

In some embodiments, a presence, absence or a level of a biomarker is measured in the biological sample. In some embodiments, the biomarker is a marker of inflammation, such as a cytokine (e.g., interleukin 6). In some embodiments, the presence, absence or level of multiple biomarkers is measured in addition to the total antibodies and neutralizing antibodies against the pathogen. In some embodiments, the pathogen is SARS-CoV-2, or a variant thereof.

### Neutralizing Antibody (Neutralization) Titer

Described, in some embodiments, are methods for measuring neutralizing antibody titer for determining sufficient quantity of neutralizing antibody to confer adaptive immunity against any one of the pathogens described herein. In some embodiments, a biological sample of neutralizing antibody to be tested is serial-diluted and mixed with a viral suspension. The mixture is applied to known number of cells for inoculation. In some embodiments, the neutralizing antibody titer is determined by calculating the highest dilution of biological sample of neutralizing antibody that prevents infection of at least 30%, 40%, 50%, 60%, 70%, 80%, 90%, 95%, 99% or more of replicate inoculations. In some embodiments, the neutralization titer is determined by calculating the highest dilution of the biological sample of neutralizing antibody that prevents infection at 50% of replicate inoculation. In some cases, the method of measuring neutralization titer comprises the use of viral plaque assay, fluorescent focus assay, and endpoint dilution assay. Each of these three assays relies on serial viral dilutions added to cells to measure neutralization titer. Other exemplary measurements for determining neutralization titer include qPCR or ELISA for quantifying the amount of pathogen genome or particle necessary to infect a set number of cells in the presence of the neutralizing antibody. In some embodiments, the quantity of neutralizing antibody detected in a biological sample is sufficient in inducing adaptive immunity when the concentration of the neutralizing antibody in the biological sample prevents infection at 50% of replicate inoculation. In some embodiments, the quantity of neutralizing antibody detected in a biological sample is insufficient in inducing adaptive immunity when the concentration of the neutralizing antibody in the biological sample prevents infection below 50% of replicate inoculation.

### Population Screening

Described herein, in some embodiments, are methods utilizing the systems, devices, and compositions described herein for screening a population described herein for prognosing immunity or susceptibility to infection caused by any one of the pathogen described herein. In some embodiments, the method comprises detecting presence, absence, or quantity of analyte comprising the neutralizing antibody in the population. In some embodiments, the method prognoses immunity or susceptibility to the infection based on the presence, absence, or quantity of the neutralizing antibody in the biological samples obtained from the subjects in the population. In some embodiments, the method prognoses the population to have adaptive immunity against the infection when the presence or sufficient quantity of the neutralizing antibody is detected in the biological samples. In some embodiments, the method prognoses the subject to have susceptibility or inadequate immunity to the infection when the neutralizing antibody is absent or is detected at an insufficient quantity in biological samples. In some embodiments, the method identifies the subjects with absence or insufficient quantity of neutralizing antibody as subjects who are in need of vaccination. In some embodiments, the population is screened at any one of the time frames or frequencies described herein.

In some embodiments, the method determines if the population is in need of vaccination against the infection based on the presence, absence, or quantity of the neutralizing antibody in the biological samples. In some embodiments, the method determines that the population does not need to be vaccinated due to presence or sufficient quantity of the neutralizing antibody detected in the biological samples. In some embodiments, the method determines that the population does not need to be vaccinated due to herd immunity when the percentage of the subjects of the population with presence or sufficient quantity neutralizing antibody is at least 30%, 40%, 50%, 60%, 70%, 80%, 90%, 95%, or 99%. In some embodiments, the method determines that the population or subjects within the population need to be vaccinated due to absence or insufficient quantity of the neutralizing antibody detected in the biological samples.

### Confirmatory Diagnostic for a Vaccine

Described herein, in some embodiments, are confirmatory diagnostic testing devices and methods of their use to determine the efficacy or effectiveness of a vaccine against a pathogen described herein. In some embodiments, methods of determining the efficacy or effectiveness of a vaccine described herein comprise detecting presence, absence, or quantity of neutralizing antibody against the pathogen in a biological sample from a subject that was administered the vaccine.

Disclosed herein, in some embodiments are methods of determining the efficacy or effectiveness of a vaccine against a pathogen, the method comprising: (a) providing a biological sample from a subject in need thereof; (b) measuring a presence, an absence, or a level of a labeled complex between at least two of (i) a capture molecule, (ii) a peptide-conjugate comprising a peptide derived from a pathogen, and (iii) a neutralizing antibody against the pathogen; (c) classifying the biological sample as having a presence or a level of the neutralizing antibody; and (d) identifying the vaccine as effective to induce a sufficient or insufficient adaptive immune response against the pathogen in the subject. In some embodiments, the level is high relative to an index or a control. In some embodiments, the vaccine is effective, provided the presence or the level of the labeled complex between the peptide-conjugate and the neutralizing antibody against the pathogen is measured. In some embodiments, the vaccine is not effective, provided the presence or the level of the labeled complex between the peptide-conjugate and the capture molecule is measured. In some embodiments, methods further comprise measuring a presence, absence or a level of a biomarker described herein. In some embodiments, the biomarker is interleukin 6. In some embodiments, measuring a presence, an absence, or a level of a labeled complex between at least two of (i) a capture molecule, (ii) a peptide-conjugate comprising a peptide derived from a pathogen, and (iii) a neutralizing antibody against the pathogen can be performed in one step without the need for washing.

In some embodiments, methods confirm that the vaccine lacks efficacy or effectiveness in the subject, provided an absence of the neutralizing antibody is detected in the biological sample obtained from the subject that was administered the vaccine. In some embodiments, methods confirm that the vaccine lacks efficacy or effectiveness, provided the level of the neutralizing antibody detected is low relative to an index or a control level. An absence or a low level of the neutralizing antibody is identified when a presence of the labeled complex between the peptide-conjugate and the capture molecule is detected. In some embodiments, methods confirm that the vaccine is effective, provided a presence of the neutralizing antibody is detected in the biological sample obtained from the subject that was administered the vaccine. In some embodiments, methods confirm that the vaccine is effective provided a level of the neutralizing antibody that is detected is high relative to an index or a control level.

In some embodiments, the subject is part of a general population, where not everyone in the general population was or is administered the vaccine. In some embodiments, the testing devices and methods described herein confirm which subjects of the general population are in need of the vaccine by detecting the absence or the low level of the neutralizing antibody in biological samples obtained from the subjects.

In some embodiments, the testing devices and methods described herein measure neutralizing antibodies against a pathogen in a biological sample from a subject prior to enrolling the subject in the clinical trial. In some embodiments, the presence or level of the neutralizing antibody detected in the biological sample excludes the subject from participating in the clinical trial for the vaccine. The absence or level of the neutralizing antibody detected in the biological sample is an inclusion criteria for the subject to enroll in the clinical trial for the vaccine. In some embodiments, the testing devices and methods described herein measure neutralizing antibodies against a pathogen in a biological sample from a subject during the clinical trial for the investigation of the efficacy of the vaccine. In some embodiments, a biological sample from the vaccinated subject is tested using the testing devices and methods described herein when an adverse event is observed during the clinical trial. In some embodiments, the adverse event is correlated with a presence, absence or level of neutralizing antibodies in the biological sample.

In some embodiments, the testing devices and methods described herein confirm efficacy or effectiveness of the vaccine in a population of subjects. In some embodiments, the population being tested for efficacy or effectiveness of the vaccine is a group receiving the vaccine (as opposed to a different group receiving the placebo) in a clinical trial. In some embodiments, the population being tested for efficacy or effectiveness of the vaccine is a group that is outside the clinical trial context, such as after the vaccine is allowed or approved for use in preventing a disease caused by the pathogen. In some embodiments, the testing devices and methods described herein detect presence, absence, or quantity of the neutralizing antibody against a pathogen in a biological sample from each subject in the group before and after the group is administered the vaccine. In some embodiments, the testing devices and methods described herein confirm adaptive immunity in the vaccinated population based on presence or high level of the detected neutralizing antibody in the biological samples obtained from the vaccinated subjects in the population. In some embodiments, the testing devices and methods described herein confirm a lack of sufficient adaptive immunity in the vaccinated population based on absence or low level of the detected neutralizing antibody in the biological samples obtained from the vaccinated subjects in the population. In this instance, a booster of the vaccine may be recommended for at least one subject of the group, or the entire group.

In some embodiments, neutralizing antibodies induced by the vaccine are measured multiple times after administration of the vaccine to determine whether the vaccine is effective to confer immunity against the pathogen (e.g., SARS-CoV-2). In some embodiments, the neutralizing antibodies are measured after administration of the vaccine at least once every 12 hours, 24 hours, 36 hours, two days, three days, four days, five days, six days, one week, two weeks, three weeks, one month, two months, three months, four months, five months, six months, seven months, eight months, nine months, ten months, 11 months, one year, per 18 months, two years, three years, four years, five years, six years, seven years, eight years, nine years, 10 years, or a combination thereof. In some embodiments, the neutralizing antibodies are measured at least 1, 2, 3, 4, 5, 6, 7, 8, 9, 10, 11, 12, 13, 14, 15, 16, 17, 18, 19, 20 or more times. In some embodiments, the neutralizing antibodies are measured at least 1, 2, 3, 4, 5, 6, 7, 8, 9, 10, 11, or 12 months following administration of the vaccine to the subject. In some embodiments, the neutralizing antibodies are measured at least 1, 2, 3, 4, 5, 6, 7, 8, 9, or 10 years following administration of the vaccine to the subject.

### Identifying Vaccine Responders and Non-Responders

In some embodiments, methods are provided using the systems, devices, and compositions described herein for identifying those subjects that respond to a vaccine (e.g., responders) and those subjects that do not respond to a vaccine (e.g., non-responders). In some embodiments, the systems, devices, and compositions described herein may be used to test individuals in a population of individuals for the presence or absence of neutralizing antibodies to identify those individuals that responded to the vaccine (e.g., produced neutralizing antibodies), and those individuals that did not respond to the vaccine (e.g., did not produce neutralizing antibodies). The population may be a group of individuals that received a vaccine against the pathogen (e.g., SARS-CoV-2). The population may be a group of individuals that received a vaccine during a clinical trial to assess vaccine efficacy. The population may be a group of individuals that received a vaccine as part of a large-scale, mass vaccination campaign. In some embodiments, the methods provided herein assess an individual's particularized response to a vaccine. The methods provided herein identify those individuals that did not respond to a particular vaccine, inform those individuals that they did not develop immunity to the pathogen, and inform those individuals that they should seek alternative interventions (e.g., alternative vaccines) and/or start or continue safety precautions (e.g., social distancing, the use of PPE, etc.).

In some embodiments, the methods may involve administering a vaccine to a population of individuals, and testing each of those individuals for the presence or absence of neutralizing antibodies to assess whether the vaccine conferred immunity against the pathogen (e.g., SARS-CoV-2). The individuals, after receiving the vaccine, may submit one or more biological samples to be tested for the presence or absence of neutralizing antibodies as described herein (e.g., by submitting the sample to a centralized testing center). For example, each individual that receives a vaccine may be given a sample collection device (e.g., a blood spot card) for submitting a biological sample for testing. The sample collection device (e.g., blood spot card) may be given to the individual at the time of vaccination.

The individual may collect one or more biological samples (e.g., a finger prick to collect a blood sample) and provide the one or more biological samples in the sample collection device (e.g., blood card). This may be done before and after vaccine administration. For example, the one or more biological samples may be collected at a time point before the individual has been vaccinated to establish a baseline level of neutralizing antibodies. After administration of the vaccine, the one or more biological sample may be collected again in the same manner at least 1 week, at least 2 weeks, at least 3 weeks, at least 4 weeks, or at least 2 months after vaccine administration to determine whether the vaccine induced a sufficient neutralizing antibody titer to confer protection against the pathogen. Generally, the one or more biological samples are taken after sufficient time has elapsed for the individual to develop neutralizing antibodies (e.g., if the individual responds to the vaccine). To assess the durability of the vaccine, one or more biological samples may be collected in the same manner at least 3 months, 4 months, 5 months, 6 months, 7 months, 8 months, 9 months, 10 months, 11 months, 12 months, 2 years, 3 years, 4 years, 5 years, or more following the vaccine administration.

The individual may submit the one or more biological samples in the sample collection device (e.g., blood spot card) to a testing center. The testing center may test the one or more biological samples for the presence or absence of neutralizing antibodies (e.g., using the systems, devices, and compositions described herein), and may identify the individual as either a vaccine responder (e.g., the individual developed neutralizing antibodies) or as a vaccine non-responder (e.g., the individual did not develop neutralizing antibodies). The testing center may provide the individual with a report (or may otherwise inform the individual) indicating the individual's status as a vaccine responder or as a vaccine non-responder. When the individual is identified as a vaccine responder, the individual may be understood to have developed immunity (e.g., the individual responded to the vaccine) to the pathogen (e.g., SARS-CoV-2).

When the individual is identified as a vaccine non-responder (e.g., the individual did not respond to the vaccine), the individual may be understood to not have developed immunity to the pathogen (e.g., SARS-CoV-2). Alternatively, if durability of an initial response weakens over time, the individual may be understood to no longer have sufficient immunity to the pathogen. In some embodiments, an alternative intervention (e.g., an alternative vaccine), may be recommended to the individual. In some embodiments, additional precautions may be recommended to the individual, such as social distancing, sheltering-in-place, quarantining, the use of personal protective equipment (e.g., gloves, face coverings), and the like. In some embodiments, the vaccine has entered the market. In some embodiments, the vaccine is a vaccine in development or a vaccine that has not yet been approved by a regulatory entity, such as the FDA. In some cases, the vaccine is a vaccine that has been licensed for use by the regulatory entity.

### Mitigating Risk During Vaccine Usage in a Population Following Market Entry

In some embodiments, methods are provided herein using the systems, devices, and compositions described herein to mitigate or reduce the risk associated with usage of a commercialized vaccine in a population. The methods provided herein may assess the benefit and the risk of a particular vaccine by identifying the individuals who respond to the vaccine and the individuals who do not respond to the vaccine. In some embodiments, the systems, devices, and compositions described herein may be used to test individuals in a population for the presence or absence of neutralizing antibodies to identify those individuals that responded to a particular vaccine (e.g., produced neutralizing antibodies), and those individuals that did not respond to the vaccine (e.g., did not produce neutralizing antibodies). In some embodiments, the non-responders are identified and informed that they did not develop immunity, thereby removing the risk associated with a particular vaccine. The methods provided herein allow a vaccine developer to leverage the benefits associated with a particular vaccine, including vaccines with limited efficacy, while minimizing or reducing the risks associated with the vaccine. The methods provided herein may be particularly suited for emergent situations in which multiple vaccines are being developed on an accelerated timeline against a pathogen (and may not be subject to the entire FDA review process).

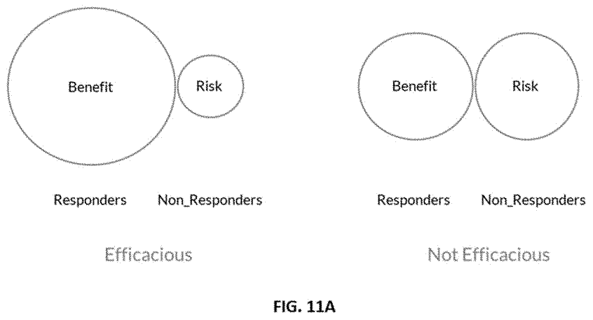

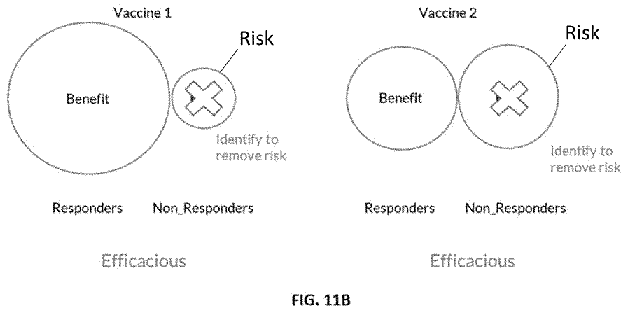

As depicted in FIG. 11A, two vaccines targeting the same pathogen (e.g., SARS-CoV-2) have been developed and licensed for use. Each of the two vaccines may have different levels of efficacy in a population. For example, 70% of the individuals in a population may respond to vaccination with Vaccine 1 (e.g., the “benefit”), whereas 30% of the individuals in the population may not respond to vaccination with Vaccine 1 (e.g., the “risk”). Vaccine 1 may be deemed “efficacious” because the benefit outweighs the risk (e.g., the number of responders is greater than the number of non-responders). Although there are some individuals for which Vaccine 1 was not effective, Vaccine 1 is developed into a vaccine for widespread use against the pathogen (e.g., SARS-CoV-2). In contrast, 40% of the individuals in a population may respond to vaccination with Vaccine 2 (e.g., the “benefit”), and 60% of the individuals in the population may not respond to vaccination with Vaccine 2 (e.g., the “risk”). In this scenario, Vaccine 2 may be deemed “not efficacious” because the risk outweighs the benefit (e.g., the number of non-responders is greater than the number of responders). Despite having a benefit for a subset of the population, Vaccine 2 is not used to prevent infection in a population by the pathogen (e.g., SARS-CoV-2).

In some embodiments, the methods provided herein may be used to identify the risk of a particular vaccine and remove the risk (e.g., by informing the non-responders of their status). As depicted in FIG. 11B, the methods provided herein identify the risk of Vaccine 1 (e.g., the 30% of the population who did not respond to Vaccine 1) and those individuals are informed that they did not develop immunity to the pathogen (SARS-CoV-2). In this scenario, Vaccine 1 is still considered to be “efficacious” and is used. Similarly, the methods provided herein identify the risk of Vaccine 2 (e.g., the 60% of the population who did not respond to Vaccine 2) and those individuals are informed that they did not develop immunity to the pathogen (e.g., SARS-CoV-2). In this scenario, the risk is removed because the non-responders are informed that they do not have immunity to the pathogen (and can take precautionary measures such as social distancing, the use of PPE, and the like). In contrast to the scenario depicted in FIG. 11A, Vaccine 2 is now considered to be “efficacious” and used. Thus, the methods provided herein reduce or remove the risk (e.g., the population of non-responders) associated with a vaccine and increase the chances that a vaccine is used.

The individual may collect one or more biological samples (e.g., a finger prick to collect a blood sample) and provide the one or more biological samples in the sample collection device (e.g., blood card). This may be done before and after vaccine administration. For example, the one or more biological samples may be collected at a time point before the individual has been vaccinated to establish a baseline level of neutralizing antibodies. After administration of the vaccine, the one or more biological sample may be collected again in the same manner at least 1 week, at least 2 weeks, at least 3 weeks, at least 4 weeks, or at least 2 months after vaccine administration to determine whether the vaccine induced a sufficient neutralizing antibody titer to confer protection against the pathogen. Generally, the one or more biological samples are taken after sufficient time has elapsed for the individual to develop neutralizing antibodies (e.g., if the individual responds to the vaccine). To assess the durability of the vaccine, one or more biological samples may be collected in the same manner at least 3 months, 4 months, 5 months, 6 months, 7 months, 8 months, 9 months, 10 months, 11 months, 12 months, 2 years, 3 years, 4 years, 5 years, or more following the vaccine administration.

The individual may submit the one or more biological samples in the sample collection device (e.g., blood spot card) to a testing center. The testing center may test the one or more biological samples for the presence or absence of neutralizing antibodies (e.g., using the systems, devices, and compositions described herein), and may identify the individual as either a vaccine responder (e.g., the individual developed neutralizing antibodies) or as a vaccine non-responder (e.g., the individual did not develop neutralizing antibodies). The testing center may provide the individual with a report (or may otherwise inform the individual) indicating the individual's status as a vaccine responder or as a vaccine non-responder.

When the individual is identified as a vaccine responder, the individual may be understood to have developed immunity (e.g., the individual responded to the vaccine) to the pathogen (e.g., SARS-CoV-2). When the individual is identified as a vaccine non-responder (e.g., the individual did not respond to the vaccine), the individual may be understood to not have developed immunity to the pathogen (e.g., SARS-CoV-2). Alternatively, if the durability of the initial response weakens over time, the individual may be understood to no longer have sufficient immunity to the pathogen. In some embodiments, an alternative intervention, such as an alternative vaccine under development, may be recommended to the individual. In some embodiments, additional precautions may be recommended to the individual, such as social distancing, sheltering-in-place, quarantining, the use of personal protective equipment (e.g., gloves, face coverings), and the like. In some embodiments, the vaccine has entered the market for use. In some embodiments, the vaccine is a vaccine in development or a vaccine that has not yet been approved by a regulatory entity (e.g., the FDA). In some cases, the vaccine is a vaccine that has been licensed for use by the regulatory entity.

### Classifying Vaccines in a Population of Individuals

In some embodiments, methods are provided using the systems, devices, and compositions described herein to classify a vaccine in a population of individuals. In some embodiments, the methods comprise (a) providing a biological sample from each individual of a population of individuals; (b) measuring a presence, an absence, or a level of a labeled complex between at least two of (i) a capture molecule, (ii) a peptide-conjugate comprising a peptide derived from a pathogen, and (iii) a neutralizing antibody against the pathogen in the biological sample; (c) classifying each biological sample as having a presence, an absence, or a level of the neutralizing antibody; and (d) classifying the vaccine based on the presence or the level of the neutralizing antibodies measured in (b). In some embodiments, the vaccine is classified by % efficacy in the population (e.g., the % of individuals in a population that responded (e.g., developed neutralizing antibodies) to the vaccine). In some embodiments, the vaccine is at least 50% efficacious in a population (e.g., at least 60%, at least 70%, at least 80%, at least 90%, at least 95%, at least 97%, at least 98%, at least 99%). In some embodiments, the classification of the vaccine is specific to a particular population class, such as gender, age, and individuals with pre-existing conditions (e.g., immune-compromised). For example, the vaccine may be 50% efficacious in children ages 12 and younger, whereas the vaccine may be 75% efficacious in adults ages 65 and older. In some embodiments, measuring a presence, an absence, or a level of a labeled complex between at least two of (i) a capture molecule, (ii) a peptide-conjugate comprising a peptide derived from a pathogen, and (iii) a neutralizing antibody against the pathogen in the biological sample can be performed in one step without the need for washing.

### Measuring Neutralizing Antibodies as a Surrogate Marker of Protection

In some embodiments, methods are provided using the systems, devices, and compositions described herein to identify the presence and/or a high level of neutralizing antibodies in an individual after administration of a vaccine. In some embodiments, the methods utilize the presence and/or high level of neutralizing antibodies as a surrogate marker of protection in the individual. In some embodiments, the presence and/or high level of neutralizing antibodies in the individual is used to inform a vaccine developer that the individual developed immunity to the pathogen (e.g., SARS-CoV-2). In some embodiments, the presence and/or high level of neutralizing antibodies in the individual is used in place of traditional methods that test efficacy of a vaccine (e.g., a randomized, placebo-controlled clinical trial).

In some embodiments, the methods comprise (a) providing a biological sample from a subject having been administered a vaccine; (b) measuring a presence, an absence, or a level of a labeled complex between at least two of (i) a capture molecule, (ii) a peptide-conjugate comprising a peptide derived from a pathogen, and (iii) a neutralizing antibody against the pathogen; (c) classifying the biological sample as having a presence, an absence, or a level of the neutralizing antibody; and (d) identifying the subject as having immunity against the pathogen based on the presence and/or a high level of the neutralizing antibody.

### Vaccines

In some embodiments, the vaccine is for the active immunization for the prevention of disease caused by a pathogen. The pathogen described herein, in some embodiments, is any bacteria, virus, or fungus that causes infection in a mammal. In some embodiments, a pathogen is a virus of any one of the virus described herein. The virus is a DNA virus or an RNA virus. A DNA virus is a single-stranded (ss) DNA virus, a double-stranded (ds) DNA virus, or a DNA virus that contains both ss and ds DNA regions. An RNA virus is a single-stranded (ss) RNA virus or a double-stranded (ds) RNA virus. A ssRNA virus can further be classified into a positive-sense RNA virus or a negative-sense RNA virus.

In some embodiments, the dsDNA virus is from the family: Myoviridae, Podoviridae, Siphoviridae, Alloherpesviridae, Herpesviridae, Malacoherpesviridae, Lipothrixviridae, Rudiviridae, Adenoviridae, Ampullaviridae, Ascoviridae, Asfaviridae, Baculoviridae, Bicaudaviridae, Clavaviridae, Corticoviridae, Fuselloviridae, Globuloviridae, Guttaviridae, Hytrosaviridae, Iridoviridae, Marseilleviridae, Mimiviridae, Nimaviridae, Pandoraviridae, Papillomaviridae, Phycodnaviridae, Plasmaviridae, Polydnaviruses, Polyomaviridae, Poxviridae, Sphaerolipoviridae, and Tectiviridae. A ssDNA virus is from the family: Anelloviridae, Bacillariodnaviridae, Bidnaviridae, Circoviridae, Geminiviridae, Inoviridae, Microviridae, Nanoviridae, Parvoviridae, and Spiraviridae. A DNA virus that contains both ss and ds DNA regions is from the group of pleolipoviruses. In some cases, the pleolipoviruses include Haloarcula hispanica pleomorphic virus 1, Halogeometricum pleomorphic virus 1, Halorubrum pleomorphic virus 1, Halorubrum pleomorphic virus 2, Halorubrum pleomorphic virus 3, and Halorubrum pleomorphic virus 6.

In some embodiments, the dsRNA virus is from the family: Bimaviridae, Chrysoviridae, Cystoviridae, Endomaviridae, Hypoviridae, Megavimaviridae, Partitiviridae, Picobimaviridae, Reoviridae, Rotavirus and Totiviridae. A positive-sense ssRNA virus is from the family: Alphaflexiviridae, Alphatetraviridae, Alvemaviridae, Arteriviridae, Astroviridae, Bamaviridae, Betaflexiviridae, Bromoviridae, Caliciviridae, Carmotetraviridae, Closteroviridae, Coronaviridae, Dicistroviridae, Flaviviridae, Gammaflexiviridae, Iflaviridae, Leviviridae, Luteoviridae, Mamaviridae, Mesoniviridae, Namaviridae, Nodaviridae, Permutotetraviridae, Picomaviridae, Potyviridae, Roniviridae, Secoviridae, Togaviridae, Tombusviridae, Tymoviridae, and Virgaviridae. A negative-sense ssRNA virus is from the family: Bomaviridae, Filoviridae, Paramyxoviridae, Rhabdoviridae, Nyamiviridae, Arenaviridae, Bunyaviridae, Ophioviridae, and Orthomyxoviridae.

In some embodiments, the vaccine described herein confers adaptive immunity against the pathogen when administered to a subject. In some embodiments, the vaccine described herein confers adaptive immunity to a subject against pathogenic (e.g., viral) infection caused by the pathogen. In some embodiments, the vaccine described herein induces neutralizing antibody against the pathogen in the subject after administration of the vaccine.

In some embodiments, the vaccine confers adaptive immunity against a coronavirus. In some instances, the coronavirus is selected from the group consisting of: alphacoronavirus, betacoronavirus, deltacoronavirus, and gammacoronavirus. Examples of alphacoronavirus include, but are not limited to, Bat coronavirus CDPHE15, Bat coronavirus HKU10, Human coronavirus 229E, Human coronavirus NL63, Miniopterus bat coronavirus 1, Miniopterus bat coronavirus HKU8, Mink coronavirus 1, Porcine epidemic diarrhoea virus, Rhinolophus bat coronavirus HKU2, and Scotophilus bat coronavirus 512. Examples of betacoronavirus include, but are not limited to, Betacoronavirus 1, Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus, Tylonycteris bat coronavirus HKU4. Examples of deltacoronavirus include, but are not limited to, Bulbul coronavirus HKU11, Common moorhen coronavirus HKU21, Coronavirus HKU15, Munia coronavirus HKU13, Night heron coronavirus HKU19, Thrush coronavirus HKU12, White-eye coronavirus HKU16, Wigeon coronavirus HKU20. Examples of gammacoronavirus include, but are not limited to, Avian coronavirus, Beluga whale coronavirus SW1. Additional examples of coronavirus include MERS-CoV, SARS-CoV, and SARS-CoV-2.

In some embodiments, the vaccine comprises an inactivated virus of any one of the virus described herein. In some embodiments, the vaccine comprises an inactivated coronavirus. In some embodiments, the vaccine comprises an inactivated SARS-CoV-2. In some embodiments, the vaccine comprises formalin-inactivated SARS-CoV-2. In some cases, the vaccine comprises a live-attenuated virus of any one of the virus described herein. In some cases, the vaccine comprises a live-attenuated coronavirus. In some cases, the vaccine comprises a live-attenuated SARS-CoV-2.

In some embodiments, antigenic peptide is derived from a protein of any one of the pathogens described herein. In some embodiments, the antigen is viral antigen derived from a viral protein, a fragment of a viral protein, or a nucleic acid encoding the viral protein or the fragment of the viral protein. In some embodiments, the viral antigen is a viral antigen of coronavirus. In some embodiments, the viral antigen is a viral antigen of SARS-CoV-2.

In some embodiments, the vaccine comprises viral antigen for inducing adaptive immunity in a subject. In some embodiments, the viral antigen is a non-mutated antigen. In some embodiments, the viral antigen is derived from publicly disclosed information on the viral genetic information. In some embodiments, the viral antigen is derived from analysis of the viral genome to predict suitable epitopes for T cell activation. In some embodiments, the viral antigen is derived from analysis of the sequence of the viral genome in a MHC-peptide presentation prediction algorithm implemented in a computer processor. In some embodiments, the viral antigen is derived from analysis of the viral sequences in an MHC-peptide presentation prediction algorithm implemented in a computer processor that has been trained by a machine learning software, which predicts the likelihood of binding and presentation of an epitope by an MHC class I or an MHC class II antigen. In some embodiments, the MHC-peptide presentation predictor is neonmhc2.

In some embodiments, the viral antigen is encoded by a nucleic acid sequence that is at least 50%, 55%, 60%, 65%, 70%, 75%, 80%, 85%, 90%, 95%, 96%, 97%, 98%, or 99% identical to SEQ ID NOS: 7-10 (SARS-CoV-2), 13-16 (SARS-CoV NL630), 17-20 (SARS CoV Tor2). In some embodiments, the viral antigen is encoded by a nucleic acid sequence that is 100% identical to any one of SEQ ID NOS: 7-10, 13-16, or 17-20. In some instances, the viral antigen comprises a peptide sequence that is at least 50%, 55%, 60%, 65%, 70%, 75%, 80%, 85%, 90%, 95%, 96%, 97%, 98%, or 99% identical to any one of SEQ ID NOS: 2-6, or 11-12. In some instances, the viral antigen comprises a peptide sequence that is 100% identical to any one of SEQ ID NOS: 2-6, 11-12, 24, 47-48, 71-72, 95-96, or 115. In some instances, the viral antigen comprises a peptide sequence that is at least 50%, 55%, 60%, 65%, 70%, 75%, 80%, 85%, 90%, 95%, 96%, 97%, 98%, or 99% identical to a fragment of any one of SEQ ID NOS: 2-6, 11-12, 24, 47-48, 71-72, 95-96, or 115. In some instances, the viral antigen comprises a peptide sequence that is 100% identical to a fragment of SEQ ID NOS: 2-4, or 11-12.

TABLE 1
Amino Acid Sequence Mutations to SEQ ID NO: 5
Amino Acid Sequence Mutations

| | | |
|---|---|---|
| H69del | D1118H | P26S |
| V70del | L18F | D138Y |
| Y144del | D80A | R190S |
| N501Y | D215G | K417T |
| A570D | R246I | H655Y |
| D614G | K417N | T1027I |
| P681H | E484K | V1176F |
| T716I | A701V | |
| S982A | T20N | |

In some embodiments, the viral antigen comprises an amino acid sequence provided in SEQ ID NO: 5, or a variant thereof. In some embodiments, the viral antigen comprises 1, 2, 3, 4, 5, 6, 7, 8, 9, 10, 11, 12, 13, 14, 15, 16, 17, 18, 19, 20, 21, 22, 23, 24, or 25 or more of the mutations to SEQ ID NO: 5 listed in Table 1.

In some embodiments, the vaccine comprises a viral antigen of SARS-CoV-2. In some embodiments, the viral antigen is selected from the group consisting of: orf1a, orf1ab, Spike glycoprotein (S protein), 3a, 3b, Envelope protein (E protein), Membrane protein (M protein), p6, 7a, 7b, 8b, 9b, Nucleocapsid protein (N protein), orf14, nsp1 (leader protein), nsp2, nsp3, nsp4, nsp5 (3C-like proteinase), nsp6, nsp7, nsp8, nsp9, nsp10 (growth-factor-like protein), nsp12 (RNA-dependent RNA polymerase, or RdRp), nsp13 (RNA 5′-triphosphatase), nsp14 (3′-to-5′ exonuclease), nsp15 (endoRNAse), and nsp16 (2′-O-ribose methyltransferase). In some embodiments, the viral antigen induces formation of neutralizing antibody against the viral antigen in a subject after administration of the vaccine.

In some embodiments, the viral antigen comprises Spike glycoprotein (S protein) or a fragment of the Spike glycoprotein. In some embodiments, the Spike glycoprotein or a fragment thereof is a monomer or a trimer. In some embodiments, the Spike glycoprotein or a fragment thereof is prefusion stabilized form of Spike glycoprotein or fragment thereof. In some embodiments, the viral antigen of the Spike glycoprotein or a fragment thereof is at least 50%, 55%, 60%, 65%, 70%, 75%, 80%, 85%, 90%, 95%, 96%, 97%, 98%, or 99% identical to any one of SEQ ID NOS: 2-6, 11-12, 24, 47-48, 71-72, 95-96, or 115. In some embodiments, the viral antigen of the Spike glycoprotein or a fragment thereof is 100% identical to any one of SEQ ID NOS: 2-6, 11-12, 24, 47-48, 71-72, 95-96, or 115. In some embodiments, the viral antigen of the Spike glycoprotein or a fragment thereof comprises 1, 2, 3, 4, 5, 6, 7, 8, 9, 10, 11, 12, 13, 14, 15, 16, 17, 18, 19, 20, 21, 22, 23, 24, or 25 or more of the mutations to SEQ ID NO: 5 listed in Table 1. In some embodiments, the viral antigen of the Spike glycoprotein or a fragment thereof comprise an amino acid length at least 5 amino acids, 10 amino acids, 20 amino acids, 25 amino acids, 50 amino acids, 100 amino acids, 200 amino acids, or more. In embodiments, the viral antigen comprising the Spike glycoprotein or a fragment thereof induces formation of neutralizing antibody against the Spike-glycoprotein or a fragment thereof in a subject after administration of the vaccine.

In some embodiments, the vaccine comprises displaying, conjugating, or complexing the viral antigen with cell-based carrier, polymer (e.g. polyester), or extra cellular vesicles such as exosomes, microvesicles, retrovirus-like particles, apoptotic bodies, apoptosomes, oncosomes, exophers, enveloped viruses, exomeres, or other very large extracellular vesicles.

In some embodiments, the vaccine comprises at least one heterologous polynucleotide encoding the viral antigen described herein. In some embodiments, the heterologous polynucleotide comprises a viral vector or a plasmid. Non-limiting examples of heterologous polynucleotides include coding or non-coding regions of a gene or gene fragment, loci (locus) defined from linkage analysis, exons, introns, messenger RNA (mRNA), self-amplifying RNA, uridine containing RNA (uRNA), self-amplifying mRNA, transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides is interrupted by non-nucleotide components. In some embodiments, the viral antigen translated from the heterologous polynucleotide induces formation of neutralizing antibody against the viral antigen in a subject after administration of the vaccine.

In some embodiments, the heterologous DNA sequence is transcribed into mRNA and then translated into the viral antigen after administration of the vaccine to the subject. In some embodiments, the viral antigen translated from the heterologous DNA sequence induces formation of neutralizing antibody against the viral antigen in a subject after administration of the vaccine. In some embodiments, the viral antigen comprising Spike glycoprotein or a fragment thereof is translated from the heterologous DNA sequence and induce formation of neutralizing antibody against the Spike glycoprotein or fragment thereof in a subject after administration of the vaccine.

In some embodiments, the vaccine comprises mRNA encoding Spike glycoprotein or a fragment thereof. In some embodiments, the vaccine comprises the heterologous mRNA, where the mRNA is a self-amplifying mRNA (saRNA). In some embodiments, the vaccine comprises the heterologous mRNA, where the mRNA comprises uridine (uRNA). In some embodiments, the heterologous RNA sequence is translated into the viral antigen after administration of the vaccine to the subject. In some embodiments, the viral antigen translated from the heterologous RNA sequence induces formation of neutralizing antibody against the viral antigen in a subject after administration of the vaccine. In some embodiments, the viral antigen comprising Spike glycoprotein or a fragment thereof is translated from the heterologous RNA sequence and induce formation of neutralizing antibody against the Spike glycoprotein or fragment thereof in a subject after administration of the vaccine. In some embodiments, the vaccine is mRNA-1273, which encodes a fill-length, prefusion stabilized Spike (S) protein. In some embodiments, the vaccine is BNT162 vaccine, comprising mRNA or modified mRNA to express the Spike (S) protein or a fragment thereof. In some instances, the BNT162 vaccine comprises nucleoside modified mRNA (modRNA), uridine containing mRNA (uRNA), or self-amplifying mRNA (saRNA),

In some embodiments, the vaccine comprising the at least one heterologous polynucleotide encoding the viral antigen comprises one or more DNA or RNA vectors. In some embodiments, the DNA or RNA vectors is plasmids. In some embodiments, the DNA or RNA vectors is viral vector. Viral vectors, and especially retroviral vectors, are engineered to contain a nucleic acid molecule with a sequence encoding any one of the viral antigens described herein and to be delivered to a target tissue. In some embodiments, the viral vectors are derived from lentivirus, poxviruses, herpes simplex virus I, adenoviruses and adeno-associated viruses, and the like. Non-limiting examples of viral vectors include retroviral vectors, adenoviral vectors, adeno-associated viral vectors (AAVs), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some embodiments, the retroviral vectors also include lentiviral vectors such as those derived from the human immunodeficiency virus (HIV) genome. In some embodiments, AAV vectors include AAV1, AAV2, AAV4, AAV5, AAV6, AAV7, AAV8, or AAV9 serotype. In some embodiments, the viral vector is a chimeric viral vector, comprising viral portions from two or more viruses. In additional embodiments, the viral vector is a recombinant viral vector. In some embodiments, the vaccine is an Ad5-nCoC vaccine comprising nucleic acid sequence encoding the Spike protein. In some embodiments, the vaccine is AZD-1222 vaccine comprising a replication-deficient chimpanzee adenovirus, ChAdOx1, which is engineered to express the Spike (S) protein. In some embodiments, the vaccine is INO-4800 vaccine comprising pGX DNA plasmid with nucleic acid encoding the Spike (S) protein.

In some embodiments, the vaccine comprises adjuvant. In some instances, the vaccine comprises immune modulator selected from any one of the biomarkers described herein.

### Companion or Complementary Diagnostic

Described herein, in some embodiments, are companion complementary diagnostic testing devices or system and methods of their use. In some embodiments, the companion diagnostic testing device or system is used to determine the efficacy or effectiveness of a vaccine against a pathogen described herein. In some embodiments, the companion diagnostic testing device or system is used to identify a person in need of the vaccine. A “companion diagnostic” as used herein, refers to a diagnostic test that may be required for administration of a therapeutic agent for the treatment or prevention of a disease caused by a pathogen disclosed herein. A “complementary diagnostic,” refers to a diagnostic test that may be optional for administration of the therapeutic agent.

In some embodiments, the method determines the efficacy or effectiveness of a vaccine described herein by detecting presence, absence, or quantity of neutralizing antibody against the pathogen in a biological sample from a subject that was administered the vaccine. In some embodiments, the companion diagnostic method confirms adaptive immunity against a pathogen in a subject or in a population of subjects. In some embodiments, the method confirms the need for vaccination against the pathogen for a subject or for a population of subjects. In some embodiments, the companion diagnostic method confirms the prevalent of infection by the pathogen in a population. In some embodiments, the companion diagnostic method identifies a subject or subjects in a population, who are most likely to benefit from being vaccinated with any one of the vaccine described herein. In some embodiments, the companion diagnostic method identifies a subject or subjects in a population, who are likely to be at increased risk for serious side effects as a result of being vaccinated. In some embodiments, the companion diagnostic method monitors response to vaccination for the purpose of adjusting the dosage or the frequency of vaccination to achieve improved safety, efficacy, or effectiveness of the vaccine. For example, based on efficacy results, subjects aged 65 or older may receive a dosage that contains two times, three times, four times, five times, six times, seven times, eight times, nine times, or ten or more times the amount of an antigen than a dosage for subjects aged 64 or younger. In some embodiments, the companion diagnostic method comprises utilizing the biomarker panel described herein.

Companion diagnostic methods provided herein comprise: (a) providing a biological sample from a subject in need thereof; (b) measuring a presence, an absence, or a level of a labeled complex between at least two of (i) a capture molecule, (ii) a peptide-conjugate comprising a peptide derived from a pathogen, and (iii) a neutralizing antibody against the pathogen; and (c) classifying the biological sample as having a presence or quantity of the labeled complex. In some embodiments, the method further comprises identifying a vaccine administered to the subject as effective to induce a sufficient or insufficient adaptive immune response against the pathogen in the subject, provided the biological sample is classified as having the presence or the quantity of the labeled complex between (ii) and (iii). In some embodiments, the method further comprises identifying the subject as in need for a vaccine against the pathogen, provided the biological sample is classified as having the presence or the quantity of the labeled complex between (i) and (ii). In some embodiments, performing (a)-(c) is required for administering the vaccine to the subject (or a population of subjects). In some embodiments, the method further comprises identifying a vaccine administered to the subject as not effective to induce a sufficient or insufficient adaptive immune response against the pathogen in the subject, provided the biological sample is classified as having the presence or the quantity of the labeled complex between (i) and (ii).

In some embodiments, the level is high relative to an index or a control. In some embodiments, the vaccine is effective, provided the presence or the level of the labeled complex between the peptide-conjugate and the neutralizing antibody against the pathogen is measured. In some embodiments, the vaccine is not effective, provided the presence or the level of the labeled complex between the peptide-conjugate and the capture molecule is measured.

In some embodiments, method described herein comprises administering a therapeutic agent to a subject in need thereof, provided the subject is identified as having an acute infection by a pathogen. In some embodiments, the therapeutic agent is effective to reduce or eliminate the acute infection by the pathogen, such as an anti-viral therapeutic agent.

In some embodiments, the companion diagnostic method is performed prior to treating the subject with the therapeutic agent. In some embodiments, performing the companion diagnostic method is required prior to treating the subject with the therapeutic agent. In some embodiments, the therapeutic agent is an anti-viral therapeutic agent. In some embodiments, the anti-viral therapeutic agent treats a disease or condition associated with SARS-CoV-2. In some embodiments, the pathogen is a virus. In some embodiments, the virus is SARS-CoV-2.

In some embodiments, the companion diagnostic method further comprises identifying the subject as having the acute infection comprises by determining the presence, absence, or a quantity of any one of the biomarkers described herein. In some embodiments, the companion diagnostic method comprises determining the presence, absence, or a quantity of both neutralizing antibody and biomarker. In some embodiments, the biomarker is interleukin 6 (IL-6).

Described herein, in some embodiments, are complementary diagnostic testing devices and methods of their use to determine to test the biological sample of the subject, where the subject does not display the symptoms of being infected by or is not exposed to any one of the pathogen described herein. In some embodiments, the complementary diagnostic method determines the benefit-risk decision-making about vaccinating the subject or vaccinating the population, where the difference in benefit-risk is clinically meaningful. In some embodiments, the complementary diagnostic method determines the need to vaccinate a subject or subjects of a population, when the status or prevalence of infection by any one of the pathogen described herein is unknown. In some embodiments, the complementary diagnostic method determines the need to vaccinate or treat the subject or subjects of a population, when the status or prevalence of infection by any one of the pathogen described herein is unknown. For example, a subject or subjects of a population have displayed symptoms of respiratory disease or disorder.

In some embodiments, the complementary diagnostic method confirms the particular pathogen that is causing the respiratory disease or disorder in the absence of a reason to test for that particular pathogen. In some embodiments, the complementary diagnostic method determines the efficacy or effectiveness of a vaccine. For example, a vaccine designed for one disease is showing unexpected protective effect against a different disease caused by a different pathogen. In some embodiments, the complementary diagnostic method identifies a subject or subjects in a population, who are most likely to benefit from being vaccinated with any one of the vaccine described herein. In some embodiments, the complementary diagnostic method identifies a subject or subjects in a population, who are likely to be at increased risk for serious side effects as a result of being vaccinated. In some embodiments, the complementary diagnostic method monitors response to vaccination for the purpose of adjusting the dosage or the frequency of vaccination to achieve improved safety, efficacy, or effectiveness of the vaccine. For example, based on response results, subjects aged 65 or older may receive a dosage that contains two times, three times, four times, five times, six times, seven times, eight times, nine times, or ten or more times the amount of an antigen than a dosage for subjects aged 64 or younger. In some embodiments, the complementary diagnostic method comprises utilizing the biomarker panel described herein.

### Biomarker Panel

Described herein, in some embodiments, are methods utilizing the systems, devices, and compositions describe herein for detecting presence, absence, quantity, or activity of a biomarker described herein. In some embodiments, the biomarkers are associated with infection by any one of the pathogens described herein. In some embodiments, the biomarkers are induced by infection by the pathogen. In some embodiments, the biomarkers are cytokines induced by infection. In some embodiments, the biomarkers are serological biomarkers. The quantity or activity of the biomarkers can increase or decrease after the infection. In some embodiments, the biomarkers may comprise a peptide or protein that is encoded by a nucleic acid sequence, or that has an amino acid sequence, that is at least 80% identical to any one of SEQ ID NOS: 21-23, 25-42, 44-46, 49-66, 68-70, 73-90, 92-94, or 97-114.

Biomarker panel methods provided herein comprise: (a) providing a biological sample from a subject in need thereof; (b) measuring a presence, an absence, level, or activity of at least one biomarker; (c) classifying the biological sample as having a presence or level of the biomarker associated or induced by an infection; and optionally (d) measuring a presence, an absence, or quantity of neutralizing antibody induced by the infection. In some embodiments, the method identifies the infection caused by the pathogen based on the quantity or activity of the biomarker. In some embodiments, the method identifies the infection caused by the pathogen based on the quantity or activity of the biomarker in a subject, who does not have neutralizing antibody against the pathogen. In some embodiments, the method confirms the infection caused by the pathogen based on the quantity or activity of the biomarker. In some embodiments, the method confirms the infection caused by the pathogen based on the quantity or activity of the biomarker in a subject, who does not have neutralizing antibody against the pathogen. In some embodiments, the method comprise prognosing an immunity or measuring susceptibility to infection based on detecting presence, absence, level, or activity of the biomarkers described herein. In some embodiments, the method comprise prognosing an immunity or measuring susceptibility to infection based on detecting presence, absence, quantity, or activity of the biomarkers in combination with detecting presence, absence, or quantity of the neutralizing antibodies described herein to confirm an infection caused by any one of the pathogen described herein.

In some embodiments, the presence, absence, level, or activity of the biomarker is detected. In some embodiments, the method detects presence absence, level, or activity of at least one, two, three, four, five, 10, 20, 30, 40, 50, 60, 70, 80, 90, 100, 200, 500, 1000 or more biomarkers of the biomarker panel in a biological sample. In some embodiments, the method detects biomarker comprising a non-peptide coding nucleic acid sequence. In some embodiments, the method detects biomarker comprising a peptide-coding nucleic acid sequence such as mRNA or cDNA. In some embodiments, the method detects biomarker comprising peptide or protein.

In some instances, the method detects presence, absence, or level, or activity of the biomarker by utilizing a nucleic acid sequence by subjecting a biological sample to a nucleic acid-based detection assay. The nucleic acid-based detection assay comprises quantitative polymerase chain reaction (qPCR), gel electrophoresis (including for e.g., Northern or Southern blot), immunochemistry, in situ hybridization such as fluorescent in situ hybridization (FISH), cytochemistry, or sequencing. The sequencing technique comprises next generation sequencing. In some embodiments, the method involves a hybridization assay such as fluorogenic qPCR (e.g., TaqMan™, SYBR green, SYBR green I, SYBR green II, SYBR gold, ethidium bromide, methylene blue, Pyronin Y, DAPI, acridine orange, Blue View or phycoerythrin), which involves a nucleic acid amplification reaction with a specific primer pair, and hybridization of the amplified nucleic acid probes comprising a detectable moiety or molecule that is specific to a target nucleic acid sequence. In some instances, a number of amplification cycles for detecting a target nucleic acid in a qPCR assay is about 5 to about 30 cycles. In some instances, the number of amplification cycles for detecting a target nucleic acid is at least about 5 cycles. In some instances, the number of amplification cycles for detecting a target nucleic acid is at most about 30 cycles. In some instances, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles. In some instances, hybridization may occur at standard hybridization temperatures, e.g., between about 35° C. and about 65° C. in a standard PCR buffer. An additional exemplary nucleic acid-based detection assay comprises the use of nucleic acid probes conjugated or otherwise immobilized on a bead, multi-well plate, or other substrate, wherein the nucleic acid probes are configured to hybridize with a target nucleic acid sequence. In some instances, the nucleic acid probe is specific to one or more genetic variants disclosed herein is used. In some instances, the nucleic acid probe specific to a SNP or SNV comprises a nucleic acid probe sequence sufficiently complementary to a risk or protective allele of interest, such that hybridization is specific to the risk or protective allele. In some instances, the nucleic acid probe specific to an indel comprises a nucleic acid probe sequence sufficiently complementary to an insertion of a nucleobase within a polynucleotide sequence flanking the insertion, such that hybridization is specific to the indel. In some instances, the nucleic acid probe specific to an indel comprises a probe sequence sufficiently complementary to a polynucleotide sequence flanking a deletion of a nucleobase within the polynucleotide sequence, such that hybridization is specific to the indel. In some instances, the nucleic acid probe specific to a biomarker comprises a nucleic acid probe sequence sufficiently complementary to the polynucleotide sequence of the biomarker. In some instances, the biomarker comprises a transcribed polynucleotide sequence (e.g., RNA, cDNA). In some embodiments, the nucleic acid probe is, for example, a full-length cDNA, or a portion thereof, such as an oligonucleotide of at least about 7, 8, 9, 10, 11, 12, 13, 14, 15, 20, 25, 30, 35, 40, 45, or 50 nucleotides in length and sufficient to specifically hybridize under standard hybridization conditions to the target nucleic acid sequence. In some embodiments, the target nucleic acid sequence is immobilized on a solid surface and contacted with a probe, for example by running the isolated target nucleic acid sequence on an agarose gel and transferring the target nucleic acid sequence from the gel to a membrane, such as nitrocellulose. In some embodiments, the probe(s) are immobilized on a solid surface, for example, in an Affymetrix gene chip array, and the probe(s) are contacted with the target nucleic acid sequence.

In some instances, the method detects presence, absence, or level, or activity of the biomarker by utilizing a protein-based assay. In some embodiments, the method detects the biomarker by an antibody-based assay, where an antibody specific to the biomarker is utilized. In some embodiments, antibody-based detection method can utilize an antibody that binds to any region of the biomarker. Another exemplary method of detecting the biomarker comprises performing an enzyme-linked immunosorbent assay (ELISA). The ELISA assay is a sandwich ELISA or a direct ELISA. Another exemplary method of detecting the biomarker comprises a single molecule array, e.g., Simoa. Other exemplary methods of detecting biomarkers can include immunohistochemistry, lateral flow assay, gel electrophoresis, capillary electrophoresis, high performance liquid chromatography (HPLC), thin layer chromatography (TLC), hyperdiffusion chromatography, or various immunological methods such as fluid or gel precipitation reactions, immunodiffusion (single or double), immunoelectrophoresis, radioimmunoassay (RIA), immunofluorescent assays, and Western blotting. In some embodiments, antibodies, or antibody fragments, is used in methods such as Western blots or immunofluorescence techniques to detect the expressed proteins. The antibody or protein is immobilized on a solid support for Western blots and immunofluorescence techniques. Suitable solid phase supports or carriers include any support capable of binding an antigen or an antibody. Exemplary supports or carriers include glass, polystyrene, polypropylene, polyethylene, dextran, nylon, amylases, natural and modified celluloses, polyacrylamides, gabbros, and magnetite.

### Data Analysis Using Personal Electronic Device

Methods disclosed herein further comprise: (a) capturing an image of a detection zone of the testing device; and (b) analyzing data from the image using one or more computer programs. In some embodiments, the one or more computer programs is run on a computing device described here. In some embodiments, capturing is performed with an imaging device described herein. In some cases, capturing and analyzing are performed by a single personal electronic device, such as a smartphone, tablet, or laptop computer. In some embodiments, capturing and analyzing are performed at the point of need (e.g., same time and place as performing the assay with the testing device).

In some embodiments, capturing comprises taking a still photograph of a liquid phase, such as a liquid composition disclosed herein. In some embodiments, capturing comprises taking a still photograph of the solid surface of the assay assembly described herein. In some embodiments, capturing comprises taking a video of the solid surface of the assay assembly.

In some embodiments, analyzing comprises detecting binding between the analyte (antibody against an antigenic peptide from the pathogen) and peptide-conjugate. In some embodiments, analyzing comprises subtracting a background signal, thereby increasing the signal to noise ratio. In some embodiments, analyzing is performed by a data analytics module of an application or computer program of the computing device. In some embodiments, the analyzing by the data analytics module comprises performing machine learning.

### Kits

Described herein, in some embodiments, are kits for obtaining a biological sample or detecting an analyte in the biological sample. In some embodiments, the kits may comprise a biological sample collection device, such as, for example, a transdermal puncture device (e.g., finger prick lance) or other sample collection device (e.g., a swab). In some embodiments, the kits may comprise a dried blood spot (DBS) card described herein for storing the biological sample. In some embodiments, the kits comprise an assay assembly described herein, such as a lateral flow assay or an ELISA that is capable of detecting neutralizing antibodies against a pathogen of interest in the biological sample.

In some embodiments, the kits described herein comprise instructions. In some embodiments, the instructions provide information about how to obtain the biological sample. In some embodiments, the instructions comprise information about how to discard the biological sample. In some embodiments, the kits comprise information about how to download a mobile application for a personal electronic device that captures and analyzes the results of the testing device. In some embodiments, the kits comprise a survey having one or more clinical questions pertaining to a symptom of an infection by a pathogen of interest. In some embodiments, the survey is on the back of the DBS card of the kit.

### Embodiments

The following non-limiting embodiments provide illustrative examples of the disclosure, but do not limit the scope of the disclosure.

Embodiment 1. A system comprising: (a) one or more capture molecules derived from an angiotensin-converting enzyme 2 (ACE2) receptor; and (b) a peptide-conjugate comprising: (i) a peptide derived from a spike glycoprotein of a coronavirus; and (ii) a detectable moiety.

Embodiment 2. The system of embodiment 1 further comprises (a) a surface; and (b) an imaging device configured to capture an image of a complex between the peptide-conjugate and the one or more capture molecules on the surface when the complex is coupled to the surface.

Embodiment 3. The system of any one of embodiments 1-2 further comprises an imaging device configured to capture an image of a complex between the peptide-conjugate and the one or more capture molecules.

Embodiment 4. The system of any one of embodiments 1-3 comprises a container comprising (a) and (b), wherein the container is portable.

Embodiment 5. The system of any one of embodiments 1-4 is a point of need system.

Embodiment 6. The system of any one of embodiments 1-5 is a point of care system.

Embodiment 7. The system of any one of embodiments 1-6, wherein the surface comprises a material selected from the group consisting of a metal, a plastic, glass, and a nitrocellulose membrane.

Embodiment 8. The system of any one of embodiments 1-7, wherein the surface is a passivated surface.

Embodiment 9. The system of any one of embodiments 1-8, wherein the passivated surface comprises a polymer layer comprising a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran.

Embodiment 10. The system of any one of embodiments 1-9, wherein the complex is coupled to the surface.

Embodiment 11. The system of any one of embodiments 1-10, wherein, the complex is coupled to the surface by a covalent bond, a linker, or a combination thereof.

Embodiment 12. The system of any one of embodiments 1-11, wherein the linker is a chemical linker, a peptide linker, or a combination thereof.

Embodiment 13. The system of any one of embodiments 1-12, wherein the one or more capture molecules comprises two or more capture molecules.

Embodiment 14. The system of any one of embodiments 1-13, wherein the one or more capture molecules is coupled to the surface.

Embodiment 15. The system of any one of embodiments 1-14, wherein the one or more capture molecules is coupled to the surface by a covalent bond, a linker, or a combination thereof.

Embodiment 16. The system of any one of embodiments 1-15, wherein the linker is a chemical linker, a peptide linker, or a combination thereof.

Embodiment 17. The system of any one of embodiments 1-16, wherein the one or more capture molecules is a fusion polypeptide.

Embodiment 18. The system of any one of embodiments 1-17, wherein the fusion polypeptide comprises at least a portion of a fragment crystallizable region (Fc) region of a monoclonal antibody.

Embodiment 19. The system of any one of embodiments 1-18, wherein the one or more capture molecules is bound by an antibody that is coupled to the surface.

Embodiment 20. The system of any one of embodiments 1-19, wherein the peptide-conjugate comprises a nanoparticle, a fluorescent dye, an enzymatic label, or a colorimetric label, or a combination thereof.

Embodiment 21. The system of any one of embodiments 1-20, wherein the nanoparticle comprises a material selected from the group consisting of a metal, agarose, acrylic, and plastic.

Embodiment 22. The system of any one of embodiments 1-21, wherein the nanoparticle is magnetic.

Embodiment 23. The system of any one of embodiments 1-22, the nanoparticle is conjugated to the peptide by a linker comprising a chemical linker, a peptide linker, or a combination thereof.

Embodiment 24. The system of any one of embodiments 1-23, wherein, the one or more capture molecules derived from the ACE2 receptor comprises an amino acid sequence that is at least 70%, 75%, 80%, 85%, 90%, 91%, 92%, 93%, 94%, 95%, 96%, 97%, 98%, 99%, 100% identical to SEQ ID NO: 1.

Embodiment 25. The system of any one of embodiments 1-24, wherein, the peptide derived from a spike glycoprotein of a coronavirus comprises at least a portion of a spike protein derived from Severe acute respiratory syndrome-associated coronavirus (SARS-CoV).

Embodiment 26. The system of any one of embodiments 1-25, wherein the SARS-CoV is SARS-CoV-2.

Embodiment 27. The system of any one of embodiments 1-26, wherein an infection in a human subject by the SARS-CoV-2 causes coronavirus disease of 2019 (COVID-19) in the human subject.

Embodiment 28. The system of any one of embodiments 1-27, wherein the at least a portion of the spike protein comprises a subunit 1 of the spike protein.

Embodiment 29. The system of any one of embodiments 1-28, wherein the at least a portion of the spike protein comprises a receptor binding domain (RBD) of the subunit 1 of the spike protein.

Embodiment 30. The system of any one of embodiments 1-29, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 2.

Embodiment 31. The system of any one of embodiments 1-30, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 3.

Embodiment 32. The system of any one of embodiments 1-31, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 4.

Embodiment 33. The system of any one of embodiments 1-32, wherein the complex between the peptide-conjugate and the one or more capture molecules on the surface is visible on the surface using color, reflectance, fluorescence, bioluminescence, or chemiluminescence.

Embodiment 34. The system of any one of embodiments 1-33, where the system further comprises a housing at least partially enclosing the surface.

Embodiment 35. The system of any one of embodiments 1-34, wherein the system further comprises a sample receptor configured to receive a biological sample from a subject.

Embodiment 36. The system of any one of embodiments 1-35, wherein the sample receptor is mechanically coupled to a housing at least partially enclosing the surface.

Embodiment 37. The system of any one of embodiments 1-36, wherein the biological sample comprises one or more antibodies specific to the peptide.

Embodiment 38. The system of any one of embodiments 1-37, wherein the biological sample does not consist of one or more antibodies specific to the peptide. Embodiment 10. the one or more antibodies belong to one or more immunoglobulin classes comprising immunoglobulin M, immunoglobulin G, immunoglobulin A, immunoglobulin E, and immunoglobulin D. Embodiment 10. the subject was, or is, exposed to the coronavirus.

Embodiment 39. The system of any one of embodiments 1-38, wherein exposure of the subject to the coronavirus is unknown.

Embodiment 40. The system of any one of embodiments 1-39, wherein the subject was administered a vaccine against the coronavirus.

Embodiment 41. The system of any one of embodiments 1-40, wherein the biological sample comprises blood, urine, saliva, or feces.

Embodiment 42. The system of any one of embodiments 1-41, wherein the blood is capillary blood.

Embodiment 43. The system of any one of embodiments 1-42, wherein the system further comprises a transdermal puncture device configured to obtain the capillary blood from the subject.

Embodiment 44. The system of any one of embodiments 1-43, wherein the sample receptor comprises a filter to separate serum from the blood.

Embodiment 45. The system of any one of embodiments 1-44, wherein the system further comprises a data store for storing data from the image that is captured by the imaging device.

Embodiment 46. The system of any one of embodiments 1-45, wherein the data store is a cloud-based or a web-based data store, or a local data store.

Embodiment 47. The system of any one of embodiments 1-46, wherein the data comprises one or more of geolocation of the imaging device, a result from the image captured by the imaging device, and external data.

Embodiment 48. The system of any one of embodiments 1-47, wherein the external data is data from an external device selected from a diagnostic device, a prognostic device, or a health or fitness tracking device.

Embodiment 49. The system of any one of embodiments 1-48, wherein the external data comprises body temperature, heart rate variability, resting heart rate, sleep quality, and sleep quantity.

Embodiment 50. The system of any one of embodiments 1-49, wherein the system further comprises an external device selected from a diagnostic device, a prognostic device, or a health or fitness tracking device.

Embodiment 51. The system of any one of embodiments 1-50, wherein the imaging device is a personal electronic device.

Embodiment 52. The system of any one of embodiments 1-51, wherein the personal electronic device is a smart phone, tablet, body camera, web camera, or personal computer.

Embodiment 53. The system of any one of embodiments 1-52, wherein the personal electronic device comprises a web-based portal.

Embodiment 54. The system of any one of embodiments 1-53, wherein the web-based portal utilizes an application.

Embodiment 55. The system of any one of embodiments 1-54, wherein the application is configured to receive data comprising a result from the image captured by the personal electronic device, a geolocation of the personal electronic device, and external data from one or more external device.

Embodiment 56. The system of any one of embodiments 1-55, wherein the application comprises a data analytics module configured to analyze the result by: (a) determining whether the result is a positive result or a negative result, wherein a positive results indicates immunity and a negative results indicates a lack of immunity; (b) determining an absolute number of complexes between the peptide-conjugate and the ACE2 receptor on the surface; or (c) determining a level of binding between the peptide-conjugate and the ACE2, wherein a high level of binding indicates a low level of immunity, and a low level of binding indicates a high level of immunity.

Embodiment 57. The system of any one of embodiments 1-56, wherein the positive or the negative result is relative to a threshold number of complexes between the peptide-conjugate and the ACE2 receptor on the surface.

Embodiment 58. The system of any one of embodiments 1-57, wherein the threshold number is predetermined relative to an index a control.

Embodiment 59. The system of any one of embodiments 1-58, wherein the data analytics module is further configured to normalize the result by subtracting background noise.

Embodiment 60. The system of any one of embodiments 1-59, wherein the data analytics module is further configured to identify a geographical location comprised of subjects for which a positive result was determined to detect a presence of herd immunity to the coronavirus, or recommend further testing.

Embodiment 61. The system of any one of embodiments 1-60, wherein the data analytics module utilizes geofencing from coordinates of the personal electronic device to identify the geographical location.

Embodiment 62. The system of any one of embodiments 1-61, wherein the data analytics module utilizes a machine learning algorithm, artificial intelligence, or both.

Embodiment 63. The system of any one of embodiments 1-62, wherein system further comprises one or more capture molecules specific to one or more antibodies against the coronavirus.

Embodiment 64. The system of any one of embodiments 1-63, wherein the one or more capture molecules specific to one or more antibodies against the coronavirus is labeled.

Embodiment 65. The system of any one of embodiments 1-64, wherein system further comprises a labeled secondary capture molecule specific to the one or more capture molecules specific to one or more antibodies against the coronavirus.

Embodiment 66. The system of any one of embodiments 1-65, wherein the one or more capture molecules specific to one or more antibodies against the coronavirus is an antibody or antigen-binding fragment. Embodiment 10. the antibody or antigen-binding fragment is monoclonal. antibody or antigen-binding fragment is polyclonal.

Embodiment 67. Aspects disclosed herein comprise system comprising: (a) one or more capture molecules derived from a spike glycoprotein of a coronavirus; (b) a peptide-conjugate comprising: (i) a peptide derived from angiotensin-converting enzyme 2 (ACE2) receptor; and (ii) a detectable moiety.

Embodiment 68. The system of embodiment 67, wherein the system further comprises (a) a surface; and (b) an imaging device configured to capture an image of a complex between the peptide-conjugate and the one or more capture molecules on the surface when the complex is coupled to the surface.

Embodiment 69. The system of any one of embodiments 67-68, wherein the system further comprises an imaging device configured to capture an image of a complex between the peptide-conjugate and the one or more capture molecules.

Embodiment 70. The system of any one of embodiments 67-69, wherein the system further comprises a container comprising (a) and (b), wherein the container is portable.

Embodiment 71. The system of any one of embodiments 67-70, wherein the system is a point of need system.

Embodiment 72. The system of any one of embodiments 67-71, wherein the point of need is a point of care system.

Embodiment 73. The system of any one of embodiments 67-72, wherein the surface comprises a material selected from the group consisting of a metal, a plastic, glass, and a nitrocellulose membrane.

Embodiment 74. The system of any one of embodiments 67-73, wherein the surface is a passivated surface.

Embodiment 75. The system of any one of embodiments 67-74, wherein the passivated surface comprises a polymer layer comprising a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran.

Embodiment 76. The system of any one of embodiments 67-75, wherein the complex is coupled to the surface.

Embodiment 77. The system of any one of embodiments 67-76, wherein the complex is coupled to the surface by a covalent bond, a linker, or a combination thereof.

Embodiment 78. The system of any one of embodiments 67-77, wherein the linker is a chemical linker, a peptide linker, or a combination thereof.

Embodiment 79. The system of any one of embodiments 67-78, wherein the one or more capture molecules comprises two or more capture molecules.

Embodiment 80. The system of any one of embodiments 67-79, wherein the one or more capture molecules is coupled to the surface.

Embodiment 81. The system of any one of embodiments 67-80, wherein the one or more capture molecules is coupled to the surface by a covalent bond, a linker, or a combination thereof.

Embodiment 82. The system of any one of embodiments 67-81, wherein the linker is a chemical linker, a peptide linker, or a combination thereof.

Embodiment 83. The system of any one of embodiments 67-82, wherein the one or more capture molecules is a fusion polypeptide.

Embodiment 84. The system of any one of embodiments 67-83, wherein the fusion polypeptide comprises at least a portion of a fragment crystallizable region (Fc) region of a monoclonal antibody.

Embodiment 85. The system of any one of embodiments 67-84, wherein the one or more capture molecules is bound by an antibody that is coupled to the surface.

Embodiment 86. The system of any one of embodiments 67-85, wherein the peptide-conjugate comprises a nanoparticle, a fluorescent dye, an enzymatic label, or a colorimetric label, or a combination thereof.

Embodiment 87. The system of any one of embodiments 67-86, wherein the nanoparticle comprises a material selected from the group consisting of a metal, agarose, acrylic, and plastic.

Embodiment 88. The system of any one of embodiments 67-87, wherein the nanoparticle is magnetic.

Embodiment 89. The system of any one of embodiments 67-88, wherein the nanoparticle is conjugated to the peptide by a linker comprising a chemical linker, a peptide linker, or a combination thereof.

Embodiment 90. The system of any one of embodiments 67-89, wherein the peptide derived from the ACE2 receptor comprises an amino acid sequence that is at least 70%, 75%, 80%, 85%, 90%, 91%, 92%, 93%, 94%, 95%, 96%, 97%, 98%, 99%, 100% identical to SEQ ID NO: 1.

Embodiment 91. The system of any one of embodiments 67-90, wherein the one or more capture molecules derived from a spike glycoprotein of a coronavirus comprises at least a portion of a spike protein derived from Severe acute respiratory syndrome-associated coronavirus (SARS-CoV).

Embodiment 92. The system of any one of embodiments 67-91, wherein the SARS-CoV is SARS-CoV-2.

Embodiment 93. The system of any one of embodiments 67-92, wherein an infection in a human subject by the SARS-CoV-2 causes coronavirus disease of 2019 (COVID-19) in the human subject.

Embodiment 94. The system of any one of embodiments 67-93, wherein the at least a portion of the spike protein comprises a subunit 1 of the spike protein.

Embodiment 95. The system of any one of embodiments 67-94, wherein the at least a portion of the spike protein comprises a receptor binding domain (RBD) of the subunit 1 of the spike protein.

Embodiment 96. The system of any one of embodiments 67-95, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 2.

Embodiment 97. The system of any one of embodiments 67-96, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 3.

Embodiment 98. The system of any one of embodiments 67-97, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NOS: 4-6, 11-12, 24, 47-48, 71-72, 95-96, or 115.

Embodiment 99. The system of any one of embodiments 67-98, wherein the complex between the peptide-conjugate and the one or more capture molecules on the surface is visible on the surface using color, reflectance, fluorescence, bioluminescence, or chemiluminescence.

Embodiment 100. The system of any one of embodiments 67-99, wherein system further comprises one or more capture molecules specific to one or more antibodies against the coronavirus. Embodiment 101. The system of any one of embodiments 67-100, wherein the one or more capture molecules specific to one or more antibodies against the coronavirus is labeled.

Embodiment 102. The system of any one of embodiments 67-101, wherein system further comprises a labeled secondary capture molecule specific to the one or more capture molecules specific to one or more antibodies against the coronavirus.

Embodiment 103. The system of any one of embodiments 67-102, wherein the one or more capture molecules specific to one or more antibodies against the coronavirus is an antibody or antigen-binding fragment.

Embodiment 104. The system of any one of embodiments 67-103, wherein the antibody or antigen-binding fragment is monoclonal. antibody or antigen-binding fragment is polyclonal.

Embodiment 105. The system of any one of embodiments 67-104, wherein system further comprises a housing at least partially enclosing the surface.

Embodiment 106. The system of any one of embodiments 67-105, wherein system further comprises a sample receptor configured to receive a biological sample from a subject.

Embodiment 107. The system of any one of embodiments 67-106, wherein the sample receptor is mechanically coupled to a housing at least partially enclosing the surface.

Embodiment 108. The system of any one of embodiments 67-107, wherein the biological sample comprises one or more antibodies specific to the peptide.

Embodiment 109. The system of any one of embodiments 67-108, wherein the biological sample does not consist of one or more antibodies specific to the peptide.

Embodiment 110. The system of any one of embodiments 67-109, wherein the one or more antibodies belong to one or more immunoglobulin classes comprising immunoglobulin M, immunoglobulin G, immunoglobulin A, immunoglobulin E, and immunoglobulin D.

Embodiment 111. The system of any one of embodiments 67-110, wherein the subject was, or is, exposed to the coronavirus.

Embodiment 112. The system of any one of embodiments 67-111, wherein exposure of the subject to the coronavirus is unknown.

Embodiment 113. The system of any one of embodiments 67-112, wherein the subject was administered a vaccine against the coronavirus.

Embodiment 114. The system of any one of embodiments 67-113, wherein the biological sample comprises blood, urine, saliva, or feces.

Embodiment 115. The system of any one of embodiments 67-114, wherein the blood is capillary blood.

Embodiment 116. The system of any one of embodiments 67-115, wherein system further comprises a transdermal puncture device configured to obtain the capillary blood from the subject.

Embodiment 117. The system of any one of embodiments 67-116, wherein the sample receptor comprises a filter to separate serum from the blood.

Embodiment 118. The system of any one of embodiments 67-117, wherein system further comprises a data store for storing data from the image that is captured by the imaging device.

Embodiment 119. The system of any one of embodiments 67-118, wherein the data store is a cloud-based or a web-based data store, or a local data store.

Embodiment 120. The system of any one of embodiments 67-119, wherein the data comprises one or more of geolocation of the imaging device, a result from the image captured by the imaging device, and external data.

Embodiment 121. The system of any one of embodiments 67-120, wherein external data is data from an external device selected from a diagnostic device, a prognostic device, or a health or fitness tracking device.

Embodiment 122. The system of any one of embodiments 67-121, wherein external data comprises body temperature, heart rate variability, resting heart rate, sleep quality, and sleep quantity.

Embodiment 123. The system of any one of embodiments 67-122, wherein system further comprises an external device selected from a diagnostic device, a prognostic device, or a health or fitness tracking device.

Embodiment 124. The system of any one of embodiments 67-123, wherein the imaging device is a personal electronic device.

Embodiment 125. The system of any one of embodiments 67-124, wherein the personal electronic device is a smart phone, tablet, body camera, web camera, or personal computer.

Embodiment 126. The system of any one of embodiments 67-125, wherein the personal electronic device comprises a web-based portal.

Embodiment 127. The system of any one of embodiments 67-126, wherein the web-based portal utilizes an application.

Embodiment 128. The system of any one of embodiments 67-127, wherein the application is configured to receive data comprising a result from the image captured by the personal electronic device, a geolocation of the personal electronic device, and external data from one or more external device.

Embodiment 129. The system of any one of embodiments 67-128, wherein the application comprises a data analytics module configured to analyze the result by: (a) determining whether the result is a positive result or a negative result, wherein a positive results indicates immunity and a negative results indicates a lack of immunity; (b) determining an absolute number of complexes between the peptide-conjugate and the ACE2 receptor on the surface; or (c) determining a level of binding between the peptide-conjugate and the ACE2, wherein a high level of binding indicates a low level of immunity, and a low level of binding indicates a high level of immunity.

Embodiment 130. The system of any one of embodiments 67-129, wherein the data analytics module is further configured to analyze the result by determining whether the result is a positive result or a negative result, wherein a positive result indicates a presence of an acute infection by the coronavirus, and a negative results indicates an absence of the acute infection by the coronavirus.

Embodiment 131. The system of any one of embodiments 67-130, wherein the positive or the negative result is relative to a threshold number of complexes between the peptide-conjugate and the ACE2 receptor on the surface.

Embodiment 132. The system of any one of embodiments 67-131, wherein the positive or the negative result is relative to a threshold number of complexes between the one or more capture molecules specific to the one or more antibodies against the coronavirus.

Embodiment 133. The system of any one of embodiments 67-132, wherein the threshold number is predetermined relative to an index a control.

Embodiment 134. The system of any one of embodiments 67-133, wherein the data analytics module is further configured to normalize the result by subtracting background noise.

Embodiment 135. The system of any one of embodiments 67-134, wherein the data analytics module is further configured to identify a geographical location comprised of subjects for which a positive result was determined to detect a presence of herd immunity to the coronavirus, or recommend further testing.

Embodiment 136. The system of any one of embodiments 67-135, wherein the data analytics module is further configured to identify a geographical location comprised of subjects for which a positive result was determined to identify regions of infection or re-infection, or to recommend further testing.

Embodiment 137. The system of any one of embodiments 67-136, wherein the data analytics module utilizes geofencing from coordinates of the personal electronic device to identify the geographical location.

Embodiment 138. The system of any one of embodiments 67-137, wherein the data analytics module utilizes a machine learning algorithm, artificial intelligence, or both.

Embodiment 139. Aspects disclosed herein provide a method of identifying adaptive immunity to a coronavirus in a subject, the method comprising: (a) obtaining a biological sample from the subject; (b) producing a mixture by introducing the biological sample with a detectable peptide derived from a spike glycoprotein of a coronavirus; (c) bringing the mixture into contact with one or more capture molecules derived from an angiotensin-converting enzyme 2 (ACE2) receptor; (d) detecting a number of binding complexes between the detectable peptide and the one or more capture molecules; (e) if the number of the binding complexes is low relative to an index or a control, then identifying the subject as being immune to an infection by the coronavirus; and (f) if the number of the binding complexes is high relative to an index or a control, then identifying the subject as not being immune to an infection by the coronavirus.

Embodiment 140. The method of embodiment 139, wherein steps (a)-(f) are performed at the point of need.

Embodiment 141. The method of any one of embodiments 139-140, wherein steps (a)-(f) are performed at the point of care.

Embodiment 142. The method of any one of embodiments 139-141, wherein the surface comprises a material selected from the group consisting of a metal, a plastic, glass, and a nitrocellulose membrane.

Embodiment 143. The method of any one of embodiments 139-142, wherein the surface is a passivated surface.

Embodiment 144. The method of any one of embodiments 139-143, wherein the passivated surface comprises a polymer layer comprising a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran.

Embodiment 145. The method of any one of embodiments 139-144, wherein the one or more capture molecules comprises two or more capture molecules.

Embodiment 146. The method of any one of embodiments 139-145, wherein the one or more capture molecules is coupled to the surface by a covalent bond, a linker, or a combination thereof.

Embodiment 147. The method of any one of embodiments 139-146, wherein the linker is a chemical linker, a peptide linker, or a combination thereof.

Embodiment 148. The method of any one of embodiments 139-147, wherein the one or more capture molecules is a fusion polypeptide.

Embodiment 149. The method of any one of embodiments 139-148, wherein the fusion polypeptide comprises at least a portion of a fragment crystallizable region (Fc) region of a monoclonal antibody.

Embodiment 150. The method of any one of embodiments 139-149, wherein the one or more capture molecules is bound by an antibody that is coupled to the surface.

Embodiment 151. The method of any one of embodiments 139-150, wherein the detectable peptide comprises a nanoparticle, a fluorescent dye, an enzymatic label, or a colorimetric label, or a combination thereof.

Embodiment 152. The method of any one of embodiments 139-151, wherein The method of any one of embodiments 139-192, wherein the nanoparticle comprises a material selected from the group consisting of a metal, agarose, acrylic, and plastic.

Embodiment 153. The method of any one of embodiments 139-152, wherein the nanoparticle is magnetic.

Embodiment 154. The method of any one of embodiments 139-153, wherein the nanoparticle is conjugated to the peptide by a linker comprising a chemical linker, a peptide linker, or a combination thereof.

Embodiment 155. The method of any one of embodiments 139-154, wherein the detectable peptide derived from the ACE2 receptor comprises an amino acid sequence that is at least 70%, 75%, 80%, 85%, 90%, 91%, 92%, 93%, 94%, 95%, 96%, 97%, 98%, 99%, 100% identical to SEQ ID NO: 1.

Embodiment 156. The method of any one of embodiments 139-155, wherein the one or more capture molecules derived from the spike glycoprotein of a coronavirus comprises at least a portion of a spike protein derived from Severe acute respiratory syndrome-associated coronavirus (SARS-CoV).

Embodiment 157. The method of any one of embodiments 139-156, wherein the SARS-CoV is SARS-CoV-2.

Embodiment 158. The method of any one of embodiments 139-157, wherein an infection in a human subject by the SARS-CoV-2 causes coronavirus disease of 2019 (COVID-19) in the human subject.

Embodiment 159. The method of any one of embodiments 139-158, wherein the at least a portion of the spike protein comprises a subunit 1 of the spike protein.

Embodiment 160. The method of any one of embodiments 139-159, wherein the at least a portion of the spike protein comprises a receptor binding domain (RBD) of the subunit 1 of the spike protein.

Embodiment 161. The method of any one of embodiments 139-160, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 2.

Embodiment 162. The method of any one of embodiments 139-161, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 3.

Embodiment 163. The method of any one of embodiments 139-162, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to any one of SEQ ID NOS: 4-6, 11-12, 24, 47-48, 71-72, 95-96, or 115.

Embodiment 164. The method of any one of embodiments 139-163, wherein the complex between the peptide-conjugate and the one or more capture molecules is visible on the surface using color, reflectance, fluorescence, bioluminescence, or chemiluminescence.

Embodiment 165. The method of any one of embodiments 139-164, wherein the one or more capture molecules derived from the ACE2 receptor comprises an amino acid sequence that is at least 70%, 75%, 80%, 85%, 90%, 91%, 92%, 93%, 94%, 95%, 96%, 97%, 98%, 99%, 100% identical to SEQ ID NO: 1.

Embodiment 166. The method of any one of embodiments 139-165, wherein the detectable peptide derived from the spike glycoprotein of a coronavirus comprises at least a portion of a spike protein derived from Severe acute respiratory syndrome-associated coronavirus (SARS-CoV).

Embodiment 167. The method of any one of embodiments 139-166, wherein the SARS-CoV is SARS-CoV-2.

Embodiment 168. The method of any one of embodiments 139-167, wherein an infection in a human subject by the SARS-CoV-2 causes coronavirus disease of 2019 (COVID-19) in the human subject.

Embodiment 169. The method of any one of embodiments 139-168, wherein the at least a portion of the spike protein comprises a subunit 1 of the spike protein.

Embodiment 170. The method of any one of embodiments 139-169, wherein the at least a portion of the spike protein comprises a receptor binding domain (RBD) of the subunit 1 of the spike protein.

Embodiment 171. The method of any one of embodiments 139-170, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 2.

Embodiment 172. The method of any one of embodiments 139-171, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 3.

Embodiment 173. The method of any one of embodiments 139-172, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to any one of SEQ ID NOS: 4-6, 11-12, 24, 47-48, 71-72, 95-96, or 115.

Embodiment 174. The method of any one of embodiments 139-173, wherein the complex between the peptide-conjugate and the one or more capture molecules is visible on the surface using color, reflectance, fluorescence, bioluminescence, or chemiluminescence.

Embodiment 175. The method of any one of embodiments 139-174, wherein method further comprises providing a web-based portal on the personal electronic device.

Embodiment 176. The method of any one of embodiments 139-175, wherein method further comprises providing an application on the web-based portal.

Embodiment 177. The method of any one of embodiments 139-176, wherein method further comprises receiving data, by the application, the data comprising a result from the image captured by the personal electronic device, a geolocation of the personal electronic device, or external data from one or more external devices.

Embodiment 178. The method of any one of embodiments 139-177, wherein method further comprises providing a data analytics module at the application.

Embodiment 179. The method of any one of embodiments 139-178, wherein method further comprises analyzing the result, by the data analytics module, to determining whether the result is a positive result or a negative result, wherein a positive results indicates immunity and a negative results indicates a lack of immunity.

Embodiment 180. The method of any one of embodiments 139-179, wherein the positive or the negative result is relative to a threshold number of complexes between the detectable peptide and the one or more capture molecules on the surface.

Embodiment 181. The method of any one of embodiments 139-180, wherein the threshold number is predetermined relative to an index a control.

Embodiment 182. The method of any one of embodiments 139-181, wherein method further comprises analyzing the result, by the data analytics module, to determining an absolute number of complexes between the detectable peptide and the one or more capture molecules on the surface.

Embodiment 183. The method of any one of embodiments 139-182, wherein method further comprises analyzing the result, by the data analytics module, to determine a level of binding between the peptide-conjugate and the ACE2, wherein a high level of binding indicates a low level of immunity, and a low level of binding indicates a high level of immunity.

Embodiment 184. The method of any one of embodiments 139-183, wherein method further comprises normalizing, by the data analytics module, the result by subtracting background noise.

Embodiment 185. The method of any one of embodiments 139-184, wherein method further comprises identifying, by the data analytics module, a geographical location comprised of subjects for which a positive result was determined to detect a presence of herd immunity to the coronavirus, or recommend further testing.

Embodiment 186. The method of any one of embodiments 139-185, wherein identifying the geographical location comprises utilizing geofencing from coordinates of the personal electronic device.

Embodiment 187. The method of any one of embodiments 139-186, wherein the data analytics module utilizes a machine learning algorithm, artificial intelligence, or both.

Embodiment 188. The method of any one of embodiments 139-187, wherein method further comprises providing a data store that is a cloud-based data store or a web-based data store, or a local data store.

Embodiment 189. The method of any one of embodiments 139-188, wherein the data comprises one or more of geolocation of the imaging device, a result from the image captured by the imaging device, and external data.

Embodiment 190. The method of any one of embodiments 139-189, wherein external data is data from an external device selected from a diagnostic device, a prognostic device, or a health or fitness tracking device.

Embodiment 191. The method of any one of embodiments 139-190, wherein external data comprises body temperature, heart rate variability, resting heart rate, sleep quality, and sleep quantity.

Embodiment 192. The method of any one of embodiments 139-191, wherein comprising receiving, from an external device, the external data, wherein the external device is selected from a diagnostic device, a prognostic device, or a health or fitness tracking device.

Embodiment 193. Aspects disclosed herein provide a method of identifying adaptive immunity to a coronavirus in a subject, the method comprising: (a) obtaining a biological sample from the subject; (b) producing a mixture by introducing the biological sample with a detectable peptide derived from an angiotensin-converting enzyme 2 (ACE2) receptor; (c) bringing the mixture into contact with one or more capture molecules derived from a spike glycoprotein of a coronavirus; (d) detecting a number of binding complexes between the detectable peptide and the one or more capture molecules; (e) if the number of the binding complexes is low relative to an index or a control, then identifying the subject as being immune to an infection by the coronavirus; and (f) if the number of the binding complexes is high relative to an index or a control, then identifying the subject as not being immune to an infection by the coronavirus.

Embodiment 194. The method of embodiment 193, wherein steps (a)-(f) are performed at the point of need.

Embodiment 195. The method of any one of embodiments 193-194, wherein steps (a)-(f) are performed at the point of care.

Embodiment 196. The method of any one of embodiments 193-195, wherein the surface comprises a material selected from the group consisting of a metal, a plastic, glass, and a nitrocellulose membrane.

Embodiment 197. The method of any one of embodiments 193-196, wherein the surface is a passivated surface.

Embodiment 198. The method of any one of embodiments 193-197, wherein the passivated surface comprises a polymer layer comprising a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran.

Embodiment 199. The method of any one of embodiments 193-198, wherein the one or more capture molecules comprises two or more capture molecules.

Embodiment 200. The method of any one of embodiments 193-199, wherein the one or more capture molecules is coupled to the surface by a covalent bond, a linker, or a combination thereof.

Embodiment 201. The method of any one of embodiments 193-200, wherein the linker is a chemical linker, a peptide linker, or a combination thereof.

Embodiment 202. The method of any one of embodiments 193-201, wherein the one or more capture molecules is a fusion polypeptide.

Embodiment 203. The method of any one of embodiments 193-202, wherein the fusion polypeptide comprises at least a portion of a fragment crystallizable region (Fc) region of a monoclonal antibody.

Embodiment 204. The method of any one of embodiments 193-203, wherein the one or more capture molecules is bound by an antibody that is coupled to the surface.

Embodiment 205. The method of any one of embodiments 193-204, wherein the detectable peptide comprises a nanoparticle, a fluorescent dye, an enzymatic label, or a colorimetric label, or a combination thereof.

Embodiment 206. The method of any one of embodiments 193-205, wherein the nanoparticle comprises a material selected from the group consisting of a metal, agarose, acrylic, and plastic.

Embodiment 207. The method of any one of embodiments 193-206, wherein the nanoparticle is magnetic.

Embodiment 208. The method of any one of embodiments 193-207, wherein the nanoparticle is conjugated to the peptide by a linker comprising a chemical linker, a peptide linker, or a combination thereof.

Embodiment 209. The method of any one of embodiments 193-208, wherein the detectable peptide derived from the ACE2 receptor comprises an amino acid sequence that is at least 70%, 75%, 80%, 85%, 90%, 91%, 92%, 93%, 94%, 95%, 96%, 97%, 98%, 99%, 100% identical to SEQ ID NO: 1.

Embodiment 210. The method of any one of embodiments 193-209, wherein the one or more capture molecules derived from the spike glycoprotein of a coronavirus comprises at least a portion of a spike protein derived from Severe acute respiratory syndrome-associated coronavirus (SARS-CoV).

Embodiment 211. The method of any one of embodiments 193-210, wherein the SARS-CoV is SARS-CoV-2.

Embodiment 212. The method of any one of embodiments 193-211, wherein an infection in a human subject by the SARS-CoV-2 causes coronavirus disease of 2019 (COVID-19) in the human subject.

Embodiment 213. The method of any one of embodiments 193-212, wherein the at least a portion of the spike protein comprises a subunit 1 of the spike protein.

Embodiment 214. The method of any one of embodiments 193-213, wherein the at least a portion of the spike protein comprises a receptor binding domain (RBD) of the subunit 1 of the spike protein.

Embodiment 215. The method of any one of embodiments 193-214, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 2.

Embodiment 216. The method of any one of embodiments 193-215, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 3.

Embodiment 217. The method of any one of embodiments 193-216, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to any one of SEQ ID NOS: 4-6, 11-12, 24, 47-48, 71-72, 95-96, or 115.

Embodiment 218. The method of any one of embodiments 193-217, wherein the complex between the peptide-conjugate and the one or more capture molecules is visible on the surface using color, reflectance, fluorescence, bioluminescence, or chemiluminescence.

Embodiment 219. The method of any one of embodiments 193-218, wherein the biological sample comprises one or more antibodies specific to the peptide.

Embodiment 220. The method of any one of embodiments 193-219, wherein the biological sample does not consist of one or more antibodies specific to the peptide.

Embodiment 221. The method of any one of embodiments 193-220, wherein the one or more antibodies belong to one or more immunoglobulin classes comprising immunoglobulin M, immunoglobulin G, immunoglobulin A, immunoglobulin E, and immunoglobulin D.

Embodiment 222. The method of any one of embodiments 193-221, wherein the subject was, or is, exposed to the coronavirus.

Embodiment 223. The method of any one of embodiments 193-222, wherein exposure of the subject to the coronavirus is unknown.

Embodiment 224. The method of any one of embodiments 193-223, wherein the subject is a plurality of subjects.

Embodiment 225. The method of any one of embodiments 193-224, wherein method further comprises identifying adaptive immunity of the plurality of subjects to the coronavirus.

Embodiment 226. The method of any one of embodiments 193-225, wherein method further comprises monitoring a spread of infection of the plurality of subjects by the coronavirus.

Embodiment 227. The method of any one of embodiments 193-226, wherein the subject was administered a vaccine against the coronavirus.

Embodiment 228. The method of any one of embodiments 193-227, wherein method further comprises determining that the vaccine is effective to substantially immunize the subject against the coronavirus, provided the number of the binding complexes is low relative to an index or a control.

Embodiment 229. The method of any one of embodiments 193-228, wherein the biological sample comprises blood, urine, saliva, or feces.

Embodiment 230. The method of any one of embodiments 193-229, wherein the blood is capillary blood.

Embodiment 231. The method of any one of embodiments 193-230, wherein the capillary blood is obtained from the subject by a prick of the subject's finger.

Embodiment 232. The method of any one of embodiments 193-231, wherein method further comprises separating serum from the blood in the biological sample.

Embodiment 233. The method of any one of embodiments 193-232, wherein detecting in (d) comprises capturing an image of the surface with an imaging device to detect a number of binding complexes between the detectable peptide and the one or more capture molecules.

Embodiment 234. The method of any one of embodiments 193-233, wherein the imaging device is a personal electronic device.

Embodiment 235. The method of any one of embodiments 193-234, wherein the personal electronic device is a smart phone, tablet, body camera, web camera, or personal computer.

Embodiment 236. The method of any one of embodiments 193-235, wherein method further comprises providing a web-based portal on the personal electronic device.

Embodiment 237. The method of any one of embodiments 193-236, wherein method further comprises providing an application on the web-based portal.

Embodiment 238. The method of any one of embodiments 193-237, wherein method further comprises receiving data, by the application, the data comprising a result from the image captured by the personal electronic device, a geolocation of the personal electronic device, or external data from one or more external devices.

Embodiment 239. The method of any one of embodiments 193-238, wherein method further comprises providing a data analytics module at the application.

Embodiment 240. The method of any one of embodiments 193-239, wherein method further comprises analyzing the result, by the data analytics module, to determining whether the result is a positive result or a negative result, wherein a positive results indicates immunity and a negative results indicates a lack of immunity.

Embodiment 241. The method of any one of embodiments 193-240, wherein the positive or the negative result is relative to a threshold number of complexes between the detectable peptide and the one or more capture molecules on the surface.

Embodiment 242. The method of any one of embodiments 193-241, wherein the threshold number is predetermined relative to an index a control.

Embodiment 243. The method of any one of embodiments 193-242, wherein method further comprises analyzing the result, by the data analytics module, to determining an absolute number of complexes between the detectable peptide and the one or more capture molecules on the surface.

Embodiment 244. The method of any one of embodiments 193-243, wherein method further comprises analyzing the result, by the data analytics module, to determine a level of binding between the peptide-conjugate and the ACE2, wherein a high level of binding indicates a low level of immunity, and a low level of binding indicates a high level of immunity.

Embodiment 245. The method of any one of embodiments 193-244, wherein method further comprises normalizing, by the data analytics module, the result by subtracting background noise.

Embodiment 246. The method of any one of embodiments 193-245, wherein method further comprises identifying, by the data analytics module, a geographical location comprised of subjects for which a positive result was determined to detect a presence of herd immunity to the coronavirus, or recommend further testing.

Embodiment 247. The method of any one of embodiments 193-246, wherein identifying the geographical location comprises utilizing geofencing from coordinates of the personal electronic device.

Embodiment 248. The method of any one of embodiments 193-247, wherein the data analytics module utilizes a machine learning algorithm, artificial intelligence, or both.

Embodiment 249. The method of any one of embodiments 193-248, wherein method comprises further comprise providing a data store that is a cloud-based data store or a web-based data store, or a local data store.

Embodiment 250. The method of any one of embodiments 193-249, wherein the data comprises one or more of geolocation of the imaging device, a result from the image captured by the imaging device, and external data.

Embodiment 251. The method of any one of embodiments 193-250, wherein external data is data from an external device selected from a diagnostic device, a prognostic device, or a health or fitness tracking device.

Embodiment 252. The method of any one of embodiments 193-251, wherein external data comprises body temperature, heart rate variability, resting heart rate, sleep quality, and sleep quantity.

Embodiment 253. The method of any one of embodiments 193-252, wherein comprising receiving, from an external device, the external data, wherein the external device is selected from a diagnostic device, a prognostic device, or a health or fitness tracking device.

Embodiment 254. Aspects disclosed herein provide a method comprising: (a) obtaining a biological sample from the subject; (b) determining whether the subject has an acute infection by a pathogen by: (i) introducing the biological sample to a mixture comprising one or more capture molecules specific to an antibody against the pathogen; (ii) detecting a number of binding complexes between the one or more capture molecules specific to the antibody against the pathogen, wherein if the number of the binding complexes is high relative to an index or a control, then identifying the subject as having the acute infection by the coronavirus; or (c) determining whether the subject has a sufficient adaptive immunity against the pathogen by: (i) introducing the biological sample with a second mixture comprising detectable peptide; (ii) bringing the second mixture into contact with one or more capture molecules specific to the detectable peptide; (iii) detecting a number of binding complexes between the detectable peptide and the one or more capture molecules specific to the peptide, wherein if the number of the binding complexes is low, then identifying the subject as being immune to an infection by the coronavirus.

Embodiment 255. The method of embodiment 254, wherein if the number of the binding complexes is high relative to an index or a control, then identifying the subject as not being immune to an infection by the coronavirus.

Embodiment 256. The method of any one of embodiments 254-255, wherein the first mixture and the second mixture is a single mixture.

Embodiment 257. The method of any one of embodiments 254-256, wherein the one or more capture molecules comprises two or more capture molecules.

Embodiment 258. The method of any one of embodiments 254-257, wherein the one or more capture molecules comprises a first antibody, a second antibody or a third antibody, wherein the first antibody is specific to an immunoglobulin G antibody against SARS-CoV-2, the second antibody is specific to an immunoglobulin M antibody against SARS-CoV-2, and the third antibody is specific to an immunoglobulin A of the SARS-CoV-2.

Embodiment 259. The method of any one of embodiments 254-258, wherein steps (a)-(c) are performed at the point of need.

Embodiment 260. The method of any one of embodiments 254-259, wherein steps (a)-(c) are performed at the point of care.

Embodiment 261. The method of any one of embodiments 254-260, wherein the one or more capture molecules specific to the antibody against the pathogen are coupled to a surface.

Embodiment 262. The method of any one of embodiments 254-261, wherein the one or more capture molecules specific to the labeled peptide are coupled to the surface.

Embodiment 263. The method of any one of embodiments 254-262, wherein the surface comprises a material selected from the group consisting of a metal, a plastic, glass, and a nitrocellulose membrane.

Embodiment 264. The method of any one of embodiments 254-263, wherein the surface is a passivated surface.

Embodiment 265. The method of any one of embodiments 254-264, wherein the passivated surface comprises a polymer layer comprising a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran.

Embodiment 266. The method of any one of embodiments 254-265, wherein the one or more capture molecules is coupled to the surface by a covalent bond, a linker, or a combination thereof.

Embodiment 267. The method of any one of embodiments 254-266, wherein the linker is a chemical linker, a peptide linker, or a combination thereof.

Embodiment 268. The method of any one of embodiments 254-267, wherein the one or more capture molecules is a fusion polypeptide.

Embodiment 269. The method of any one of embodiments 254-268, wherein the fusion polypeptide comprises at least a portion of a fragment crystallizable region (Fc) region of a monoclonal antibody.

Embodiment 270. The method of any one of embodiments 254-269, wherein the one or more capture molecules is bound by an antibody that is coupled to the surface.

Embodiment 271. The method of any one of embodiments 254-270, wherein the detectable peptide comprises a nanoparticle, a fluorescent dye, an enzymatic label, or a colorimetric label, or a combination thereof.

Embodiment 272. The method of any one of embodiments 254-271, wherein the nanoparticle comprises a material selected from the group consisting of a metal, agarose, acrylic, and plastic.

Embodiment 273. The method of any one of embodiments 254-272, wherein the nanoparticle is magnetic.

Embodiment 274. The method of any one of embodiments 254-273, wherein the nanoparticle is conjugated to the peptide by a linker comprising a chemical linker, a peptide linker, or a combination thereof.

Embodiment 275. The method of any one of embodiments 254-274, wherein the detectable peptide is derived from the ACE2 receptor and comprises an amino acid sequence that is at least 70%, 75%, 80%, 85%, 90%, 91%, 92%, 93%, 94%, 95%, 96%, 97%, 98%, 99%, 100% identical to SEQ ID NO: 1.

Embodiment 276. The method of any one of embodiments 254-275, wherein the one or more capture molecules specific to the detectable peptide is derived from the spike glycoprotein of a coronavirus and comprises at least a portion of a spike protein derived from Severe acute respiratory syndrome-associated coronavirus (SARS-CoV).

Embodiment 277. The method of any one of embodiments 254-276, wherein the SARS-CoV is SARS-CoV-2.

Embodiment 278. The method of any one of embodiments 254-277, wherein an infection in a human subject by the SARS-CoV-2 causes coronavirus disease of 2019 (COVID-19) in the human subject.

Embodiment 279. The method of any one of embodiments 254-278, wherein the at least a portion of the spike protein comprises a subunit 1 of the spike protein.

Embodiment 280. The method of any one of embodiments 254-279, wherein the at least a portion of the spike protein comprises a receptor binding domain (RBD) of the subunit 1 of the spike protein.

Embodiment 281. The method of any one of embodiments 254-280, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 2.

Embodiment 282. The method of any one of embodiments 254-281, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 3.

Embodiment 283. The method of any one of embodiments 254-282, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to any one of SEQ ID NOS: 4-6, 11-12, 24, 47-48, 71-72, 95-96, or 115.

Embodiment 284. The method of any one of embodiments 254-283, wherein the complex between the peptide-conjugate and the one or more capture molecules is visible on the surface using color, reflectance, fluorescence, bioluminescence, or chemiluminescence.

Embodiment 285. The method of any one of embodiments 254-284, wherein the one or more capture molecules specific to the detectable peptide is derived from the ACE2 receptor and comprises an amino acid sequence that is at least 70%, 75%, 80%, 85%, 90%, 91%, 92%, 93%, 94%, 95%, 96%, 97%, 98%, 99%, 100% identical to SEQ ID NO: 1.

Embodiment 286. The method of any one of embodiments 254-285, wherein the detectable peptide is derived from the spike glycoprotein of a coronavirus and comprises at least a portion of a spike protein derived from Severe acute respiratory syndrome-associated coronavirus (SARS-CoV).

Embodiment 287. The method of any one of embodiments 254-286, wherein the SARS-CoV is SARS-CoV-2.

Embodiment 288. The method of any one of embodiments 254-287, wherein an infection in a human subject by the SARS-CoV-2 causes coronavirus disease of 2019 (COVID-19) in the human subject.

Embodiment 289. The method of any one of embodiments 254-288, wherein the at least a portion of the spike protein comprises a subunit 1 of the spike protein.

Embodiment 290. The method of any one of embodiments 254-289, wherein the at least a portion of the spike protein comprises a receptor binding domain (RBD) of the subunit 1 of the spike protein.

Embodiment 291. The method of any one of embodiments 254-290, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 2.

Embodiment 292. The method of any one of embodiments 254-291, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 3.

Embodiment 293. The method of any one of embodiments 254-292, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to any one of SEQ ID NOS: 4-6, 11-12, 24, 47-48, 71-72, 95-96, or 115.

Embodiment 294. The method of any one of embodiments 254-293, wherein the complex between the detectable peptide and the one or more capture molecules specific to the detectable peptide is visible on the surface using color, reflectance, fluorescence, bioluminescence, or chemiluminescence.

Embodiment 295. The method of any one of embodiments 254-294, wherein the biological sample comprises one or more antibodies specific to the peptide.

Embodiment 296. The method of any one of embodiments 254-295, wherein the biological sample does not consist of one or more antibodies specific to the peptide.

Embodiment 297. The method of any one of embodiments 254-2%, wherein the one or more antibodies belong to one or more immunoglobulin classes comprising immunoglobulin M, immunoglobulin G, immunoglobulin A, immunoglobulin E, and immunoglobulin D.

Embodiment 298. The method of any one of embodiments 254-297, wherein pathogen is a coronavirus.

Embodiment 299. The method of any one of embodiments 254-298, wherein the subject was, or is, exposed to the coronavirus.

Embodiment 300. The method of any one of embodiments 254-299, wherein exposure of the subject to the coronavirus is unknown.

Embodiment 301. The method of any one of embodiments 254-300, wherein the subject is a plurality of subjects.

Embodiment 302. The method of any one of embodiments 254-301, wherein method further comprises identifying adaptive immunity of the plurality of subjects to the coronavirus.

Embodiment 303. The method of any one of embodiments 254-302, wherein method further comprises monitoring a spread of infection of the plurality of subjects by the coronavirus.

Embodiment 304. The method of any one of embodiments 254-303, wherein the subject was administered a vaccine against the coronavirus.

Embodiment 305. The method of any one of embodiments 254-304, wherein method further comprises determining that the vaccine is effective to substantially immunize the subject against the coronavirus, provided the number of the binding complexes between the detectable peptide and the one or more capture molecules specific to the detectable peptide is low relative to an index or a control.

Embodiment 306. The method of any one of embodiments 254-305, wherein the biological sample comprises blood, urine, saliva, or feces.

Embodiment 307. The method of any one of embodiments 254-306, wherein the blood is capillary blood.

Embodiment 308. The method of any one of embodiments 254-307, wherein the capillary blood is obtained from the subject by a prick of the subject's finger.

Embodiment 309. The method of any one of embodiments 254-308, wherein method further comprises separating serum from the blood in the biological sample.

Embodiment 310. The method of any one of embodiments 254-309, wherein detecting in (d) comprises capturing an image of the surface with an imaging device to detect a number of binding complexes between the detectable peptide and the one or more capture molecules.

Embodiment 311. The method of any one of embodiments 254-310, wherein the imaging device is a personal electronic device.

Embodiment 312. The method of any one of embodiments 254-311, wherein the personal electronic device is a smart phone, tablet, body camera, web camera, or personal computer.

Embodiment 313. The method of any one of embodiments 254-312, wherein method further comprises providing a web-based portal on the personal electronic device.

Embodiment 314. The method of any one of embodiments 254-313, wherein method further comprises providing an application on the web-based portal.

Embodiment 315. The method of any one of embodiments 254-314, wherein method further comprises receiving data, by the application, the data comprising a result from the image captured by the personal electronic device, a geolocation of the personal electronic device, or external data from one or more external devices.

Embodiment 316. The method of any one of embodiments 254-315, wherein method further comprises providing a data analytics module at the application.

Embodiment 317. The method of any one of embodiments 254-316 wherein method further comprises analyzing the result, by the data analytics module, to determining whether the result is a positive result or a negative result, wherein a positive results indicates immunity and a negative results indicates a lack of immunity.

Embodiment 318. The method of any one of embodiments 254-317, wherein the positive or the negative result (e.g., immune or not immune) is relative to a threshold number of complexes between the detectable peptide and the one or more capture molecules.

Embodiment 319. The method of any one of embodiments 254-318, wherein the threshold number is predetermined relative to an index a control.

Embodiment 320. The method of any one of embodiments 254-319, wherein the positive or the negative result (e.g., acute infection present or not present) is relative to a threshold number of complexes between the one or more capture molecules specific to the antibody against the pathogen and the antibody against the pathogen.

Embodiment 321. The method of any one of embodiments 254-320, wherein method further comprises analyzing the result, by the data analytics module, to determining an absolute number of complexes between the detectable peptide and the one or more capture molecules specific to the detectable peptide, or the antibody against the pathogen and the one or more capture molecules specific to the antibody.

Embodiment 322. The method of any one of embodiments 254-321, wherein method further comprises analyzing the result, by the data analytics module, to determine a level of binding between the detectable peptide and the one or more capture molecules specific to the detectable peptide, wherein a high level of binding indicates a low level of immunity, and a low level of binding indicates a high level of immunity.

Embodiment 323. The method of any one of embodiments 254-322, wherein method further comprises analyzing the result, by the data analytics module, to determine a level of binding between the one or more capture molecules specific to the antibody against the pathogen and the antibody against the pathogen, wherein a high level of binding indicates a presence of an acute infection by the pathogen.

Embodiment 324. The method of any one of embodiments 254-323, wherein method further comprises normalizing, by the data analytics module, the result by subtracting background noise.

Embodiment 325. The method of any one of embodiments 254-324, wherein method further comprises identifying, by the data analytics module, a geographical location comprised of subjects for which a positive result for immunity was determined to detect a presence of herd immunity to the pathogen, or recommend further testing.

Embodiment 326. The method of any one of embodiments 254-325, wherein method further comprises identifying, by the data analytics module, a geographical location comprised of subjects for which a positive result for an acute infection was determined to detect a presence of infectivity or re-infectivity by the pathogen, or recommend further testing.

Embodiment 327. The method of any one of embodiments 254-326, wherein identifying the geographical location comprises utilizing geofencing from coordinates of the personal electronic device.

Embodiment 328. The method of any one of embodiments 254-327, wherein the data analytics module utilizes a machine learning algorithm, artificial intelligence, or both.

Embodiment 329. The method of any one of embodiments 254-328, wherein method further comprises providing a data store that is a cloud-based data store or a web-based data store, or a local data store.

Embodiment 330. The method of any one of embodiments 254-329, wherein the data comprises one or more of geolocation of the imaging device, a result from the image captured by the imaging device, and external data.

Embodiment 331. The method of any one of embodiments 254-330, wherein external data is data from an external device selected from a diagnostic device, a prognostic device, or a health or fitness tracking device.

Embodiment 332. The method of any one of embodiments 254-331, wherein external data comprises body temperature, heart rate variability, resting heart rate, sleep quality, and sleep quantity.

Embodiment 333. The method of any one of embodiments 254-332, wherein comprising receiving, from an external device, the external data, wherein the external device is selected from a diagnostic device, a prognostic device, or a health or fitness tracking device.

Embodiment 334. Aspects disclosed herein provide a device comprising: (a) a liquid composition comprising a peptide-conjugate comprising: (i) a peptide derived from a spike glycoprotein of a coronavirus; and (ii) a detectable moiety; and (b) a surface submerged in the liquid composition, the surface comprising one or more capture molecules coupled to the surface, the one or more capture molecules derived from an angiotensin-converting enzyme 2 (ACE2) receptor.

Embodiment 335. The device of embodiment 334, wherein the surface is a surface of a container, wherein the container contains (a) and (b).

Embodiment 336. The device of any one of embodiments 334-335, wherein the device is portable.

Embodiment 337. The device of any one of embodiments 334-336, wherein the device is a point of need device.

Embodiment 338. The device of any one of embodiments 334-337, wherein the point of need is a point of care.

Embodiment 339. The device of any one of embodiments 334-338, wherein the surface comprises a material selected from the group consisting of a metal, a plastic, glass, and a nitrocellulose membrane.

Embodiment 340. The device of any one of embodiments 334-339, wherein the surface is a passivated surface.

Embodiment 341. The device of any one of embodiments 334-340, wherein the passivated surface comprises a polymer layer comprising a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran.

Embodiment 342. The device of any one of embodiments 334-341, wherein the one or more capture molecules comprises two or more capture molecules.

Embodiment 343. The device of any one of embodiments 334-342, wherein the one or more capture molecules is coupled to the surface by a covalent bond, a linker, or a combination thereof.

Embodiment 344. The device of any one of embodiments 334-343, wherein the linker is a chemical linker, a peptide linker, or a combination thereof.

Embodiment 345. The device of any one of embodiments 334-344, wherein the one or more capture molecules is a fusion polypeptide.

Embodiment 346. The device of any one of embodiments 334-345, wherein the fusion polypeptide comprises at least a portion of a fragment crystallizable region (Fc) region of a monoclonal antibody.

Embodiment 347. The device of any one of embodiments 334-346, wherein the one or more capture molecules is bound by an antibody that is coupled to the surface.

Embodiment 348. The device of any one of embodiments 334-347, wherein the peptide-conjugate comprises a nanoparticle, a fluorescent dye, an enzymatic label, or a colorimetric label, or a combination thereof.

Embodiment 349. The device of any one of embodiments 334-348, wherein the nanoparticle comprises a material selected from the group consisting of a metal, agarose, acrylic, and plastic.

Embodiment 350. The device of any one of embodiments 334-349, wherein the nanoparticle is magnetic.

Embodiment 351. The device of any one of embodiments 334-350, wherein the nanoparticle is conjugated to the peptide by a linker comprising a chemical linker, a peptide linker, or a combination thereof.

Embodiment 352. The device of any one of embodiments 334-3514, wherein the one or more capture molecules derived from the ACE2 receptor comprises an amino acid sequence that is at least 70%, 75%, 80%, 85%, 90%, 91%, 92%, 93%, 94%, 95%, 96%, 97%, 98%, 99%, 100% identical to SEQ ID NO: 1.

Embodiment 353. The device of any one of embodiments 334-352, wherein the peptide derived from the spike glycoprotein of a coronavirus comprises at least a portion of a spike protein derived from Severe acute respiratory syndrome-associated coronavirus (SARS-CoV).

Embodiment 354. The device of any one of embodiments 334-353, wherein the SARS-CoV is SARS-CoV-2.

Embodiment 355. The device of any one of embodiments 334-354, wherein an infection in a human subject by the SARS-CoV-2 causes coronavirus disease of 2019 (COVID-19) in the human subject.

Embodiment 356. The device of any one of embodiments 334-355, wherein the at least a portion of the spike protein comprises a subunit 1 of the spike protein.

Embodiment 357. The device of any one of embodiments 334-356, wherein the at least a portion of the spike protein comprises a receptor binding domain (RBD) of the subunit 1 of the spike protein.

Embodiment 358. The device of any one of embodiments 334-357, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 2.

Embodiment 359. The device of any one of embodiments 334-358, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 3.

Embodiment 360. The device of any one of embodiments 334-359, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to any one of SEQ ID NOS: 4-6, 11-12, 24, 47-48, 71-72, 95-96, or 115.

Embodiment 361. The device of any one of embodiments 334-360, wherein the complex between the peptide-conjugate and the one or more capture molecules receptor on the surface is visible on the surface using color, reflectance, fluorescence, bioluminescence, or chemiluminescence.

Embodiment 362. The device of any one of embodiments 334-361, wherein the one or more capture molecules is specific to one or more antibodies against the coronavirus.

Embodiment 363. The device of any one of embodiments 334-362, wherein the one or more capture molecules specific to one or more antibodies against the coronavirus is labeled.

Embodiment 364. The device of any one of embodiments 334-363, wherein device further comprise a labeled secondary capture molecule specific to the one or more capture molecules specific to one or more antibodies against the coronavirus.

Embodiment 365. The device of any one of embodiments 334-364, wherein the one or more capture molecules specific to one or more antibodies against the coronavirus is an antibody or antigen-binding fragment.

Embodiment 366. The device of any one of embodiments 334-365, wherein the antibody or antigen-binding fragment is monoclonal. antibody or antigen-binding fragment is polyclonal.

Embodiment 367. The device of any one of embodiments 334-366, wherein device further comprises a housing at least partially enclosing the surface.

Embodiment 368. The device of any one of embodiments 334-367, wherein device further comprises a sample receptor configured to receive a biological sample from a subject.

Embodiment 369. The device of any one of embodiments 334-368, wherein the sample receptor is mechanically coupled to a housing at least partially enclosing the surface.

Embodiment 370. The device of any one of embodiments 334-369, wherein the biological sample comprises one or more antibodies specific to the peptide.

Embodiment 371. The device of any one of embodiments 334-370, wherein the biological sample does not consist of one or more antibodies specific to the peptide.

Embodiment 372. The device of any one of embodiments 334-371, wherein the one or more antibodies belong to one or more immunoglobulin classes comprising immunoglobulin M, immunoglobulin G, immunoglobulin A, immunoglobulin E, and immunoglobulin D.

Embodiment 373. The device of any one of embodiments 334-372, wherein the subject was, or is, exposed to the coronavirus.

Embodiment 374. The device of any one of embodiments 334-373, wherein exposure of the subject to the coronavirus is unknown.

Embodiment 375. The device of any one of embodiments 334-374, wherein the subject was administered a vaccine against the coronavirus.

Embodiment 376. The device of any one of embodiments 334-375, wherein the biological sample comprises blood, urine, saliva, or feces.

Embodiment 377. The device of any one of embodiments 334-376, wherein the blood is capillary blood.

Embodiment 378. The device of any one of embodiments 334-377, wherein device further comprises a transdermal puncture device configured to obtain the capillary blood from the subject.

Embodiment 379. The device of any one of embodiments 334-378, wherein the sample receptor comprises a filter to separate serum from the blood.

Embodiment 380. The device of any one of embodiments 334-379, wherein the device is a single integrated device.

Embodiment 381. Aspects disclosed herein provide a device comprising: (a) a liquid composition comprising a peptide-conjugate comprising: (i) a peptide derived from an angiotensin-converting enzyme 2 (ACE2) receptor; and (ii) a detectable moiety; and (b) a surface submerged in the liquid composition, the surface comprising one or more capture molecules coupled to the surface, the one or more capture molecules derived from a spike glycoprotein of a coronavirus.

Embodiment 382. The device of embodiment 381, wherein the surface is a surface of a container, wherein the container contains (a) and (b).

Embodiment 383. The device of any one of embodiments 381-382, wherein the device is portable.

Embodiment 384. The device of any one of embodiments 381-383, wherein the device is a point of need device.

Embodiment 385. The device of any one of embodiments 381-384, wherein the point of need is a point of care.

Embodiment 386. The device of any one of embodiments 381-385, wherein the surface comprises a material selected from the group consisting of a metal, a plastic, glass, and a nitrocellulose membrane.

Embodiment 387. The device of any one of embodiments 381-386, wherein the surface is a passivated surface.

Embodiment 388. The device of any one of embodiments 381-387, wherein the passivated surface comprises a polymer layer comprising a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran.

Embodiment 389. The device of any one of embodiments 381-388, wherein the one or more capture molecules comprises two or more capture molecules.

Embodiment 390. The device of any one of embodiments 381-389, wherein the one or more capture molecules is coupled to the surface by a covalent bond, a linker, or a combination thereof.

Embodiment 391. The device of any one of embodiments 381-390, wherein the linker is a chemical linker, a peptide linker, or a combination thereof.

Embodiment 392. The device of any one of embodiments 381-391, wherein the one or more capture molecules is a fusion polypeptide.

Embodiment 393. The device of any one of embodiments 381-392, wherein the fusion polypeptide comprises at least a portion of a fragment crystallizable region (Fc) region of a monoclonal antibody.

Embodiment 394. The device of any one of embodiments 381-393, wherein the one or more capture molecules is bound by an antibody that is coupled to the surface.

Embodiment 395. The device of any one of embodiments 381-394, wherein the peptide-conjugate comprises a nanoparticle, a fluorescent dye, an enzymatic label, or a colorimetric label, or a combination thereof.

Embodiment 3%. The device of any one of embodiments 381-395, wherein the nanoparticle comprises a material selected from the group consisting of a metal, agarose, acrylic, and plastic.

Embodiment 397. The device of any one of embodiments 381-3%, wherein the nanoparticle is magnetic.

Embodiment 398. The device of any one of embodiments 381-397, wherein the nanoparticle is conjugated to the peptide by a linker comprising a chemical linker, a peptide linker, or a combination thereof.

Embodiment 399. The device of any one of embodiments 381-398, wherein the peptide derived from the ACE2 receptor comprises an amino acid sequence that is at least 70%, 75%, 80%, 85%, 90%, 91%, 92%, 93%, 94%, 95%, %%, 97%, 98%, 99%, 100% identical to SEQ ID NO: 1.

Embodiment 400. The device of any one of embodiments 381-399, wherein the one or more capture molecules derived from the spike glycoprotein of a coronavirus comprises at least a portion of a spike protein derived from Severe acute respiratory syndrome-associated coronavirus (SARS-CoV).

Embodiment 401. The device of any one of embodiments 381-400, wherein the SARS-CoV is SARS-CoV-2.

Embodiment 402. The device of any one of embodiments 381-401, wherein an infection in a human subject by the SARS-CoV-2 causes coronavirus disease of 2019 (COVID-19) in the human subject.

Embodiment 403. The device of any one of embodiments 381-402, wherein the at least a portion of the spike protein comprises a subunit 1 of the spike protein.

Embodiment 404. The device of any one of embodiments 381-403, wherein the at least a portion of the spike protein comprises a receptor binding domain (RBD) of the subunit 1 of the spike protein.

Embodiment 405. The device of any one of embodiments 381-404, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 2.

Embodiment 406. The device of any one of embodiments 381-405, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to SEQ ID NO: 3.

Embodiment 407. The device of any one of embodiments 381-406, wherein the at least a portion of the spike protein comprises an amino acid sequence that is at least 70%, 75%, 80%, 85%, 90%, 91%, 92%, 93%, 94%, 95%, 96%, 97%, 98%, 99%, 100% identical to any one of SEQ ID NOS: 4-6, 11-12, 24, 47-48, 71-72, 95-96, or 115.

Embodiment 408. The device of any one of embodiments 381-407, wherein the complex between the peptide-conjugate and the one or more capture molecules receptor on the surface is visible on the surface using color, reflectance, fluorescence, bioluminescence, or chemiluminescence.

Embodiment 409. The device of any one of embodiments 381-408, wherein the one or more capture molecules is specific to one or more antibodies against the coronavirus.

Embodiment 410. The device of any one of embodiments 381-409, wherein the one or more capture molecules specific to one or more antibodies against the coronavirus is labeled.

Embodiment 411. The device of any one of embodiments 381-410, wherein device further comprises a labeled secondary capture molecule specific to the one or more capture molecules specific to one or more antibodies against the coronavirus.

Embodiment 412. The device of any one of embodiments 381-411, wherein the one or more capture molecules specific to one or more antibodies against the coronavirus is an antibody or antigen-binding fragment.

Embodiment 413. The device of any one of embodiments 381-412, wherein the antibody or antigen-binding fragment is monoclonal. antibody or antigen-binding fragment is polyclonal.

Embodiment 414. The device of any one of embodiments 381-413, wherein device further comprises a housing at least partially enclosing the surface.

Embodiment 415. The device of any one of embodiments 381-414, wherein device further comprises a sample receptor configured to receive a biological sample from a subject.

Embodiment 416. The device of any one of embodiments 381-415, wherein the sample receptor is mechanically coupled to a housing at least partially enclosing the surface.

Embodiment 417. The device of any one of embodiments 381-416, wherein the biological sample comprises one or more antibodies specific to the peptide.

Embodiment 418. The device of any one of embodiments 381-417, wherein the biological sample does not consist of one or more antibodies specific to the peptide.

Embodiment 419. The device of any one of embodiments 381-418, wherein the one or more antibodies belong to one or more immunoglobulin classes comprising immunoglobulin M, immunoglobulin G, immunoglobulin A, immunoglobulin E, and immunoglobulin D.

Embodiment 420. The device of any one of embodiments 381-419, wherein the subject was, or is, exposed to the coronavirus.

Embodiment 421. The device of any one of embodiments 381-420, wherein exposure of the subject to the coronavirus is unknown.

Embodiment 422. The device of any one of embodiments 381-421, wherein the subject was administered a vaccine against the coronavirus.

Embodiment 423. The device of any one of embodiments 381-422, wherein the biological sample comprises blood, urine, saliva, or feces.

Embodiment 424. The device of any one of embodiments 381-423, wherein the blood is capillary blood.

Embodiment 425. The device of any one of embodiments 381-424, wherein device further comprises a transdermal puncture device configured to obtain the capillary blood from the subject.

Embodiment 426. The device of any one of embodiments 381-425, wherein the sample receptor comprises a filter to separate serum from the blood.

Embodiment 427. The device of any one of embodiments 381-426, wherein the device is a single integrated device.

Embodiment 428. Aspects disclosed herein provide a kit comprising: (a) a lateral flow assay assembly comprising: a composition comprising: a first peptide or protein derived from an ACE2, a portion thereof; or a second peptide or protein derived from a spike glycoprotein of SARS-CoV-2 or variant thereof, or a portion thereof, the first peptide or protein or the second peptide or protein comprising a detectable moiety; and a porous membrane comprising: (1) a test zone, wherein the test zone comprises: one or more capture molecules coupled to the porous membrane at the test zone, the one or more capture molecules comprising: (i) a third peptide or protein derived from the spike glycoprotein of the SARS-CoV-2 or variant thereof, or a portion thereof; (ii) a fourth peptide or protein derived from the ACE2, or portion thereof; or (iii) a primary capture molecule specific to (i), (ii), or a protein tag conjugated thereto; and (b) instructions for assaying a biological sample with the lateral flow assay assembly to detect a presence, an absence, or a quantity of neutralizing antibodies against SARS-CoV-2 or variant thereof in the biological sample, or a component thereof.

Embodiment 429. The kit of embodiment 428, wherein the kit further comprises a sample receptor mechanically coupled to the porous membrane, the sample receptor configured to contain the biological sample.

Embodiment 430. The device of any one of embodiments 428-429, wherein the kit further comprises instructions for: obtaining the biological sample; assaying the biological sample with the lateral flow assay assembly to detect a presence, an absence, or a quantity of a binding complex between the composition and the one or more capture molecules in the biological sample, or component thereof; and classifying the biological sample as having the presence, the absence, or the quantity of the neutralizing antibodies against the SARS-CoV-2 or variant thereof.

Embodiment 431. The device of any one of embodiments 428-430, wherein the kit further comprises instructions for downloading a mobile application on a personal electronic device, the mobile application configured to analyze image data from an image of the test zone of the lateral flow assay assembly to classify the biological sample as having the presence, the absence, or the quantity of neutralizing antibodies against the SARS-CoV-2 or variant thereof.

Embodiment 432. The device of any one of embodiments 428-431, wherein the kit further comprises: (a) an second primary capture molecule specific to one or more antibodies against the SARS-CoV-2 or variant thereof, wherein the one or more antibodies against SARS-CoV-2 or variant thereof comprises an immunoglobulin G, an immunoglobulin M, an immunoglobulin A, or a combination thereof; and (b) instructions for: detecting a presence, an absence, or a quantity of a binding complex between the second primary capture molecules and the one or more antibodies against the SARS-CoV-2 or variant thereof in the biological sample or a component thereof, by bringing the biological sample or component thereof into contact with the second primary capture molecule in (a); and classifying the biological sample as having a presence, an absence, or a quantity of the one or more antibodies against the SARS-CoV-2 or variant thereof in the biological sample or component thereof based on the presence, the absence or the quantity of the binding complex that is detected.

Embodiment 433. Aspects disclosed herein provide a method comprising: step (a) providing a biological sample from a subject; step (b) analyzing the biological sample, or first component thereof, to detect a presence, an absence, or a quantity of neutralizing antibodies against a severe acute respiratory syndrome coronavirus 2 (SARS-CoV-2) or variant thereof by: contacting the first component of the biological sample to a first peptide or protein derived from a spike protein of the SARS-CoV-2 or variant thereof, or a portion thereof, in the presence of a second peptide or protein derived from an angiotensin converting enzyme 2 (ACE2), or a portion thereof, wherein the first peptide or protein or the second peptide or protein comprises a detectable moiety; and detecting the presence, the absence, or the quantity of one or more neutralizing antibodies in the biological sample that functionally block binding between the first peptide or protein and the second peptide or protein by detecting a binding complex between the first peptide or protein and the second peptide or protein in the presence of the biological sample; step (c) classifying the biological sample, or first component thereof, as having the presence, the absence, or the quantity of neutralizing antibodies to the SARS-CoV-2 or variant thereof, based, at least partially, on analyzing in step (b); step (d) analyzing the biological sample, or second component thereof, to detect a presence, an absence, or a quantity of one or more antibodies against SARS-CoV-2 or variant thereof by: contacting the biological sample, or second component thereof, to a capture molecule specific to the one or more antibodies against SARS-CoV-2 or variant thereof, wherein the capture molecule comprises a detectable moiety; and detecting the presence, the absence, or the quantity of a binding complex between the capture molecule and the one or more antibodies; and step (e) classifying the biological sample, or second component thereof, as having the presence, the absence, or the quantity of the one or more antibodies against SARS-CoV-2 or variant thereof, at least partially, on analyzing in step (d).

Embodiment 434. The method of embodiment 433, wherein the classifying in step (c) comprises classifying the biological sample from the subject as positive for the presence of neutralizing antibodies against the SARS-CoV-2 or variant thereof, provided an absence of the binding complex between the first peptide or protein and the second peptide or protein is detected.

Embodiment 435. The method of any one of embodiments 433-434, wherein the classifying in step (e) comprises classifying the biological sample from the subject as positive for exposure to SARS-CoV-2 or variant thereof, provided the presence of the binding complex between the capture molecule and the one or more antibodies is detected, or providing the quantity of the binding complex between the capture molecule and the one or more antibodies is above an index or a control.

Embodiment 436. The method of any one of embodiments 433-435, wherein the steps (a)-(e) are performed at a point of need or a point of care.

Embodiment 437. The method of any one of embodiments 433-436, wherein step (b) does not utilize an immortalized cell or an immortalized cell culture.

Embodiment 438. The method of any one of embodiments 433-437, wherein the biological sample of the method comprises whole blood, blood plasma, or serum.

Embodiment 439. The method of any one of embodiments 433-428, wherein the analyzing in step (b) and analyzing in step (d) are performed with a single integrated device.

Embodiment 440. The method of any one of embodiments 433-439, wherein the steps (c) and (e) are performed by an application that runs on an electronic device, the application comprising a mobile application or a web application.

Embodiment 441. The method of any one of embodiments 433-440, wherein the application of the method is configured to generate a report comprising a status of the biological sample, the status comprising the presence, the absence, or the quantity of the neutralizing antibodies against SARS-CoV-2 or variant thereof in the biological sample.

Embodiment 442 The method of any one of embodiments 433-441, wherein the application of the method is configured to generate a report comprising a status of the biological sample, the status comprising the presence, the absence, or the quantity of the one or more antibodies against the SARS-CoV-2 or variant thereof, wherein the one or more antibodies against SARS-CoV-2 comprises immunoglobulin G, immunoglobulin M, immunoglobulin A, or a combination thereof.

Embodiment 443. Aspects disclosed herein provide a system comprising: a first testing device or module to detect a presence, an absence, or a quantity of neutralizing antibodies against SARS-CoV-2 or variant thereof, the first testing device or module comprising: a composition comprising: a first peptide or protein derived from an ACE2, or portion thereof; and a second peptide or protein derived from a spike glycoprotein of the SARS-CoV-2 or variant thereof, or a portion thereof, the first peptide or protein or the second peptide or protein comprises a detectable moiety; and a test zone for visualization of the detectable moiety; and a second testing device or module to measure a presence, an absence, or a quantity of one or more antibodies against SARS-CoV-2, or variant thereof, the second testing device or module comprising: a first surface; and one or more capture molecules coupled to a region of the first surface, the one or more capture molecules comprising: a binding domain specific to one or more antibodies against SARS-CoV-2 or variant thereof; and a detectable moiety.

Embodiment 444. The system of embodiment 443, wherein the system further comprises an imaging device operatively coupled to the first device, the imaging device configured to capture an image of the test zone.

Embodiment 445. The system of any one of embodiments 443-444, wherein the system further comprises an imaging device operatively coupled to the second device, the imaging device configured to capture an image of the region of the first surface.

Embodiment 446. The system of any one of embodiments 443-445, wherein the system further comprises an electronic device comprising one or more processors operatively coupled to the imaging device configured to run an algorithm to generate a first classification of the biological sample as having the presence, the absence, or the quantity of the neutralizing antibodies to the SARS-CoV-2 or variant thereof.

Embodiment 447. The system of any one of embodiments 443-446, wherein the system further comprises an electronic device comprising one or more processors operatively coupled to the imaging device configured to run an algorithm to generate a second classification of the biological sample as having the presence, the absence, or the quantity of the one or more antibodies against the SARS-CoV-2 or variant thereof, wherein the one or more antibodies against SARS-CoV-2 or variant thereof comprises immunoglobulin G, immunoglobulin M, immunoglobulin A, or a combination thereof.

Embodiment 448. The system of any one of embodiments 443-447, wherein the system further comprises an application that runs on an electronic device, the application configured to display on a graphical user interface a report comprising a status of the biological sample, the status comprising one or more of: the presence, the absence, or the quantity of the neutralizing antibodies against SARS-CoV-2 or variant thereof; and the presence, the absence, or the quantity of the one or more antibodies against SARS-CoV-2 or variant thereof, wherein the one or more antibodies against SARS-CoV-2 or variant thereof comprises immunoglobulin G, immunoglobulin M, immunoglobulin A, or a combination thereof.

Embodiment 449. The system of any one of embodiments 443-448, wherein the test zone of the system is positioned at a second surface and wherein either of the first peptide or protein and the second peptide or protein is coupled to the second surface at the test zone directly or indirectly.

Embodiment 450. The system of any one of embodiments 443-449, wherein the first testing module and the second testing module of the system are in a single integrated device, and wherein the first surface and the second surface are the same surface.

Embodiment 451. The system of any one of embodiments 443-450, wherein the first testing device and the second testing device are portable.

Embodiment 452. The system of any one of embodiments 443-451, wherein the first testing device or module does not consist of an immortalized cell or an immortalized cell culture.

Embodiment 453. Aspects disclosed herein provide a system for point of need or point of care comprising a testing device to detect neutralizing antibodies against a SARS-CoV-2 or variant thereof to aid in the diagnosis of a disease or a condition caused by SARS-CoV-2 or variant thereof, the testing device comprising a composition comprising: a first peptide or protein derived from an ACE2, or portion thereof; and a second peptide or protein derived from a spike glycoprotein of the SARS-CoV-2 or variant thereof, or portion thereof, wherein at least one of the first peptide or protein and the second peptide or protein is labeled with a detectable moiety.

Embodiment 454. The system of embodiment 453, wherein the system further comprises an application configured to run on the electronic device, the application configured to generate a classification of a biological sample as having a presence, an absence, or a quantity of neutralizing antibodies against the SARS-CoV-2 or variant thereof based on a presence, an absence, or a quantity of a binding complex between the first peptide or protein and the second peptide or protein in the presence of the biological sample.

Embodiment 455. The system of any one of embodiments 453-454, wherein the classification of the biological sample is indicative of at least one of: (a) a diagnosis related to the disease or the condition caused by SARS-CoV-2; (b) a prognosis related to adaptive immunity of the subject against an infection by the SARS-CoV-2 or variant thereof, or the disease or the condition caused by the SARS-CoV-2; and (c) a measure of susceptibility of the subject to an infection by the SARS-CoV-2.

Embodiment 456. The system of any one of embodiments 453-455, wherein the system further comprises a vaccine composition against the SARS-CoV-2 or variant thereof, wherein the testing device is capable of identifying the subject as being in need of treatment with the vaccine composition based on any one of (a)-(c).

Embodiment 457. The system of any one of embodiments 453-456, wherein the testing device is a point of need or a point of care device.

Embodiment 458. The system of any one of embodiments 453-457, wherein the system does not consist of an immortalized cell or an immortalized cell culture.

Embodiment 459. The system of any one of embodiments 453-458, wherein the electronic device of the system is a personal electronic device comprising a smartphone, a tablet, or a personal computer.

Embodiment 460. Aspects disclosed herein provide a system comprising a lateral flow assay assembly comprising: (i) a composition comprising: a first peptide or protein derived from an ACE2, a portion thereof; or a second peptide or protein derived from a spike glycoprotein of SARS-CoV-2 or variant thereof, or a portion thereof, the first peptide or protein or the second peptide or protein comprising a detectable moiety; and (ii) a porous membrane comprising a test zone, wherein the test zone comprises one or more capture molecules coupled to the porous membrane at the test zone, the one or more capture molecules comprising: a third peptide or protein derived from the spike glycoprotein of the SARS-CoV-2 or variant thereof, or a portion thereof; a fourth peptide or protein derived from the ACE2, or portion thereof; or a primary capture molecule specific to (i), (ii), or a protein tag conjugated thereto.

Embodiment 461. The system of embodiment 460, wherein the lateral flow assay assembly of the system is portable.

Embodiment 462. The system of any one of embodiments 460-461, wherein the system further comprises an application configured to run on a personal electronic device, the personal electronic device comprising a camera to capture an image of the test zone before or after a biological sample obtained from a subject is applied to the porous membrane, wherein the application is configured to generate a classification of the biological sample as having a presence, an absence, or a quantity of neutralizing antibodies against the SARS-CoV-2 or variant thereof based on a presence, an absence, or a quantity of a binding complex between the composition and the one or more capture molecules detected in the biological sample using the lateral flow assay assembly.

Embodiment 463. The system of any one of embodiments 460-462, wherein the one or more capture molecules of the system comprises the primary capture molecule specific to (i) the third peptide or protein or the protein tag conjugated thereto, or (ii) the fourth peptide or protein or the protein tag conjugated thereto.

Embodiment 464. The system of any one of embodiments 460-463, wherein the composition of the system comprises the second peptide or protein, and wherein the one or more capture molecules comprises (i) the fourth peptide or protein, or (ii) the primary capture molecule specific to the fourth peptide or protein, or the protein tag conjugated thereto.

Embodiment 465. The system of any one of embodiments 460-464, wherein the system further comprises a labeled second primary capture molecule specific to one or more antibodies against the SARS-CoV-2 or variant thereof, the one or more antibodies comprising an immunoglobulin G, immunoglobulin M, an immunoglobulin A, or a combination thereof.

Embodiment 466. The system of any one of embodiments 460-465, wherein the labeled second primary capture molecule is coupled to the porous membrane at the test zone.

Embodiment 467. Aspects disclosed herein provide a system comprising: (a) a first testing device or module to detect a presence, an absence, or a quantity of neutralizing antibodies against SARS-CoV-2 or variant thereof, the first testing device or module comprising: (i) a composition comprising: (1) a first peptide or protein derived from an ACE2, or portion thereof; and (2) a second peptide or protein derived from a spike glycoprotein of the SARS-CoV-2 or variant thereof, or a portion thereof, the first peptide or protein or the second peptide or protein comprises a detectable moiety; and (ii) a test zone for visualization of the detectable moiety; and (b) a second testing device or module to measure a presence, an absence, or a quantity of one or more antibodies against SARS-CoV-2, or variant thereof, the second testing device or module comprising: a first surface; and one or more capture molecules coupled to a region of the first surface, the one or more capture molecules comprising: a binding domain specific to one or more antibodies against SARS-CoV-2 or variant thereof; and a detectable moiety.

Embodiment 468. The system of embodiment 467, wherein the system further comprises an application that runs on an electronic device, the application configured to generate a classification of the biological sample, the classification comprising one or more of: the presence, the absence, or the quantity of the neutralizing antibodies against SARS-CoV-2 or variant thereof; and the presence, the absence, or the quantity of the one or more antibodies against SARS-CoV-2 or variant thereof, wherein the one or more antibodies against SARS-CoV-2 or variant thereof comprises immunoglobulin G, immunoglobulin M, immunoglobulin A, or a combination thereof.

Embodiment 469. The system of any one of embodiments 467-468, wherein the test zone is positioned at a second surface and wherein either of the first peptide or protein and the second peptide or protein is coupled to the second surface at the test zone directly or indirectly.

Embodiment 470. The system of any one of embodiments 467-469, wherein the first testing module and the second testing module are in a single integrated device, and wherein the first surface and the second surface are the same surface.

Embodiment 471. The system of any one of embodiments 467-470, wherein the first testing device and the second testing device are portable.

Embodiment 472. The system of any one of embodiments 467-471, wherein the first testing device or module does not consist of an immortalized cell or an immortal cell culture.

Embodiment 473. Aspects disclosed herein provide a system for point of need or point of care comprising a testing device to detect neutralizing antibodies against a SARS-CoV-2 or variant thereof to aid in the diagnosis of a disease or a condition caused by SARS-CoV-2 or variant thereof, the testing device comprising a composition comprising: a first peptide or protein derived from an ACE2, or portion thereof; and a second peptide or protein derived from a spike glycoprotein of the SARS-CoV-2 or variant thereof, or portion thereof, wherein at least one of the first peptide or protein and the second peptide or protein is labeled with a detectable moiety.

Embodiment 474. The system of embodiment 473, wherein the system further comprises an application configured to run on the electronic device, the application configured to generate a classification of a biological sample as having a presence, an absence, or a quantity of neutralizing antibodies against the SARS-CoV-2 or variant thereof based on a presence, an absence, or a quantity of a binding complex between the first peptide or protein and the second peptide or protein in the presence of the biological sample.

Embodiment 475. The system of any one of embodiments 473-474, wherein the classification of the biological sample is indicative of at least one of: (a) a diagnosis related to the disease or the condition caused by SARS-CoV-2; (b) a prognosis related to adaptive immunity of the subject against an infection by the SARS-CoV-2 or variant thereof, or the disease or the condition caused by the SARS-CoV-2; and (c) a measure of susceptibility of the subject to an infection by the SARS-CoV-2.

Embodiment 476. The system of any one of embodiments 473-475, wherein the system further comprises a vaccine composition against the SARS-CoV-2 or variant thereof, wherein the testing device is capable of identifying the subject as being in need of treatment with the vaccine composition based on any one of (a)-(c).

Embodiment 477. The system of any one of embodiments 473-476, wherein the testing device is a point of need or a point of care device.

Embodiment 478. The system of any one of embodiments 473-477, wherein the system does not consist of an immortalized cell or an immortalized cell culture.

Embodiment 479. The system of any one of embodiments 473-478, wherein the electronic device of the system is a personal electronic device comprising a smartphone, a tablet, or a personal computer.

Embodiment 480. Aspects disclosed herein provide a system comprising a lateral flow assay assembly comprising: (i) a composition comprising: a first peptide or protein derived from an ACE2, a portion thereof; or a second peptide or protein derived from a spike glycoprotein of SARS-CoV-2 or variant thereof, or a portion thereof, the first peptide or protein or the second peptide or protein comprising a detectable moiety; and (ii) a porous membrane comprising a test zone, wherein the test zone comprises one or more capture molecules coupled to the porous membrane at the test zone, the one or more capture molecules comprising: a third peptide or protein derived from the spike glycoprotein of the SARS-CoV-2 or variant thereof, or a portion thereof; a fourth peptide or protein derived from the ACE2, or portion thereof; or a primary capture molecule specific to (i), (ii), or a protein tag conjugated thereto.

Embodiment 481. The system of embodiment 480, wherein the lateral flow assay assembly of the system is portable.

Embodiment 482. The system of any one of embodiments 480-481, wherein the system further comprises an application configured to run on a personal electronic device, the personal electronic device comprising a camera to capture an image of the test zone before or after a biological sample obtained from a subject is applied to the porous membrane, wherein the application is configured to generate a classification of the biological sample as having a presence, an absence, or a quantity of neutralizing antibodies against the SARS-CoV-2 or variant thereof based on a presence, an absence, or a quantity of a binding complex between the composition and the one or more capture molecules detected in the biological sample using the lateral flow assay assembly.

Embodiment 483. The system of any one of embodiments 480-482, wherein the one or more capture molecules comprises the primary capture molecule specific to (i) the third peptide or protein or the protein tag conjugated thereto, or (ii) the fourth peptide or protein or the protein tag conjugated thereto.

Embodiment 484. The system of any one of embodiments 480-483, wherein the composition comprises the second peptide or protein, and wherein the one or more capture molecules comprises (i) the fourth peptide or protein, or (ii) the primary capture molecule specific to the fourth peptide or protein, or the protein tag conjugated thereto.

Embodiment 485. The system of any one of embodiments 480-484, wherein the system further comprises a labeled second primary capture molecule specific to one or more antibodies against the SARS-CoV-2 or variant thereof, the one or more antibodies comprising an immunoglobulin G, immunoglobulin M, an immunoglobulin A, or a combination thereof.

Embodiment 486. The system of any one of embodiments 480-485, wherein the labeled second primary capture molecule is coupled to the porous membrane at the test zone.

Embodiment 487. Aspects disclosed herein provide a system comprising: (a) a first testing device or module to detect a presence, an absence, or a quantity of neutralizing antibodies against SARS-CoV-2 or variant thereof, the first testing device or module comprising: (i) a composition comprising: (1) a first peptide or protein derived from an ACE2, or portion thereof; and (2) a second peptide or protein derived from a spike glycoprotein of the SARS-CoV-2 or variant thereof, or a portion thereof, the first peptide or protein or the second peptide or protein comprises a detectable moiety; and (ii) a test zone for visualization of the detectable moiety; and (b) a second testing device or module to measure a presence, an absence, or a quantity of one or more antibodies against SARS-CoV-2, or variant thereof, the second testing device or module comprising: a first surface; and one or more capture molecules coupled to a region of the first surface, the one or more capture molecules comprising: a binding domain specific to one or more antibodies against SARS-CoV-2 or variant thereof; and a detectable moiety.

Embodiment 488. The system of embodiment 487, wherein the system further comprises an application that runs on an electronic device, the application configured to generate a classification of the biological sample, the classification comprising one or more of: the presence, the absence, or the quantity of the neutralizing antibodies against SARS-CoV-2 or variant thereof; and the presence, the absence, or the quantity of the one or more antibodies against SARS-CoV-2 or variant thereof, wherein the one or more antibodies against SARS-CoV-2 or variant thereof comprises immunoglobulin G, immunoglobulin M, immunoglobulin A, or a combination thereof.

Embodiment 489. The system of any one of embodiments 487-488, wherein the test zone is positioned at a second surface and wherein either of the first peptide or protein and the second peptide or protein is coupled to the second surface at the test zone directly or indirectly.

Embodiment 490. The system of any one of embodiments 487-489, wherein the first testing module and the second testing module are in a single integrated device, and wherein the first surface and the second surface are the same surface.

Embodiment 491. The system of any one of embodiments 487-490, wherein the first testing device and the second testing device are portable.

Embodiment 492. The system of any one of embodiments 487-491, wherein the first testing device or module does not consist of an immortalized cell or an immortalized cell line.

Embodiment 493. Any of the preceding embodiments, wherein an age of the subject is at least or about 50 years old.

Embodiment 494. Any of the preceding embodiments, wherein the subject is administered a commercialized vaccine.

Embodiment 495. Any of the preceding embodiments, wherein detecting a number of binding complexes between the detectable peptide and the one or more capture molecules is performed in less than 20 minutes from the time the biological sample from the subject is contacted with the detectable peptide and the one or more capture molecules.

Embodiment 496. Any of the preceding embodiments, wherein the biological sample comprises a volume of less than 1 milliliter.

Embodiment 497. Any of the preceding embodiments, wherein the systems and methods are computer-automated.

Embodiment 498. Any of the preceding embodiments, wherein detecting a number of binding complexes between the detectable peptide and the one or more capture molecules can be performed in a single step without the need for washing.

Embodiment 499. Any of the preceding embodiments, wherein detecting a number of binding complexes between the detectable peptide and the one or more capture molecules specific to the peptide can be performed in a single step without the need for washing.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. Ibis applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, “allowed” or “allowance” in connection with a vaccine composition refers to a vaccine composition that is legally permitted by a relevant regulatory entity to be marketed for use to prevent a disease caused by a pathogen in an intended subject. In some embodiments, an allowed vaccine is approved or licensed for human use. In some embodiments, an allowed vaccine is approved, conditionally approved, or indexed for animal use. In some embodiments, the intended subject is a human. In some embodiments, the intended subject is an animal, such as a household pet or a farm animal.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Any systems, methods, software, compositions, and platforms described herein are modular and not limited to sequential steps. Accordingly, terms such as “first” and “second” do not necessarily imply priority, order of importance, or order of acts.

The term “analyte” refers to a substance whose chemical constituents or activity is measured. In some embodiments, the analyte comprises an activity of a neutralizing antibody. In some embodiments, the activity is blocking binding between a pathogen of interest (e.g., SARS-CoV-2) and a cognate receptor (e.g., ACE2). In some embodiments, the analyte is a complex comprising the spike protein bound to the neutralizing antibody at the receptor binding region of the spike protein.

The term “cloud” refers to shared or sharable storage of electronic data. The cloud may be used for archiving electronic data, sharing electronic data, and analyzing electronic data.

As used herein, the terms, “clinic,” “clinical setting,” “laboratory” or “laboratory setting” refer to a hospital, a clinic, a pharmacy, a research institution, a pathology laboratory, or other commercial business setting where trained personnel are employed to process and/or analyze biological and/or environmental samples. These terms are contrasted with point of care, a remote location, a home, a school, and otherwise non-business, non-institutional setting.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing is relative or absolute. “Detecting the presence of” can include determining the amount of something present in addition to determining whether it is present or absent depending on the context.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease is, for example, at least 10%, at least 20%, at least 30%, at least 40% or more, and is preferably down to a level accepted as within the range of normal for an individual without a given disease.

The terms “subject,” “individual,” or “patient” are often used interchangeably herein. A “subject” is a biological entity containing expressed genetic materials. The biological entity is a plant, animal, or microorganism, including, for example, bacteria, viruses, fungi, and protozoa. The subject is tissues, cells and their progeny of a biological entity obtained in vivo or cultured in vitro. The subject is a mammal. The mammal is a human. The subject may be diagnosed or suspected of being at high risk for a disease. In some cases, the subject is not necessarily diagnosed or suspected of being at high risk for the disease. In some embodiments, the subject is a child. In some embodiments, the subject is an adolescent. In some embodiments, the subject is an adult. In some embodiments, the age of the subject is 1, 2, 3, 4, 5, 6, 7, 8, 9, 10, 11, 12, 13, 14, 15, 16, 17, or 18 years. In some embodiments, the age of the subject is between 18 and 50 years. In some embodiments, the subject is more than or equal to about 50 years old (e.g., 65 years old). In some embodiments, the subject is between 50-100, 55-95, 60-90, 65-85, or 70-80 years old.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “adaptive immune response” or “adaptive immunity” as used herein refers to the components of the immune response that respond in an antigen-restricted way and encompasses cellular immune responses attributable to T lymphocytes and humoral or antibody response attributable to B cells and plasma cells. In some embodiments, the antibody response comprises neutralizing antibody response. A “cellular immune response” is indicated by any one or more of the following: cytokine/chemokine release by T cells; T-cell homing to secondary lymphoid organs; T-cell proliferation; and cytotoxic T-cell responses. Several methods are suitable to verify an antigen-specific cellular immune response, including ex vivo antigen stimulation assays of T lymphocytes and in vivo assays, such as tetramer staining of T lymphocytes. An “antibody response” is indicated at least by any one or more of the following: B cell proliferation, B-cell cytokine/chemokine release, B-cell homing to secondary lymphoid organs, antibody secretion, isotype switching to IgG type antibodies, or plasma cell differentiation. An antibody response may be verified by several methods, but a predominant method is the detection of antigen-specific antibodies in the serum or plasma of an individual. In some embodiments, the antibody comprises neutralizing antibody.

An “adjuvant” as described herein refers to a substance that, in combination with an antigen, promotes an adaptive immune response to the antigen. In some embodiments, the adjuvant is an immune stimulatory compound. In some embodiments, the adjuvant comprises analgesic adjuvants. In some embodiments, the adjuvant comprises inorganic compounds such as alum, aluminum hydroxide, aluminum phosphate, or calcium phosphate hydroxide. In some embodiments, the adjuvant comprises mineral oil or paraffin oil. In some embodiments, the adjuvant comprises bacterial products such as inactivated Bordetella pertussis, Mycobacterium bovis, tor oxoids. In some embodiments, the adjuvant comprises nonbacterial organics like squalene. In some embodiments, the adjuvant comprises the use of delivery systems such as detergents (Quil A). In some embodiments, the adjuvant comprises plant saponins such as saponin derived from Quillaja, soybean, or Polygala senega. In some embodiments, the adjuvant comprises Freund's complete adjuvant or Freund's incomplete adjuvant. In some embodiments, the adjuvant comprises food-based oil like peanut oil.

An “immune stimulatory compound” refers to a substance that specifically interacts with the innate immune system to initiate a “danger signal” that ultimately leads to the development of the adaptive components of the immune response (e.g., B cell, T cells). Immune stimulatory compounds include, without limitation, pathogen-associated molecular patterns (PAMPs) such as dsRNA, lipopolysaccharide, and CpG DNA, either naturally occurring or synthetic. Immune stimulatory compounds may be agonists of various innate immune receptors including Toll-like receptors (TLRs), NOD-like receptors, RIG-1 or MDA-5 receptors, C-type lectin receptors, or the STING pathway.

The section headings used herein are for organizational purposes only and are not to be construed as limiting the subject matter described.

### EXAMPLES

The following examples are included for illustrative purposes only and are not intended to limit the scope of the invention.

### Example 1: Detecting Adaptive Immunity in a Healthcare Worker

A healthcare worker, responding to an urgent public health crisis, needs to know whether she is immune to an infection of a pathogen of interest so that she may serve others in the community at risk for infection. In this example, the pathogen of interest is SARS-CoV-2, and the healthcare worker is exposed to SARS-CoV-2. Optionally, the healthcare worker has recovered from coronavirus disease of 2019 (COVID-19).

While at home, the healthcare worker utilizes the point of need testing device described herein to obtain capillary blood by pricking her finger. She applies the capillary blood to the sample receptor component of the testing device, where it separates the blood serum (containing the analyte of interest) and other blood components. The serum flows downstream from the sample receptor to a test zone of the testing device, where the serum is mixed with a fluid composition comprising a peptide-conjugate. In this example, the peptide comprises an amino acid sequence that is at least 95% identical to any one of SEQ ID NOS: 2-6, 11-12, 24, 47-48, 71-72, 95-96, or 115. The peptide-conjugate comprises a detectable moiety comprising a gold microsphere. Within 10-20 minutes a signal develops in the detectable zone of the testing device.

The healthcare worker takes a picture of the detectable zone using her smartphone, which is equipped with a mobile application that will interpret the results from the testing device. The mobile application displays the result via the graphical user interface of the smartphone and indicates to the healthcare worker that she is sufficiently immune to the pathogen. She returns to work immediately.

### Example 2: Detecting Adaptive Immunity to Pathogen in Blood or Blood Plasma

A donation of blood or blood plasma is tested for a presence of neutralizing antibodies that functionally block binding between a pathogen of interest and an pathogen recognition receptor. In this example, the test is performed at the point of need (e.g., a blood bank) by a technician. In this example, the pathogen of interest is SARS-CoV-2.

At the blood donation site, the technician utilizes the point of need testing device described herein to test a sample of the blood. The technician applies the blood to the sample receptor component of the testing device, where it separates the blood serum (containing the analyte of interest) and other blood components. The serum flows downstream from the sample receptor to a test zone of the testing device, where the serum is mixed with a fluid composition comprising the peptide-conjugate from Example 1. Within 10-20 minutes a signal develops in the detectable zone of the testing device.

The technician takes a picture of the detectable zone using a tablet, which is equipped with a mobile application that will interpret the results from the testing device. The mobile application displays the result via the graphical user interface of the tablet and indicates to the technician that the blood sample contains neutralizing antibodies that block binding between the spike protein of SARS-CoV-2 and human ACE2.

The technician sends the blood sample to a research laboratory to determine whether the serum isolated from this blood (containing the neutralizing antibodies) is suitable for use in convalescent plasma therapy.

### Example 3. Vaccine Development Tool and Methods of Use

A pharmaceutical company is developing a vaccine to a pathogen of interest. In this example, the pathogen of interest is SARS-CoV-2. The pharmaceutical company wants to know whether the vaccine induces the production of antibodies which block the interaction between the spike protein of SARS-Co-V-2 and the human ACE2 receptor, that mediates infection in vivo. The pharmaceutical company utilizes the testing device described herein to test the vaccine by testing a biological sample from an animal (e.g., mammal) inoculated with the vaccine.

A biological sample is obtained from a mammal that is exposed to SARS-Co-V-2, and is administered the vaccine. A researcher at the pharmaceutical company applies the biological sample to the sample receptor component of the testing device, where it separates the blood plasma/serum (containing the analyte of interest) and other blood components. The serum flows downstream from the sample receptor to a test zone of the testing device, where the serum is mixed with a fluid composition comprising the peptide-conjugate from Example 1. Within 10-20 minutes a signal develops in the detectable zone of the testing device.

The researcher takes an image of the detectable zone using an imaging device, which is equipped with an application that will interpret the results from the testing device. The application displays the result via the graphical user interface of the tablet and indicates to the researcher that biological sample contains antibodies that block the interaction between the spike protein of SARS-Co-V-2 and the human ACE2 receptor, which means that vaccine was effective. The vaccine moves on for further research and development.

### Example 4. Identifying Herd Immunity Using Artificial Intelligence

A governmental agency, responding to a global pandemic, is monitoring a population of citizens to determine whether a threshold adaptive immunity neutralizing a pathogen of interest is present in the population, such that incidences of infection are drastically reduced (also referred to as “herd immunity”). The agency provides citizens testing devices, such as those described herein; and a free mobile App that is downloaded to their mobile device. Citizens in a given population utilize the testing device, take a picture of the testing zone of the testing device using their mobile device.

The data is anonymized and uploaded into the cloud. Data includes GPS data from the mobile devices and the data from the picture that was taken for each citizen. The data is analyzed by cloud-computing by machine learning, and results are accessible to the agency via a web-portal. The results are used to identify populations of citizens that are immune from SARS-CoV-2. Geofencing is used to create geographical boundaries around where those populations reside. When a threshold number of citizens with adaptive immunity neutralizing SARS-CoV-2 is detected conferring herd immunity.

### Example 5. Ranid Deployment of Neutralization Assay for Newly Discovered Virus

A new strain of SARS-CoV-2 is discovered that has an amino acid sequence encoding the RBD comprising two amino acid substitutions, relative to SEQ ID NO: 3, 5, or 11. Referring to FIG. 8A-8B, components of the testing device described herein that were previously assembled include a surface comprising a capture molecule coupled to the surface, and a peptide-conjugate comprising a peptide derived from the ACE2 receptor and a detectable moiety (e.g., ACE2-Au).

Within weeks of the discovery of the new strain of SARS-CoV-2, a second peptide-conjugate is manufactured, the second peptide-conjugate comprising the variant RBD of the new strain of SARS-CoV-2 and conjugated to an epitope tag. The second peptide-conjugate is coupled to the surface via the surface-bound capture molecules specific to the epitope tag.

An individual in need of the testing device buys the testing device online, and downloads the mobile application on her iPhone. The testing device is mailed to the individual, and includes instructions for how to use the device and the testing device. Per the instructions provided, the individual pricks her finger to obtain capillary blood, places the drop of blood in the sample receptor of the testing device, and tilts the testing device sufficient to permit the fluid in the sample device to flow from the sample receptor to the opposite end of the testing device. If the individual has developed sufficient immunity to SARS-CoV-2, then no signal will be observed (FIG. 8B). If the individual has not developed sufficient immunity to SARS-CoV-2, then a signal will be observed (FIG. 8A).

### Example 6. Screening for a Population at Risk of Infection

A population comprising a plurality of subjects is at risk of a SARS-CoV-2 infection due to an outbreak. Accordingly, immunity of the population is determined by utilizing the systems, devices, compositions, and methods described herein. Blood cards are distributed to the subjects for collection of biological sample comprising capillary blood. The healthcare workers perform finger-prick to drop the capillary blood into each slot of the dried blood spot card. After collection of biological samples, the dried blood spot cards are sent by mail to a centralized processing and testing facility, where the blood cards are processed by automated robotics. The blood samples are tested for presence or quantity of neutralizing antibody against at least one antigen of SARS-CoV-2. Most of the subjects are determined to have sufficient quantity of neutralizing antibody, which indicates that most the subjects can carry on with normal daily activities. Some of the subjects have absence or insufficient quantity of neutralizing antibody. These subjects are asked to take shelter-in-place measure and will be vaccinated with any one of the vaccines described herein to induce neutralizing antibody.

In addition to determining the immunity due to the SARS-CoV-2 outbreak, the population is routinely screened at a time interval of once every three months. Such routine screening of the population identifies the percentage of the subjects within the population with sufficient quantity of neutralizing antibody against SARS-CoV-2. If the percentage of the subjects with sufficient quantity of neutralizing antibody is above the percentage of the herd immunity (e.g. over 70%), then the subjects with absence or insufficient quantity of neutralizing antibody do not need to be vaccinated.

The routine screening also identifies if any of the subjects are infected. The infected subjects can be asymptomatic but contagious. The routine screening is also conducted after the subjects are exposed to SARS-CoV-2. Those exposed subjects with an initial absence of neutralizing antibody against SARS-CoC-2 are retested at least one week, at least one month, or at least one year after the last exposure. Screening of the subjects for neutralizing antibody for SARS-CoV-2 identifies the subjects with the neutralizing antibody who should undergo an evaluation for assessing the symptoms or severity of the SARS-CoV-2 infection.

### Example 7. Longitudinal System for Screening

Efficacy and durability of a vaccine allowed or approved for the prevention of a disease caused by SARS-CoV-2 is assessed by assaying a biological sample from a subject who was administered the vaccine with the confirmatory diagnostic testing device described herein. The confirmatory diagnostic testing devices measures neutralizing antibodies in biological samples obtained from the subject at various time points following vaccine administration.

A biological sample is obtained from the subject prior to vaccine administration. The biological may be obtained by the subject (e.g., finger prick) and deposited on a biological sample medium (e.g., a dried blood spot card) to be stored and transported to a laboratory for processing. In this instance, the biological sample is obtained by finger pricking the subject for the biological sample comprising capillary blood. The biological may be obtained by the subject (e.g., finger prick) and deposited on a biological sample medium (e.g., a dried blood spot card) to be stored and transported to a laboratory for processing. The biological sample is tested for neutralizing antibody against antigen of SARS-CoV-2. The confirmatory diagnostic confirms absence of the neutralizing antibody in the biological sample and the subject needs to be vaccinated against SARS-CoV-2.

One month after vaccination, a biological sample comprising the capillary blood is obtained from the subject finger pricking at home. The biological sample is sent back to the laboratory for testing via the use of a blood card. The confirmatory diagnostic confirms sufficient quantity of neutralizing antibody in the biological sample, which indicates that the subject has developed adaptive immunity against SARS-CoV-2. The confirmatory diagnostic also confirms the efficacy and effectiveness of the vaccine by detecting presence and sufficient quantity of neutralizing antibody in the biological sample.

The subject is tested again at two months after vaccination, at four months after vaccination, at six months after vaccination, at one year after vaccination, at two years after vaccination, at three years after vaccination, at four years after vaccination to confirm that the vaccinated subject has maintained the sufficient quantity of the neutralizing antibody for adaptive immunity against SARS-CoV-2.

At the testing at five years after vaccination, the test reveals that vaccinated subject now has insufficient quantity of the neutralizing antibody and is in need of a booster vaccine against SARS-CoV-2.

### Example 8. Vaccine Confirmatory Diagnostic—Vaccine Comprising mRNA-1273

A clinical trial is being conducted to test efficacy and effectiveness of a SARS-CoV-2 vaccine comprising an mRNA encoding the full-length, prefusion stabilized Spike (S) protein (mRNA-1273). Subjects are enrolled into different cohorts to receive different dosages of the vaccine comprising the mRNA-1273. Subjects will receive the vaccine on Days 1 and 29 in the deltoid muscle and will be followed through 12 months post second vaccination (Day 394). Follow-up visits will occur 1, 2, and 4 weeks post each vaccination (Days 8, 15, 29, 36, 43, and 57), as well as 3, 6, and 12 months post second vaccination (Days 119, 209, and 394). The primary objective is to evaluate the safety and reactogenicity of a 2-dose vaccination schedule of mRNA-1273, given 28 days apart, across 5 dosages in healthy adults.

Primary outcome measures: solicited local and systemic adverse reactions (ARs) [Time Frame: 7 days post-vaccination]; unsolicited adverse events (AEs) [Time Frame: 28 days post-vaccination]; medically-attended adverse events (MAAEs) [Time Frame: Month 0 through Month 13]; serious adverse events (SAEs) [Time Frame: Month 0 through Month 13]; change in the measure of clinical safety laboratory values in Cohort 2 from baseline [Time Frame: Through 1 month after last vaccination]; the number and percentage of participants with abnormalities in blood pressure, temperature, HR or respiratory rate will be assessed. [Time Frame: Through 1 year after last vaccination]; the number and percentage of participants with abnormalities in physical examinations will be assessed [Time Frame: Through 1 year after last vaccination]; evaluate immunogenicity of mRNA-1273 by titer of SARS-CoV-2-specific binding antibody (bAb) measured by enzyme-linked immunosorbent assay (ELISA) [Time Frame: Through 1 year after the final dose].

Secondary outcome measures: titer of SARS-CoV-2-specific neutralizing antibody (nAb) using the testing devices described herein [Time Frame: Through 1 year post last vaccination]; and seroconversion as measured by an increase of SARS-CoV-2-specific neutralizing antibody (nAb) titer using the testing devices described herein [Time Frame: Through 1 year post last vaccination].

Secondary outcome measures for this clinical study show that the mRNA-1273 vaccine induces neutralizing antibodies against SARS-CoV-2, indicating that mRNA-1273 is effective to confer adaptive immunity against SARS-CoV-2. This result is strongly correlative of protection against a future infection by SARS-CoV-2.

Durability of the mRNA-1273 vaccine is measured continuously throughout 1 year post the last vaccination. Seroconversion measured using the testing devices described show consistent neutralizing antibodies levels over time, indicating that the mRNA-1273 vaccine is durable. The efficacy and durability of the mRNA-1273 vaccine is continuously monitored using the testing devices described herein during Phase 0, Phase I, Phase II, Phase III, or Phase IV or after Phase IV of the clinical trial.

Upon approval of the mRNA-1273 vaccine, the vaccine will be administered to subjects in the population. Confirmatory diagnostic utilizing the systems, devices, methods, and compositions described herein will be performed to determine if the vaccinated subjects will develop and maintain sufficient quantity of neutralizing antibody. Confirmatory diagnostic will be performed at one month, two months, six months, one year, two years, three years, and four years after vaccination. Subjects with insufficient quantity of neutralizing antibody will be advised to receive a booster vaccine.

### Example 9. Vaccine Confirmatory Diagnostic—Vaccine Comprising BCG

A clinical trial is being conducted to test efficacy and effectiveness of a SARS-CoV-2 vaccine comprising Bacille Calmette-Gudrin (BCG), a vaccine originally developed for tuberculosis. Studies have shown its ability to induce potent protection against other infectious diseases: the so called non-specific effects (NSEs). A favorable in vitro or in vivo effect is observed in studies for distinct viral pathogens, e.g. respiratory syncytial virus, yellow fever, herpes simplex virus; human papilloma virus. Based on the capacity of BCG to reduce the incidence of respiratory tract infections in children, to exert antiviral effects in experimental models; and to reduce viremia in an experimental human model of viral infection, the hypothesis is that BCG vaccination induces (partial) protection against susceptibility to and/or severity of Covid-19 infection. This study evaluates the efficacy of BCG to improve the clinical course of Covid-19 infection and to prevent absenteeism in order to safeguard continuous patient care.

This randomized controlled trial is designed as a pragmatic study with a highly feasible primary endpoint, which is unplanned absenteeism, that is continuously measured on a bi-weekly basis). This allows for the most rapid identification of a beneficial outcome that would allow other subjects to also benefit from the intervention if and as soon as it is demonstrated to be effective.

Primary outcome measures health care workers absenteeism [Time Frame: Maximum of 180 days].

Secondary outcome measures: the cumulative incidence of documented COVID-19 [Time Frame: Maximum of 180 days]; the cumulative incidence of Hospital Admission due to documented COVID-19 [Time Frame: Maximum of 180 days]; the number of days of unplanned absenteeism, because of documented COVID-19 [Time Frame: Maximum of 180 days]; the cumulative incidence of self-reported acute respiratory symptoms or fever [Time Frame: Maximum of 180 days]; the cumulative incidence of death due to documented COVID-19 [Time Frame: Maximum of 180 days]; the cumulative incidence of Intensive Care Admission due to documented COVID-19 [Time Frame: Maximum of 180 days]; the number of days of absenteeism, because of imposed quarantine as a result of exposure to COVID-19 [Time Frame: Maximum of 180 days]; the number of days of absenteeism, because of imposed quarantine as a result of having acute respiratory symptoms, fever or documented COVID-19 [Time Frame: Maximum of 180 days]; the number of days of unplanned absenteeism because of self-reported acute respiratory symptoms [Time Frame: Maximum of 180 days]; the number of days of self-reported fever (?38 gr C) [Time Frame: Maximum of 180 days]; the cumulative incidence of self-reported fever (238 gr C) [Time Frame: Maximum of 180 days]; the number of days of self-reported acute respiratory symptoms [Time Frame: Maximum of 180 days]; the cumulative incidence of self-reported acute respiratory symptoms [Time Frame: Maximum of 180 days]; the cumulative incidence of death for any reason [Time Frame: Maximum of 180 days]; the cumulative incidence of Intensive Care Admission for any reason [Time Frame: Maximum of 180 days]; the cumulative incidence of Hospital Admission for any reason [Time Frame: Maximum of 180 days], and the cumulative incidence and magnitude of plasma/serum antibodies (IgA,M,G) and SARS-CoV-2-specific antibodies at 12 weeks after vaccination and at the end of the study period [Time Frame: Maximum of 180 days]

In addition, the subjects are scheduled to for periodic follow-up visit during and after the time frames disclosed herein. During each visit, confirmatory diagnostic will be performed to analyze a biological sample. The biological sample comprises capillary blood obtained from the subject. The biological sample is analyzed with the systems, devices, compositions, and methods described herein to detect the presence, absence, or quantity of neutralizing antibody recognizing antigen of SARS-CoV-2. Depending on the underlying pathophysiological mechanism, the efficacy or effectiveness of the vaccine is confirmed when the presence or sufficient quantity or when the absence of the neutralizing antibody recognizing an antigen of SARS-CoV-2 is detected in the biological sample. Once the confirmatory diagnostic confirms the efficacy and effectiveness of BCG, BCG progresses into the next stage of development. Confirmatory diagnostic of the efficacy and the effectiveness of the BCG vaccine is continuously performed by utilizing the systems, devices, compositions, and methods described herein during Phase 0, Phase I, Phase II, Phase III, or Phase IV or after Phase IV of the clinical trial.

Upon approval of the vaccine in this example, the vaccine will be administered to subjects in the population. Confirmatory diagnostic utilizing the systems, devices, methods, and compositions described herein will be performed to determine if the vaccinated subjects will develop and maintain sufficient quantity of neutralizing antibody. Confirmatory diagnostic will be performed at one month, two months, six months, one year, two years, three years, and four years after vaccination. Subjects with insufficient quantity of neutralizing antibody will be advised to receive a booster vaccine.

### Example 10. Vaccine Confirmatory Diagnostic—Vaccine Comprising Ad5-nCoV

A clinical trial is being conducted to test efficacy and effectiveness of a SARS-CoV-2 vaccine comprising an adenoviral vector comprising nucleic acid sequence the Spike (S) protein of SARS-CoV-2. The Ad5-nCoV vaccine is delivered via the use of lipid nanoparticle. The clinical trial is designed to assess the safety, reactogenicity and immunogenicity of Recombinant Novel Coronavirus Vaccine (Adenovirus Type 5 Vector). One hundred and eight subjects will be enrolled into one of three cohorts and will receive an intramuscular (IM) injection of experimental vaccine or placebo on Day 1 in the deltoid muscle. In the low-dose group, subjects received one dose of 5E10 vp Ad5-nCoV. In the middle-dose group, subjects received one dose of 1E11 vp Ad5-nCoV. In the high-dose group, subjects received one dose of 1.5E11vp Ad5-nCoV. Primary outcome measures safety indexes of adverse reactions [Time Frame: 0-7 days post-vaccination].

Secondary outcome measures: titer of SARS-CoV-2-specific neutralizing antibody (nAb) using the testing devices described herein [Time Frame: Through 1 year post last vaccination]; and seroconversion as measured by an increase of SARS-CoV-2-specific neutralizing antibody (nAb) titer using the testing devices described herein [Time Frame: Through 1 year post last vaccination]; safety indexes of adverse events [Time Frame: 0-28 days post-vaccination], occurrence of adverse events post-vaccination; safety indexes of SAE [Time Frame: 0-28 days, within 6 months post-vaccination], occurrence of serious adverse events post-vaccination; safety indexes of lab measures [Time Frame: pre-vaccination, day 7 post-vaccination], occurrence of abnormal changes of laboratory safety examinations; immunogenicity indexes of GMT (ELISA) [Time Frame: day 14, 28, month 3, 6 post-vaccination]geometric mean titer GMT) of S-specific antibodies against 2019 novel coronavirus tested by ELISA in serum; immunogenicity indexes of GMT (pseudoviral neutralization test method) [Time Frame: day 14, 28, month 6 post-vaccination], geometric mean titer (GMT) of S-specific antibodies against 2019 novel coronavirus tested by pseudoviral neutralization test method in serum; immunogenicity indexes of seropositivity rates (ELISA) [Time Frame: day 14, 28, month 3, 6 post-vaccination], the seropositivity rates of S-specific antibodies against 2019 novel coronavirus tested by ELISA in serum; immunogenicity indexes of seropositivity rates (pseudoviral neutralization test method) [Time Frame: day 14, 28, month 6 post-vaccination], the seropositivity rates of S-specific antibodies against 2019 novel coronavirus tested by pseudoviral neutralization test method in serum; immunogenicity indexes of GMI (ELISA) [Time Frame: day 14, 28, month 3, 6 post-vaccination], geometric mean fold increase (GMI) of S-specific antibodies against 2019 novel coronavirus tested by ELISA in serum; immunogenicity indexes of GMI (pseudoviral neutralization test method) [Time Frame: day 14, 28, month 6 post-vaccination], geometric mean fold increase (GMI) of S-specific antibodies against 2019 novel coronavirus tested by pseudoviral neutralization test method in serum; immunogenicity indexes of GMC (Ad5 vector) [Time Frame: day 14, 28 month 3, 6 post-vaccination], geometric mean concentration (GMC) of anti-Ad5 vector neutralizing antibody responses; immunogenicity indexes of GMI (Ad5 vector) [Time Frame: day 14, 28, month 3, 6 post-vaccination], geometric mean fold increase (GMI) of anti-Ad5 vector neutralizing antibody responses; and immunogenicity indexes of cellular immune [Time Frame: day 14, 28, month 6 post-vaccination], specific cellular immune responses. Other outcome measures: consistency analysis (ELISA and pseudoviral neutralization test method) [Time Frame: day, 14, 28, month 6 post-vaccination], consistency analysis of S-specific antibodies against 2019 novel coronavirus tested by ELISA against those tested by pseudoviral neutralization test method; dose-response relationship (Humoral immunity) [Time Frame: day 14, 28, month 3, 6 post-vaccination], relationship between Geometric mean titer (GMT) of S protein-specific antibodies against 2019 novel coronavirus and vaccine dose among study groups; persistence analysis of anti-S protein antibodies [Time Frame: day 14, 28, month 3, 6 post-vaccination], persistence analysis of anti-S protein antibodies among study groups; time-dose-response relationship (Humoral immunity) [Time Frame: day 14, 28, month 3, 6 post-vaccination], relationship between the appearance time of S-specific antibodies against 2019 novel coronavirus and the vaccination dose; dose-response relationship (cellular immunity) [Time Frame: day 14, 28, month 6 post-vaccination], relationship between cellular immune levels against 2019 novel coronavirus and vaccine dose among study groups; persistence analysis of cellular immune [Time Frame: day 14, 28, month 6 post-vaccination], persistence analysis of specific cellular immune response; and time-dose-response relationship cellular immunity) [Time Frame: day 14, 28, month 6 post-vaccination], relationship between the appearance time of cellular immunity against 2019 novel coronavirus and the vaccination dose.

Durability of the Ad5-nCoV vaccine is measured continuously throughout 1 year post the last vaccination. Seroconversion measured using the testing devices described show consistent neutralizing antibodies levels over time, indicating that the Ad5-nCoV vaccine is durable. The efficacy and durability of the Ad5-nCoV vaccine is continuously monitored using the testing devices described herein during Phase 0, Phase I, Phase II, Phase III, or Phase IV or after Phase IV of the clinical trial.

Upon approval of the Ad5-nCoV vaccine, the vaccine will be administered to subjects in the population. Confirmatory diagnostic utilizing the systems, devices, methods, and compositions described herein will be performed to determine if the vaccinated subjects will develop and maintain sufficient quantity of neutralizing antibody. Confirmatory diagnostic will be performed at one month, two months, six months, one year, two years, three years, and four years after vaccination. Subjects with insufficient quantity of neutralizing antibody will be advised to receive a booster vaccine.

### Example 11. Vaccine Confirmatory Diagnostic—Vaccine Comprising AZD1222 (ChAdOx1 nCoV-19)

A clinical trial is being conducted to test efficacy and effectiveness of a SARS-CoV-2 vaccine comprising a replication-deficient chimpanzee adenovirus, ChAdOx1, which is engineered to express the Spike (S) protein of SARS-CoV-2. There will be 4 study groups to receive various dose of the ChAdOx1 nCoV-19. Volunteers will participate in the study for approximately 6 months, with the option to come for an additional follow up visit at Day 364. Primary outcome measures: assess efficacy of the candidate ChAdOx1 nCoV-19 against COVID-19: Number of virologically confirmed (PCR positive) symptomatic cases [Time Frame: 6 months], number of virologically confirmed (PCR positive) symptomatic cases of COVID-19; and assess the safety of the candidate vaccine ChAdOx1 nCoV: occurrence of serious adverse events (SAEs) [Time Frame: 6 months], occurrence of serious adverse events (SAEs) throughout the study duration.

Secondary outcome measures: titer of SARS-CoV-2-specific neutralizing antibody (nAb) using the testing devices described herein [Time Frame: Through 1 year post last vaccination], seroconversion as measured by an increase of SARS-CoV-2-specific neutralizing antibody (nAb) titer using the testing devices described herein [Time Frame: Through 1 year post last vaccination], assess the safety, tolerability and reactogenicity profile of the candidate vaccine ChAdOx1 nCoV: Occurrence of solicited local reactogenicity signs and symptoms [Time Frame: 7 days following vaccination], occurrence of solicited local reactogenicity signs and symptoms for 7 days following vaccination; assess the safety, tolerability and reactogenicity profile of the candidate vaccine ChAdOx1 nCoV: occurrence of solicited systemic reactogenicity signs and symptoms [Time Frame: 7 days following vaccination], occurrence of solicited systemic reactogenicity signs and symptoms for 7 days following vaccination; assess the safety, tolerability and reactogenicity profile of the candidate vaccine ChAdOx1 nCoV: Occurrence of unsolicited adverse events (AEs) [Time Frame: 28 days following vaccination], occurrence of unsolicited adverse events (AEs) for 28 days following vaccination; assess the safety, tolerability and reactogenicity profile of the candidate vaccine ChAdOx1 nCoV through standard blood tests [Time Frame: 6 months], change from baseline for safety laboratory measures (haematology and biochemistry blood results); assess the safety, tolerability and reactogenicity profile of the candidate vaccine ChAdOx1 nCoV by measuring the number of disease enhancement episodes [Time Frame: 6 months], occurrence of disease enhancement episodes; assess efficacy of the candidate ChAdOx1 nCoV-19 against severe and non-severe COVID-19 [Time Frame: 6 months], number of deaths associated with COVID-19; assess efficacy of the candidate ChAdOx1 nCoV-19 against severe and non-severe COVID-19 [Time Frame: 6 months], umber of hospital admissions associated with COVID-19; assess efficacy of the candidate ChAdOx1 nCoV-19 against severe and non-severe COVID-19 [Time Frame: 6 months], number of intensive care unit admissions associated with COVID-19; assess efficacy of the candidate ChAdOx1 nCoV-19 against severe and non-severe COVID-19 by measuring seroconversion rates [Time Frame: 6 months], proportion of people who become seropositive for non-Spike SARS-CoV-2 antigens during the study, assess cellular and humoral immunogenicity of ChAdOx1 nCoV-19 through ELISpot assays [Time Frame: 6 months], interferon-gamma (IFN-γ) enzyme-linked immunospot (ELISpot) responses to SARS-CoV-2 spike protein; and assess cellular and humoral immunogenicity of ChAdOx1 nCoV-19 [Time Frame: 6 months], quantify antibodies against SARS-CoV-2 spike protein (seroconversion rates). Other outcome measures: assess cellular and humoral immunogenicity of ChAdOx1 nCoV-19 through Virus neutralizing antibody assays [Time Frame: 6 months], virus neutralizing antibody (NAb) assays against live and/or pseudotype SARS-CoV-2 virus; and to assess safety, reactogenicity, immunogenicity and efficacy endpoints, for participants receiving prophylactic paracetamol [Time Frame: 6 months], all safety, reactogenicity, immunogenicity and efficacy endpoints.

Durability of the AZD1222 vaccine is measured continuously throughout 1 year post the last vaccination. Seroconversion measured using the testing devices described show consistent neutralizing antibodies levels over time, indicating that the AZD1222 vaccine is durable. The efficacy and durability of the AZD1222 vaccine is continuously monitored using the testing devices described herein during Phase 0, Phase I, Phase II, Phase III, or Phase IV or after Phase IV of the clinical trial.

Upon approval of the AZD1222 vaccine, the vaccine will be administered to subjects in the population. Confirmatory diagnostic utilizing the systems, devices, methods, and compositions described herein will be performed to determine if the vaccinated subjects will develop and maintain sufficient quantity of neutralizing antibody. Confirmatory diagnostic will be performed at one month, two months, six months, one year, two years, three years, and four years after vaccination. Subjects with insufficient quantity of neutralizing antibody will be advised to receive a booster vaccine.

### Example 12. Vaccine Confirmatory Diagnostic—Vaccine Comprising INO-400

A clinical trial is being conducted to test efficacy and effectiveness of a SARS-CoV-2 vaccine comprising pGX DNA plasmid with nucleic acid encoding the Spike (S) protein of SARS-CoV-2 as the insert. The plasmid will be administered intramuscularly via electroporation. There will be 2 study groups to receive various dose of the pGX plasmid. The subjects will be tasked for follow-up visits for up to 52 weeks. Primary outcome measures: percentage of participants with Adverse Events (AEs) [Time Frame: Baseline up to Week 52]; percentage of participants with administration (Injection) site reactions [Time Frame: Day 0 up to Week 52]; percentage of participants with Adverse Events of Special Interest (AESIs) [Time Frame: Baseline up to Week 52]; change from baseline in antigen-specific binding antibody titers [Time Frame: Baseline up to Week 52]; and change from baseline in antigen-specific Interferon-Gamma (IFN-γ) cellular immune response [Time Frame: Baseline up to Week 52].

Secondary outcome measures: titer of SARS-CoV-2-specific neutralizing antibody (nAb) using the testing devices described herein [Time Frame: Through 1 year post last vaccination]; and seroconversion as measured by an increase of SARS-CoV-2-specific neutralizing antibody (nAb) titer using the testing devices described herein [Time Frame: Through 1 year post last vaccination].

Durability of the INO-400 vaccine is measured continuously throughout 1 year post the last vaccination. Seroconversion measured using the testing devices described show consistent neutralizing antibodies levels over time, indicating INO-400 vaccine is durable. The efficacy and durability of the INO-400 vaccine is continuously monitored using the testing devices described herein during Phase 0, Phase I, Phase II, Phase III, or Phase IV or after Phase IV of the clinical trial.

Upon approval of the INO-400 vaccine, the vaccine will be administered to subjects in the population. Confirmatory diagnostic utilizing the systems, devices, methods, and compositions described herein will be performed to determine if the vaccinated subjects will develop and maintain sufficient quantity of neutralizing antibody. Confirmatory diagnostic will be performed at one month, two months, six months, one year, two years, three years, and four years after vaccination. Subjects with insufficient quantity of neutralizing antibody will be advised to receive a booster vaccine.

### Example 13. Vaccine Confirmatory Diagnostic—Vaccine Comprising BNT162

A clinical trial is being conducted to test efficacy and effectiveness of a SARS-CoV-2 anti-viral RNA vaccine comprising mRNA or modified mRNA to express the Spike (S) protein or a fragment thereof of SARS-CoV-2. There are four vaccine candidates being tested. Two of the four vaccine candidates include a nucleoside modified mRNA (modRNA), one includes a uridine containing mRNA (uRNA), and the fourth vaccine candidate utilizes self-amplifying mRNA (saRNA). Each mRNA format is combined with a lipid nanoparticle (LNP) formulation. The larger spike sequence is included in two of the vaccine candidates, and the smaller optimized receptor binding domain (RBD) from the spike protein is included in the other two candidates. The RBD-based candidates contain the piece of the spike that is thought to be most important for eliciting antibodies that inactivate the virus. The trial has two parts: a dose-finding part (Part A) with four dose cohorts (treatment groups) for each vaccine and one pre-defined and one optional dose level for a de-escalation approach and, a second part (Part B) dedicated to recruit expansion cohorts with dose levels which are selected from data generated in Part A. The vaccines BNT162a1, BNT162b1, and BNT162b2 will be administered using a Prime/Boost (P/B) regimen. The vaccine BNT162c2 will be administered using a Single dose (SD) regimen.

Primary outcome measures: solicited local reactions at the injection site (pain, tenderness, erythema/redness, induration/swelling) recorded up to 7±1 days after each immunization. [Time Frame: up to 7 days following each dose administration]; solicited systemic reactions (nausea, vomiting, diarrhea, headache, fatigue, myalgia, arthralgia, chills, loss of appetite, malaise, and fever) recorded up to 7±1 days after each immunization. [Time Frame: up to 7 days following each dose administration]; the proportion of subjects with at least 1 unsolicited treatment emergent adverse event (TEAE): [Time Frame: 21 days following dose administration], for BNT162a1, BNT162b1, BNT162b2 (P/B): occurring up to 21±2 days after the prime immunization; and the proportion of subjects with at least 1 unsolicited treatment emergent adverse event (TEAE): [Time Frame: 28 days following dose administration], for BNT162a1, BNT162b1, BNT162b2 (P/B): occurring up to 28±4 days after the boost immunization, and for BNT162c2 (SD): The proportion of subjects with at least 1 unsolicited TEAE occurring up to 28±4 days after the immunization.

Secondary outcome measures: titer of SARS-CoV-2-specific neutralizing antibody (nAb) using the testing devices described herein [Time Frame: Through 1 year post last vaccination], seroconversion as measured by an increase of SARS-CoV-2-specific neutralizing antibody (nAb) titer using the testing devices described herein [Time Frame: Through 1 year post last vaccination], for BNT162a1, BNT162b1, BNT162b2 (P/B): [Time Frame: up to 162 days following dose administration], functional antibody responses at 7±1 days and 21±2 days after primary immunization and at 21±2 days, 63±5 days, and 162±7 days after the boost immunization; for BNT162a1, BNT162b1, BNT162b2 (P/B): [Time Frame: up to 162 days following dose administration], fold increase in functional antibody titers 7±1 days and 21±2 days after primary immunization and at 21±2 days, 63±5 days, and 162±7 days after the boost immunization; for BNT162a1, BNT162b1, BNT162b2 (P/B): [Time Frame: up to 162 days following dose administration], number of subjects with seroconversion defined as a minimum of 4-fold increase of functional antibody titers as compared to baseline at 7±1 days and 21±2 days after primary immunization and at 21±2 days, 63f5 days, and 162±7 days after the boost immunization; for BNT162c2 (SD): [Time Frame: up to 183 days following dose administration], functional antibody responses at 7±1 days, 21±2 days, 42±3 days, 84±5 days, and 183±7 days after the primary immunization; for BNT162c2 (SD): [Time Frame: up to 183 days following dose administration], old increase in functional antibody titers at 7±1 days, 21±2 days, 42±3 days, 84±5 days, and 183±7 days after the primary immunization; and for BNT162c2 (SD): [Time Frame: up to 183 days following dose administration], number of subjects with seroconversion defined as a minimum of 4-fold increase of functional antibody titers as compared to baseline at 7±1 days, 21±2 days, 42±3 days, 84±5 days, and 183±7 days after the primary immunization.

Durability of the BNT162 vaccine is measured continuously throughout 1 year post the last vaccination. Seroconversion measured using the testing devices described show consistent neutralizing antibodies levels over time, indicating that the BNT162 vaccine is durable. The efficacy and durability of the BNT162 vaccine is continuously monitored using the testing devices described herein during Phase 0, Phase I, Phase II, Phase III, or Phase IV or after Phase IV of the clinical trial.

Upon approval of the BNT162 vaccine, the vaccine will be administered to subjects in the population. Confirmatory diagnostic utilizing the systems, devices, methods, and compositions described herein will be performed to determine if the vaccinated subjects will develop and maintain sufficient quantity of neutralizing antibody. Confirmatory diagnostic will be performed at one month, two months, six months, one year, two years, three years, and four years after vaccination. Subjects with insufficient quantity of neutralizing antibody will be advised to receive a booster vaccine.

### Example 14. Vaccine Confirmatory Diagnostic—Vaccine Comprising Inactivated Virus

A clinical trial is being conducted to test efficacy and effectiveness of a SARS-CoV-2 vaccine comprising an inactivated virus of SARS-CoV-2. Primary outcome measures incidence of adverse reactions or adverse events between 0-7 days after each doe of vaccination.

Secondary outcome measures: titer of SARS-CoV-2-specific neutralizing antibody (nAb) using the testing devices described herein [Time Frame: Through 1 year post last vaccination], seroconversion as measured by an increase of SARS-CoV-2-specific neutralizing antibody (nAb) titer using the testing devices described herein [Time Frame: Through 1 year post last vaccination], incidence of abnormal indicators of liver and kidney function, blood routine and urine routine on the 4th day after each dose of vaccination in phase I; rate of adverse reaction and adverse event at 8-28/30 days, 0-28/30 days after inoculation of each dose of vaccine; Incidence of Serious Adverse Events (SAE) within 12 months from first vaccination to completion of full vaccination schedule; four-fold growth rate and antibody level (GMT, GMI) of serum antibody against COVID-19 at 28 days after full vaccination; four-fold growth rate and antibody level (GMT, GMI) of COVID-19 antibody as determined in the 18-59 age group, bleeding shall be done at 4 and 14 days before and after each dose of vaccination, 21 days after the first dose of vaccination, 28, 90, 180 days after full vaccination completion, and 360 days after full vaccination in phase I; four-fold growth rate and antibody level (GMT, GMI) of COVID-19 antibody as determined in >=60 years old and 6-17 years old groups, bleeding shall be done before and 4 days after each dose of vaccination, 14 days after the first and second doses of vaccination, at 28 days and 90 days and 180 days and 360 days after full vaccination in phase I; cellular immunity as determined for the group at 18 years old and above, bleeding shall be done before and after first and second vaccination, before third vaccination and at 28 days, 180 days and 360 days after full vaccination in phase I; incidence of adverse reactions/events at 8-14/21/28/30 days, 0-14/21/28/30 days after inoculation of each dose of vaccine in phase II; four-fold growth rate and antibody level (GMT, GMI) of COVID-19 antibody as determined in 18-59 age group with 1 dose, bleeding shall be done before vaccination and at 28 days post full-vaccination completion in phase II; four-fold growth rate and antibody level (GMT, GMI) of COVID-19 antibody as determined in 3-doses group, bleeding shall be done before each dose and at 28 days post full vaccination in phase II; and four-fold growth rate and antibody level (GMT, GMI) of COVID-19 antibody as determined 90 days, 180 days and 360 days after full vaccination in phase II.

Durability of the vaccine comprising an inactivated virus of SARS-CoV-2 is measured continuously throughout 1 year post the last vaccination. Seroconversion measured using the testing devices described show consistent neutralizing antibodies levels over time, indicating that vaccine comprising an inactivated virus of SARS-CoV-2 is durable. The efficacy and durability of the vaccine comprising an inactivated virus of SARS-CoV-2 is continuously monitored using the testing devices described herein during Phase 0, Phase I, Phase II, Phase III, or Phase IV or after Phase IV of the clinical trial.

Upon approval of the vaccine comprising an inactivated virus of SARS-CoV-2 vaccine, the vaccine will be administered to subjects in the population. Confirmatory diagnostic utilizing the systems, devices, methods, and compositions described herein will be performed to determine if the vaccinated subjects will develop and maintain sufficient quantity of neutralizing antibody. Confirmatory diagnostic will be performed at one month, two months, six months, one year, two years, three years, and four years after vaccination. Subjects with insufficient quantity of neutralizing antibody will be advised to receive a booster vaccine.

### Example 15. Vaccine Confirmatory Diagnostic—Vaccine Comprising PiCoVacc

A clinical trial is being conducted to test efficacy and effectiveness of a SARS-CoV-2 vaccine comprising a formalin-inactivated SARS-CoV-2 virus, PiCoVacc, obtained from vero cell culture. Primary outcome measures: safety indexes of adverse reactions [Time Frame: From the beginning of the vaccination to 28 days after the whole schedule vaccination], occurrence of adverse reactions post vaccination; immunogenicity indexes of neutralizing-antibody seroconversion rates for the emergency vaccination schedule (day 0, 14) [Time Frame: The 14th day after two doses of vaccination], the seroconversion rates of neutralizing antibodies against 2019 novel coronavirus tested by micro-neutralization assay in serum; and immunogenicity indexes of neutralizing-antibody seroconversion rates for the routine vaccination schedule (day 0, 28) [Time Frame: The 28th day after two doses of vaccination], the seroconversion rates of neutralizing antibodies against 2019 novel coronavirus tested by micro-neutralization assay in serum.

Secondary outcome measures: titer of SARS-CoV-2-specific neutralizing antibody (nAb) using the testing devices described herein [Time Frame: Through 1 year post last vaccination], and seroconversion as measured by an increase of SARS-CoV-2-specific neutralizing antibody (nAb) titer using the testing devices described herein [Time Frame: Through 1 year post last vaccination], safety indexes of adverse reactions [Time Frame: 0˜7 days after each dose injection], occurrence of adverse reactions post vaccination; occurrence of abnormal changes of laboratory safety examinations (hemoglobin, WBCs, platelets, ALT, AST, total bilirubin, creatinine, creatine phosphokinase, urine protein, urine sugar, urinary erythrocyte) [Time Frame: The 3rd day after each dose injection], safety index, abnormal changes will be defined as any one of the lab indexes experiencing changes out of clinical reference value range; safety indexes of serious adverse events (SAEs) [Time Frame: From the beginning of the vaccination to 6 months after two doses of vaccination], occurrence of SAEs post vaccination; immunogenicity indexes of neutralizing-antibody seroconversion rates [Time Frame: 7, 14, 21, 42 days after the first dose injection for emergency vaccination schedule and 28, 35, 42 days after the first dose injection for the routine vaccination schedules], the seroconversion rates of neutralizing antibodies against 2019 novel coronavirus tested by micro-neutralization assay in serum; immunogenicity indexes of IgG antibody seropositivity rates [Time Frame: 7, 14, 21, 28, 42 days after the first dose injection for emergency vaccination schedule and 28, 35, 42, 56 days after the first dose injection for the routine vaccination schedule], the seropositivity rates of IgG antibody tested by ELISA serum; immunogenicity indexes IgM antibody seropositivity rates [Time Frame: 7, 14, 21, 28, 42 days after the first dose injection for emergency vaccination schedule and 28, 35, 42, 56 days after the first dose injection for the routine vaccination schedule], the seropositivity rates of IgM antibody tested by ELISA serum; immunogenicity indexes of GMT of neutralizing-antibody [Time Frame: 7, 14, 21, 28, 42 days after the first dose injection for emergency vaccination schedule and 28, 35, 42, 56 days after the first dose injection for the routine vaccination schedule], the GMT of neutralizing antibody against 2019 novel coronavirus tested by micro-neutralization assay in serum; and immunogenicity indexes of GMR of neutralizing-antibody [Time Frame: 7, 14, 21, 28, 42 days after the first dose injection for emergency vaccination schedule and 28, 35, 42, 56 days after the first dose injection for the routine vaccination schedule], the GMR of neutralizing antibody against 2019 novel coronavirus tested by micro-neutralization assay in serum. Other outcome measures: immunogenicity indexes of cellular immune [Time Frame: The 14th day after each dose vaccination], specific cellular immune responses; immunogenicity indexes of neutralizing-antibody persistence [Time Frame: 6 months after two doses of vaccination], the seropositivity rates of neutralizing antibodies against 2019 novel coronavirus tested by micro-neutralization assay in serum; immunogenicity indexes of neutralizing-antibody GMT [Time Frame: 6 months after two doses of vaccination], the GMT of neutralizing antibody against 2019 novel coronavirus tested by micro-neutralization assay in serum; safety indexes-Seropositivity rates of antinuclear antibody [Time Frame: The 7th day after each dose injection], seropositivity rates of antinuclear antibody in serum; and safety indexes-level of inflammatory factors [Time Frame: The 7th day after each dose injection], level of inflammatory factors in serum.

Durability of the PiCoVacc vaccine is measured continuously throughout 1 year post the last vaccination. Seroconversion measured using the testing devices described show consistent neutralizing antibodies levels over time, indicating that the PiCoVacc vaccine is durable. The efficacy and durability of the PiCoVacc vaccine is continuously monitored using the testing devices described herein during Phase 0, Phase I, Phase II, Phase III, or Phase IV or after Phase IV of the clinical trial.

Upon approval of the PiCoVacc vaccine, the vaccine will be administered to subjects in the population. Confirmatory diagnostic utilizing the systems, devices, methods, and compositions described herein will be performed to determine if the vaccinated subjects will develop and maintain sufficient quantity of neutralizing antibody. Confirmatory diagnostic will be performed at one month, two months, six months, one year, two years, three years, and four years after vaccination. Subjects with insufficient quantity of neutralizing antibody will be advised to receive a booster vaccine.

### Example 16. Vaccine Confirmatory Diagnostic—Vaccine Comprising bacTRL-Spike

A clinical trial is being conducted to test efficacy and effectiveness of a SARS-CoV-2 vaccine (bacTRL-Spike) comprising bifidobacterial engineered to express the Spike protein of SARS0-CoV-2. Primary outcome measures frequency of adverse events [Time Frame: Up to 12 months post-vaccination], adverse events (specifically including incidence of gastrointestinal-associated events) following administration of oral bacTRL-Spike.

Secondary outcome measures: titer of SARS-CoV-2-specific neutralizing antibody (nAb) using the testing devices described herein [Time Frame: Through 1 year post last vaccination], and seroconversion as measured by an increase of SARS-CoV-2-specific neutralizing antibody (nAb) titer using the testing devices described herein [Time Frame: Through 1 year post last vaccination], SARS-CoV-2 antibodies [Time Frame: Baseline (pre-vaccination), and 1, 3 and 12 months post-vaccination], antibody against SARS-CoV-2 Spike protein; incidence of COVID-19 infection [Time Frame: Up to 12 months post-vaccination], incidence and clinical phenotype of confirmed and probable COVID-19 infection among vaccinated participants, based on current public health definitions; bacTRL-Spike in stool post-vaccination [Time Frame: Days 7, 14, 21, and 1 and 3 months post-vaccination], isolation of viable bacTRL-Spike from stool post-vaccination; seroconversion of circulating anti-Spike IgG antibodies & stability of serum IgG titers [Time Frame: Up to 12 months post-vaccination], collection of biological samples for future studies to understand immunity against SARS-CoV-2; and effectiveness of intestinal colonization of the probiotic-based bacTRL-Spike oral vaccine [Time Frame: Up to 12 months post-vaccination], collection of biological samples for future studies to understand immunity against SARS-CoV-2.

Durability of the bacTRL-Spike vaccine is measured continuously throughout 1 year post the last vaccination. Seroconversion measured using the testing devices described show consistent neutralizing antibodies levels over time, indicating that the bacTRL-Spike vaccine is durable. The efficacy and durability of the bacTRL-Spike vaccine is continuously monitored using the testing devices described herein during Phase 0, Phase I, Phase II, Phase III, or Phase IV or after Phase IV of the clinical trial.

Upon approval of the bacTRL-Spike vaccine, the vaccine will be administered to subjects in the population. Confirmatory diagnostic utilizing the systems, devices, methods, and compositions described herein will be performed to determine if the vaccinated subjects will develop and maintain sufficient quantity of neutralizing antibody. Confirmatory diagnostic will be performed at one month, two months, six months, one year, two years, three years, and four years after vaccination. Subjects with insufficient quantity of neutralizing antibody will be advised to receive a booster vaccine.

### Example 17. Vaccine Confirmatory Diagnostic—Vaccine Comprising NVX-CoV2373

A clinical trial is being conducted to test efficacy and effectiveness of a SARS-CoV-2 vaccine comprising a multiple recombinant nanoparticle vaccine comprising a prefusion form of the Spike protein of SARS-CoV-2. Primary outcome measures: subjects with solicited AEs—Phase 1 [Time Frame: 28 days], numbers and percentages (with 95% CIs) of subjects with solicited AEs (local, systemic) for 7 days following each primary vaccination (Days 0, 21) by severity score, duration, and peak intensity. In the case of no reactogenicity, a toxicity score of zero (0) will be applied; safety Laboratory Values (serum chemistry, hematology)—Phase 1 [Time Frame: 28 days], safety laboratory values (serum chemistry, hematology) by FDA toxicity scoring (absolute and change from baseline where identified) at 7 days after each vaccination; and serum IgG antibody levels specific for the SARS-CoV-2 rS protein antigen(s)—Phase 1 [Time Frame: 35 days], serum IgG antibody levels specific for the SARS-CoV-2 rS protein antigen(s) as detected by ELISA at Day 21 and Day 35. Derived/calculated endpoints based on these data will include geometric mean ELISA units, geometric mean fold rise, and seroconversion rate (proportion of subjects with >4-fold rises in ELISA units).

Secondary outcome measures: titer of SARS-CoV-2-specific neutralizing antibody (nAb) using the testing devices described herein [Time Frame: Through 1 year post last vaccination]; and seroconversion as measured by an increase of SARS-CoV-2-specific neutralizing antibody (nAb) titer using the testing devices described herein [Time Frame: Through 1 year post last vaccination].

Durability of the NVX-CoV2373 vaccine is measured continuously throughout 1 year post the last vaccination. Seroconversion measured using the testing devices described show consistent neutralizing antibodies levels over time, indicating that the NVX-CoV2373 vaccine is durable. The efficacy and durability of the NVX-CoV2373 vaccine is continuously monitored using the testing devices described herein during Phase 0, Phase I, Phase II, Phase III, or Phase IV or after Phase IV of the clinical trial.

Upon approval of the NVX-CoV2373 vaccine, the vaccine will be administered to subjects in the population. Confirmatory diagnostic utilizing the systems, devices, methods, and compositions described herein will be performed to determine if the vaccinated subjects will develop and maintain sufficient quantity of neutralizing antibody. Confirmatory diagnostic will be performed at one month, two months, six months, one year, two years, three years, and four years after vaccination. Subjects with insufficient quantity of neutralizing antibody will be advised to receive a booster vaccine.

### Example 18. Confirmatory Diagnostic for Vaccine Licensed for Use—mRNA1273

After approval of the mRNA1272 vaccine, the vaccine is administered to a population of subjects to confer adaptive immunity against SARS-CoV-2 in the population. After vaccination, the population is routinely screened with the confirmatory diagnostic testing system and devices described herein. One month after vaccination, the subjects are asked to submit biological samples comprising capillary blood. The subjects perform finger-prick at home to drop the blood droplets onto a blood card. The blood card contains multiple slots for multiple assays to be performed. The subjects drop the blood droplets onto each slot of the blood card and mail the blood card to a centralized facility. The blood cards are processed and assayed with the systems, devices, methods, and compositions described herein at the centralized facility to detect presence, absence, and quantity of neutralizing antibody induced by the mRNA1273 vaccine. At one month after vaccination, 100% of the tested subjects are confirmed to have sufficient quantity of neutralizing antibody against SARS-CoV-2 infection.

The subjects of the population are recommended to be tested using the confirmatory diagnostic testing device or system at two months after vaccination, at six months after vaccination, at one year after vaccination, at two years after vaccination, at three years after vaccination, and at every year after to ensure that the subjects maintain sufficient quantity of neutralizing antibody. The confirmatory diagnostic also identifies the subjects with insufficient quantity of neutralizing antibody at any of the time point for which the confirmatory diagnostic is performed. These subjects are advised to receive a booster vaccine.

In addition to determining the presence, absence, or quantity of neutralizing antibody in the vaccinated subjects over time, the confirmatory diagnostic also determines the percentage of the vaccinated subjects who have sufficient neutralizing antibody. 100% of the tested subjects are confirmed to have sufficient quantity of neutralizing antibody after one year. At two years after vaccination, 93% of the tested subjects are confirmed to have sufficient quantity of neutralizing antibody. 93% is above a threshold of herd immunity. Accordingly, other than advising the 7% of the subjects without sufficient quantity of neutralizing antibody to receive a booster vaccine, no additional step is taken. At three years after vaccination, only 53% of the tested subjects are confirmed to have sufficient neutralizing antibody, which is now below the threshold of herd immunity. Accordingly, in addition to strongly urging the subjects with insufficient quantity of neutralizing antibody to receive a booster vaccine, all subjects are asked to observe social distancing rules until the percentage of the population with sufficient quantity of neutralizing antibody is above the threshold of herd immunity again.

### Example 19. Identifying Population in Need of Vaccination

A population of subjects is in need of a vaccine against a pathogen (e.g., SARS-CoV-2), but there is a limited capacity to vaccinate each subject at the same time. The testing device or system described herein is used to identify subjects that lack a sufficient adaptive immune response to the pathogen, thereby identifying the subjects as being in need of the vaccine. Optionally, individuals who belong to a high risk category (e.g., preexisting conditions, old age, and the like) may be prioritized for vaccination with the vaccine.

### Example 20. Vaccine Confirmatory Diagnostic—Vaccine Comprising AV-COVID-19

A clinical trial is being conducted to test efficacy and effectiveness of a SARS-CoV-2 vaccine consisting of autologous dendritic cells loaded with antigens from SARS-CoV-2 to prevent COVID-19 in adults. Primary outcome measures: Confirm safety [Time Frame: 6 months], confirm safety of AV-COVID-19 by adverse event monitoring.

Secondary outcome measures: titer of SARS-CoV-2-specific neutralizing antibody (nAb) using the testing devices described herein [Time Frame: Through 1 year post last vaccination]; and seroconversion as measured by an increase of SARS-CoV-2-specific neutralizing antibody (nAb) titer using the testing devices described herein [Time Frame: Through 1 year post last vaccination]; Suggestion of efficacy [Time Frame: 6 months], measurement of IgG and IgM in subject blood; Optimal dose of SARS-CoV-2 antigen [Time Frame: 6 months]; measurement of IgG and IgM in subject blood; Advantage of administering vaccine admixed with GM-CSF [Time Frame: 6 months], measurement of IgG and IgM in subject blood; Frequency of detecting IgG against SARS-CoV-2 in blood after vaccination [Time Frame: 6 months], measurement of IgG and IgM in subject blood.

Durability of the AV-COVID-19 vaccine is measured continuously throughout 1 year post the last vaccination. Seroconversion measured using the testing devices described show consistent neutralizing antibodies levels over time, indicating that the AV-COVID-19 vaccine is durable. The efficacy and durability of the AV-COVID-19 vaccine is continuously monitored using the testing devices described herein during Phase 0, Phase I, Phase II, Phase III, or Phase IV or after Phase IV of the clinical trial.

Upon approval of the AV-COVID-19 vaccine, the vaccine will be administered to subjects in the population. Confirmatory diagnostic utilizing the systems, devices, methods, and compositions described herein will be performed to determine if the vaccinated subjects will develop and maintain sufficient quantity of neutralizing antibody. Confirmatory diagnostic will be performed at one month, two months, six months, one year, two years, three years, and four years after vaccination. Subjects with insufficient quantity of neutralizing antibody will be advised to receive a booster vaccine.

### Example 21. Veterinary Use of Confirmatory Diagnostic Test

A veterinarian wishes to know whether an animal subject (e.g., a farm animal or a household pet) responded to administration of a new vaccine against SARS-CoV-2. A biological sample is obtained from the animal subject before administration of the vaccine to assess the baseline level of neutralizing antibodies against SARS-CoV-2. A biological sample is obtained from the animal subject at least 2-4 weeks following the administration of the vaccine. The testing device and systems described herein are used to measure a presence or requisite level of neutralizing antibodies against SARS-CoV-2 as compared to the baseline level of neutralizing antibodies measured before administration of the vaccine.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 22. Automated and Centralized Sample Processing and Assaying

Biological samples are received at a testing facility equipped with a centralized and automated system for assaying the biological samples. Each biological sample is contained in a biological sample medium (BSM), such as a dried blood spot card. The automated system, as shown in FIG. 13, comprises a Biological Sample Processor 1302 and an Assay Assembly Block 1309, which modules and submodules are operatively connected with a mechanical arm that is controlled by a centralizing computing system programmed to input the BSMs into the biological sample processor 1302 and transfer the purified biological sample to the Assay Assembly Block 1309 without human intervention. The biological sample receiver 1303 receives BSM and a barcode on the BSM is optionally scanned by the biological sample scanner 1304. The patient data (e.g., name, preexisting condition, address, healthcare insurance provider, etc.) is stored in a database and later optionally associated with the Assay Results 1312 by one or more computer processors in the centralized computing system. The biological sample separator 1305 (e.g., a mechanical hole punch) punches a hole in the BSM to remove a segment of the biological sample, which segment falls into a well in a 96 well plate (“elution plate”). The biological sample imager 1306 is an optical apparatus that images the bottom (or sides) of the well to ensure that the segment is correctly placed into the well. The biological sample extractor 1307 adds elution buffer (phosphate-buffered saline) to the well. The 96 well plate is gently agitated at a temperature of around 4 degrees Celsius for a period of time to elute the biological sample from the segment. The biological sample is optionally purified by the biological sample purifier 1308, such as isolating blood plasma or serum from a whole blood sample. The eluent is transferred to the Assay Assembly Block 1309, where the eluent from each well of the 96 well plate are diluted 4 times (e.g., 1:80, 1:160, 1:320, 1:640) and transferred into a 384 well plate (“source plate”) by the biological sample diluter 1310. The 1536 well plate is transferred to the biological sample assay conductor 1311, which runs the assay described herein (e.g., FRET, ELISA, etc.) to measure neutralizing antibodies against the coronavirus in dilutions. A higher signal intensity correlates with absence or low levels of neutralizing antibodies in the biological sample. The assay results 1312 are stored in a database and analyzed by one or more computer processors in the centralizing computing system. The one or more computer processors are configured to receive the results from the 4 dilutions per biological sample, averages the normalized results from the 4 dilutions per biological sample, and compares the average to a predetermined cutoff for neutralizing antibody titers indicative of CoVS-ACE2 binding inhibition (as measured, for example, using a cellularly based assay as demonstrated in Tan et al., A SARS-CoV-2 surrogate virus neutralization test based on antibody-mediated blockage of ACE2-spike protein-protein interaction, Nature Biotechnology 38, 1073-1078 (2020)), and optionally associates the results from the test to the patient data.

### Example 23: Longitudinal Study of Elderly Subjects in Assisted Living Facilities

A study group of elderly subjects in assisted living facilities, ages 75 and above, will be monitored over a period of time to measure the durability and efficacy of a vaccine against a coronavirus (e.g., SARS-CoV-2). In addition to the elderly subjects who may be residents of the assisted living facility, the study may also include the staff and visitors of the assisted living facility. The environment of the study is chosen as an assisted living facility, as the subjects have high exposure to one another and are all in a patient population with lower immunity to the coronavirus due to immunosenescence. The study will determine if the vaccine has induced a sufficient level of neutralizing antibodies (nAbs) to protect the study subjects from later infection of the coronavirus, or provide a reduced severity of symptoms from the coronavirus if infection were to occur.

Before the subjects are administered a vaccine, a baseline titer of coronavirus-specific nAbs will be determined for each subject using the testing devices described herein. The subjects will then be administered a vaccine against the coronavirus and tested again to determine a titer of nAbs. Once the vaccine is administered, the subjects will then be tested regularly in monthly intervals to determine a titer of nAbs present in the patients' system as time passes.

For each test, the subjects will be asked to submit biological samples comprising capillary blood. Blood cards will be distributed to the subjects for collection of the biological samples. The subjects, or healthcare workers, will perform a finger-prick to drop the capillary blood into each slot of the dried blood spot card. After collection of biological samples, the dried blood spot cards will then be sent by mail to a centralized processing and testing facility, where the blood cards are processed, as described in Example 22.

The data in the study will show the efficacy of the vaccine to protect elderly subjects against the coronavirus. The data will show that, for elderly subjects, confidence intervals are wider, and total nAb titers are lower, indicating that patients in this elderly subgroup are in need of close monitoring. The data in this study is also applicable for patients ages 50 and older.

The data will also show that, for elderly patients ages 65 and older, an increased dose of the coronavirus vaccine is desired to provide adequate protection against the coronavirus. For example, the increased dose of the vaccine may include four times the antigen as a coronavirus vaccine for patients ages 64 and younger.

The level of nAbs is also correlated to negative PCR test results. The study will show that a high nAbs titer in the subjects is indicative of a negative PCR test result.

### Example 24: Study Showing the Effects of Obesity on Patients Diagnosed with a Coronavirus

A study group of obese subjects will be monitored over a period of time to measure the durability and efficacy of a vaccine against a coronavirus (e.g., SARS-CoV-2) for obese patients. The study will determine the effect obesity has on patients diagnosed with a coronavirus.

Before the subjects are administered a vaccine, a baseline titer of coronavirus-specific nAbs will be determined for each subject using the testing devices described herein. The subjects will then be administered a vaccine against the coronavirus and tested again to determine a titer of nAbs. Once the vaccine is administered, the subjects will then be tested regularly in monthly intervals to determine a titer of nAbs present in the patients' system as time passes.

For each test, the subjects will be asked to submit biological samples comprising capillary blood. Blood cards will be distributed to the subjects for collection of the biological samples. The subjects, or healthcare workers, will perform a finger-prick to drop the capillary blood into each slot of the dried blood spot card. After collection of biological samples, the dried blood spot cards will then be sent by mail to a centralized processing and testing facility, where the blood cards are processed, as described in Example 22.

The data will show that obese patients diagnosed with a coronavirus have an increased risk of death as compared to diagnosed patients who are not obese. The data will also show that obesity causes an increased risk of death for a coronavirus patient, and that the increased risk of death applies to all obese coronavirus patients, irrespective of the patient's race.

### Example 25: Study of Subject with a High Risk of Exposure to a Coronavirus

A study group of subjects with a high risk of exposure to a coronavirus will be monitored over a period of time to measure the durability and efficacy of a vaccine against a coronavirus (e.g., SARS-CoV-2). The subjects may be at a high risk of exposure to a coronavirus because of congregate living circumstances. For example, the subjects may include the staff, guests, and visitors living on a cruise ship, in dormitories, or other congregate living environments. The study may also include subjects with a high risk of exposure due to their working environment. The study may follow healthcare workers, meat processing workers, food industry workers, public school workers, retail workers, and workers in other high-population-density work environments. The environment of the study is chosen as congregate living and/or high-population-density work environments, as the subjects have high exposure to one another. The study will determine if the vaccine has induced a sufficient level of neutralizing antibodies (nAbs) to protect the study subjects from later infection of the coronavirus, or provide a reduced severity of symptoms from the coronavirus if infection were to occur.

Before the subjects are administered a vaccine, a baseline titer of coronavirus-specific nAbs will be determined for each subject using the testing devices described herein. The subjects will then be administered a vaccine against the coronavirus and tested again to determine a titer of nAbs. Once the vaccine is administered, the subjects will then be tested regularly in monthly intervals to determine a titer of nAbs present in the subjects' system as time passes.

For each test, the subjects will be asked to submit biological samples comprising capillary blood. Blood cards will be distributed to the subjects for collection of the biological samples. The subjects, or healthcare workers, will perform a finger-prick to drop the capillary blood into each slot of the dried blood spot card. After collection of biological samples, the dried blood spot cards will then be sent by mail to a centralized processing and testing facility, where the blood cards are processed, as described in Example 22.

The data in the study will show the efficacy of the vaccine to protect subjects, with a high risk of exposure, against the coronavirus. The data will show a minimum nAbs titer necessary to protect subjects in such congregate living and high-population-density work environments.

The level of nAbs is also correlated to negative PCR test results. The study will show that a high nAbs titer in the subjects is indicative of a negative PCR test result.

### Example 26. Performance of Neutralizing Antibody Test

To determine whether the neutralizing antibody test described herein can measure neutralizing antibodies against SARS-CoV-2 accurately, binding inhibition was measured using the neutralizing antibody test and results were compared to a sample having a known IC50. The neutralizing antibody test was performed by performing a time-resolved fluorescence resonance energy transfer (TR-FRET) reaction by the following steps:

Equal volumes (5 μl) of the ACE2 receptor protein complexed with EU, the SARS-CoV-2 Spike protein S1 subunit complexed with a FRET-acceptor for EU, and inhibitor were mixed, and were incubated for 1 hour at room temperature. In this example, the inhibitor is any solution containing neutralizing antibodies that inhibit the binding of the Spike protein S1 subunit to the ACE2 receptor protein. Specifically, multiple dilutions of a solution containing a known neutralizing antibody with an IC50 of 4 nM was used as inhibitor. Following incubation, the fluorescent signal of the test mixture was then determined as shown in Table 3, and IC50 calculated. Read TR-FRET signal in a microtiter-plate reader under settings described below in Table 2 (settings may need optimization depending on the instrument).

TABLE 2
Settings for TR-FRET Imaging

| Channel | Variable | Recommended Value |
|---|---|---|
| 1 | Excitation wavelength (nm) | 340 +/− 20 |
| | Emission wavelength (nm) | 620 +/− 10 |
| | Lag time (μs) | 60 |
| | Integration time (μs) | 500 |
| 2 | Excitation wavelength (nm) | 340 +/− 20 |
| | Emission wavelength (nm) | 665 +/− 10 |
| | Lag time (μs) | 60 |
| | Integration time (μs) | 500 |

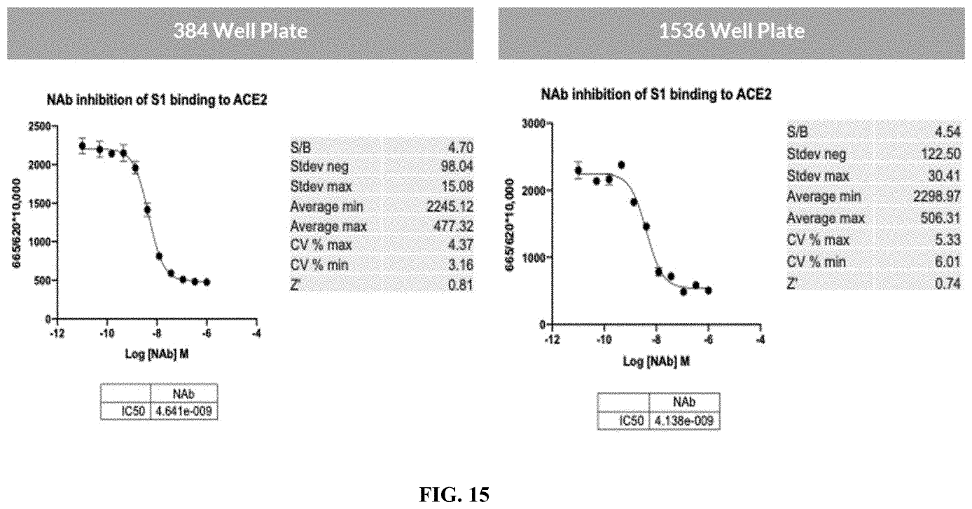

As shown in FIG. 15, the above protocol was performed on a 384 well plate (left) and a 1536 well plate (right), and in each case, the neutralizing antibody test accurately calculated the IC50 for the known neutralizing antibody. This demonstrates that the neutralizing antibody test described herein can measure inhibition of binding between the SARS-CoV-2 S protein and human ACE2 with accuracy. The signal to baseline (“S/B”) ratio of 4.70 and 4.54 demonstrate over 4 logs of separation between the baseline and maximum signal, indicating a large dynamic range for the neutralizing antibody test. The range in percent coefficient of variation (CV %) of 3.16-4.37 and 5.33-6.01 for the 384 well plate (left) and a 1536 well plate (right), respectively, are significantly lower than industry standard for a “good” test with respect to repeatability and reproducibility. Moreover, the Z′ score of 0.81 and 0.74 for the 384 well plate (left) and a 1536 well plate (right), respectively, indicate that the neutralizing antibody test is highly reproduceable. Together, these results show that the neutralizing antibody test described herein is accurate and highly reproduceable. In addition, the neutralizing antibody test described herein is scalable at least because of the small reagent requirements (<20 μl) and because the assay does not require a “wash” step.

### Example 27. The Neutraliziny Antibody Test can Detect Varvinr Neutraliziny Antibody Responses in Human Serum Samoes

Vaccines against SARS-CoV-2 have been shown elicit a strong neutralizing antibody response in some, but not all, individuals, including vaccines that are licensed for commercial use. Measuring the binding inhibition between the SARS-CoV-2 Spike (S) protein and human ACE2 is a surrogate for measuring neutralizing antibody titers in a human subject, as shown in, as for example, Tan et al., A SARS-CoV-2 surrogate virus neutralization test based on antibody-mediated blockage of ACE2-spike protein-protein interaction, Nat Biotechnol. 2020 September; 38(9): 1073-1078, which is hereby incorporated by reference in its entirety. Since neutralizing antibody titers correlate with vaccine efficacy, measuring binding inhibition between the SARS-CoV-2 S protein and human ACE2 can evaluate vaccine efficacy for individuals in a given population. Earle, et al. Evidence for antibody as a protective correlate for COVID-19 vaccines, medRxiv preprint doi: 10.1101.2021.03.17.20200246 (Mar. 20, 2021). The following experimental design is aimed at confirming that the neutralizing antibody test described herein can quantitatively measure binding inhibition between SARS-CoV-2 S protein and human ACE2 as a surrogate for neutralizing antibody titers in human samples obtained from vaccinated subjects.

Three human blood serum samples are obtained that do not contain neutralizing antibodies against SARS-CoV-2. The samples are spiked with different known quantities of neutralizing antibodies against SARS-CoV-2 S protein, thereby mimicking human serum samples obtained from individuals in a given population vaccinated against SARS-CoV-2 having varying neutralizing antibody responses. The positive control that will be used contains a buffer with a known quantity of neutralizing antibodies against SARS-CoV-2 S protein. The negative control that will be used contains the buffer without any neutralizing antibodies against SARS-CoV-2 S protein.

Equal volumes of the ACE2 receptor protein complexed with EU, the SARS-CoV-2 Spike protein S1 subunit complexed with a FRET-acceptor for EU, and inhibitor are mixed, and are incubated for 1 hour at room temperature. In this example, the inhibitor is any solution containing neutralizing antibodies that inhibit the binding of the Spike protein S1 subunit to the ACE2 receptor protein. Specifically, multiple dilutions of a solution containing a known neutralizing antibody with an IC50 of 4 nM is used as inhibitor. Following incubation, the fluorescent signal of the test mixture was then determined as shown in Table 2, and IC50 calculated. The above protocol is performed in a 384 well plate and a 1536 well plate. In each case, the results expected to be achieved are as follows:

It is expected that the neutralizing antibody test will accurately calculate the IC50 for the known neutralizing antibody for those samples. The CV %, Z′ score, and S/B are expected to be similar to the results described in Example 26. Together, the results are expected to show that the neutralizing antibody test described herein is not only accurate, highly reproducible, and scalable, but can measure binding inhibition between SARS-CoV-2 S protein and human ACE2 as a surrogate for neutralizing antibody titers in complex human serum samples that mimic samples obtained from vaccinated human subjects.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed:

1. A method for processing a biological sample, the method comprising:
(a) bringing a capillary blood sample into contact with (i) a first polypeptide comprising an Angiotensin-converting enzyme 2 (ACE2) polypeptide or a portion thereof capable of binding to a spike protein of a coronavirus and (ii) a second polypeptide comprising the spike protein of the coronavirus or an ACE2-binding portion of the spike protein under conditions sufficient cause the first polypeptide and the second polypeptide to form a detectable binding complex; and
(b) detecting the detectable binding complex, which detecting is indicative of an absence or a low level of neutralizing antibodies against the spike protein relative to a known level of the neutralizing antibodies, wherein the neutralizing antibodies block binding between the spike protein or the ACE2-binding portion thereof and the ACE2 polypeptide or the portion thereof under conditions otherwise suitable for binding.

2. The method of claim 1, wherein the capillary blood sample has a volume comprising less than 1 milliliter (mL).

3. The method of claim 2, wherein the volume comprises less than 100 microliters (μl).

4. The method of claim 1, wherein the coronavirus is a severe acute respiratory syndrome (SARS) coronavirus.

5. The method of claim 4, wherein the SARS coronavirus is SARS coronavirus 2 (SARS-CoV-2).

6. The method of claim 1, wherein the capillary blood sample is obtained from a human subject.

7. The method of claim 1, wherein the method does not comprise a wash step whereby the first polypeptide and the second polypeptide that is unbound following (a) is substantially removed prior to detecting in (b).

8. The method of claim 1, wherein detecting the binding complex in (b) comprises measuring a fluorescence signal.

9. The method of claim 8, wherein the fluorescence signal is inversely correlated with a level of neutralizing antibodies in the capillary blood sample.

10. The method of claim 1, wherein the binding complex is immobilized to a solid support.

11. A method for processing a biological sample, the method comprising:
(a) receiving a biological sample from a subject, wherein the biological sample was obtained from the subject by one or more finger pricks;
(b) bringing the biological sample into contact with (i) a first polypeptide comprising an Angiotensin-converting enzyme 2 (ACE2) polypeptide or a portion thereof capable of binding to a spike protein of a coronavirus and (ii) a second polypeptide comprising the spike protein of the coronavirus or an ACE2-binding portion of the spike protein under conditions sufficient cause the first polypeptide and the second polypeptide to form a detectable binding complex; and
(c) detecting the detectable binding complex, which detecting is indicative of an absence or a low level of neutralizing antibodies against the spike protein relative to a known level of the neutralizing antibodies, wherein the neutralizing antibodies block binding between the spike protein or the ACE2-binding portion thereof and the ACE2 polypeptide or the portion thereof under conditions otherwise suitable for binding.

12. The method of claim 11, wherein the biological sample has a volume comprising less than 1 milliliter (mL).

13. The method of claim 12, wherein the volume comprises less than 100 microliters (μl).

14. The method of claim 11, wherein the coronavirus is a severe acute respiratory syndrome (SARS) coronavirus.

15. The method of claim 14, wherein the SARS coronavirus is SARS coronavirus 2 (SARS-CoV-2).

16. The method of claim 11, wherein the subject is a human subject.

17. The method of claim 11, wherein the method does not comprise a wash step whereby the first polypeptide and the second polypeptide that is unbound following (a) is substantially removed prior to detecting in (c).

18. The method of claim 11, wherein detecting the binding complex in (b) comprises measuring a fluorescence signal.

19. The method of claim 18, wherein the fluorescence signal is inversely correlated with a level of neutralizing antibodies in the biological sample.

20. The method of claim 11, wherein the binding complex is immobilized to a solid support.

21. A method for processing a biological sample, the method comprising:
(a) obtaining less than 1 milliliter (mL) of a biological sample from a subject;
(b) bringing the biological sample into contact with (i) a first polypeptide comprising an Angiotensin-converting enzyme 2 (ACE2) polypeptide or a portion thereof capable of binding to a spike protein of a coronavirus and (ii) a second polypeptide comprising the spike protein of the coronavirus or an ACE2-binding portion of the spike protein under conditions sufficient cause the first polypeptide and the second polypeptide to form a detectable binding complex; and
(c) detecting the detectable binding complex, which detecting is indicative of an absence or a low level of neutralizing antibodies against the spike protein relative to a known level of the neutralizing antibodies, wherein the neutralizing antibodies block binding between the spike protein or the ACE2-binding portion thereof and the ACE2 polypeptide or the portion thereof under conditions otherwise suitable for binding.

22. The method of claim 21, wherein the biological sample comprises a volume comprising less than 500 μl when it is obtained in (a).

23. The method of claim 22, wherein the volume comprises less than 100 μl.

24. The method of claim 21, wherein the coronavirus is a severe acute respiratory syndrome (SARS) coronavirus.

25. The method of claim 24, wherein the SARS coronavirus is SARS coronavirus 2 (SARS-CoV-2).

26. The method of claim 21, wherein the subject is a human subject.

27. The method of claim 21, wherein the method does not comprise a wash step whereby the first polypeptide and the second polypeptide that is unbound following (a) is substantially removed prior to detecting in (c).

28. The method of claim 21, wherein detecting the binding complex in (c) comprises measuring a fluorescence signal.

29. The method of claim 28, wherein the fluorescence signal is inversely correlated with a level of neutralizing antibodies in the biological sample.

30. The method of claim 21, wherein the binding complex is immobilized to a solid support.
